(19) United States
(12) Patent Application Publication
(10) Pub. No.: US 2026/0091047 A1
(43) Pub. Date: Apr. 2, 2026

(54) THERAPEUTIC COMBINATIONS FOR MOVEMENT DISORDERS

(71) Applicant: Native Code Bio, LLC, Bainbridge Island, WA (US)

(72) Inventors: Adam R. Nelson, Piedmont, CA (US); Stephen Y. Hung, Bainbridge Island, WA (US)

(21) Appl. No.: 19/110,400

(22) PCT Filed: Sep. 11, 2023
(86) PCT No.: PCT/US2023/032444

§ 371 Date: Mar. 10, 2025

Related U.S. Application Data
(60) Provisional application No. 63/405,430, filed on Sep. 10, 2022.

(51) Int. Cl. A61K 31/675; A61K 31/00; A61K 31/37; A61K 31/4045; A61K 36/04; A61K 36/07; A61K 36/185; A61K 36/48; A61P 25/28

(52) U.S. Cl. CPC A61K 31/675; A61K 31/37; A61K 31/4045; A61K 31/658; A61K 36/04; A61K 36/078; A61K 36/3482; A61K 36/48; A61P 25/28; A61K 2236/15; A61K 2236/333; A61K 2236/37

(57) ABSTRACT

Provided are therapeutic combinations, pharmaceutical compositions, and pharmaceutical kits comprising bioactive molecules from fungi, plants, and algae. In some embodiments, the fungi are from any psilocybin-producing fungi, such as from Psilocybe spp. fungi. In some embodiments, the plants are from either or both of the Cannabis and Dipteryx genera. In some embodiments, the algae may be marine algae, such as from the family Bangicacae, including the Pyropia and Porphyra genera. Methods of producing the disclosed combinations, compositions, and kits are also provided, such as using natural, biosynthetic, or synthetic means. Further provided are methods of using the disclosed combinations, compositions, and kits in treatment, and in particular for movement disorders, such as for Parkinson's disease, and the disclosed combinations are demonstrated to provide significant advantages in the treatment thereof.

### CROSS-REFERENCE

Priority is claimed under PCT Art. 8 (1) and Rule 4.10 to U.S. Prov. App. No. 63/405,430, filed Sep. 10, 2022, and incorporated by reference for all purposes as if fully set forth herein.

### FIELD OF THE INVENTION

Provided are therapeutic combinations of bioactive molecules from fungi, plants, and algae, pharmaceutical compositions and kits comprising such molecules, and methods of their use in medicine, and in particular to treat movement disorders, such as Parkinson's disease.

### BACKGROUND OF THE INVENTION

Nearly 40 million people in the United States suffer from a movement disorder (Morishita, 2013). Most movement disorders are not curable, and treatment generally attempts only to ease symptoms or slow their progression. Treatment is typically incapable of restoring function to patients once it is lost, and insufficient to appreciably slow the progression of most disorders. Current therapies also can be invasive, such as Deep Brain Stimulation (DBS) which uses a surgically implanted device to send electrical impulses to the brain (Kringelbach et al., 2007). Moreover, current therapies are unable to treat most non-motor symptoms of movement disorders, such as those involving thinking, memory, behavior, and mood. Novel treatments that can reduce the severity of present symptoms, prevent the development of new symptoms, and broadly reverse the progression of disease, are therefore sorely needed.

Provided herein are therapeutic combinations, pharmaceutical compositions and kits, and methods of their use to treat movement disorders, to meet these needs and others, and having such advantages and improvements over prior art treatment options as will be readily appreciated.

### BRIEF SUMMARY OF THE INVENTION

The following presents a simplified summary of some embodiments of the invention in order to provide a basic understanding thereof. This summary is not an extensive overview, nor is it intended to identify every key or critical element of the invention or to delineate the complete scope of the invention. Its sole purpose is to present some exemplary embodiments in a simplified form as a prelude to the more detailed description below.

In some aspects are disclosed therapeutic combinations useful to prevent or treat a movement disorder, comprising: a fungal portion; a first plant portion; optionally, a second plant portion; and an algal portion. In some further aspects are disclosed therapeutic combinations comprising: a fungal portion; a first plant portion; a second plant portion; and an algal portion.

In some embodiments, the fungal portion is from a psilocybin-producing species. In some embodiments, the psilocybin-producing species is from any of the genera Athelia, Conocybe, Copelandia, Fibularhizoctonia, Galerina, Gymnopilus, Inocybe, Mycena, Panaeolus, Pholiotina, Pluteus, and Psilocybe. In some embodiments, the psilocybin-producing species is from the genus Psilocybe. In some embodiments, the psilocybin-producing species from the genus Psilocybe is any of P. azurescens, P. bohemica, P. semilanceata, P. baeocystis, P. cyanescens, P. tampanensis, P. cubensis, P. weilii, P. hoogshagenii, P. stuntzii, P. cyanofibrillosa, and P. liniformans. In some embodiments, the therapeutic combination comprises: a fungal portion from a species in the Psilocybe genus; a first plant portion; a second plant portion; and an algal portion.

In some embodiments, the first plant portion is from a species in the Cannabis genus. In some embodiments, the species in the Cannabis genus is any of Cannabis sativa, Cannabis indica, and Cannabis ruderalis. In some embodiments, the therapeutic combination comprises: a fungal portion; a first plant portion from a species in the Cannabis genus; a second plant portion; and an algal portion. In some embodiments, the therapeutic combination comprises: a fungal portion from a species in the Psilocybe genus; a first plant portion from a species in the Cannabis genus; a second plant portion; and an algal portion.

In some embodiments, the second plant portion is from a species in the Dipteryx genus. In some embodiments, the species in the Dipteryx genus is Dipteryx odorata. In some embodiments, the therapeutic combination comprises: a fungal portion; a first plant portion; a second plant portion from a species in the Dipteryx genus; and an algal portion. In some embodiments, the therapeutic combination comprises: a fungal portion from a species in the Psilocybe genus; a first plant portion from a species in the Cannabis genus; a second plant portion from a species in the Dipteryx genus; and an algal portion.

In some embodiments, the algal portion is from a species of marine algae. In some embodiments, the species of marine algae is from the family Bangiaceae. In some embodiments, the species of marine algae is from the genera Pyropia and Porphyra. In some embodiments, the species of marine algae is any of Pyropia yezoensis, Pyropia perforata, and Porphyra umbilicalis. In some embodiments, the therapeutic combination comprises: a fungal portion; a first plant portion; a second plant portion; and an algal portion from a species in the Pyropia or Porphyra genera. In some embodiments, the therapeutic combination comprises: a fungal portion from a species in the Psilocybe genus; a first plant portion from a species in the Cannabis genus; a second plant portion from a species in the Dipteryx genus; and an algal portion from a species in the Pyropia or Porphyra genera.

In some embodiments, the fungal portion comprises a fungal extract from the psilocybin-producing species. In some embodiments, the fungal extract is from P. azurescens, P. bohemica, P. semilanceata, P. baeocystis, P. cyanescens, P. tampanensis, P. cubensis, P. weilii, P. hoogshagenii, P. stuntzii, P. cyanofibrillosa, or P. liniformans. In some embodiments, the fungal extract is obtained by ultrasonic extraction or Soxhlet extraction. In some embodiments, the fungal extract comprises a 2:1 mixture of a fungal extract obtained by ultrasonic extraction and a fungal extract obtained by Soxhlet extraction.

In some embodiments, the first plant portion comprises a Cannabis plant extract from a species in the Cannabis genus. In some embodiments, the Cannabis plant extract is from Cannabis sativa, Cannabis indica, or Cannabis ruderalis. In some embodiments, the Cannabis plant extract is obtained by Soxhlet extraction.

In some embodiments, the second plant portion comprises a Dipteryx plant extract from a species in the Dipteryx genus. In some embodiments, the Dipteryx plant extract is from Dipteryx odorata. In some embodiments, the Dipteryx plant extract is obtained by anhydrous ethanol percolation.

In some embodiments, the algal portion comprises an algal extract from a species of marine algae. In some embodiments, the algal extract is from Pyropia yezoensis, Pyropia perforata, or Porphyra umbilicalis. In some embodiments, the algal extract is obtained by ultrasonic extraction.

In some embodiments, the therapeutic combination comprises: a fungal extract from a species in the Psilocybe genus; a Cannabis plant extract from a species in the Cannabis genus; a Dipteryx plant extract from a species in the Dipteryx genus; and an algal extract from a species in the Pyropia or Porphyra genera.

In some embodiments, the therapeutic combination comprises: a 2:1 mixture of a fungal extract obtained by ultrasonic extraction and a fungal extract obtained by Soxhlet extraction; a Cannabis plant extract obtained by Soxhlet extraction; a Dipteryx plant extract obtained by anhydrous ethanol percolation; and an algal extract obtained by ultrasonic extraction.

In some embodiments, the therapeutic combination comprises: a fungal extract comprising psilocybin and psilocin; a Cannabis plant extract comprising Δ⁹-THC (THC) and cannabidiol (CBD); a Dipteryx plant extract comprising coumarin; and an algal extract comprising porphyran.

In some embodiments, the therapeutic combination comprises: a fungal portion comprising psilocybin and psilocin; a first plant portion comprising Δ⁹-THC (THC) and cannabidiol (CBD); a second plant portion comprising coumarin; and an algal portion comprising porphyran.

In some embodiments, the fungal portion comprises a bioactive molecule from a fungus. In some embodiments, the bioactive molecule from a fungus is a primary bioactive molecule from a psilocybin-producing species. In some embodiments, the primary bioactive molecule from a psilocybin-producing species is one or more tryptamines or one or more beta-carbolines. In some embodiments, the one or more tryptamines is any of psilocybin, psilocin, baeocystin, norbaeocystin, norpsilocin, and aeruginascin. In some embodiments, the one or more tryptamines is psilocybin or psilocin. In some embodiments, the one or more tryptamines is psilocybin and psilocin. In some embodiments, the psilocybin and psilocin are in a weight ratio of about 5:3. In some embodiments, the one or more beta-carbolines is any of harmane, harmine, harmol, pinoline, harmaline, cordysinin C, cordysinin D, norharmane, and perlolyrine. In some embodiments, the bioactive molecule from a fungus is a secondary bioactive molecule from a psilocybin-producing species. In some embodiments, the secondary bioactive molecule from a psilocybin-producing species is any of a polysaccharide, a peptide, a terpene, a phenolic compound, a mineral, a vitamin, an amino acid, a lipid, choline, and a lactone.

In some embodiments, the first plant portion comprises a bioactive molecule from Cannabis. In some embodiments, the bioactive molecule from Cannabis is a primary bioactive molecule from a Cannabis species. In some embodiments, the primary bioactive molecule from a Cannabis species is one or more cannabinoids. In some embodiments, the one or more cannabinoids is any of a Δ⁹-THC-type cannabinoid, a Δ⁸-THC-type cannabinoid, a CBG-type cannabinoid, a CBD-type cannabinoid, a CBND-type cannabinoid, a CBE-type cannabinoid, a CBL-type cannabinoid, a CBC-type cannabinoid, a CBN-type cannabinoid, a CBT-type cannabinoid, and a miscellaneous-type cannabinoid. In some embodiments, the one or more cannabinoids is Δ⁹-THC (THC) or cannabidiol (CBD). In some embodiments, the one or more cannabinoids is THC and CBD. In some embodiments, the THC and CBD are in a weight ratio of about 1:1. In some embodiments, the bioactive molecule from Cannabis is a secondary bioactive molecule from a Cannabis species. In some embodiments, the secondary bioactive molecule from a Cannabis species is any of a flavone or flavonoid, a terpene or terpenoid, a carbohydrate, a fatty acid or a fatty acid esters, an amide, an amine, a phytosterol, and a phenolic compound.

In some embodiments, the second plant portion comprises a bioactive molecule from Dipteryx. In some embodiments, the bioactive molecule from Dipteryx is a primary bioactive molecule from Dipteryx odorata. In some embodiments, the primary bioactive molecule from Dipteryx odorata is coumarin. In some embodiments, the bioactive molecule from Dipteryx is a secondary bioactive molecule from Dipteryx odorata. In some embodiments, the secondary bioactive molecule from Dipteryx odorata is any of cumaru, a coumarin derivative, an isoflavone, a lupeol derivative, a fatty acid ester, (±)-balanophonin, (−)-lariciresinol, 3′-hydroxyretusin-8-methyl-ether, 5-methoxyxanthocercin A, 6,4′-dihydroxy-3′-methoxyaurone, 7-hydroxy-chromone, 7,3′-dihydroxy-8,4′-dimethoxyisoflavone, betulin, butin, coumaric-acid-beta-glucoside, dipteryxin, dipteryxic acid, eriodictyol, ferulic-acid, isoliquiritigenin, lupeol, melilotoside, melilotoside-1-p-coumaryl-beta-d-glucose, methyl-linolenate, methyl-oleate, O-coumaricacid, O-hydroxycoumaric-acid, odoratin, P-hydroxy-benzoic-acid, retusin, retusin-8-methyl-ether, sulfuretin, salicylic-acid, afrormisin, castinin, linoleic acid, oleic acid, 3′,4′,7′-trihydroxyflavone, luteolin, and umbelliferone.

In some embodiments, the algal portion comprises a bioactive molecule from algae. In some embodiments, the bioactive molecule from algae is a primary bioactive molecule from Pyropia or Porphyra. In some embodiments, the primary bioactive molecule from Pyropia or Porphyra is any of porphyran, an oligo-porphyran, a polysaccharide, an oligo-polysaccharide, a monosaccharide, a peptide, a phycobiliprotein, a mycosporine-like amino acid, an essential amino acid, a nonessential amino acid, a carotene, an intermediate carotenoid, a glycoprotein, an amino sulfonic acid, and taurine. In some embodiments, the primary bioactive molecule from Pyropia or Porphyra is porphyran. In some embodiments, the bioactive molecule from algae is a secondary bioactive molecule from Pyropia or Porphyra. In some embodiments, the secondary bioactive molecule from Pyropia or Porphyra is any of a mineral, a vitamin, a lipid, a phenolic compound, and a phlorotannin.

In some embodiments, the therapeutic combination further comprises a flavorant or colorant. In some embodiments, the flavorant is ginger or bay laurel.

In some embodiments, the therapeutic combination further comprises an additional active agent. In some embodiments, the additional active agent is any of levodopa, carbidopa, carbidopa-levodopa, entacapone, carbidopa-levodopa-entacapone, tolcapone, opicapone, pramipexole, pramipexole, ropinirole, apomorphine, rotigotine, selegiline, rasagiline, safinamide, amantadine, istradefylline, trihexyphenidyl, benztropine, procyclidine, trihexyphenidyl, orphenadrine, and buntanetap.

In some embodiments, at least one of the fungal portion, the first plant portion, the second plant portion, or the algal portion further comprises a non-naturally occurring carrier, diluent, or excipient. In some embodiments, at least two, at least three, or all four of the fungal portion, the first plant portion, the second plant portion, and the algal portion further comprises a non-naturally occurring carrier, diluent, or excipient.

In some embodiments, the therapeutic combination comprises: a bioactive molecule from a species in the Psilocybe genus; a bioactive molecule from a species in the Cannabis genus; a bioactive molecule from a species in the Dipteryx genus; and a bioactive molecule from a species in the Pyropia or Porphyra genera.

In some embodiments, the therapeutic combination comprises: a primary bioactive molecule from a species in the Psilocybe genus; a primary bioactive molecule from a species in the Cannabis genus; a primary bioactive molecule from a species in the Dipteryx genus; and a primary bioactive molecule from a species in the Pyropia or Porphyra genera.

In some embodiments, the therapeutic combination comprises: one or more tryptamines from a species in the Psilocybe genus; one or more cannabinoids from a species in the Cannabis genus; coumarin; and porphyran.

In some embodiments, the therapeutic combination comprises: psilocybin and psilocin; THC and CBD; coumarin; and porphyran.

In some embodiments, the therapeutic combination comprises: psilocybin and psilocin in a weight ratio of 5:3; THC and CBD in a weight ratio of 1:1; coumarin; and porphyran.

In some embodiments, the therapeutic combination further comprises: a secondary bioactive molecule from a species in the Psilocybe genus; a secondary bioactive molecule from a species in the Cannabis genus; a secondary bioactive molecule from a species in the Dipteryx genus; and a secondary bioactive molecule from a species in the Pyropia or Porphyra genera.

In some embodiments, one of the bioactive molecules, two of the bioactive molecules, three of the bioactive molecules, four of the bioactive molecules, five of the bioactive molecules, six of the bioactive molecules, at least one of the bioactive molecule, at least two of the bioactive molecules, at least three of the bioactive molecules, at least four of the bioactive molecules, at least five of the bioactive molecules, at least six of the bioactive molecules, all of the bioactive molecules, six or fewer of the bioactive molecules, five or fewer of the bioactive molecules, four or fewer of the bioactive molecules, three or fewer of the bioactive molecules, two or fewer of the bioactive molecules, or none of the bioactive molecules are from or are in an extract, are isolated molecules, are pure or substantially pure molecules, or are synthetic molecules.

In some embodiments, a single dose of the therapeutic combination comprises: 250 μg of psilocybin; 150 μg of psilocin; 1 mg of CBD; 1 mg of THC; 1 mg of coumarin; 8 mg of an extract of Pyropia; optionally, 8 mg of a flavorant or colorant.

In some embodiments, the flavorant or colorant comprises ginger or bay leaf. In some embodiments, the flavorant or colorant comprises ginger and bay leaf.

In some embodiments, the therapeutic combination further comprises a diluent. In some embodiments, the diluent is water.

In some embodiments, of the therapeutic combination: the fungal portion constitutes about 45% by volume of the total combination; the first plant portion constitutes about 15% by volume of the total combination; the second plant portion constitutes about 2% by volume of the total combination; the algal portion constitutes about 15% by volume of the total combination; a flavorant or colorant constitutes about 15% by volume of the total combination; and a diluent constitutes the remainder of the total combination.

In some embodiments, of the therapeutic combination: the fungal portion comprises a 2:1 mixture of a fungal extract obtained by ultrasonic extraction and a fungal extract obtained by Soxhlet extraction; the first plant portion comprises a Cannabis plant extract obtained by Soxhlet extraction; the second plant portion comprises a Dipteryx plant extract obtained by anhydrous ethanol percolation; the algal portion comprises an algal extract obtained by ultrasonic extraction; the flavorant or colorant comprises ethanol infused with ginger and bay leaf; and the diluent comprises water.

In some embodiments, of the therapeutic combination: the fungal portion is a 2:1 mixture of a fungal extract obtained by ultrasonic extraction and a fungal extract obtained by Soxhlet extraction; the first plant portion is a Cannabis plant extract obtained by Soxhlet extraction; the second plant portion is a Dipteryx plant extract obtained by anhydrous ethanol percolation; the algal portion is an algal extract obtained by ultrasonic extraction; the flavorant or colorant is ethanol infused with ginger and bay leaf; and the diluent is water.

In some embodiments, the diluent comprises water and at least one non-naturally occurring diluent.

In some aspects are disclosed methods of preparing a disclosed therapeutic combination, comprising: obtaining the fungal extract by ultrasonic extraction and/or Soxhlet extraction; obtaining the Cannabis plant extract by Soxhlet extraction; obtaining the Dipteryx plant extract by anhydrous ethanol percolation; obtaining the algal extract by ultrasonic extraction; analyzing the concentration of at least one bioactive molecule in each extract; calculating a compounding percentage to achieve a target dose for each of the at least one bioactive molecule in each extract; blending a calculated amount of each extract based on the compounding percentage into a mixture; optionally, homogenizing the mixture; optionally, adding a flavorant or colorant; optionally, adding a diluent to obtain a target volume.

In some embodiments, the target dose for each of the at least one bioactive molecule in each extract comprises: 250 μg of psilocybin; 150 μg of psilocin; 1 mg of CBD; 1 mg of THC; and 1 mg of coumarin.

In some embodiments, the calculated amount of each extract based on the compounding percentage comprises: 45% by volume of the total combination for the fungal extract; 15% by volume of the total combination for the Cannabis plant extract; 2% by volume of the total combination for the Dipteryx plant extract; 15% by volume of the total combination for the algal extract; 15% by volume of the total combination for the flavorant or colorant; and 8% by volume of the total combination for the diluent.

In some aspects are disclosed pharmaceutical compositions comprising a disclosed therapeutic combination and a pharmaceutically acceptable carrier, diluent, or excipient. In some embodiments, the pharmaceutically acceptable carrier, diluent, or excipient is non-naturally occurring.

In some embodiments, the pharmaceutical composition is suitable for enteral or parenteral administration. In some embodiments, the pharmaceutical composition is prepared as any of a tincture formulation, oral spray formulation, oral mucosal spray formulation, soft mist inhaler formulation, vaporizer formulation, tablet formulation, scorable double-strength tablet formulation, capsule formulation, capsule formulation with additional active agent, suspension formulation, intravenous solution formulation, injectable solution formulation, topical formulation for transdermal administration, cut matrix sublingual or buccal tablet formulation, individually formed sublingual or buccal lozenge formulation, intranasal delivery formulation. In some embodiments, the pharmaceutical composition is prepared as any of a tincture formulation, oral spray formulation, oral mucosal spray formulation, or soft mist inhaler formulation.

In some embodiments, the pharmaceutical composition is a formulation, comprising: 45% by volume of the total combination for the fungal extract; 15% by volume of the total combination for the Cannabis plant extract; 2% by volume of the total combination for the Dipteryx plant extract; 15% by volume of the total combination for the algal extract; 15% by volume of the total combination for the flavorant or colorant; and 8% by volume of the total combination for the diluent.

In some embodiments, a single dose of the formulation comprises: 250 μg of psilocybin; 150 μg of psilocin; 1 mg of CBD; 1 mg of THC; 1 mg of coumarin; 8 mg of an extract of Pyropia; optionally, 8 mg of a flavorant or colorant.

In some embodiments, a single dose of psilocybin is between about 0.5 μg to about 200 mg, between about 5 μg to about 5 mg, or between about 100 μg to about 600 μg. In some embodiments, a single dose of psilocybin is 250 μg. In some embodiments, a single dose of psilocin is between about 0.5 μg to about 200 mg, between about 5 μg to about 5 mg, or between about 100 μg to about 600 μg. In some embodiments, a single dose of psilocin is 150 μg.

In some embodiments, a single dose of CBD is between about 0.5 μg to about 200 mg, between about 0.01 mg to about 75 mg, or between about 0.5 mg to about 15 mg. In some embodiments, a single dose of CBD is 1 mg. In some embodiments, a single dose of THC is between about 0.5 μg to about 200 mg, between about 0.01 mg to about 75 mg, or between about 0.5 mg to about 15 mg. In some embodiments, a single dose of THC is 1 mg.

In some embodiments, a single dose of coumarin is between about 0.5 μg to about 200 mg, between about 0.01 mg to about 75 mg, or between about 0.5 mg to about 15 mg. In some embodiments, a single dose of coumarin is 1 mg.

In some embodiments, a single dose includes a Pyropia yezoensis, Pyropia perforata, or Porphyra umbilicalis whole extract, in an amount of between about 0.5 mg to about 100 mg, between about 1 mg to about 50 mg, or between about 5 mg to about 20 mg. In some embodiments, a single dose includes a Pyropia whole extract in an amount of 8 mg.

In some embodiments, a single dose includes ethanol infused with ginger and bay leaf, in an amount of between about 0.5 mg to about 100 mg, between about 1 mg to about 50 mg, or between about 5 mg to about 20 mg. In some embodiments, a single dose includes ethanol infused with ginger and bay leaf in an amount of 8 mg.

In some aspects are disclosed pharmaceutical kits comprising a first pharmaceutical composition, and a second pharmaceutical composition, wherein: the first pharmaceutical composition comprises at least one portion of the therapeutic combination of claim 21, and a pharmaceutically acceptable carrier, diluent, or excipient; and the second pharmaceutical composition comprises the remaining portions of the therapeutic combination of claim 21, and a pharmaceutically acceptable carrier, diluent, or excipient.

In some embodiments, the first pharmaceutical composition is prepared as a tincture formulation, oral spray formulation, oral mucosal spray formulation, or soft mist inhaler formulation; and the second pharmaceutical composition is prepared as a tincture formulation, oral spray formulation, oral mucosal spray formulation, or soft mist inhaler formulation.

In some aspects are disclosed methods of preventing or treating a movement disorder, comprising administering to a patient in need thereof, a disclosed therapeutic combination, a disclosed pharmaceutical composition, or a disclosed pharmaceutical kit.

In some embodiments, the movement disorder is any one or more of ataxia, an ataxic disorder, a certain specified movement disorder, cervical dystonia, chorea, a choreiform disorder, dystonia, a dystonic disorder, essential tremor, Friedreich's ataxia, a functional movement disorder, hemifacial spasm, hereditary spastic paraplegia, Huntington's disease, L-dopa induced dyskinesia, multiple system atrophy (MSA), myoclonus, a myoclonic disorder, Parkinson's disease, atypical Parkinson's, Parkinsonism, Secondary Parkinsonism, progressive supranuclear palsy (PSP), restless legs syndrome, Rett syndrome, a sleep-related movement disorder, spasticity, tardive dyskinesia (TD), tourette syndrome, a tic disorder, a disorder associated with tremor, and Wilson's disease.

In some embodiments, the pharmaceutical composition is administered between 1 and 8 times per day. In some embodiments, the patient experiences an improvement related to the movement disorder. In some embodiments, the improvement is a reduction in the severity of at least one symptom of the movement disorder.

In some embodiments, the at least one symptom of the movement disorder is a motor symptom. In some embodiments, the motor symptom is any of stooped posture, masked facial expression, forward tilt of trunk, flexed elbows and wrists, reduced arm swinging, flexed hips and knees, trembling of extremities, shuffling gait, short-stepped gait, uncoordinated or clumsy balance, altered speech, involuntary limb movements, irregular motor movement, long-lasting contractions, intermittent contractions of neck muscles, causing the head to turn in different ways; repetitive, irregular, involuntary movements involving the face, mouth, trunk, and limbs; twisting, repetitive movements; jerking of muscles or groups of muscles, tremors, stiffness, finger tapping, toe tapping, poor posture, slow, decreased movement or imbalance; difficulties walking, random involuntary eye movement, involuntary blinking, involuntary grimacing, unpleasant, abnormal feelings in limbs which may be relieved by movement; involuntary vocal sounds, and rhythmic shaking of parts of the body, commonly the hands and/or head. In some embodiments, the motor symptom is any of issues with speech, overproducing saliva and drooling, problems with chewing and swallowing, difficulty eating, dressing, maintaining proper hygiene, handwriting, doing hobbies and other activities, turning in bed, getting out of bed, a car, or a deep chair; trouble walking and maintaining balance, experiencing tremor, and freezing in place.

In some embodiments, the at least one symptom of the movement disorder is a non-motor symptom. In some embodiments, the non-motor symptom is any of cognitive impairment, hallucinations and psychosis, depressed mood, anxious mood, apathy, features of dopamine dysregulation syndrome, sleep problems, daytime sleepiness, pain and other sensations, urinary problems, constipation problems, lightheadedness on standing, and fatigue. In some embodiments, the non-motor symptom is a mood symptom. In some embodiments, the mood symptom is any of feelings of depression, anxiety, irritability, mood swings, impaired judgment, loss of empathy, aggression, impulsivity, delusions, and paranoia.

In some embodiments, the reduction in the severity of at least one symptom of the movement disorder occurs less than about 75 days from the first administration of the pharmaceutical composition. In some embodiments, the reduction in the severity of at least one symptom of the movement disorder occurs less than about 35 days from the first administration of the pharmaceutical composition. In some embodiments, the reduction in the severity of at least one symptom of the movement disorder lasts for at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 36 months, at least 48 months, or at least 60 months.

In some embodiments, the improvement is an improvement in motor control. In some embodiments, the improvement in motor control is an improvement of any of balance, frequency of involuntary movements, amplitude of involuntary movements, strength, endurance, and physical capacity. In some embodiments, the improvement in motor control occurs less than about 75 days from the first administration of the pharmaceutical composition. In some embodiments, the improvement in motor control occurs less than about 35 days from the first administration of the pharmaceutical composition.

In some embodiments, the improvement is to a clinical outcome assessment. In some embodiments, the clinical outcome assessment is any of the Movement Disorder Society-Unified Parkinson's Disease Rating Scale (MDS-UPDRS), the Movement Disorder Society Non-Motor Rating Scale (MDS-NMS), the Cortical Basal Ganglia Functional Scale (SBFS), the Gastrointestinal Dysfunction Scale for Parkinson's Disease (GIDS-PD), the Global Assessment Scale for Wilson's Disease (GAS for WD), the Global Dystonia Severity Rating Scale (GDS), the Modified Bradykinesia Rating Scale (MBRS), the Non-Motor Symptoms Questionnaire (NMSQ), the Non-Motor Symptoms Scale for Parkinson's Disease (NMSS), the Pantothenate Kinase-Associated Neurodegeneration Disease Rating Scale (PKAN-DRS), the Progressive Supranuclear Palsy Clinician Deficits Scale (PSP-CDS), the Quality of Life in Essential Tremor Questionnaire, the Rating Scale for Psychogenic Movement Disorders, the Rush Dyskinesia Rating Scale (RDRS), the Rush Video-Based Tic Rating Scale (RVBTRS), Scales for Outcomes in Parkinson's Disease-Autonomic Dysfunction (SCOPA-AUT), Scales for Outcomes in Parkinson's Disease-Diary Card (SCOPA-DC), Scales for Outcomes in Parkinson's Disease-Psychiatric Complications (SCOPA-PC), Scales for Outcomes in Parkinson's Disease-Psychosocial Functioning (SCOPA-PS), Scales for Outcomes in Parkinson's Disease-Sleep (SCOPA-Sleep; SCOPA-S), Scales for Outcomes in Parkinson's Disease-Cognition (SCOPA-COG), Short Parkinson's Evaluation Scale (SPES)/Scales for Outcomes in Parkinson's Disease-Motor Function (SPES/SCOPA-Motor), The Non-Motor Fluctuation Assessment (NoMoFA) Questionnaire, the UFMG Sydenham's Chorea Rating Scale (USCRS), Unified Dyskinesia Rating Scale (UDysRS), Unified Dystonia Rating Scale (UDRS), Unified Multiple System Atrophy Rating Scale (UMSARS), and the 8-item Unified Parkinson's Disease Rating Scale (UPDRS-8). In some embodiments, the clinical outcome assessment is the MDS-UPDRS, the UPDRS, or the UPDRS-8. In some embodiments, the clinical outcome assessment is the MDS-UPDRS.

In some embodiments, the improvement in the MDS-UPDRS is an improvement in the nM-EDL. In some embodiments, the improvement in the nM-EDL is to any of cognitive impairment, hallucinations and psychosis, depressed mood, anxious mood, apathy, features of dopamine dysregulation syndrome, sleep problems, daytime sleepiness, pain and other sensations, urinary problems, constipation problems, lightheadedness on standing, and fatigue.

In some embodiments, the improvement in the MDS-UPDRS is an improvement in the M-EDL. In some embodiments, the improvement in the M-EDL is to any of speech, saliva and drooling, chewing and swallowing, eating tasks, dressing, hygiene, handwriting, doing hobbies and other activities, turning in bed, tremor, getting out of bed, a car, or a deep chair; walking and balance, and freezing.

In some embodiments, the improvement in the MDS-UPDRS is an improvement in motor examination. In some embodiments, the improvement in motor examination is to any of speech, facial expression, rigidity, finger tapping, hand movements, pronation-supination movements of hands, toe tapping, leg agility, arising from chair, gait, freezing of gait, postural stability, posture, global spontaneity of movement (body bradykinesia), postural tremor of the hands, kinetic tremor of the hands, rest tremor amplitude, constancy of rest tremor, Hoehn and Yahr stage, time spent with dyskinesias, functional impact of dyskinesias, time spent in the off state, functional impact of fluctuations, complexity of motor fluctuations, and painful off-state dystonia.

In some embodiments, the improvement is a reduction in score. In some embodiments, the reduction in score is by at least 1 point, at least 2 points, at least 3 points, or at least 4 points.

In some embodiments, the improvement is an improvement in the UPDRS.

In some embodiments, the improvement in the UPDRS is an improvement in mentation, behavior, and mood. In some embodiments, the improvement in mentation, behavior, and mood is any of intellectual impairment, thought disorder, depression, and motivation/initiative.

In some embodiments, the improvement in the UPDRS is an improvement in ADL. In some embodiments, the improvement in ADL is to any of speech, salivation, swallowing, handwriting, cutting food and handling utensils, dressing, hygiene, turning in bed and adjusting bed clothes, falling, freezing when walking, walking, tremor, and sensory complaints related to parkinsonism.

In some embodiments, the improvement in the UPDRS is an improvement in motor examination. In some embodiments, the improvement in motor examination is to any of speech, facial expression, tremor at rest, action or postural tremor of hands, rigidity, finger taps, hand movements, rapid alternating movements of hands, leg agility, arising from chair, posture, gait, postural stability, and body bradykinesia and hypokinesia.

In some embodiments, the improvement in the UPDRS is an improvement in complications of therapy. In some embodiments, the improvement in complications of therapy is to any of daily duration of dyskinesias, severity of disability from dyskinesias, painful dyskinesias, and the proportion of the waking day the patient is “off” on average.

In some embodiments, the improvement is an improvement in score. In some embodiments, the improvement in score is by at least 1 point, at least 2 points, at least 3 points, or at least 4 points. In some embodiments, the improvement in the UPDRS is a reduction in stage of the modified Hoehn and Yahr staging session. In some embodiments, the reduction is a reduction in at least 1 stage. In some embodiments, the improvement in the UPDRS is a reduction in the Schwab and England A DL scale percentage. In some embodiments, the reduction is a reduction of between about 10% and about 100%. In some embodiments, the improvement in the UPDRS is a change in the binary yes and no questions.

In some embodiments, the improvement occurs less than about 75 days from the first administration of the pharmaceutical composition. In some embodiments, the improvement occurs less than about 35 days from the first administration of the pharmaceutical composition. In some embodiments, the improvement lasts for at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 36 months, at least 48 months, or at least 60 months.

The foregoing has outlined broadly some pertinent features of certain exemplary embodiments of the present disclosure so that the detailed description that follows may be better understood and the contribution to the art more fully appreciated. Additional features of the invention will be described hereinafter which form the subject of the claims. It will be appreciated by those in the art that the disclosed specific formulations and methods may be readily utilized as a basis for modifying or designing other formulations and methods for carrying out the same purposes of the disclosure. It also will be realized that such equivalent formulations and methods do not depart from the spirit and scope of the invention as set forth in the claims. Hence, this summary is made with the understanding that it will be considered as a brief and general synopsis of only some of the aspects and embodiments herein, is provided solely for the benefit and convenience of the reader, and is not intended to limit in any manner the scope, or range of equivalents, to which the claims are lawfully entitled.

### BRIEF DESCRIPTION OF THE FIGURES

To further clarify various aspects of the invention, a more particular description is rendered by reference to certain exemplary embodiments illustrated in the figures. It will be appreciated that these figures depict only illustrated embodiments of the invention and should not be considered limiting of its scope. They are merely provided as exemplary illustrations of certain concepts of some embodiments of the invention. Certain aspects of the invention are therefore further described and explained with additional specificity and detail, but still by way of example only, with reference to the accompanying figures in which:

FIG. 1 illustrates by way of summary and in the aggregate certain observed repeated patterns of therapeutic activity and benefit by individuals utilizing a disclosed combination.

FIG. 2 illustrates hand motion improvements via software-assisted motion tracking.

FIG. 3 illustrates by way of summary certain observed data for the treatment of Parkinson's disease, in a study conducted according to some embodiments of the invention, regarding the disease footprint, using the 8-item version of the Unified Parkinson's Disease Rating Scale (UPDRS-8) scoring (see, e.g., Hauser, Lyons, and Pahwa, 2012), pre- and post-treatment with the composition referred to herein as ABS-108, and defined below.

FIG. 4 illustrates by way of summary certain observed data for the treatment of Parkinson's disease, in a study conducted according to some embodiments of the invention, by time to ABS-108 motor response relative to disease progression.

FIG. 5 illustrates the UPDRS-8 scoring estimate, for each of the domains of non-motor, motor, and motor complications, for patient A2 (as further described below), as a change from baseline, pre- and post-treatment with ABS-108.

FIG. 6 illustrates the UPDRS-8 scoring estimate for patient A3.

FIG. 7 illustrates the UPDRS-8 scoring estimate for patient A4.

FIG. 8 illustrates the UPDRS-8 scoring estimate for patient A5.

FIG. 9 illustrates the UPDRS-8 scoring estimate for patient A6.

FIG. 10 illustrates the UPDRS-8 scoring estimate for patient A9.

FIG. 11 illustrates the UPDRS-8 scoring estimate for patient A10.

FIG. 12 illustrates the UPDRS-8 scoring estimate for patient A11.

FIG. 13 illustrates the UPDRS-8 scoring estimate for patient A12.

FIG. 14 illustrates the UPDRS-8 scoring estimate for patient A13.

FIG. 15 illustrates the UPDRS-8 scoring estimate for patient A14.

FIG. 16 illustrates the UPDRS-8 scoring estimate for patient A16.

FIG. 17 illustrates the UPDRS-8 scoring estimate for patient A17.

FIG. 18 illustrates the UPDRS-8 scoring estimate for patient A18.

FIG. 19 is a spider chart with UPDRS-8 scores for each of the 8 items, pre- and post-treatment with ABS-108, according to the methods of EXAMPLE 16, for patient A2.

FIG. 20 is a spider chart with UPDRS-8 scores pre- and post-treatment for patient A3.

FIG. 21 is a spider chart with UPDRS-8 scores pre- and post-treatment for patient A4.

FIG. 22 is a spider chart with UPDRS-8 scores pre- and post-treatment for patient A5.

FIG. 23 is a spider chart with UPDRS-8 scores pre- and post-treatment for patient A6.

FIG. 24 is a spider chart with UPDRS-8 scores pre- and post-treatment for patient A9.

FIG. 25 is a spider chart with UPDRS-8 scores pre- and post-treatment for patient A10.

FIG. 26 is a spider chart with UPDRS-8 scores pre- and post-treatment for patient A11.

FIG. 27 is a spider chart with UPDRS-8 scores pre- and post-treatment for patient A12.

FIG. 28 is a spider chart with UPDRS-8 scores pre- and post-treatment for patient A13.

FIG. 29 is a spider chart with UPDRS-8 scores pre- and post-treatment for patient A14.

FIG. 30 is a spider chart with UPDRS-8 scores pre- and post-treatment for patient A16.

FIG. 31 is a spider chart with UPDRS-8 scores pre- and post-treatment for patient A17.

FIG. 32 is a spider chart with UPDRS-8 scores pre- and post-treatment for patient A18.

FIG. 33 illustrates the production of an exemplary formulation according to the methods of certain described embodiments.

FIG. 34 illustrates an example analysis of a formulation via liquid chromatography-mass spectrometry (LC-MS) testing, including chromatograms showing the various concentrations of bioactive molecules contained in a formulation with parameters disclosed in TABLE 6.

FIG. 35 illustrates an example 1H NMR spectrum of a disclosed composition having parameters described in TABLE 6.

FIG. 36 illustrates an example analysis of a formulation via multiple reaction monitoring (MRM) LC-MS testing, including a chromatogram showing the concentration of bioactive molecules contained in a formulation with parameters disclosed in TABLE 6.

FIG. 37 illustrates total antioxidant capacity of Cannabis Stock, measured via Folin-Ciocalteu assay, wherein the calculated Gallic Acid Equivalents (GAEs) of Cannabis Stock in milligrams (mg) are plotted per milliliter/liter (mL/L) of a given solution.

FIG. 38 illustrates total antioxidant capacity of Psilocybe Stock, using Folin-Ciocalteu assay, with the calculated GAEs of Psilocybe Stock in mg plotted per mL/L of a given solution.

FIG. 39 illustrates total antioxidant capacity of Pyropia Stock, using Folin-Ciocalteu assay, with the calculated GAEs of Pyropia Stock in mg plotted per mL/L of a given solution.

FIG. 40 illustrates total antioxidant capacity of Tonka Concentrate, using Folin-Ciocalteu assay, with calculated GAEs of Tonka Concentrate in mg plotted per mL/L of a given solution.

FIG. 41 illustrates the total antioxidant capacity of Cannabis Stock (Ca Stock), Psilocybe Stock (Ps Stock), and the 2-ingredient blend of Cannabis/Psilocybe (Ca/Ps), using (i.e., obtained via) Folin-Ciocalteu assay. FIG. 41 shows calculated GAEs for Ca Stock, for Ps Stock, and for Ca/Ps in mg per mL/L of solution utilized. The table under the graph shown in FIG. 41 depicts the statistical significance of Ca Stock to Ca/Ps versus Ps Stock to Ca/Ps, and is indicated by asterisks (when P<0.10: (*), P<0.05: * and P<0.01: **). The 2-ingredient blend of Cannabis and Psilocybe had a higher antioxidant capacity than each ingredient alone, and this data suggested an additive effect, where the two ingredients contributed to the capacity of the 2-ingredient blend.

FIG. 42 illustrates the total antioxidant capacity of Cannabis Stock (Ca Stock), Pyropia Stock (Py Stock), and the 2-ingredient blend of Cannabis/Pyropia (Ca/Py), obtained via Folin-Ciocalteu assay. FIG. 42 shows the calculated GAEs for Ca Stock, for Py Stock, and for Ca/Py in mg per mL/L of solution utilized. The table under the graph shown in FIG. 42 depicts the statistical significance of Ca Stock to Ca/Py versus Py Stock to Ca/Py, and is indicated by asterisks (when P<0.10: (*), P<0.05: * and P<0.01: **). The 2-ingredient blend of Cannabis and Pyropia had a higher antioxidant capacity than each ingredient alone, and this data suggested a synergistic effect, since the antioxidant capacity of the 2-ingredient blend was more than what would be expected by adding the numbers for the two ingredients together.

FIG. 43 illustrates the total antioxidant capacity of Cannabis Stock (Ca Stock), Tonka Stock (To Stock), and the 2-ingredient blend of Cannabis/Tonka (Ca/To), obtained via Folin-Ciocalteu assay. FIG. 43 shows the calculated GAEs for Ca Stock, for To Stock, and for Ca/To in mg per mL/L of solution utilized. The table under the graph shown in FIG. 43 depicts the statistical significance of Ca Stock to Ca/To versus To Stock to Ca/To, and is indicated by asterisks (when P<0.10: (*), P<0.05: * and P<0.01: **). The 2-ingredient blend of Cannabis and Tonka had a similar antioxidant capacity as the Cannabis ingredient. Tonka provided a slight enhancement of the antioxidant capacity, despite its relatively low proportion in the blend.

FIG. 44 illustrates the total antioxidant capacity of Psilocybe Stock (Ps Stock), Pyropia Stock (Py Stock), and the 2-ingredient blend of Psilocybe/Pyropia (Ps/Py), obtained via Folin-Ciocalteu assay. FIG. 44 shows the calculated GAEs for Ps Stock, for Py Stock, and for Ps/Py in mg per mL/L of solution utilized. The table under the graph shown in FIG. 44 depicts the statistical significance of Ps Stock to Ps/Py versus Py Stock to Ps/Py, and is indicated by asterisks (when P<0.10: (*), P<0.05: * and P<0.01: **). The 2-ingredient blend of Psilocybe and Pyropia had slightly lower antioxidant capacity than Psilocybe alone. This suggests that compounds in the Pyropia stock solution interacted in a way that reduced the antioxidant capacity, i.e., the ability to donate electrons to the chemical reaction in the assay.

FIG. 45 illustrates the total antioxidant capacity of Psilocybe Stock (Ps Stock), Tonka Stock (To Stock), and the 2-ingredient blend of Psilocybe/Tonka (Ps/To), obtained via Folin-Ciocalteu assay. FIG. 45 shows the calculated GAEs for Ps Stock, for To Stock, and for Ps/To in mg per mL/L of solution utilized. The table under the graph shown in FIG. 45 depicts the statistical significance of Ps Stock to Ps/To versus To Stock to Ps/To, and is indicated by asterisks (when P<0.10: (*), P<0.05: * and P<0.01: **). The 2-ingredient blend of Psilocybe and Tonka had a lower antioxidant capacity than Psilocybe alone. This suggests that compounds in Tonka interacted with compounds in Psilocybe in a way that reduced the antioxidant capacity, i.e., the ability to donate electrons to the chemical reaction in the assay.

FIG. 46 illustrates the total antioxidant capacity of Pyropia Stock (Py Stock), Tonka Stock (To Stock), and the 2-ingredient blend of Pyropia/Tonka (Py/To), using Folin-Ciocalteu assay. FIG. 46 shows the calculated GAEs for Py Stock, for To Stock, and for Py/To in mg per mL/L of solution utilized. The table under the graph shown in FIG. 46 depicts the statistical significance of Py Stock to Py/To versus To Stock to Py/To, and is indicated by asterisks (when P<0.10: (*), P<0.05: * and P<0.01: **). The 2-ingredient blend of Pyropia and Tonka had a similar antioxidant capacity as Pyropia alone. Tonka's effects alone were undetectable at the dose range tested.

FIG. 47 illustrates the total antioxidant capacity for the blend of Cannabis/Psilocybe/Pyropia/Tonka (Ca/Ps/Py/To) compared to matching doses of Cannabis Stock (Ca Stock), Psilocybe Stock (Ps Stock), Pyropia Stock (Py Stock), and Tonka Stock (To Stock), obtained via Folin-Ciocalteu assay. FIG. 47 shows the calculated GAEs for Ca/Ps/Py/To, for Ca Stock, for Ps Stock, for Py Stock, and for To Stock in mg per mL/L of solution utilized. The table under the graph shown in FIG. 47 depicts the statistical significance of Ca/Ps/Py/To to Ca Stock versus Ca/Ps/Py/To to Ps Stock versus Ca/Ps/Py/To to Py Stock versus Ca/Ps/Py/To to To Stock, and is indicated by asterisks (when P<0.10: (*), P<0.05: * and P<0.01: **).

FIG. 48 illustrates the total antioxidant capacity for the blend of Cannabis/Psilocybe/Pyropia/Tonka (Ca/Ps/Py/To) compared to matching doses of each of the 2-ingredient blends, including Cannabis/Psilocybe (Ca/Ps), Cannabis/Pyropia (Ca/Py), Cannabis/Tonka (Ca/To), Psilocybe/Pyropia (Ps/Py), Psilocybe/Tonka (Ps/To), and Pyropia/Tonka (Py/To), obtained via Folin-Ciocalteu assay. FIG. 48 shows the calculated GAEs for Ca/Ps/Py/To, for Ca/Ps, for Ca/Py, for Ca/To, for Ps/Py, for Ps/To, and for Py/To in mg per mL/L of solution utilized. The table under the graph shown in FIG. 48 depicts the statistical significance of Ca/Ps/Py/To to Ca/Ps versus Ca/Ps/Py/To to Ca/Py versus Ca/Ps/Py/To to Ca/To versus Ca/Ps/Py/To to Ps/Py versus Ca/Ps/Py/To to Ps/To versus Ca/Ps/Py/To to Py/To, and is indicated by asterisks (when P<0.10: (*), P<0.05: * and P<0.01: **).

FIG. 49 illustrates the total antioxidant capacity of the 4-ingredient blend of Cannabis/Psilocybe/Pyropia/Tonka (Ca/Ps/Py/To) in comparison to Applicant's “4+” Blend (i.e., Applicant's final product disclosed herein as ABS-108 and NIM-01, and as the formulation of EXAMPLE 1) obtained via Folin-Ciocalteu assay. FIG. 49 shows the calculated GAEs for Ca/Ps/Py/To and for 4+ Blend in mg per mL/L of solution utilized. FIG. 49 depicts data as the average #standard deviation of duplicate data points for each dose. FIG. 49 describes the statistical significance of Ca/Ps/Py/To versus 4+ Blend, and is indicated by asterisks (when P<0.10: (*), P<0.05: * and P<0.01: **). The 4+ blend showed a higher antioxidant capacity than the 4-ingredient blend of Cannabis/Psilocybe/Pyropia/Tonka. This shows that the additional ingredients in the 4+ blend contribute to the antioxidant properties of the final product.

### DETAILED DESCRIPTION OF THE INVENTION

While various aspects and features of certain embodiments are summarized above, the following detailed description illustrates some exemplary embodiments in further detail to enable one having ordinary skill in the art to which the invention belongs (“one of skill”) to practice such embodiments and to make and use the full scope of the invention claimed.

Many modifications, substitutions, changes, and variations in the described examples, embodiments, applications, and details of the invention illustrated herein can be made by one of skill without departing from the spirit of the invention, or the scope of the invention as described in the appended claims, and the general principles defined herein may be applied to a wide range of aspects. Thus, the invention is not intended to be limited to the aspects presented, but is to be accorded the widest scope consistent with the principles and features disclosed. The description below is designed to make such embodiments apparent to one of skill, in that the embodiments shall be both readily cognizable and readily creatable without undue experimentation, solely using the teachings herein together with the general knowledge in the art.

Among the aspects of the invention are therapeutic combinations comprising bioactive molecules from a fungus, a plant, and/or an algae. Among other aspects of the invention are pharmaceutical compositions comprising the therapeutic combinations. Among other aspects are methods of using the therapeutic combinations and pharmaceutical compositions to treat movement disorders, such as Parkinson's disease.

### A. General Definitions and Terms

As used in this specification and the appended claims, the singular forms “a,” “an,” and “the” include plural referents unless the context clearly dictates otherwise. Thus, for example, reference to “an excipient” includes reference to a combination of one or more excipients, and reference to “a bioactive molecule” includes reference to a combination of one or more bioactive molecules. Similarly, “a bioactive molecule” includes reference to bioactive molecule(s) within another substance, such as an extract, unless the bioactive molecule(s) are indicated expressly or by context as being purified or isolated therefrom, and even then may include another substance as long as the bioactive molecule(s) are, as one example, within the degree of purity indicated.

The terms “comprising,” “including,” “such as,” and “having” are intended to be inclusive and not exclusive (i.e., there may be other elements in addition to the recited elements). Thus, the term “including” as used herein means, and is used interchangeably with, the phrase “including but not limited to.” The term “or” is used herein to mean, and is used interchangeably with, the term “and/or,” unless context clearly indicates otherwise, and the specific use of the term “and/or” does not signify that any uses of “or” are disjunctive only; rather, such use simply underscores the possibility that the term “and/or” may be conjunctive in particular embodiments, but otherwise may be disjunctive, like “or.” The term “and” will be understood to be conjunctive.

Compositions “consisting of” specific bioactive molecules may include only the bioactive molecules recited, and those “consisting essentially of” specific bioactive molecules may include the recited bioactive molecules, optionally together with additional compounds or other elements that do not materially affect the basic and novel characteristic(s) of the claimed combination, composition, or method. While the term “one or more” may be used, its absence (or its replacement by the singular) does not signify the singular only; rather, such use simply underscores the possibility of multiple agents or ingredients in particular embodiments.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. It is also understood that unless otherwise indicated or otherwise evident from the context and understanding of one of skill, values that are expressed as ranges can assume any specific value or subrange within the stated ranges in different embodiments, to the tenth of the unit of the lower limit of the range, unless the context clearly dictates otherwise. It is further understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment in which a numerical value is prefaced by “about” or “approximately,” the invention includes an embodiment in which the exact value is recited. For any embodiment in which a numerical value is not prefaced by “about” or “approximately,” the invention includes an embodiment in which the value is prefaced by “about” or “approximately.” Unless otherwise indicated, all numbers expressing quantities of ingredients, properties such as concentration, reaction conditions, and so forth, used to describe and claim certain embodiments of the invention, therefore are to be understood as being modified in some instances by the term “about.” “Approximately” or “about” is intended to encompass numbers that fall within a range of +10% of a number, in some embodiments within +5% of a number, in some embodiments (including in some preferred embodiments) within +2% of a number, in some embodiments within +1% of a number, in some embodiments within +0.5% of a number, in some embodiments within +0.1% of a number unless otherwise stated or otherwise evident from the context (except where such number would impermissibly exceed 100% of a possible value).

In some embodiments, the numerical parameters set forth in the description and claims are approximations that can vary depending upon the desired properties sought to be obtained by a particular embodiment (and as would be understood to one of skill). The term “substantially,” where it is applied to modify a feature or limitation herein, will be read in the context of the invention and in light of the knowledge in the art to provide the appropriate certainty, e.g., by using a standard that is recognized in the art for measuring the meaning of “substantially” as a term of degree, or by ascertaining the scope as would one of skill. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in embodiments may contain certain errors necessarily resulting from the standard deviation found in their respective testing measurements.

A comprehensive list of the abbreviations utilized by organic chemists of ordinary skill appears in the first issue of each volume of the Journal of Organic Chemistry; this list is typically presented in a table entitled Standard List of Abbreviations. The current list as of the date of this filing is hereby incorporated by reference as if fully set forth herein.

Unless explicitly defined otherwise, all technical and scientific terms herein have the meaning as commonly understood by one of skill. Further definitions that may assist a reader in understanding the disclosed and exemplary embodiments are below; however, it will be appreciated that such definitions are not intended to limit the scope of the invention, which shall be properly interpreted and understood by reference to the full specification (as well as any plain meaning known to one of skill) in view of the language used in the claims. Terminology herein is for the purpose of describing particular embodiments only and is not intended to be limiting.

Terms having a specific meaning within the regulatory law of a jurisdiction in which this application is filed or may be in force generally should be given such meaning unless context dictates otherwise. For example, “Botanical Drug Substance” may refer to the term as defined through FDA implementing rules and regulations, and as described in the Guidance for Industry Botanical Drug Products, December 2016 (Docket No. FDA-2000-D-0103), U.S. Department of Health and Human Services, Food and Drug Administration Center for Drug Evaluation and Research (“2016 FDA Botanical Drug Guidance”). Other such terms, including “Botanical Drug Product” and “Botanical Raw Materials,” shall be known similarly.

For avoidance of doubt, even though “botanical” is commonly used to mean “relating to plants,” for purposes herein “botanical,” as in a “botanical” drug substance or drug product, will be understood to include compounds, substances, and products (e.g., extracts) obtained or derived from fungal material as well, or present therein, such as psilocybin-containing or other fungi, such as defined by the FDA. A “botanical” drug substance or drug product also will be understood to include compounds, substances, and products (e.g., extracts) obtained or derived from algal material, or present therein, wherein “algal material” refers broadly to any material from the polyphyletic group of diverse photosynthetic eukaryotic organisms that is referred to or understood in the field as “algae,” and especially those parts comprising the bioactive molecules of interest, as known by those of skill.

Botanical drug substances and drug products, and compositions comprising them, may be available by prescription or over-the-counter (“OTC”), or as nutritional or dietary supplements, or under any other regulatory regime; they also may be unregulated (e.g., “natural products”).

Herein the term “plant material” encompasses whole plants and also parts thereof which contain the bioactive molecules sought, for example the aerial parts of a plant or isolated leaves, stems, flowers, fruits, roots, or combinations of any of the foregoing. With cannabis plant material, it will be understood that the parts used primarily shall be the inflorescences (“buds”) of the flowering female plant, which generally comprise the greatest concentration of bioactive molecules, such as terpenoids and cannabinoids. Other plant parts however also shall be able to be used in the disclosed compositions and methods, as will be appreciated by those of skill.

Where fungal material is used (e.g., in an extraction process), it may be from any part of fungal fruiting bodies (“mushrooms”), from fungal sclerotia (“truffles”), as well as from mycelia or other fungal material (e.g., bioreactor biomass), unless context indicates otherwise, and any or all such parts, as well as combinations thereof, may be referred to as “fungal material.”

Generally, the nomenclature used and procedures performed herein are those known in fields relating to an aspect of the invention, such as biology, chemistry, natural products extraction, botany, mycology, phycology, pharmacology, and medicine, and are those that will be well known and commonly employed in such fields. Standard techniques and procedures will be those generally performed according to conventional methods in the art.

It will be appreciated that the headings in this document are used only to expedite its review by a reader. They should not be construed as limiting the invention in any manner.

### B. Primary and Secondary Bioactive Molecules

In some aspects, provided therapeutic combinations are useful to prevent or treat a movement disorder, such as Parkinson's disease (PD). In some embodiments, a therapeutic combination comprises a fungal portion, a plant portion, and an algal portion. In some embodiments, a therapeutic combination comprises a fungal portion, a first plant portion, an optional second plant portion, and an algal portion. In embodiments, a therapeutic combination comprises a fungal portion, a first plant portion, a second plant portion, and an algal portion.

In some embodiments, a fungal, a plant, or an algal portion comprises an extract (i.e., a fungal, plant, or algal extract, respectively). In embodiments, a therapeutic combination comprises a fungal extract, a plant portion, and an algal portion. In some embodiments, a therapeutic combination comprises a fungal extract, a first plant portion, an optional second plant portion, and an algal portion. In some embodiments, a therapeutic combination comprises a fungal extract, a first plant portion, a second plant portion, and an algal portion. In some embodiments, a therapeutic combination comprises a fungal portion, a first plant extract, an optional second plant extract, and an algal portion. In some embodiments, a therapeutic combination comprises a fungal portion, a first plant extract, a second plant extract, and an algal portion. In some embodiments, a therapeutic combination comprises a fungal portion, a first plant portion, an optional second plant portion, and an algal extract. In some embodiments, a therapeutic combination comprises a fungal portion, a first plant portion, a second plant portion, and an algal extract. In some embodiments, a therapeutic combination comprises a fungal extract, a first plant extract, an optional second plant extract, and an algal portion. In some embodiments, a therapeutic combination comprises a fungal extract, a first plant extract, a second plant extract, and an algal portion. In some embodiments, a therapeutic combination comprises a fungal extract, a first plant portion, an optional second plant portion, and an algal extract. In some embodiments, a therapeutic combination comprises a fungal extract, a first plant portion, a second plant portion, and an algal extract. In some embodiments, a therapeutic combination comprises a fungal extract, a plant extract, and an algal extract. In some embodiments, a therapeutic combination comprises a fungal extract, a first plant extract, an optional second plant extract, and an algal extract. In some embodiments, a therapeutic combination comprises a fungal extract, a first plant extract, a second plant extract, and an algal extract.

In some embodiments, the therapeutic combination comprises a bioactive molecule from a fungus, a plant, and/or an algae. In some embodiments, a therapeutic combination comprises a bioactive molecule from a fungus, and a bioactive molecule from a plant. In some embodiments, a therapeutic combination comprises a bioactive molecule from a plant, and a bioactive molecule from an algae. In some embodiments, a therapeutic combination comprises a bioactive molecule from a fungus, a bioactive molecule from a plant, and a bioactive molecule from an algae. In some embodiments, a therapeutic combination comprises a bioactive molecule from a fungus, a bioactive molecule from a plant, a bioactive molecule from an algae, and a bioactive molecule from a fungus, a plant, and/or an algae. In some embodiments, a therapeutic combination comprises a bioactive molecule from a fungus, a first bioactive molecule from a plant, a second bioactive molecule from a plant, and a bioactive molecule from an algae.

In some embodiments, a therapeutic combination comprises a bioactive molecule from Psilocybe cubensis, a bioactive molecule from Cannabis sativa, a bioactive molecule from Pyropia yesoensis, and a bioactive molecule from Dypterix odorata. In some embodiments, a disclosed therapeutic combination is a botanical formulation, such as prepared in embodiments herein, comprising whole extracts of Psilocybe cubensis fungi, Cannabis sativa plant, Pyropia yesoensis algae, and Dypterix odorata bean. As will be further appreciated, a bioactive molecule may be provided in an extract, such as in a whole extract, in a fraction or subfraction thereof, or may be provided as an isolated compound, a substantially purified compound, or a purified compound, including one produced by biosynthetic or synthetic means, and combinations thereof.

In some embodiments, a bioactive molecule from a fungus is not also a bioactive molecule from a plant, nor is it a bioactive molecule from an algae (i.e., the bioactive molecule from a fungus is only found in fungi). In some embodiments, a bioactive molecule from a plant is not also a bioactive molecule from a fungus, nor is it a bioactive molecule from an algae (i.e., the bioactive molecule from a plant is only found in plants). In some embodiments, a bioactive molecule from an algae is not also a bioactive molecule from any fungus, nor is it a bioactive molecule from any plant (i.e., the bioactive molecule from an algae is only found in algae).

In some embodiments, a bioactive molecule from a fungus is also either a bioactive molecule from a plant, or a bioactive molecule from an algae (i.e., it is also found in plants or algae, but not both). In some embodiments, a bioactive molecule from a plant is also either a bioactive molecule from any fungus, or a bioactive molecule from an algae (i.e., it is also found in fungi or algae, but not both). In some embodiments, a bioactive molecule from an algae is also either a bioactive molecule from any fungus, or a bioactive molecule from a plant (i.e., it is also found in fungi or plants, but not both).

In some embodiments, a bioactive molecule from a fungus is also a bioactive molecule from a plant, or a bioactive molecule from an algae (i.e., it is also found in plants or algae). In some embodiments, a bioactive molecule from a plant is also a bioactive molecule from a fungus, or a bioactive molecule from an algae (i.e., it is also found in fungi or algae). In some embodiments, a bioactive molecule from an algae is also a bioactive molecule from a fungus, or a bioactive molecule from a plant (i.e., it is also found in fungi or plants).

In some embodiments, a bioactive molecule from a fungus is also a bioactive molecule from a plant, and a bioactive molecule from an algae (i.e., it is also found in plants and algae). In some embodiments, a bioactive molecule from a plant is also a bioactive molecule from a fungus, and a bioactive molecule from an algae (i.e., it is also found in fungi and algae). In some embodiments, a bioactive molecule from an algae is also a bioactive molecule from a fungus, and a bioactive molecule from a plant (i.e., it is also found in fungi and plants).

The term “secondary,” as in “secondary bioactive,” is used herein to mean, and may be used interchangeably with, the terms “second” or “additional,” and is not intended to necessarily signify any specific degree of priority when compared to the term “primary,” as in “primary bioactive,” and hence should not necessarily imply a lower degree of importance or significance, such as importance or significance in any specific composition, or for any specific disclosed use.

a. Fungi

Fungi are organisms that, unlike plants, do not contain chlorophyll and are saprophytic (they feed on dead plant and animal material), parasitic (they feed off a living host), or symbiotic (they share a mutually beneficial relationship with another organism). Fungi reproduce both sexually and asexually through spores developed in various ways. Exemplary fungi include the classes Phycomycetes, Ascomycetes, and Basidiomycetes. Fungi may include what are commonly referred to as yeasts, rusts, smuts, mildews, molds, and mushrooms. Herein, “fungi” should be appreciated to include both filamentous and non-filamentous fungi species, and the term will be understood to include all forms of the word, including “fungal” and “fungus.”

In some exemplary embodiments, the fungi are psilocybin-producing fungi. A “psilocybin-producing” fungus (or psilocybin-producing fungi) is any fungus that produces or is capable of producing psilocybin. Over 100 species in the Psilocybe genus of fungi produce psilocybin. Psilocybin-producing species also can be found in a number of other genera, including Athelia, Conocybe, Copelandia, Fibularhizoctonia, Galerina, Gymnopilus, Inocybe, Mycena, Panaeolus, Pholiotina, and Pluteus. In some embodiments, the psilocybin-producing species is from any of these genera. In embodiments, the psilocybin-producing species is from any of the genera Copelandia, Galerina, Gymnopilus, Inocybe, Panaeolus, Pholiotina, Pluteus, and Psilocybe. In embodiments, it is from Psilocybe. Different species of psilocybin mushrooms, and different strains thereof, will be readily known or readily identifiable to those in the art.

In some embodiments, the psilocybin-producing fungus is a Psilocybe spp. fungus. In some embodiments, the Psilocybe spp. fungus is any of a P. acutipilea, P. allenii, P. alutacea, P. angulospora, P. antioquiensis, P. araucariicola, P. atlantis, P. aquamarina, P. armandii (Mexicana), P. aucklandiae, P. aztecorum, P. azurescens, P. baeocystis, P. banderillensis, P. bispora, P. brasiliensis, P. brunneocystidiata, P. caeruleoannulata, P. caerulescens, P. caerulipes, P. callosa, P. carbonaria, P. caribaea, P. chuxiongensis, P. collybioides, P. columbiana, P. congolensis, P. cordispora, P. cubensis, P. cyanescens, P. cyanofibrillosa, P. dumontii, P. egonii, P. eximia, P. fagicola, P. farinacea, P. fimetaria, P. fuliginosa, P. furtadoana, P. galindoi, P. gallaeciae, P. graveolens, P. guatapensis, P. heimii, P. herrerae, P. hispanica, P. hoogshagenii, P. inconspicua, P. indica, P. isabelae, P. jacobsii, P. jaliscana, P. kumaenorum, P. laurae, P. lazoi, P. liniformans, P. mexicana, P. mairei, P. makarorae, P. mammillata, P. medullosa, P. meridensis, P. meridionalis, P. mescaleroensis, P. moseri, P. muliercula, P. naematoliformis, P. natalensis, P. natarajanii, P. neorhombispora, P. neoxalapensis, P. ovoideocystidiata, P. papuana, P. paulensis, P. pelliculosa, P. pintonii, P. pleurocystidiosa, P. plutonia, P. portoricensis, P. pseudoaztecorum, P. puberula, P. quebecensis, P. rickii, P. rostrate, P. rzedowskii, P. samuiensis, P. schultesii, P. semilanceata, P. septentrionalis, P. serbica, P. sierrae, P. sylvatica, P. singer, P. strictipes, P. stuntzii, P. subacutipilea, P. subaeruginascens, P. subaeruginosa, P. subcaerulipes, P. subcubensis, P. subpsilocybioides, P. subtropicalis, P. tampanensis, P. thaicordispora, P. thaiaerugineomaculans, P. thaiduplicatocystidiata, P. uruguayensis, P. uxpanapensis, P. venenata, P. villarrealiae, P. weilii, P. weldenii, P. weraroa, P. wrightii, P. yungensis, P. zapotecoantillarum, P. zapotecocaribaea, or P. zapotecorum species, including strains thereof.

In some embodiments, the psilocybin-producing fungus is not a Psilocybe spp. fungus. Other psilocybin-producing fungi, not of the Psilocybe genus, will be readily known to those in the art. Non-limiting examples include Conocybe siligineoides, Conocybe velutipes, Copelandia tropica, Inocybe aeruginascens, Inocybe caerulata, Inocybe coelestium, Inocybe corydalina, Inocybe haemacta, Inocybe tricolor, Galerina steglichii, Gymnopilus aeruginosas, Gymnopilus braendlei, Gymnopilus cyanopalmicola, Gymnopilus dilepis, Gymnopilus dunensis, Gymnopilus intermedius, Gymnopilus lateritius, Gymnopilus luteofolius, Gymnopilus luteoviridis, Gymnopilus luteus, Gymnopilus palmicola, Gymnopilus purpuratus, Gymnopilus subpurpuratus, Gymnopilus subspectabilis, Gymnopilus validipes, Gymnopilus viridans, Panaeolus venezolanus, Panaeolus tropicalis, Panaeolus tirunelveliensis, Panaeolus rubricaulis, Panaeolus olivaceus, Panaeolus moellerianus, Panaeolus microsporus, Panaeolus lentisporus, Panaeolus fimicola, Panaeolus cyanescens, Panaeolus cinctulus, Panaeolus chlorocystis, Panaeolus cambodginiensis, Panaeolus bisporus, Panaeolus axfordii, Panaeolus africanus, Panaeolus affinis, Pholiotina cyanopus, Pholiotina smithii, Pluteus albostipitatus, Pluteus americanus, Pluteus cyanopus, Pluteus glaucus, Pluteus glaucotinctus, Pluteus nigroviridis, Pluteus phaeocyanopus, Pluteus salicinus, Pluteus saupei, Pluteus velutinornatus, and Pluteus villosus.

In some aspects, disclosed combinations, compositions, and methods comprising a fungal portion, a fungal extract, or a bioactive molecule from a fungus affect CNS signaling capabilities. In some embodiments, disclosed combinations, compositions, and methods comprising a fungal portion, a fungal extract, or a bioactive molecule from a fungus (e.g., psilocybin and/or psilocin) increase extracellular dopamine and serotonin in animals such as mammals, including humans, or in rodents, such as in rats. In some embodiments, disclosed combinations, compositions, and methods comprising a fungus, a fungal portion, a fungal extract, or a bioactive molecule from a fungus (e.g., psilocybin and/or psilocin) increase synaptic density, as measured by, e.g., synaptic vesicle glycoprotein 2A (SV2A), such as in animal studies (Sakashita et al., 2015; and Raval et al., 2021). In some embodiments, disclosed combinations, compositions, and methods comprising a fungus, a fungal portion, a fungal extract, or a bioactive molecule from a fungus (e.g., psilocybin and/or psilocin) promote structural and functional plasticity, for example by way of 5-HT, mTOR, and other various pathways, which can be measured by known methods such as illustrated in Ly et. al., 2018.

In some aspects, disclosed combinations, compositions, and methods comprising a fungus, a fungal portion, a fungal extract, or a bioactive molecule from a fungus regulate proinflammatory cycles. In some embodiments, disclosed combinations, compositions, and methods comprising a fungus, a fungal portion, a fungal extract, or a bioactive molecule from a fungus (e.g., psilocybin and/or psilocin) reverse LPS-induced pro-inflammatory cytokines (e.g., TNF-α, IL-1β). In some embodiments, disclosed combinations, compositions, and methods inhibit concentrations of LPS-induced pro-inflammatory cytokines (e.g., TNF-α, IL-1β) in a dose-dependent manner. In some embodiments, disclosed combinations, compositions, and methods comprising a fungus, a fungal portion, a fungal extract, or a bioactive molecule from a fungus (e.g., psilocybin and/or psilocin) lower IL-6 and/or COX-2 concentrations, and in some embodiments without lowering the concentration of the anti-inflammatory IL-10, e.g., as described by Nkadimeng, Steinmann, and Eloff, 2021. In some embodiments, disclosed combinations, compositions, and methods comprising a fungus, a fungal portion, a fungal extract, or a bioactive molecule from a fungus (e.g., psilocybin and/or psilocin) show a reduction of TNF-α, IL-1β, and ROS, e.g., as described by Nkadimeng, Steinmann, and Eloff, 2020.

ii. Primary and Secondary Bioactive Molecules from Fungi

In some embodiments, a therapeutic combination comprises a fungal portion. In some embodiments, the fungal portion comprises a fungal extract. In some embodiments, the fungal portion comprises a bioactive molecule from a fungus. In some embodiments, the fungal extract comprises a bioactive molecule from a fungus. For example, in embodiments, the fungal portion comprises a primary bioactive molecule and/or a secondary bioactive molecule from a fungus. In embodiments, the fungal extract comprises a primary bioactive molecule and/or a secondary bioactive molecule from a fungus. Hence, in embodiments, a disclosed therapeutic combination comprises a primary bioactive molecule and/or a secondary bioactive molecule from a fungi.

In some embodiments (equivalently herein, and simply as shorthand, “in embodiments”), the fungal portion is from a psilocybin-producing fungus. The fungus can be any psilocybin-producing fungus known or ascertainable by those of skill, including, as non-limiting examples, certain species from the genera Athelia, Conocybe, Copelandia, Fibularhizoctonia, Galerina, Gymnopilus, Inocybe, Mycena, Panaeolus, Pholiotina, Pluteus, and Psilocybe. Different species of psilocybin-producing fungi, and different strains thereof, are described herein and readily known or readily identifiable to those in the art.

In embodiments wherein the fungal portion is described as being “from” a fungus, it will be understood that the fungal portion comprises matter derived from the fungus, including, as non-limiting examples, fungal material such as raw (i.e., unprocessed) fungal biomass, a fungal extract (e.g., extracts described herein, such as aqueous and/or ethanolic extracts), or a molecule that is naturally occurring in the fungus (e.g., a primary or secondary bioactive molecule described in embodiments herein), whether or not said molecule is actually obtained by isolating the molecule from the fungus, or another means (e.g., by chemical synthesis).

In some embodiments, the fungal portion of a therapeutic combination is from a psilocybin-producing species. In some embodiments, the fungal portion of a therapeutic combination is from a species of the genera Athelia, Conocybe, Copelandia, Fibularhizoctonia, Galerina, Gymnopilus, Inocybe, Mycena, Panaeolus, Pholiotina, Pluteus, and Psilocybe. In some embodiments, the fungal portion comprises a fungal extract from a psilocybin-producing species. In some embodiments, the fungal portion comprises a fungal extract from a species of the genera Athelia, Conocybe, Copelandia, Fibularhizoctonia, Galerina, Gymnopilus, Inocybe, Mycena, Panaeolus, Pholiotina, Pluteus, and Psilocybe. In some embodiments, the fungal portion comprises a bioactive molecule from a psilocybin-producing species. In some embodiments, the fungal portion comprises a bioactive molecule from a species of the genera Athelia, Conocybe, Copelandia, Fibularhizoctonia, Galerina, Gymnopilus, Inocybe, Mycena, Panaeolus, Pholiotina, Pluteus, and Psilocybe. In some embodiments, the fungal portion is from a species of the genera Copelandia, Galerina, Gymnopilus, Inocybe, Panaeolus, Pholiotina, Pluteus, and Psilocybe. In some embodiments, the fungal portion comprises a fungal extract from a species of the genera Copelandia, Galerina, Gymnopilus, Inocybe, Panaeolus, Pholiotina, Pluteus, and Psilocybe. In embodiments, the fungal portion comprises a bioactive molecule from a species of the genera Copelandia, Galerina, Gymnopilus, Inocybe, Panaeolus, Pholiotina, Pluteus, and Psilocybe.

In some embodiments, the fungi is a Psilocybe spp. fungi. In some embodiments, the fungal portion is from a Psilocybe spp. fungi. In some embodiments, the fungal portion is from the species P. azurescens, P. bohemica, P. semilanceata, P. baeocystis, P. cyanescens, P. tampanensis, P. cubensis, P. weilii, P. hoogshagenii, P. stuntzii, P. cyanofibrillosa, and/or P. liniformans. In some embodiments, the fungal portion comprises a fungal extract from a Psilocybe spp. fungi. In some embodiments, the fungal portion comprises a fungal extract from the species P. azurescens, P. bohemica, P. semilanceata, P. baeocystis, P. cyanescens, P. tampanensis, P. cubensis, P. weilii, P. hoogshagenii, P. stuntzii, P. cyanofibrillosa, and/or P. liniformans. In some embodiments, the fungal portion comprises a fungal extract from a Psilocybe spp. fungi. In embodiments, the fungal portion comprises a bioactive molecule from the species P. azurescens, P. bohemica, P. semilanceata, P. baeocystis, P. cyanescens, P. tampanensis, P. cubensis, P. weilii, P. hoogshagenii, P. stuntzii, P. cyanofibrillosa, and/or P. liniformans.

In embodiments, the fungal portion comprises any of psilocybin, psilocin, baeocystin, norbaeocystin, norpsilocin, and aeruginascin. In embodiments, the fungal portion comprises a fungal extract comprising any of psilocybin, psilocin, baeocystin, norbaeocystin, norpsilocin, and aeruginascin. In embodiments, the fungal portion comprises a primary bioactive molecule from a fungus that is selected from the group consisting of psilocybin, psilocin, baeocystin, norbaeocystin, norpsilocin, and aeruginascin. In embodiments, the fungal portion comprises psilocybin. In embodiments, the fungal portion comprises psilocin. In embodiments, the fungal portion comprises baeocystin. In embodiments, the fungal portion comprises norbaeocystin. In embodiments, the fungal portion comprises norpsilocin. In embodiments, the fungal portion comprises aeruginascin. In embodiments, the fungal portion comprises a fungal extract comprising psilocybin. In embodiments, the fungal portion comprises a fungal extract comprising psilocin. In embodiments, the fungal portion comprises a fungal extract comprising baeocystin. In embodiments, the fungal portion comprises a fungal extract comprising norbaeocystin. In embodiments, the fungal portion comprises a fungal extract comprising norpsilocin. In embodiments, the fungal portion comprises a fungal extract comprising aeruginascin. In embodiments, the fungal portion comprises a primary bioactive molecule from a fungus, wherein the molecule is psilocybin. In embodiments, the fungal portion comprises a primary bioactive molecule from a fungus, wherein the molecule is psilocin. In embodiments, the fungal portion comprises a primary bioactive molecule from a fungus, wherein the molecule is baeocystin. In embodiments, the fungal portion comprises a primary bioactive molecule from a fungus, wherein the molecule is norbaeocystin. In embodiments, the fungal portion comprises a primary bioactive molecule from a fungus, wherein the molecule is norpsilocin. In embodiments, the fungal portion comprises a primary bioactive molecule from a fungus, wherein the molecule is aeruginascin.

In some embodiments, the fungal portion comprises both psilocybin and psilocin. In some embodiments, the fungal portion comprises a fungal extract comprising both psilocybin and psilocin. In some embodiments, the fungal portion comprises, as primary bioactive molecules from a fungus, both of psilocybin and psilocin. In some embodiments, the fungal portion comprises both psilocybin and psilocin in a w/w or molar ratio of between 100:1 and 1:100, between 50:1 and 1:50, between 10:1 and 1:10, between 5:1 and 1:5, between 2:1 and 1:2, between 5:3 and 3:5, or between 3:2 and 2:3, all ranges inclusive. In some embodiments, the fungal portion comprises both psilocybin and psilocin in a w/w or molar ratio of about 100:1, 50:1, 10:1, 5:1, 2:1, 5:3, 3:2, or 1:1. In some embodiments, the fungal portion comprises both psilocybin and psilocin in a w/w or molar ratio of about 1:100, 1:50, 1:10, 1:5, 1:2, 3:5, 2:3, or 1:1. In some embodiments, the fungal portion comprises both psilocybin and psilocin in a w/w or molar ratio of about 5:3, about 3:2, about 1:1, about 2:3, or about 3:5. In some embodiments, the fungal portion comprises a fungal extract comprising both psilocybin and psilocin, including in any of the above w/w or molar ratios. In some embodiments, the fungal portion comprises, as primary bioactive molecules from a fungus, both of psilocybin and psilocin, including in any of the above w/w or molar ratios.

In some embodiments, psilocybin and psilocin are in a ratio of about 5:3, about 3:2 or about 1:1. In embodiments, psilocybin and psilocin are in a ratio of about 5:3 or a ratio of 5:3.

In some embodiments, the fungal portion comprises a B-carboline (beta-carboline). In some embodiments, the fungal portion comprises a fungal extract comprising a β-carboline. In some embodiments, the fungal portion comprises a primary bioactive molecule from a fungus, wherein the primary bioactive molecule is a B-carboline. In embodiments, the B-carboline is harmane, harmine, harmol, harmalol, harmaline, tetrahydroharmine, pinoline, cordysinin C, cordysinin D, norharmane, perlolyrine, B-carboline (9H-pyrido[3,4-b]indole), or another L-tryptophan-derived B-carboline. Although “B-carboline” therefore may refer both to the individual compound and to the class of related compounds, use of the term herein shall be understood as referring to the class of compounds (including, but not limited to, when recited as “a β-carboline”) unless context demands otherwise.

In embodiments, the secondary bioactive molecule from a fungus is a polysaccharide (including a and B-glucans and polysaccharide-protein complexes), a peptide (including proteins such as lectins), a terpene or terpenoid (including mono and sesquiterpene oils, diterpenes, triterpenoids and sterols, and carotenoid pigments), a phenolic compound (including phenolic acids, hydroxycinnamic acids, hydroxybenzoic acids, ligans, tannins, flavonoids, stilbenes, and oxidized polyphenols), a mineral (including potassium, phosphorous, sodium, calcium, magnesium, copper, selenium, iron, and zinc), a vitamin (including ascorbic acid, vitamin D, riboflavin, folate, thiamine, pantothenic acid, and niacin), an amino acid (including the essential amino acids, and including histidine, isoleucine, leucine, lysine, methionine, phenylalanine, taurine, threonine, tryptophan, and valine), a lipid (including saturated, monounsaturated, and polyunsaturated fatty acids such as oleic, linoleic, and linolenic acids), a choline, or a lactone.

In embodiments, a bioactive molecule from a fungus is any of those in Venturella, 2021; Anusiya et al., 2021; Thu et al., 2020; Muszynska, 2018; and Mishraki-Berkowitz et al., 2020.

b. Plants

In some embodiments, a therapeutic combination comprises a plant portion. In some embodiments, the plant portion comprises a plant extract. In some embodiments, the plant portion comprises a bioactive molecule from a plant. In some embodiments, the fungal extract comprises a bioactive molecule from a plant. For example, in embodiments, the plant portion comprises a primary bioactive molecule and/or a secondary bioactive molecule from a plant. In embodiments, the plant extract comprises a primary bioactive molecule and/or a secondary bioactive molecule from a plant. Hence, in some embodiments, a disclosed therapeutic combination comprises a primary bioactive molecule and/or a secondary bioactive molecule from a plant.

In embodiments wherein the plant portion is described as being “from” a plant, it will be understood that the plant portion comprises matter derived from the plant, including, as non-limiting examples, plant material such as raw (i.e., unprocessed) plant biomass, a plant extract (e.g., extracts described herein, such as aqueous and/or ethanolic extracts), or a molecule that is naturally occurring in the plant (e.g., a primary or secondary bioactive molecule described in embodiments herein), whether or not said molecule is actually obtained by isolating the molecule from the plant, or another means (e.g., by chemical synthesis).

Plants are photosynthetic eukaryotes of the kingdom Plantae. In some embodiments, a therapeutic combination comprises a plant extract. In some embodiments, a therapeutic combination comprises a primary bioactive molecule and/or a secondary bioactive molecule from a plant. In some embodiments, a therapeutic combination comprises a Cannabis extract and/or Dipteryx extract. In some embodiments, a therapeutic combination comprises a Cannabis extract. In some embodiments, a therapeutic combination comprises a Dipteryx extract. In some embodiments, a therapeutic combination comprises both a Cannabis extract and a Dipteryx extract. In some embodiments, the therapeutic combination comprises a primary bioactive molecule and/or a secondary bioactive molecule from a plant in the genera Cannabis and/or Dipteryx. In some embodiments, the therapeutic combination comprises a primary bioactive molecule and/or a secondary bioactive molecule from a plant in the genera Cannabis.

ii. Cannabis

Cannabis is a genus of flowering plant in the family Cannabaceae. The number of species in the genus is disputed, and some recognize three separate species, Cannabis sativa, Cannabis indica, and Cannabis ruderalis; some include C. ruderalis within C. sativa; some include C. sativa, C. indica, and C. ruderalis as subspecies of a single species, C. sativa; and some treat C. sativa L as a single undivided species. Herein, “cannabis” refers to all such encompassed species, subspecies, cultivars, varieties, variants, strains, chemovars, and the like, comprising the genus Cannabis, independent of any such terminology. Simply for shorthand herein, and without taking a position on the above dispute, the term “Cannabis” will refer to C. sativa, C. indica, and C. ruderalis, and will further include any genetic crosses, self-crosses, and hybrids thereof.

In some embodiments, the plant portion of a therapeutic combination is from Cannabis. In embodiments wherein the plant portion is described as being “from” Cannabis, it will be understood that the plant portion comprises matter derived from Cannabis, including, as non-limiting examples, Cannabis material such as raw (i.e., unprocessed) Cannabis biomass, a Cannabis extract (e.g., extracts described herein, such as aqueous and/or ethanolic extracts), or a molecule that is naturally occurring in Cannabis (e.g., a primary or secondary bioactive molecule described in embodiments herein), whether or not said molecule is actually obtained by isolating the molecule from Cannabis, or another means (e.g., by chemical synthesis).

In some embodiments, a therapeutic combination comprises a Cannabis extract. In some embodiments, a therapeutic combination comprises a primary bioactive molecule from Cannabis.

In some embodiments, a combination comprises a secondary bioactive molecule from Cannabis.

a. Primary and Secondary Bioactive Molecules from Cannabis

In some embodiments, a therapeutic combination comprises a primary and/or secondary bioactive molecule from Cannabis.

II. Primary Bioactive Molecules from Cannabis

In some embodiments, the primary bioactive molecule from Cannabis is a cannabinoid. Cannabinoids are a diverse class of small molecules grouped together because of their ability to act on cannabinoid receptors. Cannabinoid receptors are found in the brain and throughout the central and peripheral nervous systems of humans and other mammals. There are two major types of cannabinoid receptors, known as the cannabinoid receptor 1 (CB₁) and cannabinoid receptor 2 (CB₂). CB₁ receptors are primarily found in the central nervous system (i.e., the brain and spinal cord), as well as in the lungs, liver, and kidneys. CB₁ receptor-mediated signaling plays a critical role in neural circuitry that mediates mood, motivation, and emotional behaviors. CB₂ receptors are primarily found in the immune system and hematopoietic stem and progenitor cells, and also may be found in neurons.

Cannabinoids have been shown to increase the activity and expression of tyrosine hydroxylase (Bonnin et al, 1996; Hernandez, et al 1997). Consistent with this, Bloom (1982) and Maitre et al (1970) have shown that THC increases the synthesis of dopamine. THC has also been shown to inhibit dopamine uptake (Banerjee et al 1975). Szabo et al (2002) also suggest that CB₁ receptor activation inhibits gabaergic neurotransmission in the VTA by a presynaptic mechanism. Depression of the gabaergic inhibitory effect on dopaminergic neurons would increase their firing rate in vivo, with a resultant increase in dopamine in the NAc. Moreover, cannabinoids regulate the release and uptake of various neurotransmitters, including acetylcholine, glutamate, noradrenaline, and GABA by way of N and P/Q type calcium currents and potassium A-currents (Gifford et al., 2000).

Cannabinoids have also been shown to regulate pro-inflammatory cycles. In LPS-stimulated neuroinflammatory tests, THC and CBD decreased IL-1β and IL-6, while CBD suppressed the secretion of IL-1β and inhibited the NF-κB signaling pathway (Kozela et al., 2010; Dos-Santos Pereira et al., 2020). CBD was found to increase NRF2 expression, leading to decreased ROS, which in turn may have partially suppressed NLRP3 inflammasome activation by reducing NF-κB levels (Jastrząb, Gęgotek, and Skrzydlewska, 2019). In one study, CBD increased NRF2 expression, while actively suppressing NF-κB by way of promoters p 65 and p 52 (Jastrząb, Gęgotek, and Skrzydlewska, 2019). CBD also activates PPAR-y networks and directly represses NF-κB transduction through CB₁ and CB₂ receptors (Nichols and Kaplan, 2020).

Cannabinoids will be known to those in the art and some are also set forth and described by Radwan et al. in Cannabinoids, Phenolics, Terpenes and Alkaloids of Cannabis, Molecules, 26 (9), 2774 (2021), which is incorporated by reference as if fully set forth herein. Without being bound by theory, cannabinoids according to the categorization of Radwan 2021 include compounds with a characteristic C21 terpenophenolic backbone that are part of one of 11 cannabinoid sub-classes, namely: cannabichromene (CBC)-type, cannabidiol (CBD) type, cannabielsoin (CBE) type, cannabigerol (CBG) type, cannabicyclol (CBL) type, cannabinol (CBN) type, cannabinodiol (CBND) type, cannabitriol (CBT) type, (−)-48-trans-tetrahydrocannabinol (Δ⁸-THC) type, (−)-Δ⁹-trans-tetrahydrocannabinol (Δ⁹-THC) type, and miscellaneous-type cannabinoids. Non-limiting examples of such cannabinoids, all of which will be understood to be useful in the practice of the invention, are known by reference to the disclosure of Radwan 2021 and the below. In embodiments, the primary bioactive molecule from Cannabis is any such cannabinoid.

In embodiments, the primary bioactive molecule from Cannabis is a Δ⁹-THC-type cannabinoid, Δ⁸-THC-type cannabinoid, CBG-type cannabinoid, CBD-type cannabinoid, CBND-type cannabinoid, CBE-type cannabinoid, CBL-type cannabinoid, CBN-type cannabinoid, CBC-type cannabinoid, CBT-type cannabinoid, or miscellaneous-type cannabinoid. Examples of Δ⁹-THC-type cannabinoids include Δ⁹-THC-C5, 49-THCAA-C5, Δ⁹-THCAB-C5, 49-THC-CH, Δ⁹-THCAA-CH, Δ⁹-THCV, Δ⁹-THCVAA, Δ⁹-THCO, Δ⁹-THCOAA, Δ⁹-THC-aldehyde, β-fenchyl(−)-Δ⁹-trans-tetrahydrocannabinolate, α-fenchyl(−)-Δ⁹-trans-tetrahydrocannabinolate, epi-bornyl(−)-Δ⁹-trans-tetrahydrocannabinolate, bornyl(−)-Δ⁹-trans-tetrahydrocannabinolate, α-terpenyl(−)-Δ⁹-trans-tetrahydrocannabinolate, γ-terpenyl(−)-Δ⁹-trans-tetrahydrocannabinolate, α-cadinyl(−)-Δ⁹-trans-tetrahydrocannabinolate, γ-eudesmyl(−)-Δ⁹-trans-tetrahydrocannabinolate, 8α-hydroxy-(−)-Δ⁹-trans-tetrahydro-cannabinol, 8β-hydroxy-(−)-Δ⁹-trans-tetrahydrocannabinol, 11-acetoxy-(−)-Δ⁹-trans-tetrahydro-cannabinolic acid A, 8-oxo-(−)-Δ⁹-trans-tetrahydrocannabinol, cannabisol, (−)-Δ⁹-trans-tetrahydrocannabiphorol, and (−)-Δ⁹-trans-tetrahydrocannabihexol.

Examples of Δ⁸-THC-type cannabinoids include Δ⁸-THC, A8-THCA, 10α-OH-Δ⁸-THC, 10β-OH-Δ⁸-THC, and 10a-α-hydroxy-10-oxo-Δ⁸-THC.

In embodiments, CBG-type cannabinoids include (E)CBG, (E)CBGA, (E)CBGG, (F)CBGAM, (E)CBGV, (E)CBGVA, (Z)CBGA, 5-acetyl-4-hydroxy-cannabigerol, (±)-6,7-trans-epoxycannabigerolic acid, (±)-6,7-cis-epoxycannabigerolic acid, (±)-6,7-cis-epoxycannabigerol, (±)-6,7-trans-epxoycannabigerol, camagerol, and sesquicannabigerol.

Examples of CBD-type cannabinoids include CBD-C₅, CBDA-C₅, CBDM-C₅, CBD-C₄, CBDV, CBDVA, CBD-C₁, CBDH, CBDP, and CBDD. Examples of CBND-type cannabinoids include CBND-C₃ and CBND-C₅. Examples of CBE-type cannabinoids include CBE-C₅, CBEAA-C₅, CBEAB-C₅, CBE-C₃, and CBEAB-C₃. Examples of CBL-type cannabinoids include CBL, CBLA, and CBLV. Examples of CBC-type cannabinoids include CBC, CBCA, ±CBCV, ±CBCV, CBCVA, 4-acetoxy-CBC, (±)-3″-hydroxy-44″-cannabichromene, (−)-7-hydroxy-canna-bichromene, and CBC-C₃. Examples of CBN-type cannabinoids include CBN-C₅, CBNA-C₅, CBN-C₄, CBN-C₃, CBN-C₂, CBN-C₁, CBNM-C₅, 8-OH-CBN, 8-OH-CBNA, 1'S—OH-CBN, and γ-terpenyl-cannabinolate. Examples of CBT-type cannabinoids include (−)-trans-CBT-C₅, (±)-trans-CBT-C₅, (±)-cis-CBT-C₅, (±)-trans-CBT-C₃, CBT-C₃-homologue, (−)-trans-CBT-OEt-C₅, (−)-trans-CBT-OEt-C₃, 8,9-Di-OH-CBT-C₅, CBDA-C₅, and 9-OH-CBT-C₃ ester.

Examples of miscellaneous-type cannabinoids include DCBF-C₅, CBF-C₅, OH-iso-HHCV-C; OTHC, cannabicitran, cis-Δ⁹-THC, CBCON-C₅, CBR, CBTT, CBCN-C₅, CBCN-C₃, cis-iso-47-THCV, trans-iso-47-THCV, trans-iso-47-THC, CBCNB, CBCNC, CBCND, (−)-(7R)-cannabicoumarononic acid, 4-acetoxy-2-geranyl-5-hydroxy-3-n-pentylphenol, 2-gera-nyl-5-hydroxy-3-n-pentyl-1,4-benzoquinone, 5-acetoxy-6-geranyl-3-n-pentyl-1,4-benzoquinone, CBM, CBX, 10α-hydroxy-49.11-hexahydrocannabinol, 9β, 10β-epoxyhexahydrocannabinol, 9α-hydroxyhexahydrocannabinol, 7-oxo-9α-hydroxyhexa-hydrocannabinol, 10α-hydroxyhexa-hydrocannabinol, 10αR-hydroxyhexahydrocannabinol, and 9α-hydroxy-10-oxo-Δ^{6a,10a}-THC.

In embodiments, the primary bioactive molecule from Cannabis is Δ⁹-THC-C₅, Δ⁹-THCAA-C₅, Δ⁹-THCAB-C₅, Δ⁹-THC-C₄, Δ⁹-THCAA-C₄, Δ⁹-THCV, Δ⁹-THCVAA, Δ⁹-THCO, Δ⁹-THCOAA, Δ⁹-THC-aldehyde, β-fenchyl(−)-Δ⁹-trans-tetrahydrocannabinolate, α-fenchyl(−)-Δ9-trans-tetrahydrocannabinolate, epi-bornyl(−)-Δ9-trans-tetrahydrocannabinolate, bornyl(−)-Δ9-trans-tetrahydrocannabinolate, α-terpenyl(−)-Δ9-trans-tetrahydrocannabinolate, γ-terpenyl(−)-Δ9-trans-tetrahydrocannabinolate, α-cadinyl(−)-Δ9-trans-tetrahydrocannabinolate, γ-eudesmyl(−)-Δ9-trans-tetrahydrocannabinolate, 8α-hydroxy-(−)-Δ9-trans-tetrahydro-cannabinol, 8β-hydroxy-(−)-Δ9-trans-tetrahydrocannabinol, 11-acetoxy-(−)-Δ9-trans-tetrahydrocannabinolic acid A, 8-oxo-(−)-Δ9-trans-tetrahydrocannabinol, cannabisol, (−)-Δ9-trans-tetrahydro-cannabiphorol, (−)-Δ9-trans-tetrahydrocannabihexol, Δ⁸-THC, Δ⁸-THCA, 10α-OH-Δ⁸-THC, 10β-OH-Δ⁸-THC, 10a-α-hydroxy-10-oxo-Δ⁸-THC, (E)CBG, (E)CBGA, (E)CBGG, (F)CBGAM, (E)CBGV, (E)CBGVA, (Z)CBGA, 5-acetyl-4-hydroxy-cannabigerol, (±)-6,7-trans-epoxycannabigerolic acid, (±)-6,7-cis-epoxycannabigerolic acid, (±)-6,7-cis-epoxycannabigerol, (±)-6,7-trans-epxoycannabigerol, camagerol, sesquicannabigerol, CBD-C₅, CBDA-C₅, CBDM-C₅, CBD-C₄, CBDV, CBDVA, CBD-C₁, CBDH, CBDP, CBDD, CBND-C₃, CBND-C₅, CBE-C₅, CBEAA-C₅, CBEAB-C₅, CBE-C₃, CBEAB-C₃, CBL, CBLA, CBLV, CBC, CBCA, ±CBCV, ±CBCV, CBCVA, 4-acetoxy-CBC, (±)-3″-hydroxy-44″-cannabichromene, (−)-7-hydroxy-cannabichromane, CBC-C₃, CBN-C₅, CBNA-C₅, CBN-C₄, CBN-C₃, CBN-C₂, CBN-C₁, CBNM-C₅, 8-OH-CBN, 8-OH-CBNA, 1'S—OH-CBN, γ-terpenyl-cannabinolate, (−)-trans-CBT-C₅, (±)-trans-CBT-C₅, (±)-cis-CBT-C₅, (±)-trans-CBT-C₃, CBT-C₃-homologue, (−)-trans-CBT-OEt-C₅, (−)-trans-CBT-OEt-C₃, 8,9-Di-OH-CBT-C₅, and CBDA-C₅, and 9-OH-CBT-C₅ ester, DCBF-C₅, CBF-C₅, OH-iso-HHCV-C₃ OTHC, cannabicitran, cis-Δ⁹-THC, CBCON-C₅, CBR, CBTT, CBCN-C₅, CBCN-C₃, cis-iso-47-THCV, trans-iso-A7-THCV, trans-iso-47-THC, CBCNB, CBCNC, CBCND, (−)-(7R)-cannabicoumarononic acid, 4-acetoxy-2-geranyl-5-hydroxy-3-n-pentylphenol, 2-geranyl-5-hydroxy-3-n-pentyl-1,4-benzo-quinone, 5-acetoxy-6-geranyl-3-n-pentyl-1,4-benzoquinone, CBM, CBX, 10α-hydroxy-49.11-hexahydrocannabinol, 9β,10β-epoxyhexahydrocannabinol, 9-hydroxyhexahydrocannabinol, 7-oxo-9α-hydroxyhexahydrocannabinol, 10α-hydroxyhexahydrocannabinol, 10αR-hydroxyhexa-hydrocannabinol, or 9α-hydroxy-10-oxo-Δ^{6a,10a}-THC. In embodiments, a “cannabinoid” includes any of the cannabinoid carboxylic acids and their carboxylate salts (see U.S. Pat. No. 9,376,367).

Each cannabinoid also will be understood to include its isomers, such as structural isomers and stereoisomers (including enantiomers), the -A and -B isomers for each cannabinoid, double bond isomers, and other such isomers known to those of skill. Thus, “THC” will, in embodiments, be understood to include THC-A and THC-B. Reference to a cannabinoid will include the various alkyl chain lengths associated therewith, as illustrated by Cn, wherein “n” refers to the number of carbon atoms in each alkyl chain. Thus, “THC” also includes THC-C1, THC-C2, THC-C3, THC-C4, THC-C5, THC-C6, and THC-C7. Reference to a given cannabinoid also will include all possible isomers, such as but not limited to its -A and -B isomers, together with all possible combinations of alkyl chain lengths, including chains comprised of 1, 2, 3, 4, 5, 6, or 7 carbon atoms. So, herein, reference to “THC” will include THC-C1 A, THC-C1 B, THC-C2 A, THC-C2 B, THC-C3 A, THC-C3 B, THC-C4 A, THC-C4 B, THC-C5 A, THC-C5 B, THC-C6 A, THC-C6 B, THC-C7 A, and THC-C7 B. As will be apparent to one of skill, such logic applies to all cannabinoids disclosed herein; THC is merely used to illustrate such logic, and should not be construed as limiting. In embodiments, a cannabinoid may further comprise an additional chemical moiety substituted thereon, including methyl, alkyl, alkenyl, methoxy, alkoxy, acetyl, carboxyl, carbonyl, oxo, ester, hydroxyl, aryl, heteroaryl, cycloalkyl, cycloalkenyl, cycloalkylalkenyl, cycloalkenylalkyl, cycloalkenylalkenyl, heterocyclylalkenyl, heteroarylalkenyl, arylalkenyl, heterocyclyl, aralkyl, cycloalkylalkyl, heterocyclylalkyl, heteroarylalkyl, and the like. In embodiments, a cannabinoid includes a “synthetic cannabinoid.”

In some embodiments, the plant portion comprises both THC and CBD. In some embodiments, the plant portion comprises a plant extract comprising both THC and CBD. In some embodiments, the plant portion comprises, as primary bioactive molecules from a plant (e.g., Cannabis), both THC and CBD. In some embodiments, the plant portion comprises both THC and CBD in a w/w or molar ratio of between 100:1 and 1:100, between 50:1 and 1:50, between 10:1 and 1:10, between 5:1 and 1:5, between 2:1 and 1:2, between 5:3 and 3:5, or between 3:2 and 2:3, all ranges inclusive. In some embodiments, the plant portion comprises both THC and CBD in a w/w or molar ratio of about 100:1, 50:1, 10:1, 5:1, 2:1, 5:3, 3:2, or 1:1. In some embodiments, the plant portion comprises THC and CBD in a w/w or molar ratio of about 1:100, 1:50, 1:10, 1:5, 1:2, 3:5, 2:3, or 1:1. In embodiments, the plant portion comprises both THC and CBD in a w/w or molar ratio of about 1:1. In embodiments, the plant portion comprises a plant extract (e.g., a Cannabis extract) comprising both THC and CBD, including in any of the above w/w or molar ratios. In embodiments, the plant portion comprises, as primary bioactive molecules from a plant, both THC and CBD, including in any of the above w/w or molar ratios.

II. Secondary Bioactive Molecules from Cannabis

In some embodiments, a therapeutic combination comprises a secondary bioactive molecule from Cannabis.

In some embodiments, the secondary bioactive molecule from Cannabis is a flavone and/or flavonoid, a terpene and/or terpenoid, a carbohydrate, a fatty acid or fatty acid ester, an amide, an amine, a phytosterol, or a phenolic compound.

Flavonoids are a broad class of water-soluble polyphenolic molecules (i.e., comprising a phenyl group (—C₆H₅) bonded to a hydroxy group (—OH)), of which about 20 are widely found in cannabis. A primary function of flavonoids is to provide color pigmentation to plants, particularly flowers. In Cannabis, deep purple strains owe their coloration to the flavonoids, anthocyanins and anthoxanthins. Besides providing color, flavonoids also have been shown to provide health benefits through modulation of cell signaling pathways and through various anti-inflammatory, antioxidant, anti-fungal, anti-cancer, and other effects. For example, the Cannabis flavonoid apigenin has potent anti-anxiety, anti-inflammatory, and anti-cancer properties; butin has been shown to reduce oxidative stress-related cell dysfunction. Other bioactive flavonoids found in cannabis include cannaflavins, kaempferol, orientin, luteolin, quercetin, silymarin, and vitexin.

In embodiments, flavonoids include those broadly described in Radwan 2021, including orientin, vitexin, isovitexin, apigenin, luteolin, kaempferol, and quercetin flavonoids; which, in embodiments, may be methylated, glycosylated, prenylated, or geranylated.

In embodiments, the flavonoid is orientin, orientin-O-glucoside, orientin-7-O-glucoside, orientin-7-O-rhamnoglucoside, vitexin, vitexin-O-glucoside, vitexin-7-O-glucoside, vitexin-7-O-rhamnoglucoside, cytisoside, cytisoside-glucoside, isovitexin, isovitexin-O-glucoside, isovitexin-7-O-glucoarbinoside, isovitexin-7-O-rhamnoglucoside, apigenin-7-O-glucoside, apigenin-7-O-glucuronoid, apigenin-7-O′P-coumaroylglucoside, 6-prenylapigenin, apigenin-6,8-di-gluco-pyranoside, luteolin-C-glucuronide, luteolin-7-O-glucuronide, canniflavin A, canniflavin B, canniflavin C, chrysoeriol, kaempferol-3-O-diglucoside, quercetin-3-O-glucoside, quercetin-3-O-diglucoside, kaempferol-3-O-sophoroside, quercetin-3-O-sophoroside, rutin, quercetin, naringenin, or naringin.

Terpenes are a large class of organic hydrocarbon compounds containing one or more repeating units of a five-carbon building block known as an isoprene unit (i.e., 2-methyl-1,3-butadiene, having the molecular formula C₅H₈). Being isoprene polymers, terpenes as a group are also often referred to as “isoprenoids.” Isoprene units may be linked together end-to-end to form linear chains, or may be arranged so as to form rings (thus having the molecular formula (CH₈)ₙ, where n is the number of linked isoprene units). Terpenes are classified in families according to the number of isoprene units from which they are constituted: as hemiterpenes (one unit), monoterpenes (two), sesquiterpenes (three), diterpenes (four), sesterterpenes (five), triterpenes (six), sesquarterpenes (seven), tetraterpenes (eight), and polyterpenes (nine or more).

Terpenes may be referred to as “terpenoids” when they have experienced oxidation and have an additional oxygen-containing functional group (for instance, after cannabis is cured and dried), or when they are otherwise modified by addition or removal of a functional group (e.g., a methyl group (—CH₃)). The presence and particular combination of terpenes gives different plants (and different cannabis strains) their distinctive smells and tastes. In Cannabis, terpenes are the largest group of phytochemicals, with at least 120 identified molecules. Terpenes generally make up between 10-20 percent of the total oil content produced by Cannabis resin glands. Terpenes also constitute the majority of chemicals in the heated or vaporized smoke of Cannabis flowers, often consisting of greater than 50%, with cannabinoids normally accounting for 10-20%.

Although cannabinoids are more popularly understood to be responsible for the mental and physical effects of Cannabis, terpenes have demonstrated a wide array of such effects as well (see, e.g., Russo, 2011). For example, the terpene β-myrcene has been shown to have a sedative effect, and is believed to be responsible for a heavy “body high.” β-myrcene has also demonstrated the ability to reduce inflammation and block hepatic carcinogenesis, and act as an analgesic and muscle relaxant. The terpenes linalool, nerolidol, and pulegone have also shown sedative effects. Others, such as limonene and terpinolene have, by contrast, shown stimulating effects. Additional terpenes show yet different effects. For example, α-humulene acts as an appetite suppressant. Limonene exhibits anti-cancer, anxiolytic, and immunostimulating properties, and nerolidol also has anti-cancer properties. β-caryophyllene possesses anti-inflammatory and gastric cytoprotector effects. Pentacyclic triterpenes, such as β-amyrin and cycloartenol, show anti-bacterial, anti-fungal, anti-inflammatory, and anti-cancer properties.

Terpenes may cause effects when consumed because of the modulation of neurotransmitter systems in the brain, as terpenes easily cross the blood-brain barrier (BBB). Linalool, for example, is shown to modulate the glutamatergic and gabaminergic neurotransmitter systems, which may explain its analgesic, anti-anxiety, anti-inflammatory, and anticonvulsant properties. α-Pinene is an acetylcholinesteral inhibitor, and may thereby aid memory. And phytol, a diterpene, increases gabaminergic expression. Other terpenes have been shown to affect serotonergic and dopaminergic neurotransmitter systems.

Some terpenes interact directly with the ECS. For example, β-caryophyllene selectively binds to CB₂ receptors as a functional CB₂ agonist, supporting β-caryophyllene as having an anxiolytic and antidepressant effect. Terpenes also have been shown to alter the permeability of cell membranes and thereby modulate the effects of THC and other cannabinoids. Since terpenes are lipophilic, they interact with lipid membranes, ion channels, a variety of different receptors (including both G protein-coupled odorant and neurotransmitter receptors), and enzymes. Through these and other mechanisms, the terpenes in Cannabis do not only cause effects individually, and in combination with other terpenes, but may also modulate the effects of the different cannabinoids that are present.

In embodiments, the secondary bioactive molecule from Cannabis may be any of a hemiterpene, a monoterpene, a sesquiterpene, a diterpene, a sesterterpene, a triterpene, a sesquarterpene, a tetraterpene, a polyterpene, a carbohydrate, a fatty acid and its esters, an amide, an amine, a phytosterol, or a phenolic compound.

Examples of monoterpenes include myrcene, cis-β-ocimene, trans-β-ocimene, p-cymene, α-terpinene, β-phellandrene, γ-terpinene, α-terpinolene, α-phellandrene, 3-phenyl-2-methyl-prop-1-ene, α-pinene, β-pinene, camphene, Δ³-carene, Δ⁴-carene, sabinene, α-thujene, linalool, citral B, nerol, geraniol, ipsienol, citronellol, 2-methyl-2-heptene-6-on, geranyl acetone, m-mentha-1,8-(9)-dien-5-ol, carvacrol, carvone, α-terpineol, terpinene-4-ol, pulegone, dihydrocarvone, β-terpineol, dihydrocarveyl acetate, p-cymene-8-ol, β-cyclocitral, safranal, cis-linalool oxide, perillene, sabinol, thujyl alcohol, linalool oxide, cis-carveol, cis-sabinene hydrate, sabinene hydrate, 8-cineol, 1,4-cineol, piperitone oxide, piperitenone oxide, fenchyl alcohol, fenchone, borneol, bornyl acetate, camphor, camphene hydrate, α-pinene oxide, pinocarveol, and pinocarvone.

Examples of sesquiterpenes include α-caryophyllene, β-caryophyllene, caryophyllene oxide, curcumene, α-trans-bergamotene, α-selinene, β-farnesene, longifolene, humulene epoxide I, humulene epoxide II, caryophyllene alcohol (caryophyllenol), β-bisabolene, allo-aromadendrene, calamenene, α-copaene, nerolidol, α-gurjunene, iso-caryophyllene, β-selinene, selina-3,7 (11)-diene, selina-4 (14),7 (11)-diene, α-bisabolol, α-cedrene, α-cubebene, δ-cadinene, epi-β-santalene, farnesol, γ-cadinene, γ-elemene, γ-eudesmol, guaiol, ledol, trans-trans-α-farnesene, (Z)-β-farnesene, farnesyl acetone, α-cadinene, α-cis-bergamotene, α-eudesmol, α-guaiene, α-longipinene, α-ylangene, β-elemene, β-eudesmol, epi-α-bisabolol, γ-cis-bisabolene, γ-curcumene, γ-muurolene, γ-trans-bisabolene, viridiflorene, germacrene-B, and clovandiol. Examples of diterpenes include phytol and neophytadiene. Examples of triterpenes include friedeline and epifriedelanol. Examples of miscellaneous terpenes include vomifoliol, dihydrovomifoliol, β-ionone, and dihydroactinidiolide.

Examples of phenolic compounds, in addition to those disclosed as part of, e.g., terpenes and flavonoids, include lignans, spiro-indans, dihydrostilbenes, dihydrophenanthrene derivatives, stilbenoids, cannabispirans, denbinobin, catechin, chlorogenic acid, caffeic acid, epicatechin, luteolin-7-O-glucoside, p-coumaric acid, caffeoyl, tyramine, ferulic acid, quercetin-3-glucoside, kaempferols, apigenin-7-glucoside, luteolin, cannabisins, and apigenin.

In embodiments, the secondary bioactive molecule from Cannabis may be any of a flavonoid, including orientin, orientin-O-glucoside, orientin-7-O-glucoside, orientin-7-O-rhamnoglucoside, vitexin, vitexin-O-glucoside, vitexin-7-O-glucoside, vitexin-7-O-rhamnoglucoside, cytisoside, cytisoside-glucoside, isovitexin, isovitexin-O-glucoside, isovitexin-7-O-glucoarbinoside, isovitexin-7-O-rhamnoglucoside, apigenin-7-O-glucoside, apigenin-7-O-glucuronoid, apigenin-7-O′P-coumaroylglucoside, 6-prenylapigenin, luteolin-C-glucuronide, luteolin-7-O-glucuronide, canniflavin A, canniflavin B, canniflavin C, chrysoeriol, kaempferol-3-O-diglucoside, quercetin-3-O-glucoside, quercetin-3-O-diglucoside, kaempferol-3-O-sophoroside, quercetin-3-O-sophoroside, rutin, quercetin, naringenin, and naringin; a terpene, including myrcene, cis-β-ocimene, trans-β-ocimene, p-cymene, α-terpinene, β-phellandrene, γ-terpinene, α-terpinolene, α-phellandrene, 3-phenyl-2-methyl-prop-1-ene, α-pinene, β-pinene, camphene, 43-carene, Δ⁴-carene, sabinene, α-thujene, linalool, citral B, nerol, geraniol, ipsienol, citronellol, 2-methyl-2-heptene-6-on, geranyl acetone, m-mentha-1,8-(9)-dien-5-ol, carvacrol, carvone, α-terpineol, terpinene-4-ol, pulegone, dihydrocarvone, β-terpineol, dihydrocarveyl acetate, p-cymene-8-ol, β-cyclocitral, safranal, cis-linalool oxide, perillene, sabinol, thujyl alcohol, linalool oxide, cis-carveol, cis-sabinene hydrate, sabinene hydrate, 8-cineol, 1,4-cineol, piperitone oxide, piperitenone oxide, fenchyl alcohol, fenchone, borneol, bornyl acetate, camphor, camphene hydrate, α-pinene oxide, pinocarveol, pinocarvone, α-caryophyllene, β-caryophyllene, caryophyllene oxide, curcumene, α-trans-bergamotene, α-selinene, β-farnesene, longifolene, humulene epoxide I, humulene epoxide II, caryophyllene alcohol (caryophyllenol), β-bisabolene, allo-aromadendrene, calamenene, α-copaene, nerolidol, α-gurjunene, iso-caryophyllene, β-selinene, selina-3,7 (11)-diene, selina-4 (14),7 (11)-diene, α-bisabolol, α-cedrene, α-cubebene, δ-cadinene, epi-β-santalene, farnesol, γ-cadinene, γ-elemene, γ-eudesmol, guaiol, ledol, trans-trans-α-farnesene, (Z)-β-farnesene, farnesyl acetone, α-cadinene, α-cis-bergamotene, α-eudesmol, α-guaiene, α-longipinene, α-ylangene, β-elemene, β-eudesmol, epi-α-bisabolol, γ-cis-bisabolene, γ-curcumene, γ-muurolene, γ-trans-bisabolene, viridiflorene, germacrene-B, clovandiol, phytol, neophytadiene, friedeline, epifriedelanol, vomifoliol, dihydrovomifoliol, β-ionone, dihydroactinidiolide, a carbohydrate, a fatty acid and its esters, an amide, an amine, a phytosterol, and a phenolic compound, including lignans, spiro-indans, dihydrostilbenes, dihydrophenanthrene derivatives, stilbenoids, cannabispirans, denbinobin, catechin, chlorogenic acid, caffeic acid, epicatechin, luteolin-7-O-glucoside, p-coumaric acid, caffeoyl, tyramine, ferulic acid, quercetin-3-glucoside, kaempferols, apigenin-7-glucoside, luteolin, cannabisins, and apigenin.

In embodiments, bioactive molecules from Cannabis additionally include those outlined in Russo, 2011; Gertsch, Pertwee, and Di Marzo, 2010; Tahir et al., 2021; Thomas and ElSohly, 2016; De Backer et al., 2009; and Hazekamp et al., 2004.

ii. Dipteryx

In some embodiments, the plant portion of a therapeutic combination is from Dipteryx. In embodiments wherein the plant portion is described as being “from” Dipteryx, it will be understood that the plant portion comprises matter derived from Dipteryx, including, as non-limiting examples, Dipteryx material such as raw (i.e., unprocessed) Dipteryx biomass, a Dipteryx extract (e.g., extracts described herein, such as aqueous and/or ethanolic extracts), or a molecule that is naturally occurring in Dipteryx (e.g., a primary or secondary bioactive molecule described in embodiments herein), whether or not said molecule is actually obtained by isolating the molecule from Dipteryx, or another means (e.g., by chemical synthesis).

In some embodiments, a therapeutic combination comprises a Dipteryx extract. In some embodiments, the therapeutic combination comprises a primary bioactive molecule and/or a secondary bioactive molecule from a species in the genus Dipteryx.

Although in some preferred embodiments, the primary bioactive molecule and/or secondary bioactive molecule are from Dipteryx, in other embodiments, the primary bioactive molecule and/or secondary bioactive molecule are from another genus from the tribe Dipterygeae, which further includes the genera Monopteryx, Pterodon, and Taralea.

In embodiments, the primary and/or secondary bioactive molecules are from Dipteryx, including from D. odorata, also herein referred to as the “Cumaru tree” or “Cumaru.”

Although in some preferred embodiments a bioactive molecule such as a coumarin is from a plant of the genus Dipteryx, such as Dipteryx odorata, i.e., Cumaru, in other embodiments a bioactive molecule such as a coumarin is from a plant of another genus, including any genera in the tribe Amburaneae, such as Amburana, Cordyla, Dupuya, Dussia, Mildbraediodendron, Myrocarpus, Myrospermum, Myroxylon, and Petaladenium. Accordingly, in some embodiments, species from such other genera, and extracts and bioactive molecules therefrom, will be considered as equivalents of like extracts and bioactive molecules from Dipteryx.

Dipteryx is a genus of large trees belonging to the family Fabaceae, native to South and Central America and the Caribbean, and previously known as the genus (oumarouna.

In some embodiments, the therapeutic combinations comprise primary and/or secondary bioactive molecules from the species Dipteryx odorata.

Dipteryx odorata is known by many names, including (oumarouna odorata, Cumaru tree (Brazil), Tonka bean tree, Brazilian teak, Tonquin bean, rumara, Kumaru (Guyana), cumaruzeiro (Portuguese), charapilla (Peru), charapilla del murciélago (Peru), shihuahuaco (Peru), and sarapia (Venezuela, Columbia), all of which may be used interchangeably or viewed equivalently according to the disclosure herein. Dipteryx odorata is a large rainforest canopy tree that grows up to 30 meters in height in the Amazon. It can be found in Brazil, Venezuela, Guyana, French Guiana, Suriname, Nigeria, Peru and Colombia. Historically, the seed and bark of Dipteryx odorata have been employed by local Amazonians, the seeds fermented in rum and used for snakebites, cuts, contusions, coughs, rheumatism, and as shampoo; the seed oil used to prevent earaches and ear infections, and bark to create a bath for fever patients.

The seeds (also referred to as the beans) produced by the Cumaru tree (“tonka beans”), containing the bioactive molecule coumarin, have a pleasant vanilla-like odor and have been used in perfumery, soaps, and as a flavoring agent in foods and tobacco products. For their preparation, the beans are usually fermented in a local rum and then air-dried. This results in the formation of coumarin crystals dusting the outside of the seeds, making them appear frosted.

In some embodiments, a disclosed combination comprising a bioactive molecule from Dipteryx, such as a coumarin, can exhibit antispasmodic, emmenagogue, cardiotonic, and antiasthmatic effects.

In some aspects, a disclosed combination comprising a bioactive molecule from Dipteryx, such as a coumarin, modulates a serotonin, a dopamine (including the upregulation of its synthesis and release), and/or an adrenergic receptor, and can exhibit neuroprotective effects, e.g., as disclosed in Ostrowska, 2020 and Abdelhafez et al., 2011. In some embodiments, a disclosed combination comprising a bioactive molecule from Dipteryx, such as a coumarin, inhibits MAO-B, thereby allowing dopamine to remain active, e.g., as disclosed in Huang et al., 2015.

In some aspects, a disclosed combination comprising a bioactive molecule from Dipteryx, such as a coumarin, decreases the production of nitric oxide (NO), tumor necrosis factor alpha (TNF α), and/or interleukin-1 beta (IL-1β) in a dose-dependent manner. In some aspects, a disclosed combination comprising a bioactive molecule from Dipteryx, such as a coumarin, inhibits LPS-induced protein and mRNA expression levels of nitric oxide synthase (iNOS) and cyclooxygenase-2 (COX-2) in RAW264.7 cells. In some aspects, a disclosed combination comprising a bioactive molecule from Dipteryx, such as a coumarin, activates PPAR-y, counter-regulating inflammatory activity in a tissue-specific way; and transcription factor NRf2, further modulating NF-kB activity and exerting useful cytoprotective mechanisms, e.g., as disclosed in Hassanein et al., 2020; and Di Stasi, 2021.

ii. Primary and Secondary Bioactive Molecules from Dipteryx

In embodiments, a therapeutic combination comprises a primary and/or secondary bioactive molecule from Dipteryx.

In embodiments, the primary bioactive molecule from Dipteryx is coumarin (2H-chromen-2-one; 2H-1-benzopyran-2-one), a colorless crystalline solid that gives tonka beans a characteristic vanilla-like aroma and flavor, and which may also act as a chemical defense against predators.

Coumarin may be obtained from Dipteryx and from myriad other plant species, including vanilla grass (Anthoxanthum odoratum), sweet woodruff (Galium odoratum), sweet grass (Hierochloe odorata), sweet-clover (genus Melilotus), cinnamon, including ceylon cinnamon or “true cinnamon” ((′innamomum verum), Chinese cinnamon or Chinese cassia (C. cassia), Indonesian cinnamon or Padang cassia (C. burmannii), Saigon cinnamon or Vietnamese cassia (C. loureiroi), deertongue ((′arphephorus odoratissimus), Tilo (Justicia pectoralis), Mullein (genus Verbascum), many cherry blossom tree varieties of the genus Prunus, and in trace amounts in strawberries, black currants, apricots, and cherries (Ananthakrishnan, et al., 2018; Wang et al., 2013; Khan and Ehab, 2010; Ieri, Pinelli, and Romani, 2012; and National Center for Biotechnology Information, 2022). In some embodiments, a coumarin is extracted, isolated, or otherwise obtained from any of the above or other such species, instead of from Dipteryx.

In some embodiments, a bioactive molecule is a compound derived from coumarin (e.g., a phenylpropanoid, a coumarin, or a coumarinoid) and which may be used in disclosed combinations, compositions, and methods; such compounds include, e.g., the bioactive molecules umbelliferone, aesculetin, herniarin, psoralen, dicoumarol, imperatorin, brodifacoum, bromadiolone, difenacoum, auraptene, ensaculin, phenprocoumon, PSB-SB-487, PSB-SB-1202, scopoletin, and warfarin (see, e.g., Laposata, Van Cott, and Lev, 2007; Syah, et al., 2009).

In embodiments, the secondary bioactive molecule from Dipteryx is any of cumaru, a coumarin derivative, an isoflavone, a lupeol derivative, a fatty acid ester, (±)-balanophonin, (−)-lariciresinol, 3′-hydroxyretusin-8-methyl-ether, 5-methoxyxanthocercin A, 6,4′-dihydroxy-3′-methoxyaurone, 7-hydroxychromone, 7,3′-dihydroxy-8,4′-dimethoxy-isoflavone, betulin, butin, coumaric-acid-beta-glucoside, dipteryxin, dipteryxic acid, eriodictyol, ferulic-acid, isoliquiritigenin, lupeol, melilotoside, melilotoside-1-p-coumaryl-beta-d-glucose, methyl-linolenate, methyl-oleate, O-coumaricacid, O-hydroxycoumaric-acid, odoratin, P-hydroxy-benzoic-acid, retusin, retusin-8-methyl-ether, sulfuretin, salicylic-acid, afrormisin, castinin, linoleic acid, oleic acid, 3′,4′,7′-trihydroxyflavone, luteolin, and umbelliferone.

In embodiments, bioactive molecules from Dipteryx additionally include those in Trincone, 2019; Jofre et al., 2020; or Gomez-Zavaglia et al., 2019.

c. Algae

In some embodiments, a therapeutic combination comprises an algal portion. In some embodiments, the algal portion comprises an algal extract. In some embodiments, the algal portion comprises a bioactive molecule from an algae. In some embodiments, the algal extract comprises a bioactive molecule from an algae. For example, in some embodiments, the algal portion comprises a primary bioactive molecule and/or a secondary bioactive molecule from an algae. In some embodiments, the algal extract comprises a primary bioactive molecule and/or a secondary bioactive molecule from an algae. Hence, in some embodiments, a disclosed therapeutic combination comprises a primary bioactive molecule and/or a secondary bioactive molecule from an algae.

In embodiments wherein the algae portion is described as being “from” an algae, it will be understood that the algae portion comprises matter derived from the algae, including, as non-limiting examples, algae material such as raw (i.e., unprocessed) algae biomass, an algae extract (e.g., extracts described herein, such as aqueous and/or ethanolic extracts), or a molecule that is naturally occurring in the algae (e.g., a primary or secondary bioactive molecule described in embodiments herein), whether or not said molecule is actually obtained by isolating the molecule from the algae, or another means (e.g., by chemical synthesis).

Algae broadly refers to a large, polyphyletic group of photosynthetic eukaryotic organisms. Generally, no definition of algae is universally accepted; however, one means of describing the multiple clades encompassing “algae” is that they have chlorophyll as their primary photosynthetic pigment and lack a sterile covering of cells around their reproductive cells (Lee, 2008). Algae includes varieties capable of living in freshwater, and/or salt water, and generally includes Euglenophyta (Euglenoids), Chrysophyta (golden-brown algae and diatoms), Pyrrophyta (fire algae), Chlorophyta (green algae), Rhodophyta (red algae), Phaeophyta (brown algae), and Xanthophyta (yellow-green algae) (Bailey, 2018).

In embodiments, the algae useful in the therapeutic combinations of the invention is a marine algae. In embodiments, the marine algae may be any of a brown algae (Phaeophyta), a green algae (Chlorophyta), and a red algae (Rhodophyta). In embodiments, the marine algae is a marine red algae. In embodiments, the marine red algae is from the family Bangiacea. In embodiments, the marine red algae is from the genus Pyropia or Porphyra.

Culturally, Porphyra has been cultivated in East Asia and utilized in the making of “nori” and other forms of edible seaweed such as used in sushi to bind and secure rice to other proteins, such as fish. Some species of Porphyra also are harvested in Western Europe to produce laverbread, a traditional food consumed primarily in Wales as part of local traditional cuisine (Harford, n.d.).

In embodiments, the red algae is Porphyra umbilicalis. Broadly, umbilicalis is a membranous, single-layered algae with centrally attached irregularly lobed, olive to brown fronds (up to 200 mm long) that split from a central holdfast and generally have a lettuce-like appearance. Texturely, umbilicalis is often smooth and gelatinous, lacking rigidity (Harford, n.d). Like other Porphyra species, umbilicalis may be found growing on rocks and sometimes mussels in midtidal to splash zones and are distributed in abundance in spring and summer. In embodiments, the red algae is Pyropia perforata. Perforata is generally purple/greenish in color, has lobed blades, and can reach 30 cm in width and length. Perforata may be found growing on rocks, or as epiphytes on other species of algae. In embodiments, the red algae is Pyropia yezoensis. Yezoensis has a discoid holdfast and short stipe, and folded blades which are membranous and monostromatic, coming in red, brown, and dark green colorations.

a. Primary and Secondary Bioactive Molecules from Algae

In embodiments, a therapeutic combination comprises a primary and/or secondary bioactive molecule from algae.

In embodiments, the primary bioactive molecule from an algae is a primary bioactive molecule from a genus in the family Bangiaceae. In embodiments, the primary bioactive molecule from an algae is a primary bioactive molecule from a species in the genus Pyropia or Porphyra. In embodiments, the primary bioactive molecule from an algae is a primary bioactive molecule from Porphyra umbilicalis. In embodiments, the primary bioactive molecule from an algae is a primary bioactive molecule from Pyropia yezoensis. In embodiments, the primary bioactive molecule from an algae is a primary bioactive molecule from Pyropia perforata.

In embodiments, the primary bioactive molecule from Pyropia or Porphyra is any of porphyran and/or oligo-porphyran, a polysaccharide, oligo-polysaccharide, and/or monosaccharide; a peptide, a phycobiliprotein, mycosporine-like amino acid, an essential amino acid, a nonessential amino acid, a carotene, an intermediate carotenoid, a glycoprotein, and an amino sulfonic acid such as taurine.

In some embodiments, disclosed combinations, compositions, and methods comprising bioactive molecules from algae such as oligosaccharides from Pyropia will be shown to increase the synthesis of dopamine by inhibiting the loss of tyrosine hydroxylase, such as according to the methods disclosed in Liu et al., 2019. In some embodiments, disclosed combinations, compositions, and methods comprising bioactive molecules from algae such as porphyran will be shown to provide neuroprotective effects. In some embodiments, disclosed combinations, compositions, and methods comprising bioactive molecules from algae such as taurine will be shown to protect dopaminergic neurons and elevate dopamine, such as shown in mouse models in the methods disclosed in Liu et al., 2018; Che et al., 2018; and Ericson et al., 2006.

In some embodiments, disclosed combinations, compositions, and methods comprising bioactive molecules from algae such as taurine will be shown to reduce neuropathy through down-regulation of NF-κB and activation of Nrf2 signaling cascades. In some embodiments, taurine will be shown to reduce serum levels of IL-6 in TBI patients, such as according to the methods disclosed in Agca et al., 2014; and Vahdat et al., 2021. In embodiments, taurine will be shown to biostabilize membranes, encourage redox homeostasis, and scavenge oxidative factors, such as according to the methods disclosed in Yildirim et al., 2007; and Thirupathi et al., 2020.

In embodiments, phycobiliproteins include phycoerythrin, phycoerythrobilin, phycocyanin, and allophycocyanin. In embodiments, mycosporine-like amino acids include porphyra-334 and shinorine. In embodiments, amino acids include isoleucine, leucine, threonine, methionine, phenylalanine, lysine, histidine, valine, arginine, and cysteine. In embodiments, nonessential amino acids include aspartic acid, glutamic acid, glycine, tyrosine, serine, alanine, and proline. In embodiments, peptides include monopeptides, dipeptides, tripeptides, and proteins. In embodiments, carotenes include lutein, zeoxanthin, α-carotene, β-carotene, and astaxanthin. In embodiments, intermediate carotenoids include α-cryptoxanthin, zeinoxanthin and β-cryptoxanthin.

In embodiments, the primary bioactive molecule from Pyropia or Porphyra is any of porphyran and/or oligo-porphyran, polysaccharides, peptides, including monopeptides, dipeptides, tripeptides, and proteins; phycobiliproteins, including phycoerythrin, phycoerythrobilin, phycocyanin, and allophycocyanin; mycosporine amino acids, including porphyra-334 and shinorine; essential amino acids, including isoleucine, leucine, threonine, methionine, phenylalanine, lysine, histidine, valine, arginine and cysteine; nonessential amino acids, including aspartic acid, glutamic acid, glycine, tyrosine, serine, alanine, and proline; carotenes, including lutein, zeoxanthin, α-carotene, β-carotene, and astaxanthin; intermediate carotenoids, including α-cryptoxanthin, zeinoxanthin, and β-cryptoxanthin; and taurine.

In embodiments, a therapeutic combination comprises a bioactive molecule from an algae, wherein the bioactive molecule, in embodiments, is a secondary bioactive molecule from an algae. In embodiments, the secondary bioactive molecule from an algae is a secondary bioactive molecule from a genus in the family Bangiaceae. In embodiments, the secondary bioactive molecule from an algae is a secondary bioactive molecule from a species in the genus Pyropia or Porphyra. In embodiments, the secondary bioactive molecule from an algae is a secondary bioactive molecule from Porphyra umbilicalis. In embodiments, the secondary bioactive molecule from an algae is a secondary bioactive molecule from Pyropia Yezoensis.

In embodiments, the secondary bioactive molecule from Pyropia or Porphyra is any of a mineral, a vitamin, a lipid, a phenolic compound, and a phlorotannin.

In embodiments, minerals include potassium, phosphorus, magnesium, sodium, calcium, manganese, iron, copper, and zinc. In embodiments, vitamins include vitamin K, ascorbic acid, folate, and cobalamin. In embodiments, lipids include fatty acids, such as eicosapentaenoic acid and palmitic acid. In embodiments, phlorotannins include phlorotannin A.

In embodiments, the secondary bioactive molecule from Pyropia or Porphyra is any of minerals, including potassium, phosphorous, magnesium, sodium, calcium, manganese, iron, copper, and zinc; vitamins, including vitamin K, ascorbic acid, folate, and cobalamin; lipids, including fatty acids such as eicosapentaenoic acid and palmitic acid; phenolic compounds, and phlorotannins, including phlorotannin A.

### C. Obtaining Bioactive Molecules

In embodiments, the primary and secondary bioactive molecules disclosed herein may be obtained via any of, as non-limiting examples: extraction, synthesis, biosynthesis methods, as a whole plant, such as a Cannabis flower or Cannabis biomass, a fruiting fungal body, fungal mycelium mass, bioreactor-produced fungal biomass, a truffle (a fungal sclerotia), a cumaru seed, or whole Porphyra or Pyropia algae, isolated, and/or gathered as fractions.

In some embodiments, a bioactive molecule of a therapeutic combination will be commercially available, and will be commercially sourced (e.g., Cayman Chemical Company, Ann Arbor, MI; Sigma-Aldrich, Burlington, MA).

While these methods will be known to those of skill, exemplary means of obtaining primary and secondary bioactive molecules are disclosed herein.

a. Extraction

In some embodiments, extracts used in a disclosed therapeutic combination (e.g., a fungal extract, plant extract, or algal extract) is obtained through the use of extraction techniques known to those of skill in the art. In some embodiments, a disclosed primary and secondary bioactive molecule is provided in the form of an extract produced according to extraction techniques known to those of skill in the art.

Broadly, but without being bound by theory, an extraction system works by introducing a material, such as a fungal material, a plant material, and/or an algal material (e.g., containing a desired primary and/or secondary bioactive molecule), to a solvent capable of separating desired primary and/or secondary bioactive molecules from the material to form a solution containing the solvent and the primary and/or secondary bioactive molecules (an extract).

An extract may be used directly in a disclosed combination, composition, or method, or may be first further processed, such as by further extraction, filtration, distillation, fractionation, subfractionation, isolation, and/or purification, and other such methods known in the art, and including combinations thereof.

Various types of extraction systems exist, utilizing different methods, and having different parameters (e.g., temperature, pressure, solvent) which may be tailored to the desired end product, for example the desired primary and/or secondary bioactive molecules. Such properties will be known to those in the art. For example, and without being bound by theory, extraction systems generally follow the rule “like dissolves like.” Thus, when extracting a polar bioactive molecule, a polar solvent may be utilized; and when extracting a nonpolar bioactive molecule, a nonpolar solvent may be utilized (Lowery and Richardson, 1987).

Methods of extracting primary and/or secondary bioactive molecules, of creating plant and fungal extracts thereof, and of generally obtaining purified products containing desired compounds free from undesired plant or fungal matter, chemicals, and other impurities are known in the art, see e.g., U.S. Pat. Nos. 6,403,126, 8,846,409, 8,895,078, 10,059,684, 10,239,808, 10,246,431, 10,300,494, 10,307,447, 10,406,453, 10,413,845, and 10,414,709; and U.S. App. Nos. 2003/0017216A1 and 2016/0038437A1, and references cited, all of which are incorporated.

Exemplary extraction methods and systems disclosed in such references and otherwise herein should not be construed as being limiting, and many variations will be appreciated by those of skill. While exemplary extraction methods, including those described herein, are disclosed as a series of steps, other extraction methods useful in the practice of the invention and in obtaining bioactive molecules therefore may deviate from such steps, including by the modification to, removal of, addition of, or rearrangement of any such steps, as will be appreciated by those of skill, in view of this disclosure and the general knowledge in the art.

Herein, “extract” may refer to a botanical extract (e.g., a fungal, plant, Cannabis, Dipteryx, algal, or other extract containing a primary and/or secondary bioactive molecule disclosed herein) prepared, for example, from a botanical source (as “botanical” is defined herein). In some embodiments, an extract undergoes further extraction, filtration, fractionation, subfractionation, partial purification, substantial purification, or complete purification, or other processing to obtain a specific bioactive molecule(s) separate from other constituents, wherein such bioactive molecule(s) are found in the filtrate, fraction, subfraction, partially purified product, substantially purified product, completely purified product, isolate, or the like, and in some cases may be obtained separated from all other components as a single component or as single components. In some embodiments, an extract does not undergo fractionation, subfractionation, partial purification, substantial purification, or complete purification, or other processing to obtain a specific bioactive molecule(s) separate from other constituents, and is obtained as a whole plant, whole fungal, or whole algal extract, which may be referred to herein as a “whole extract.” In alternative embodiments of any exemplary disclosed embodiment herein, a whole extract is substituted with a fraction, subfraction, partially purified product, substantially purified product, completely purified product, isolate, or the like, as well as by a single bioactive molecule or one or more molecule(s) from an extract (including in an extract), or by molecule(s) produced synthetically, such as by partial or complete chemical synthesis, or by biosynthesis.

Extracts thus may include purified extracts. “Purified extract” may refer to a botanical extract that has undergone further processing after preparation, as will be understood by those in the art. In some exemplary embodiments, purified extracts may be the product of, e.g., soaking or heating the preparation in water and/or alcohol (e.g., depending on whether and to what degree the bioactive molecules sought are water soluble), agitating, cooling the resulting liquid, straining, filtering, and removing unwanted products (repeating if necessary), and then evaporating sufficient liquid solvent to obtain a desired concentration (or entirely, to obtain a crystalline precipitate), or using a spray dryer to create a purified dried powder. Other purification techniques will be known to those of skill, and in general, extraction and purification techniques for obtaining high purity bioactive compounds will be known in the art.

A starch or other carrier can be added to a purified extract to maintain the purified dried powder as a free-flowing powder that is easy to work with during formulation, for instance if the dried powder will be added to capsules; however, when referring to the weight of a “purified extract,” any such carrier, diluent, or excipient is excluded from the total amount.

In embodiments, extracts may be “standardized,” which refers to extracts that include a primary and/or secondary bioactive molecule in a specific concentration. Methods to produce standardized extracts, such as by quantifying the concentration of an extract and then adding a carrier, diluent, or excipient to dilute the extract to a standardized concentration, or further concentrating an extract to increase its concentration, will be known to those of skill.

“Natural” may refer to a substance which was isolated, extracted, or otherwise obtained from a natural source, such as a fungus, plant, or algae. A natural primary or secondary bioactive molecule, such as a natural cannabinoid, therefore may be isolated or extracted from a Cannabis plant. Thus, in various disclosed embodiments, the compositions shall be derived from botanical sources including fungi, plants, and algae, as extracts or by other means, and shall comprise a botanical drug substance or a Cannabis-derived drug substance. In embodiments, such compositions shall comprise a botanical drug product or a Cannabis-derived drug product. In other embodiments, such compositions shall comprise a botanical drug product or a Cannabis-derived drug product, which is substantially free from impurities.

In some embodiments, wherein a disclosed combination or composition comprises multiple extracts (e.g., fungal extracts, plant extracts, algal extracts) the concentration of bioactive molecules contained therein is quantified (e.g., according to methods disclosed herein and otherwise known to one of skill). The concentrations of bioactive molecules in the extracts may vary, based on the concentration of bioactive molecules in the corresponding source material (e.g., raw fungal, plant, or algal biomass). Accordingly, the concentrations of bioactive molecules in disclosed compositions may vary from batch to batch, based on variability between batches of extracts. In conforming with FDA rules (2016, USHHS, FDA, CDER, Botanical Drug Development Guidance for Industry), bioactive molecules (e.g., a primary and/or secondary bioactive molecule as disclosed herein) may be added to an extract disclosed herein (e.g., a fungal, plant, or algal extract); or to a disclosed combination or composition comprising said extracts. This may be conducted, e.g., to standardize the disclosed combination or composition and ensure a fixed concentration of a bioactive molecule between batches, even when the source materials or extracts thereof may vary in their concentrations of bioactive molecules.

### i. Exemplary Extraction of Molecules from Fungi

In embodiments, a therapeutic combination comprises a primary and/or secondary bioactive molecule from a fungus. In embodiments, the primary and/or secondary bioactive molecule may be obtained via extraction of fungal material.

Without being bound by theory, fungal extraction methods can be generally described as follows. First, the fungal material comprising the desired primary and/or secondary bioactive molecules is obtained, optionally dried (using, e.g., means such as a low-temperature oven, food dehydrator, and/or commercial drier), and optionally ground, pulverized, macerated, and/or milled to form a substantially fine powder, which is optionally then sieved, e.g., for consistency.

The fungal material is then combined with a solvent capable of extracting the desired primary and/or secondary bioactive molecules (e.g., methanol, ethanol, water, or a mixture thereof) to form a slurry, and the slurry is agitated to facilitate extraction for a specified duration of time, for example about 24 hours. The solvent is then filtered and collected. In embodiments, filters may include a cheesecloth, filter paper, and/or a filtration system. Optionally, the extract may be evaporated, using any of ambient evaporation, rotary evaporation, vacuum evaporation, and evaporation methods wherein heat is applied, such as those utilizing an oven, to create a more concentrated extract. In embodiments, the fungal material may be re-saturated with the same or different solvent after the initial filtration to complete a secondary, tertiary, or further extraction. Extraction methods and variations thereto may be chosen using ordinary skill.

In embodiments, heat extraction is utilized. In embodiments, heat extraction follows a process as generally described above, except that for example the solvent is heated, the agitation is completed in a heated environment, or the extraction otherwise takes place at a raised temperature relative to ambient temperature. In embodiments, extraction is completed at temperatures at which psilocybin is substantially dephosphorylated to psilocin. In embodiments, heat extraction is completed in temperatures exceeding about 70° C. In embodiments, extraction occurs at temperatures below which there is substantial dephosphorylation of psilocybin.

In embodiments, ultrasonic extraction is utilized. In some embodiments, mushrooms or other fungal matter is dried and cut and/or pulverized prior to ultrasonic extraction. In embodiments, an ultrasonic extraction machine is utilized to expose fungal material to ultrasonic vibrations via an ultrasonic probe. In embodiments, the ultrasonic probe vibrates at 20 kHz or more. Without being bound by theory, as ultrasonic waves travel through a liquid, they create alternating high-pressure (compression) and low-pressure (rarefaction or expansion) cycles (Hielscher, 2022). During the low-pressure vacuum cycle, minute vacuum bubbles or cavities occur in the liquid, which grow over several pressure cycles. During the compression phase of the liquid and bubbles, the pressure is positive, while the rarefaction phase produces a vacuum (negative pressure) (Hielscher, 2022). During the compression-expansion cycles, the cavities in the liquid grow until they reach a size at which they cannot absorb more energy, at which point they implode. The implosion of those cavities results in various highly energetic sonomechanical effects, known as coustic/ultrasonic cavitation (Hielscher, 2022), during which bubbles are created in the sonicated liquid that disrupt cell walls of plant tissues and release the intracellular compounds, including the primary and/or secondary bioactive molecules.

In some embodiments, extraction with a Soxhlet extractor is utilized. In some embodiments, Soxhlet extraction is conducted using an ethanol and water solvent (e.g., 80% ethanol and 20% water). In some embodiments, the solvent is heated to reflux, and the solvent vapor travels up a distillation arm and floods into the chamber housing the thimble of fungal material. The condenser at the top of the device ensures that any solvent vapor cools, and drips back down into the chamber housing the fungal material. As a result, the chamber containing the fungal material slowly fills with warm solvent. When the Soxhlet chamber is almost full, the chamber is emptied by the siphon, and the solvent is returned to the distillation flask. In some embodiments, using standard equipment known to those of skill, Soxhlet extraction is conducted for between about 6 and 10 hours, including about 6 hours, about 7 hours, about 8 hours, about 9 hours, or about 10 hours. In embodiments, Soxhlet extraction is conducted for about 7 hours.

Extracts of fungal material (comprising a primary and/or secondary bioactive molecule) produced according to different methods described herein or otherwise known to one of skill may be combined to provide various advantages. For example, fungal extracts produced according to different techniques may contain different concentrations of primary and/or secondary bioactive molecules, and combining such extracts in a disclosed therapeutic combination may confer advantageous properties.

In some embodiments, the fungal portion of a disclosed therapeutic combination comprises a combination of fungal extracts. In some embodiments, the fungal portion of a disclosed therapeutic combination comprises an ultrasonic extract and a Soxhlet extract. In some embodiments, the combination comprises the ultrasonic extract and the Soxhlet extract in an ultrasonic: Soxhlet ratio (e.g., a volume ratio (v/v) or weight ratio (w/w) of the extract solutions, or a molar ratio, such as a molar ratio of the constituent bioactive molecule(s) in the extracts) of between about 0.5:1 to 10:1, including 0.5:1, 1:1, 1.25:1, 1.5:1, 1.75:1, 2:1, 2.5:1, 3:1, 3.5:1, 4:1, 5:1, 6:1, 7:1, 8:1, 9:1, and 10:1, including ranges in between these values. In some embodiments, the combination comprises the ultrasonic extract and the Soxhlet extract in an ultrasonic: Soxhlet volume ratio of between about 0.5:1 to 10:1, including 0.5:1, 1:1, 1.25:1, 1.5:1, 1.75:1, 2:1, 2.5:1, 3:1, 3.5:1, 4:1, 5:1, 6:1, 7:1, 8:1, 9:1, and 10:1, including ranges in between these values. In some embodiments, the combination comprises a mixture of the ultrasonic extract and the Soxhlet extract in an ultrasonic: Soxhlet ratio of about 2:1 (v/v).

Advantages of combining different fungal extracts in disclosed combinations can include, for example, improved solution stability. For example, certain compositions disclosed herein comprise, as a component of a fungal portion, psilocin and/or psilocybin. The solution instability of psilocybin and especially psilocin has long been recognized, but the exact chemistry of the mechanisms and products of decomposition remains a matter of ongoing research. Studies of Psilocybe mushrooms that have been damaged to expose its alkaloids to environmental oxygen have revealed that psilocybin is dephosphorylated to psilocin, whose hydroxy group is subsequently oxidized to produce 4-oxo degradation products (Lenz et al., 2020). Research into possible solution decomposition mechanisms has revealed that oxidative dimerization may also contribute to solution instability of psilocin (Lenz et al., 2021). In some embodiments, combining fungal extracts (e.g., as in disclosed combinations wherein the fungal portion comprises both an ultrasonic extract and a Soxhlet extract) protects the resulting combination against oxidative decomposition. Without being bound by theory, the differing levels of primary and/or secondary bioactive molecules in the different fungal extracts, which may include naturally occurring stabilizers and antioxidants, may in combination produce additive or synergistic effects that stabilize the resulting formulations against decomposition pathways, such as oxidative dimerization. In some embodiments, oxidative decomposition of a disclosed composition can be monitored quantitatively (e.g., by chemical analysis methods, such as those disclosed in Lenz et al., 2020 and Lenz et al., 2021; or otherwise known to one of skill) or qualitatively (e.g., by visual monitoring of the characteristic “blueing” reaction of psilocybin- and/or psilocin-containing extracts and materials).

In some embodiments, disclosed combinations wherein the fungal portion comprises multiple fungal extracts may also possess other advantageous properties, such as increased mixability with the other portions of the combination (e.g., plant and/or algal portions). By way of example, certain compositions disclosed herein comprise components with high water solubility (e.g., a fungal extract comprising psilocybin and/or psilocin, or a fungal extract comprising these compounds) and low water solubility (e.g., a plant portion comprising a cannabinoid or Cannabis extract). During the preparation and storage of such compositions, precipitation may occur due to mutual incompatibility between primary and/or secondary bioactive molecules and the solvent system. Without being bound by theory, the differing levels of primary and/or secondary bioactive molecules in the different fungal extracts may produce additive or synergistic effects that stabilize the resulting formulations against precipitation.

In some embodiments, disclosed combinations wherein the fungal portion comprises multiple different fungal extracts possess improved stability (e.g., against oxidation and/or precipitation) such that additional stabilizing excipients (e.g., antioxidants, stabilizers) are not necessary for the combination to have sufficient stability under ambient conditions. Reducing or eliminating the need for such additional stabilizing excipients may result in further advantages, such as improved bioavailability, as these ingredients may interfere with absorption of a primary and/or secondary bioactive molecule.

a. Determining the Concentration of a Primary and or Secondary Bioactive Molecule

It will be appreciated that determining the concentration of a primary and/or secondary bioactive molecule within an extract or within fungi, plant, and/or algae material is within the ability of one of skill. Several such means are disclosed below, and may be used in embodiments.

As a non-limiting example of such means, the concentration of primary bioactive molecules from various Psilocybe species are presented in TABLE 1 below (obtained from Mahmood, 2013, and organized by psilocybin content; see also Stamets, 1996) showing the w/w % of psilocybin, psilocin, and baeocystin in dried mushrooms (containing negligible water weight, or sometimes referred to as “cracker dry”), as is additionally discussed further herein:

TABLE 1
Psilocybin, Psilocin, & Bacocystin Content
of Exemplary Psilocybe Species by %

| Psilocybe Species | Psilocybin % | Psilocin % | Baeocystin % |
|---|---|---|---|
| P. azurescens | 1.70 | 0.38 | 0.35 |
| P. bohemica | 1.34 | 0.11 | 0.02 |
| P. semilanceata | 0.98 | 0.02 | 0.36 |
| P. baeocystis | 0.85 | 0.59 | 0.10 |
| P. cyanescens | 0.85 | 0.36 | 0.03 |
| P. tampanensis | 0.68 | 0.32 | n/a |
| P. cubensis | 0.63 | 0.60 | 0.025 |
| P. weilii | 0.61 | 0.27 | 0.05 |
| P. hoogshagenii | 0.60 | 0.10 | n/a |
| P. stuntzii | 0.36 | 0.12 | 0.02 |
| P. cyanofibrillosa | 0.21 | 0.04 | n/a |
| P. liniformans | 0.16 | n/a | 0.005 |

Thus, an extract of 100 mg P. cubensis may in some embodiments contain approximately 0.63 mg of psilocybin. An extract of 275 mg P. azurenscens may in some embodiments contain approximately 4.895 mg.

One will readily appreciate that the growing conditions of organisms such as fungi (as well as plants and algae) may influence the concentration of bioactive molecules found therein. The concentration of bioactive molecules also may differ depending on the organism part from which they are obtained (e.g, cap vs. stipe or mycelium, flower vs. leaves or stalk, etc.), as well as other variables known to those of skill. One will nevertheless understand how to select such growing conditions, organism parts, and the like, and understand how to determine the concentration of bioactive molecules with disclosed methods or those generally known in the art.

In embodiments, the concentration of a primary and/or secondary bioactive molecule in fungi, plants, and/or algae is determined utilizing liquid chromatography, such as high-performance liquid chromatography (HPLC). Broadly, HPLC works by using pumps to pass a pressurized liquid solvent containing the sample mixture (in this case, a fungal extract) through a column filled with a solid adsorbent material. As each individual component in the sample interacts differently with the adsorbent material, it causes different flow rates for the different components, leading to the separation of the components as they flow out of the column (Freshminds, 2021).

In embodiments, the concentration of a primary and/or secondary bioactive molecule in fungi, plants, and/or algae is determined utilizing reversed-phase HPLC and single-wavelength detection, as disclosed in Samuelsson et al., which is herein incorporated by reference. Broadly, reverse-phase HPLC generally proceeds in the same manner as the exemplary HPLC method disclosed above, but has a hydrophobic rather than a hydrophilic stationary phase. Meaning, hydrophobic molecules will absorb to the column packing, while hydrophilic molecules will be eluted and detected first (Mehta, 2012; Molnár and Horváth, 1976).

In embodiments, the concentration of a primary and/or secondary bioactive molecule in fungi, plants, and/or algae is determined utilizing liquid chromatography and mass spectrometry (LC/MS), as illustrated by Oetjen et al., 2020, which is herein incorporated by reference.

In embodiments, the concentration of a primary and/or secondary bioactive molecule in fungi, plants, and/or algae is determined utilizing hydrophilic interaction liquid chromatography (HILIC), described in Jiang et al., 2020, and incorporated by reference. Broadly, HILIC is a normal phase HPLC technique that utilizes reversed-phase type eluents. Thus, the column has a hydrophilic stationary phase, and the eluent contains water, a buffer, and a high concentration of water-miscible organic solvent (“Hydrophilic Interaction Liquid Chromatography,” 2022).

In embodiments, the concentration of a primary and/or secondary bioactive molecule in fungi, plants, and/or algae is determined utilizing a rapid personal quantification means usable for determination of concentration, one example of which for fungi is the PSILO-QTest (Miraculix; Jena, Germany), useful for determining the concentration of psilocybin. The PSILO-QTest uses chemical color reaction to detect concentration, with color intensity proportional to the concentration of the bioactive molecule (Mandrake, 2021). In some embodiments, characterization of the fungal, plant, and/or algal portion of a disclosed combination (e.g., characterization of the primary and/or secondary bioactive molecules) comprises characterizing free and/or bound amino acids using known chromatographic techniques (e.g., LC-MS, HPLC). In some embodiments, the characterization comprises determining the concentration of taurine in the fungal, plant, and/or algal portion.

### ii. Exemplary Extraction of Molecules from Cannabis

In embodiments, a therapeutic combination comprises a primary and/or secondary bioactive molecule from a Cannabis species. In embodiments, the primary and/or secondary bioactive molecule may be obtained via extraction of Cannabis plant material.

In embodiments, an exemplary extraction system is as follows: First, one may obtain cannabis plant material which, in embodiments, has been substantially dried. In embodiments, the dried cannabis plant material may then be ground, and optionally pulverized to yield a fine powder, which may optionally be sieved. As elsewhere, grinding and pulverization may increase the surface area available for a solvent to interact with a material and, thus, may increase the yield of desired primary and/or secondary bioactive molecules within the material. In embodiments, the dried, and optionally ground and/or pulverized plant material is then placed into an extraction system generally containing a loop wherein chilled ethanol (kept at between −30° C. and −40° C.) is circulated. Evaporation is used to remove solvent, followed by distillation and filtration to further concentrate the extract and remove impurities.

In such an exemplary extraction system, the high temperature and pressure would yield decarboxylation (which occurs at roughly 110° C. at standard pressure). If, as an example, one did not wish to obtain the intoxicating effects of THC, one may choose an extraction process with temperatures and pressures below the aforementioned threshold. Likewise, one may utilize, e.g., column chromatography, to remove the THC after extraction.

It will be appreciated that, while such an extraction system is disclosed as a series of steps, deviation from the steps is also within the scope of the invention, as is modification to, removal of, and addition of various steps at any point during the extraction process.

Moreover, ethanol extraction is just one extraction process capable of producing an extract useful in a therapeutic combination. In embodiments, other extraction methods and solvents may be utilized, non-limiting examples of which include subcritical and supercritical CO₂ extraction, hydrocarbon extraction such as with butane and/or propane, methanol extraction, isopropyl alcohol extraction, and other such extraction methods known to those of skill. While any such extraction methods may be utilized, it should be appreciated that certain extraction methods may provide specific benefits, depending on the desired composition of the extract.

As an example, supercritical extraction, such as supercritical CO₂ extraction, is useful for the extraction of cannabinoids, and preservation of terpenoids; warm alcohol extraction is useful in extracting and decarboxylating cannabinoids, but also extracts various pigments and impurities; cold alcohol extraction is more successful at isolating terpenes and cannabinoids from impurities than warm ethanol extraction, but is less efficient; hydrocarbon extraction, on the other hand, including butane and propane extraction, is effective in reducing impurities and pigments in the extract, and may be more efficient than cold alcohol extraction.

Primary and/or secondary bioactive molecules from Cannabis may be obtained via extraction of Cannabis plant material, and may be used to prepare Cannabis-derived drug substances and Cannabis-derived drug products.

“Cannabis-derived drug substance” may refer to a botanical drug substance which is derived from Cannabis plants (including plant parts, plant part biomass, and plant exudates), as non-limiting, purely illustrative examples, primary extracts prepared by processes including maceration, percolation, extraction with solvents such as C1-C5 alcohols (e.g., ethanol), hydrocarbons (e.g., propane, butane), and subcritical or supercritical carbon dioxide.

“Cannabis-derived drug product” may refer to a primary Cannabis extract that is further purified, for example by distillation or chromatography. It will be known to those of skill that when certain solvents are used to prepare primary extracts, the resultant extract may contain non-specific lipid-soluble material. Those of skill will know that such impurities can be removed by a variety of processes including winterization (e.g., chilling to −20° C. followed by filtration to remove waxy ballast), further extraction or filtration, and distillation.

### iii. Exemplary Extraction of Molecules from Dipteryx

In embodiments, a therapeutic combination comprises a primary and/or secondary bioactive molecule from Dipteryx. In embodiments, the primary and/or secondary bioactive molecule may be obtained via extraction of cumaru (tonka) beans.

As mentioned, a primary bioactive molecule in Dipteryx is coumarin, which may, in embodiments, be extracted from cumaru beans via extraction with a polar solvent, such as but not limited to water, alcohols, including ethanol and methanol; and various ethers. Below, an exemplary extraction system utilizing ethanol is described.

In embodiments, ethanol extraction may be utilized to extract coumarin from cumaru beans. In embodiments, the ethanol extraction may be ethanol percolation which, in embodiments, uses anhydrous ethanol. In embodiments, the ethanol percolation extraction system functions by first crushing the cumaru beans so that the ethanol solvent is capable of entering and exiting the beans. Preferably, the seeds are sufficiently ground to increase surface area for extraction, forming a collection of plant matter. The seed matter is then placed in a filter sitting atop a collection device. Ethanol is then poured over the seed matter, which then passes through the filter, and is collected in the collection device. In embodiments, this is completed until the color of the ethanol indicates substantially no additional bioactive molecules are being collected.

Optionally, the excess ethanol is then evaporated. This may be completed by applying heat to the resultant solution, or utilizing ambient evaporation.

Other extraction methods also may be used, as would be known to those of skill. Additionally, while coumarin is directly mentioned, it should be readily appreciated that, e.g., secondary bioactive molecules from cumaru may additionally be extracted in the same or a similar manner, and may be present in the extract when the exemplary ethanol extraction method is utilized. Moreover, the concentration of a primary and/or secondary bioactive molecule may be determined as disclosed herein.

b. Synthesis

In embodiments, the primary and secondary bioactive molecules disclosed herein may be synthetic, where “synthetic” herein may refer to a substance which is manufactured in a laboratory, by means of chemical synthesis (e.g., by a series of chemical processes or reactions using chemical substrates, reagents, and optionally a catalyst) or biosynthesis, discussed herein, (e.g., from a bioengineered organism, and thus including those compounds also referred to as “biosynthetic” or as involving “synthetic biology” or “synbio”).

Generally, methods for chemical synthesis are well known in the art, including for the bioactive molecules disclosed herein. Methods for synthesis of the bioactive molecules and/or starting materials therefore will be readily apparent to the skilled artisan in view of general references known in the art (see, e.g., Greene et al., 1991; Harrison et al., 1971-1996; “Beilstein Handbook of Organic Chemistry,” Beilstein Institute of Organic Chemistry, Frankfurt, Germany; Feiser et al, 1967-1994; Trost et al., 1991; “Theilheimer's Synthetic Methods of Organic Chemistry,” 1991; March 1991; Larock, 1989; Paquette, 1995) and may be used to synthesize the bioactive molecules, where not otherwise obtained, such as by extraction. In general, the approaches used for similar compounds (Shulgin & Shulgin, 1992; Shulgin & Shulgin, 1997; Glennon et al., 1986; Nichols et al., 1991; Kedrowski et al., 2007; Heravi & Zadsirjan, 2016; Keri et al., 2017; Pérez-Silanes et al., 2001; and references therein), such adaptation being that known and understood to those of ordinary skill.

In embodiments, the disclosed primary and secondary bioactive molecules may be obtained via biosynthesis. Biosynthesis refers to the production of molecules, such as the primary and/or secondary bioactive molecules utilized in the therapeutic combinations, within a cell or a cell-free system. In embodiments, the primary and/or secondary bioactive molecules useful in the therapeutic combinations may be produced via biosynthesis.

Methods for biosynthesis of the bioactive molecules will be readily apparent to the skilled artisan in view of general references known in the art (see, e.g., PCT Pub. Nos. WO2021/052989, WO2019/173797, WO2021/086513, and WO2019/180309). Non-limiting examples of known biosynthetic pathways in the art include those for cannabinoids, flavonoids, carotenoids, psilocybin, psilocin, and other indole alkaloids (such as the primary and secondary bioactive molecules found in fungi), amino acids, and peptides (including monopeptides, dipeptides, tripeptides, and proteins).

In embodiments, the primary bioactive molecule and/or secondary bioactive molecule from a fungus are produced by biosynthesis. In embodiments, the primary bioactive molecule and/or secondary bioactive molecule from a plant are produced by biosynthesis. In embodiments, the primary bioactive molecule and/or secondary bioactive molecule from algae are produced by biosynthesis.

c. Isolation, Fractionation, and Purification

In embodiments, the primary and/or secondary bioactive molecules may be obtained via fractionation, isolation, and/or purification. Fractionation refers to a separation process wherein a certain quantity of a mixture is divided during a phase transition into a number of smaller quantities termed fractions, the composition of which varying according to a gradient.

In embodiments, fractions containing target primary and/or secondary bioactive molecules may be obtained via fractional distillation, column chromatography, fractional crystallization, fractional freezing, and bioassay-guided fractionation.

In embodiments, isolation of primary and/or secondary bioactive molecules from extracts containing the primary and/or secondary bioactive molecules may be completed. Numerous means of isolating compounds from extracts are known to those of skill in the art. However, exemplary means include thin layer chromatography, column chromatography, flash chromatography, sephadex chromatography, and high performance liquid chromatography (Sasidharan et al., 2011).

In embodiments, extracts may be further purified to isolate a particular primary and/or secondary bioactive molecule. In embodiments, such purification techniques will be those known to those of skill, but may include HPLC and centrifugal partition chromatography (CPC).

d. Compounds

In embodiments, the disclosed primary and/or secondary bioactive molecules are pure or substantially pure. The terms “pure” or “substantially pure,” as used herein, refer to material that is substantially or essentially free from components that normally accompany the material when the material is synthesized, manufactured, or otherwise produced. A “pure” or “substantially pure” preparation of a primary and/or secondary bioactive molecule is accordingly defined as a preparation having a chromatographic purity (of the desired bioactive molecule) of greater than 90%, more preferably greater than 95%, more preferably greater than 96%, more preferably greater than 97%, more preferably greater than 98%, more preferably greater than 99%, more preferably greater than 99.5%, and most preferably greater than 99.9%, as determined by area normalization of an HPLC profile or other similar detection method. Preferably the pure or substantially pure primary and/or secondary bioactive molecules used in the invention are substantially free of any other active compounds which are not intended to be administered to a subject. In this context, “substantially free” refers to the fact that no active compound(s), other than the primary and/or secondary bioactive molecules intended to be administered to a subject, are detectable by HPLC or another similar detection method, or are below a desired threshold of detection such as defined above.

The disclosed primary and/or secondary bioactive molecules may contain one or more asymmetric centers and give rise to enantiomers, diastereomers, and other stereoisomeric forms. The invention includes all such possible isomers, as well as mixtures thereof, including racemic and optically pure forms. Optically active isomers may be prepared using chiral synthons or chiral reagents, or resolved using conventional techniques. Various methods are known in the art for preparing optically active forms and determining activity.

The invention also includes primary and/or secondary bioactive molecules with at least one desired isotopic substitution of an atom, at an amount above the natural abundance of the isotope, i.e., isotopically enriched. Isotopes are atoms having the same atomic number but different mass numbers, i.e., the same number of protons but a different number of neutrons. By way of example, isotopes of hydrogen including deuterium (²H) and tritium (³H) may be used anywhere in described structures that achieves the desired result. Alternatively or in addition, isotopes of carbon, e.g., ¹³C and ¹⁴C, may be used. Isotopic substitutions, for example deuterium substitutions, can be partial or complete. Partial deuterium substitution means that at least one hydrogen is substituted with deuterium. In certain embodiments, the isotope is at least 60%, 70%, 80%, 90%, 95%, or 99% or more enriched in an isotope at any location of interest. In one embodiment, deuterium is 90%, 95%, or 99% enriched at a desired location.

The primary and/or secondary bioactive molecules of the therapeutic combinations will be understood to also encompass pharmaceutically acceptable salts of such molecules. The term “pharmaceutically acceptable salt” refers to salts prepared from pharmaceutically acceptable non-toxic acids or bases, which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable. Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like. (See Berge et al., 1977.)

Prodrugs of the primary and/or secondary bioactive molecules also will be appreciated to be within the scope of the invention. The term “prodrug” refers to a precursor of a biologically active pharmaceutical agent. Prodrugs undergo a chemical or a metabolic conversion to become a biologically active pharmaceutical agent, such as the conversion of the prodrug psilocybin to its active metabolite psilocin.

In the case of solid compositions, it is understood that the disclosed primary and/or secondary bioactive molecules may exist in different forms. For example, the primary and/or secondary bioactive molecules may exist in stable and metastable crystalline forms, isotropic and amorphous forms, milled forms and nano-particulate forms, all of which are intended to be within the scope of the present invention. In addition, the primary and/or secondary bioactive molecules include crystalline forms, also known as polymorphs. Polymorphs include the different crystal packing arrangements of the same elemental composition of a bioactive molecule. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

For any of the disclosed primary and/or secondary bioactive molecules, substitution of the primary and/or secondary bioactive molecule by its ion, free base, salt form, polymorph, hydrate or solvate form, co-crystal, or an isomer or enantiomerically enriched mixture, shall be understood to provide merely an alternative embodiment still within the scope of the invention (with modifications to the formulation and dosage amounts made according to the teachings herein and ordinary skill, if necessary or desired). Further, compositions within the scope of the invention should be understood to be open-ended and may include additional primary and/or secondary bioactive molecules, active or inactive agents, and ingredients.

In some embodiments, disclosed primary and/or secondary bioactive molecules, or pharmaceutically acceptable salts, hydrates, solvates, or prodrugs thereof, are produced and tested in compliance with Good Laboratory Practice (GLP) or Good Manufacturing Practice (GMP) requirements, and/or their equivalents and any related practices, e.g., in Canada, when referring to Cannabis plants, the Good Production Practices (GPP) of the Cannabis Regulations.

### D. Therapeutic Combinations

In some aspects are therapeutic combinations comprising the primary and/or secondary bioactive molecules from a fungus, a plant, and/or an algae.

In embodiments, a therapeutic combination comprising a primary and/or secondary bioactive molecule from a fungus and a primary and/or secondary bioactive molecule from a plant will be synergistic or have synergistic effects. In embodiments, a therapeutic combination comprising a primary and/or secondary bioactive molecule from a fungus, and a primary and/or secondary bioactive molecule from a plant will be synergistic or have synergistic effects. In embodiments, a therapeutic combination comprising a primary and/or secondary bioactive molecule from a fungus and a primary and/or secondary bioactive molecule from an algae will be synergistic or have synergistic effects. In embodiments, a therapeutic combination comprising a primary and/or secondary bioactive molecule from a plant and a primary and/or secondary bioactive molecule from an algae will be synergistic or have synergistic effects. In embodiments, a therapeutic combination comprising a primary and/or secondary bioactive molecule from a fungus, a primary and/or secondary bioactive molecule from a plant, and a primary and/or secondary bioactive molecule from an algae will be synergistic or have synergistic effects.

### E. Synergism and Synergistic Effects

In embodiments, “synergistic” may refer to a combination that is more effective than the additive effects of any two or more single primary and/or secondary bioactive molecules. A synergistic effect, for example, permits the effective treatment of a disease using lower amounts (doses) of individual therapy. This includes lower doses of a first primary and/or secondary bioactive molecule or a second primary and/or secondary bioactive molecule (“apparent one-way synergy”), or lower doses of both primary and/or secondary bioactive molecules (“two-way synergy”), than would normally be required when either primary and/or secondary bioactive molecule is used alone. In effect, the lower doses result in lower toxicity without reduced efficacy. A synergistic effect may additionally result in improved efficacy, including an improved avoidance or reduction of disease as compared to any single therapy. “Synergistic effects” will also be understood to include increases in potency, bioactivity, bioaccessibility, bioavailability, or therapeutic effect (including one or more additional therapeutic effects), greater than the additive contributions of the components acting alone, and/or are greater than the contribution of the isolated primary and/or secondary bioactive molecules on their own.

Numerous methods known to those of skill exist to determine whether there is synergy as to a particular effect, i.e., whether, when two or more components are mixed together, the effect is greater than the sum of the effects of the individual components applied alone, thereby producing “1+1>2.” Suitable methods include isobologram (or contour) analysis (Huang et al. 2019), or the equation of Loewe additivity (Loewe & Muischnek, 1926). A synergistic effect also may be calculated using methods such as the Sigmoid-Emax equation (Holford & Scheiner, 1981) and the median-effect equation (Chou & Talalay, 1984). The corresponding graphs associated with the equations above are the concentration-effect curve and combination index curve, respectively. Each equation referred to above may be applied to experimental data to generate a corresponding graph to aid in assessing the effects of the drug combination.

In embodiments, a combination comprises a primary and/or secondary bioactive molecule from a psilocybin-producing fungus and a primary and/or secondary bioactive molecule from Cannabis. In some embodiments, the combination will be synergistic or have synergistic effects.

In embodiments, a combination comprises a primary and/or secondary bioactive molecule from a psilocybin-producing fungus and a primary and/or secondary bioactive molecule from Dipteryx. In some embodiments, the combination will be synergistic or have synergistic effects.

In embodiments, a combination comprises a primary and/or secondary bioactive molecule from a psilocybin-producing fungus and a primary and/or secondary bioactive molecule from Pyropia or Porphyra. In some embodiments, the combination will be synergistic or have synergistic effects.

In embodiments, a combination comprises a primary and/or secondary bioactive molecule from Cannabis and a primary and/or secondary bioactive molecule from Dipteryx. In some embodiments, the combination will be synergistic or have synergistic effects.

In embodiments, a combination comprises a primary and/or secondary bioactive molecule from Cannabis and a primary and/or secondary bioactive molecule from Pyropia or Porphyra. In embodiments, the combination will be synergistic or have synergistic effects.

In embodiments, a combination comprising a primary and/or secondary bioactive molecule from Dipteryx and a primary and/or secondary bioactive molecule from Pyropia or Porphyra. In some embodiments, the combination will be synergistic or have synergistic effects.

In embodiments, a combination comprises a primary and/or secondary bioactive molecule from a psilocybin-producing fungus, a primary and/or secondary bioactive molecule from Cannabis, and a primary and/or secondary bioactive molecule from Dipteryx. In some embodiments, the combination will be synergistic or have synergistic effects.

In embodiments, a combination comprises a primary and/or secondary bioactive molecule from a psilocybin-producing fungus, a primary and/or secondary bioactive molecule from Cannabis, and a primary and/or secondary bioactive molecule from Pyropia or Porphyra. In some embodiments, the combination will be synergistic or have synergistic effects.

In embodiments, a combination comprises a primary and/or secondary bioactive molecule from a psilocybin-producing fungus, a primary and/or secondary bioactive molecule from Dipteryx, and a primary and/or secondary bioactive molecule from Pyropia or Porphyra. In some embodiments, the combination will be synergistic or have synergistic effects.

In embodiments, a combination comprises a primary and/or secondary bioactive molecule from Cannabis, a primary and/or secondary bioactive molecule from Pyropia or Porphyra, and a primary and/or secondary bioactive molecule from Dipteryx. In some embodiments, the combination will be synergistic or have synergistic effects.

In embodiments, a combination comprises a primary and/or secondary bioactive molecule from a psilocybin-producing fungus, a primary and/or secondary bioactive molecule from Cannabis, a primary and/or secondary bioactive molecule from Dipteryx, and a primary and/or secondary bioactive molecule from Pyropia or Porphyra. In some embodiments, the combination will be synergistic or have synergistic effects.

In some embodiments, a disclosed therapeutic combination is a botanical formulation comprising minimal or low doses of whole extracts of C. sativa plant, P. cubensis fungi, P. yesoensis algae, and D. odorata bean, relative to those that would otherwise be understood or expected in the art, and a synergy is an ability to provide an effect at the given dose amounts. Because the disclosed combinations, compositions, and methods synergistically permit the effective use of surprisingly low doses of one or more of the bioactive molecules, in some embodiments these low doses will avoid receptor exhaustion, avoid the activation of self-regulating compensatory measures, or avoid induced compensatory neurological reactions.

### F. Additional Active Agents

The therapeutic combinations may also be used together with an additional active agent, which may, for example, contribute to or provide an additional therapeutic effect, or contribute to or provide a synergistic effect.

In embodiments, the additional active agent is an agent useful in treating Parkinson's carbidopa-levodopa, disease, including carbidopa-levodopa (controlled release), carbidopa-levodopa (orally disintegrating tablet), carbidopa-levodopa (extended release capsules), carbidopa-levodopa (oral suspension), carbidopa-levodopa-entacapone (enteral suspension), levodopa, levodopa (inhalation powder), a whole extract botanical version of levodopa, e.g., velvet bean or fava bean, entacapone, tolcapone, opicapone, pramipexole, pramipexole (extended release), ropinirole, ropinirole (extended release), apomorphine (injection), apomorphine sublingual film, rotigotine (transdermal patch), selegiline, selegiline (orally disintegrating tablet), rasagiline, safinamide, amantadine, amantadine (extended release), istradefylline, trihexyphenidyl, benztropine, procyclidine, trihexyphenidyl(benzhexol), orphenadrine, and buntanetap (posiphen, ANVS-401). In embodiments, an additional active agent may be administered in separate dosage forms or as a single dosage form comprising a primary and/or secondary bioactive molecule according to the invention in combination with the additional active agent.

In embodiments, an additional active agent is a serotonergic agent. In embodiments, a “serotonergic agent” refers to any compound that binds to, blocks, or otherwise influences (e.g., via an allosteric reaction) activity at a serotonin receptor, including any serotonin receptor subtypes. In embodiments, a serotonergic agent binds to a serotonin receptor. In embodiments, a serotonergic agent indirectly affects a serotonin receptor, e.g., via interactions affecting the reactivity of other molecules at the serotonin receptor. In embodiments, a serotonergic agent is an agonist, e.g., a compound activating a serotonin receptor. In embodiments, a serotonergic agent is an antagonist, e.g., a compound binding but not activating a serotonin receptor, e.g., blocking a receptor. In embodiments, a serotonergic agent is an effector molecule, e.g., a compound binding to an enzyme for allosteric regulation. In embodiments, a serotonergic agent acts (either directly or indirectly) at more than one type of receptor, including receptors other than serotonergic or other monoaminergic receptors. In embodiments, a serotonergic agent blocks the serotonin transporter (SERT) and results in an elevation of the synaptic concentration of serotonin, and an increase of neurotransmission. In embodiments, a serotonergic agent is a serotonin uptake or reuptake inhibitor. In embodiments, a serotonergic agent acts as a reuptake modulator and inhibits the plasmalemmal transporter-mediated reuptake of serotonin from the synapse into the presynaptic neuron, leading to an increase in extracellular concentrations of serotonin and an increase in neurotransmission. In embodiments, a serotonergic agent inhibits the activity of one or both monoamine oxidase enzymes, resulting in an increase in concentrations of serotonin and an increase in neurotransmission. In embodiments, a serotonergic agent is an antidepressant or anxiolytic, such as an SSRI, serotonin-norepinephrine reuptake inhibitor (SNRI), tricyclic antidepressant (TCA), monoamine oxidase inhibitor (MAOI), or atypical antidepressant. In other embodiments, a serotonergic agent is selected from the group consisting of: (1) serotonin transport inhibitors; (2) serotonin receptor modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin receptor agonists; (11) serotonin receptor antagonists; and (12) serotonin metabolites.

In embodiments, an additional active agent may be one or more of phenolic compounds, terpenes, polysaccharides, polyphenols, lipids, organic acids, polyunsaturated fatty acids (PUFAs), and tocopherols extracted from fungi, algae, or plants. In embodiments, an additional active agent may be extracted from genera including any of Cheirolophus, Rhaponticoides, Volutaria, Zingiber, Rosmarinus, Salvia, Thymus, Origanum, Ocimum, Melissa, Mentha, Origanum, Satureja, Hyssopus, Laurus, Bacopa, Bupleurum, Camellia, Berberis, and Lathyrus. In embodiments, the additional active agent is from the genus Zingiber, such as from Zingiber officinale. In embodiments, the additional active agent is from Zingiber officinale rhizomes. In embodiments, the additional active agent is from the genus Laurus, such as from Laurus nobilis. In embodiments, the additional active agent is from Laurus nobilis leaves. In embodiments, a disclosed composition comprises an additional active agent from Zingiber, such as from Zingiber officinale rhizomes, and an additional active agent from Laurus, such as from Laurus nobilis leaves.

In embodiments, an additional active agent may be any one or more of amino acids, antioxidants, anti-inflammatory agents, analgesics, antineuropathic and antinociceptive agents, antimigraine agents, anxiolytics, antidepressants, antipsychotics, anti-PTSD agents, cannabinoids, NMDA antagonists, dissociatives, immunostimulants, anti-cancer agents, antiemetics, orexigenics, antiulcer agents, antihistamines, antihypertensives, anticonvulsants, antiepileptics, bronchodilators, neuroprotectants, nootropics, entheogens, entactogens and empathogens, psychedelics, monoamine oxidase inhibitors (including RIMAs), tryptamines, terpenes, phenethylamines, sedatives, stimulants, serotonergic agents, and vitamins. These agents may be in ion, freebase, or salt form, and may be isomers, prodrugs, derivatives (preferably physiologically functional derivatives), or analogs.

### G. Pharmaceutical Compositions

In some aspects are disclosed pharmaceutical compositions comprising a therapeutic combination. “Pharmaceutical compositions” (and including for shorthand, unless context indicates otherwise, “compositions”) comprise a therapeutic combination together in an amount (for example, in a unit dosage form) with a pharmaceutically acceptable carrier, diluent, or excipient. It will be understood that some embodiments do not have a single carrier, diluent, or excipient alone, but have multiple carriers, diluents, and/or excipients.

In some embodiments, a disclosed combination or composition will comprise a non-naturally occurring carrier, diluent, or excipient, examples of which will be known to those in the art. In some embodiments, “non-naturally occurring” refers to a carrier, diluent, or excipient used in a disclosed combination or composition that does not naturally occur in a fungus, a plant, and/or an algae, or in any of the same fungi, plants, and/or algae from which the extracts and/or the bioactive molecules of the combination or composition are or can be derived.

Compositions can be prepared by standard pharmaceutical formulation techniques such as disclosed in Remington: Science and Practice of Pharmacy, 2005; The Merck Index, 1996; Pharm. Principles of Solid Dosage Forms 1993; Ansel and Stoklosa, 2001; and Poznansky et al., 1980. “Pharmaceutically acceptable,” used in connection with an agent, means the agents are generally safe and, within the scope of sound medical judgment, suitable for use in contact with the cells of humans and other animals without undue toxicity, irritation, allergic response, or complication, and commensurate with a reasonable risk/benefit ratio.

Compositions comprising a therapeutic combination can be formulated into any suitable dosage form, such as aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilised formulations, tablets, capsules, pills, powders, patches, inhalers, nebulizers, pulsatile release formulations, multi-particulate formulations, immediate release, controlled release, sustained release, extended release, and modified release formulations, and mixed immediate release and controlled release formulations.

In embodiments, a pharmaceutical composition is formulated in a unit dosage form. “Unit dosage form” refers to a physically discrete unit suited as unitary dosages for the patient to be treated, each unit containing a predetermined quantity of bioactive molecules calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose), of the pharmaceutical composition administered. Unit dosage forms include capsules, troches, cachets, lozenges, tablets, ampules and vials, which may include a composition in a freeze-dried or lyophilised state; a sterile liquid carrier, for example, can be added prior to administration or delivery in vivo. Unit dosage forms include ampules and vials with liquid compositions disposed therein. Unit dosage forms include compounds for transdermal administration, such as “patches” that contact the epidermis of a patient for an extended or brief period of time.

In embodiments, a pharmaceutical composition is formulated in a pharmaceutically acceptable oral dosage form, including oral solid dosage forms and oral liquid dosage forms.

In embodiments, the compositions are formulated as a pharmaceutically acceptable oral solid dosage form, including lozenges, troches, tablets, capsules, caplets, powders, pellets, multiparticulates, beads, spheres, capsules, pills, and/or any combinations thereof. Oral solid dosage forms may be formulated as immediate release, controlled release, sustained release, extended release, or modified release formulations. In embodiments, solid dosage forms may comprise pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. In embodiments, solid dosage forms may comprise pharmaceutically acceptable additives such as a compatible carrier, complexing agent, ionic dispersion modulator, disintegrating agent, surfactant, lubricant, colorant, moistening agent, plasticizer, stabilizer, wetting agent, anti-foaming agent, alone or in combination, as well as supplementary active agent(s), including preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of a pharmaceutical composition, and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid (ALA).

In embodiments, the compositions are formulated as a pharmaceutically acceptable oral liquid dosage form. Non-limiting examples of oral liquid dosage forms include tinctures, drops, emulsions, syrups, elixirs, suspensions, and solutions, and the like. In embodiments, oral liquid dosage forms may be formulated with any pharmaceutically acceptable excipient known to those of skill for the preparation of liquid dosage forms, and with solvents, diluents, carriers, excipients, and the like, chosen as appropriate to the solubility and other properties of the primary and/or secondary bioactive molecules disclosed hereins and other ingredients. Non-limiting examples of solvents include, e.g., water, glycerin, simple syrup, alcohol, medium chain triglycerides (MCT), and combinations thereof.

In embodiments, oral liquid dosage forms may be monophasic or biphasic, the former being a substantially homogenous solution dissolved in water or non-aqueous solvent, while the latter refers to oral liquid dosage forms in which the bioactive molecules do not fully dissolve in common solvents. In embodiments, over time, the solid particles (i.e., the bioactive molecules) within the oral liquid dosage form may form a precipitate at the bottom of the container-requiring vigorous shaking to redisperse the bioactive molecules. Non-limiting examples of monophasic liquid forms include syrups, linctuses, spirits/essences, elixirs, and fluid extracts. Non-limiting examples of biphasic liquid forms include oral suspensions, oral emulsions, and mixtures.

Liquid dosage forms for oral administration may be prepared as liquid suspensions or solutions using a sterile liquid, such as but not limited to, an oil, water, an alcohol, combinations of pharmaceutically suitable surfactants, suspending agents, and emulsifying agents. In embodiments, liquid formulations also may be prepared as single dose or multi-dose beverages. In embodiments, suspensions may include oils. Such oils include but are not limited to peanut oil, sesame oil, cottonseed oil, corn oil, and olive oil. Suitable oils also include carrier oils such as MCT and long chain triglyceride (LCT) oils. In embodiments, a suspension preparation may also contain esters of fatty acids such as ethyl oleate, isopropyl myristate, fatty acid glycerides, and acetylated fatty acid glycerides. In embodiments, suspension formulations may include alcohols, such as ethanol, isopropyl alcohol, hexadecyl alcohol; glycerol, and propylene glycol. In embodiments, ethers, such as polyethylene glycol; petroleum hydrocarbons, such as mineral oil and petrolatum; and water may also be used in suspension formulations. In embodiments, a suspension can thus include an aqueous liquid or a non-aqueous liquid, an oil-in-water liquid emulsion, or a water-in-oil emulsion.

In addition to the primary and/or secondary bioactive molecules, the liquid dosage forms may comprise additives, such as (a) disintegrating agents, (b) dispersing agents, (c) wetting agents, (d) preservatives, (e) viscosity enhancing agents, (f) sweetening agents, and/or (g) flavoring agents. In addition to the additives listed above, the liquid formulations of the invention, in embodiments, may also comprise inert diluents commonly used in the art such as water or other solvents, solubilizing agents, emulsifiers, flavoring agents, and/or sweeteners. In embodiments, co-solvents and adjuvants also may be added to a formulation.

In embodiments of modified release formulations, the plasma half-life compared to the plasma half-life of an immediate release formulation is greater by at least 5%, at least 10%, at least 15%, at least 20%, at least 25%, at least 30%, at least 35%, at least 50%, at least 75%, at least 100%, or values in between. In embodiments of modified release formulations, the formulations are designed to result in a comparable area under the curve, or AUCO-24, and a similar safety and efficacy profile, but having a delayed time to peak concentration (tmax) of at least 5%, at least 10%, at least 15%, at least 20%, at least 25%, at least 30%, at least 35%, at least 50%, at least 75%, at least 100%, or values in between. In embodiments, a formulation is designed to be a product with a specific time course based on an optimum therapeutic window, such as less than about 30 minutes, about 30 minutes, about 45 minutes, about 60 minutes, about 90 minutes, about 2 hours, about 3 hours, about 4 hours, and greater than 4 hours, including lengths of time in between.

In embodiments, a formulation is chosen based on its area of absorption. In embodiments, one may choose a tincture, such as the tincture described in the formulation examples below, based on its pre-digestion absorption by epithelial tissue.

In some embodiments, multiple formulations are combined, and the therapeutic combination comprises two or more pharmaceutical compositions together, which may be provided as a single pharmaceutical kit. In some embodiments, one or more portions of a therapeutic combination are provided as an inhalable formulation, such as for a soft mist inhaler, and the remaining portions of the combination are provided as a tincture. In embodiments, one or more portions of a therapeutic combination are provided as an inhalable formulation, and the remaining portions of the combination are provided as an oral dissolving strip. In embodiments, one or more portions of a therapeutic combination are provided as a tincture, and the remaining portions of the combination are provided as an oral dissolving strip. In embodiments, one or more portions of a therapeutic combination are provided as an inhalable formulation, and the remaining portions of the combination are provided as an oral spray, such as an oral mucosal spray. In embodiments, one or more portions of a therapeutic combination are provided as a tincture, and the remaining portions of the combination are provided as an oral spray. In embodiments, more than two separate formulations are combined, and the therapeutic combination therefore comprises more than two pharmaceutical compositions together, which may be provided as a single pharmaceutical kit. In embodiments, more than three, more than four, more than five, or more than six separate formulations are combined, and the therapeutic combination therefore comprises more than two, more than three, more than four, more than five, or more than six pharmaceutical compositions together, which may be provided as a single pharmaceutical kit.

In embodiments, a pharmaceutical composition is formulated as an inhaled formulation. Non-limiting examples of inhaled formulations include soft mist inhaler formulations, dry powder aerosol formulations, and vaporizer formulations. In embodiments, the inhaled formulation is a soft mist inhaler formulation (“soft mist” formulation). Soft mist formulations may be produced by combining liquids containing bioactive molecules in a cartridge or syringe. The cartridge or syringe is then placed within a soft mist inhaler device, such as those described in WO 2020/167893 and U.S. Pat. No. 9,108,011. One may also choose to include a solubilizer to assist in homogenizing the formulation (e.g., polysorbate 80, also known as TWEEN-80). In embodiments, the liquids containing the bioactive molecules may be liquid extracts obtained via the methods disclosed herein, with or without a solubilizing agent. In embodiments, the inhaled formulation may be a dry powder aerosol formulation, where bioactive molecules are obtained as a powder and are aerosolized by an inhaler device, such as a metered dose inhaler (MDI).

In embodiments, the inhaled formulation is a vaporizer formulation. A vaporizer formulation may be any of a vape juice, an e-liquid, an e-juice, and the like. In embodiments, vaporizer formulations may be a liquid (including, e.g., an oil) and/or a solid (including, e.g., a wax or a dry powder). In embodiments, vaporizer formulations may be produced by combining a bioactive molecule in a suitable liquid termed a “base liquid,” the resultant formulation capable of being vaporized by an internal heating element (such as those found on vape pens, e-cigs, e-pipes, and other such e-smoking devices). Examples of suitable liquids include propylene glycol (PG), vegetable glycerin (VG), and polyethylene glycol (PEG). In embodiments, the base liquid may also contain a liquid of lower viscosity to act as a thinning agent, including water and/or ethanol (where the ethanol may be an alcoholic drink or spirit, including vodka). In embodiments, the vaporizer formulation may additionally contain a flavorant, where the flavorant is any flavor concentrate known to those of skill.

In embodiments, a pharmaceutical composition is formulated as an effervescent powder.

In embodiments, a pharmaceutical composition is formulated in a pharmaceutically acceptable transdermal application, capable of being administered transdermally. Non-limiting examples of transdermal formulations include ointments, creams, suspensions, lotions, pastes, gels, sprays, foams, oils, and the like, and any combination thereof.

In embodiments, a pharmaceutical composition is formulated for subcutaneous, intravenous, intra-arterial, intraperitoneal, intraosseous, intramuscular, intrathecal, or intracerebroventricular injection (“injectable formulations”). In embodiments, injectable formulations may be prepared by dissolving, suspending, or emulsifying the primary and/or secondary bioactive molecules in an aqueous or nonaqueous solvent, non-limiting examples of which include oils, such as vegetable oil, synthetic aliphatic acid glycerides, and esters of higher aliphatic acids or propylene glycol; and may also comprise additives such as solubilizers, stabilizers, and suspending, preserving, wetting, emulsifying, dispensing, and isotonic agents.

In some embodiments, a composition is formulated for a specific tissue or for a specific route of administration, other than for oral gastrointestinal administration, such as mucosal (e.g., sublingual, buccal, rectal, nasal), intramuscular, subcutaneous, cutaneous, intranasal, inhaled, and the like. Such administration may result in a reduction of side effects, reduced toxicity, increased efficacy, improved selectivity, enhanced bioavailability, and minimized drug-drug interactions.

Injectable formulations may comprise additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like, and may comprise isotonic agents, such as sugars, sodium chloride, and the like. Prolonged drug absorption of the injectable pharmaceutical form can be brought about by the use of agents delaying absorption, such as aluminum monostearate and gelatin. Injectable formulations designed for extended-release via SC or IM injection can avoid first-pass metabolism and lower dosages of the primary and/or secondary bioactive molecules will be necessary to maintain desired plasma levels. In such formulations, the particle size and the range of the particle sizes of the primary and/or secondary bioactive molecules can be used to control the release of the primary and/or secondary bioactive molecules by controlling the rate of dissolution in fat or muscle.

In embodiments, a composition is formulated in a pharmaceutically acceptable nanostructured formulation, such as a nanoemulsion, a nanocapsule, a nanoparticle conjugate, or a nano-encapsulated oral, sublingual, buccal, or nasal spray. In embodiments, nanostructured formulations are prepared by reference to general knowledge of the art (see, e.g., Jaiswal 2015).

In some preferred embodiments, where a therapeutic combination, pharmaceutical composition, or other formulation comprises natural compounds or compounds found in nature, at least one of the carrier(s), diluent(s), and/or excipient(s) used in the combination, composition, or formulation are non-natural or non-naturally-occurring, or other agent(s), such as additional active agent(s), in the disclosed combination, composition, or formulation are non-natural or non-naturally-occurring, i.e., the natural compound(s) are combined with non-natural ingredient(s), so that the combination, composition, or formulation comprises at least one non-natural ingredient.

By way of non-limiting and merely suggestive examples, the following formulations may be prepared, and which also may be used in the methods of the invention.

In all disclosed exemplary formulation embodiments below, the formulation may solely comprise a bioactive molecule from a fungus and a bioactive molecule from a plant (where “solely comprise” refers to no additional disclosed bioactive molecules, although a formulation may further comprise one or more carriers, diluents, or excipients, other inactive ingredients, and/or in some embodiments, additional active agents as disclosed herein). In embodiments, the formulation may solely comprise a bioactive molecule from a single genus of fungus and a bioactive molecule from a single genus of plant (that is, where the molecule(s) are extracted, isolated, derived, or otherwise obtained from a single genus, and not necessarily that they are only found in one genus). In embodiments, the formulation may solely comprise a bioactive molecule from a single species of fungus and a bioactive molecule from a single species of plant (that is, where the molecule(s) are extracted, isolated, derived, or otherwise obtained from a single species, and not necessarily that they are only found in one species). In embodiments, the formulation may solely comprise one bioactive molecule from a fungus and one bioactive molecule from a plant.

In some embodiments, the formulation may solely comprise a bioactive molecule from a psilocybin-producing species, and a bioactive molecule from a Cannabis plant. In embodiments, the formulation may solely comprise a bioactive molecule from a Psilocybe azurescens, Psilocybe bohemica, Psilocybe semilanceata, Psilocybe baeocystis, Psilocybe cyanescens, Psilocybe tampanensis, Psilocybe cubensis, Psilocybe weilii, Psilocybe hoogshagenii, Psilocybe stuntzii, Psilocybe cyanofibrillosa, or Psilocybe liniformans species, and a bioactive molecule from a Cannabis plant. In embodiments, the formulation may solely comprise one bioactive molecule from a psilocybin-producing species, and one bioactive molecule from a Cannabis plant. In embodiments, the formulation may solely comprise one bioactive molecule from a Psilocybe azurescens, Psilocybe bohemica, Psilocybe semilanceata, Psilocybe baeocystis, Psilocybe cyanescens, Psilocybe tampanensis, Psilocybe cubensis, Psilocybe weilii, Psilocybe hoogshagenii, Psilocybe stuntzii, Psilocybe cyanofibrillosa, or Psilocybe liniformans species, and one bioactive molecule from a Cannabis plant. In embodiments, a formulation may solely comprise a bioactive molecule from a Psilocybe fungus, and a bioactive molecule from a Cannabis plant. A “Psilocybe fungus” refers to a Psilocybe spp. fungus. In embodiments, a formulation may solely comprise one bioactive molecule from a Psilocybe fungus, and one bioactive molecule from a Cannabis plant.

For example, each of the below embodiments may be made with psilocybin (or psilocin) only, or both; with CBD (or THC) only, or both; and with each of coumarin, whole Pyropia extract, and ethanol and ginger (and any other disclosed active or inactive ingredients) optional, i.e., in some embodiments any one or more of coumarin, whole Pyropia extract, and ethanol and a flavorant and/or colorant such as ginger (and any other disclosed active or inactive ingredients) may not be included. Accordingly, in some embodiments, the exemplary formulation solely comprises psilocybin and CBD. In embodiments, the exemplary formulation solely comprises psilocin and CBD. In embodiments, the exemplary formulation solely comprises psilocybin, psilocin, and CBD. In embodiments, the exemplary formulation solely comprises psilocybin, THC, and CBD. In embodiments, the exemplary formulation solely comprises psilocin, THC, and CBD. In embodiments, the exemplary formulation solely comprises psilocybin, psilocin, and THC. In embodiments, the exemplary formulation solely comprises psilocybin and THC. In embodiments, the exemplary formulation solely comprises psilocin and THC. As above, “solely comprises” refers to no additional disclosed bioactive molecules, although a formulation may further comprise one or more carriers, diluents, or excipients, other inactive ingredients, and/or in some embodiments, additional active agents as disclosed herein, or as otherwise appropriate to the formulation, such as known in the art.

In some embodiments, any one or more of psilocybin, psilocin, THC, and CBD may be replaced with another bioactive molecule, such as another bioactive molecule from the same fungus or the same plant. In some embodiments, a flavorant and/or colorant (equivalently and as shorthand “flavorant/colorant”) is optional. Accordingly, any of the below examples without a flavorant/colorant is an additional embodiment. In some preferred embodiments, the “flavorant/colorant” in an example is ethanol and ginger. In some embodiments, a formulation comprises an additional active agent. Where an active and/or inactive ingredient from a disclosed exemplary formulation is not present, the formulation will be prepared as described, together with general skill and knowledge in the art, and with such modifications as will be appreciated by ordinary artisans. Where an ingredient may be provided as a solid or as a liquid, one of skill will appreciate how to convert between mass amounts and volume amounts, including for ingredients, whether as a solid or a liquid, of an alternate or variable concentration.

### Example 1: Tincture Formulation

A tincture is prepared with the proportions as follows:

| | Quantity | | |
|---|---|---|---|
| Ingredient | (units) | | Extract % |
| Psilocybin | 250 | μg | 45 (30% from sonified extraction |
| Psilocin | 150 | μg | and 15% from Soxhlet extraction) |
| CBD | 1 | mg | 15 |
| THC | 1 | mg | |
| Coumarin | 1 | mg | 2 |
| Whole Pyropia extract | 8 | mg | 15 |
| Flavorant/colorant | 8 | mg | 15 |

| | |
|---|---|
| Water | 8 |

The solution is prepared by combining the bioactive molecules from a fungus, a plant, and an algae, and/or extracts comprising them (“bioactive molecules and/or extracts comprising them”) with a flavoring agent and a solvent. In embodiments, an amount or a proportion of an ingredient, such as when expressed as a mass or as a percentage, includes the term “about.” In some embodiments, “about” a mass or “about” a percentage, including where the term “about” is implied by the context, refers to a range of ±2%. Thus for example, in embodiments where a psilocybin extract is 45% of a formulation, it will be understood that the amount in embodiments refers to a psilocybin extract of “about” 45%, and in some such embodiments, an amount of “about” 45% refers to an amount of 45%±2%, i.e., between 43% and 47% psilocybin extract, inclusive. In some other embodiments, an amount, such as expressed as a mass or a percentage, will also refer to “about” that amount, with “about” having the meaning as elsewhere described.

In embodiments, a fungal extract comprises about 45% of each dose, and contains about 400 μg of combined psilocybin and psilocin per dose; a Cannabis extract comprises about 15% of each dose, and contains about 1 mg CBD and about 1 mg THC per dose; a cumaru extract comprises about 2% of each dose, and contains about 1 mg of coumarin per dose; and an algal extract, which in embodiments is a Pyropia extract, comprises about 15% of each dose; the flavorant and/or colorant, which in some preferred embodiments is ethanol infused with ginger and bay leaf, and in some embodiments is another flavoring and/or coloring agent, which may be in ethanol, water, or another diluent, comprises about 15% of each dose; and added water constitutes about 8% of each dose. In some embodiments, a tincture or other liquid formulation herein further comprises a solubilizing agent, such as a cyclodextrin, lecithin, propylene glycol, xanthan gum, or a combination thereof.

Tinctures may be prepared by exposing fungal, plant, and/or algal matter to a solvent capable of extracting the desired primary and/or secondary bioactive molecules, combining the extracts, and then optionally adding a flavorant and/or coloring agent.

In some embodiments, the solvent is alcohol. In embodiments, the alcohol may be, e.g., 40% to 60% alcohol, or may be 80% to 90% alcohol, and then diluted to between 40% and 60% alcohol. In some embodiments, the solvent is water. In some embodiments, the solvent may be an acid, for example, acetic acid.

As an example, food coloring may be added to the tincture in order to provide a certain appearance to the solution and may contain additional compounds sufficient to improve the taste of the tincture. In embodiments, the food coloring may comprise a compound which improves the biological activity of the primary and/or secondary bioactive molecules. Examples of suitable food coloring for use with disclosed extracts, compositions and tinctures include turmeric extracts, cinnamon extracts, beetroot extracts, carrot extracts, caramel, blueberry extracts, blackberry extracts, and ginger extracts.

In some embodiments, a patient will be administered, on a daily basis, one dose of a tincture having ingredients in the amounts as formulated above, two doses of a tincture in the amounts as formulated above, three such doses, four such doses, five such doses, or greater than five such doses daily. In some preferred embodiments, a daily dosage amount is four such doses.

### Example 2: Oral Spray and Oral Mucosal Spray Formulation

An oral spray may be prepared comprising the same ingredients, quantities (units) of ingredients, and overall proportions as in EXAMPLE 1. Where an oral spray is formulated for oral mucosal (e.g., sublingual or buccal) administration, it may further comprise a penetration enhancer and/or a mucoadhesive polymer.

### Example 3: Soft Mist Inhaler Formulation

A soft mist inhaler formulation is prepared as follows, comprising the same ingredients, quantities (units) of ingredients, and overall proportions as in EXAMPLE 1 above: The solution is prepared by combining the bioactive molecules from a fungus, a plant, and an algae, and/or extracts comprising them with the flavorant/colorant and the solvent.

The bioactive molecules and/or extracts comprising them are mixed and combined. The combined bioactive molecules and/or extracts comprising them may then be combined with ethanol and/or water, and an optional added preservative, for example benzalkonium chloride, to a volume of, for example, 15 mL. Reference may be made to Anderson, 2006 and Dalby, Eicher, and Zierenberg, 2011, both of which are incorporated by reference herein in their entirety.

### Example 4: Vaporizer Formulation

A vaporizer formulation, comprising 500 μg psilocybin, 300 μg psilocin, 2 mg CBD, 2 mg THC, 2 mg coumarin, 16 mg whole Pyropia extract, 16 mg flavorant/colorant, and 10 mL 50:50 base liquid (50% PG, 50% VG), is prepared as follows: The bioactive molecules and/or extracts comprising them are mixed and combined (in solid form, and/or as part of one or more liquid extracts) with the 50:50 base liquid prepared for use with any liquid vaporization device or appliance, such as e-liquid vaporizers, e-cigs, mods, vape pens, and the like. A flavorant/colorant (e.g., comprising ginger and/or bay laurel) optionally may be added. The formulation also can be prepared for any oil, thin oil, e-juice, or e-liquid vaporizer, according to ordinary skill.

### Example 5: Tablet and Scorable Double-Strength Tablet Formulation

Tablets comprising 500 μg psilocybin, 300 μg psilocin, 1 mg CBD, 1 mg THC, 1 mg coumarin, 16 mg whole Pyropia extract, 170 mg microcrystalline cellulose, 10 mg colloidal silicon dioxide, and 7.5 mg stearic acid are prepared by blending the bioactive molecules and/or extracts comprising them together with the other ingredients, and compressing it to form tablets.

Scorable double strength tablets comprising 1000 μg psilocybin, 600 μg psilocin, 2 mg CBD, 2 mg THC, 2 mg coumarin, 32 mg whole Pyropia extract, 35 mg microcrystalline cellulose, 45 mg starch, 4.5 mg sodium carboxymethyl starch, 0.5 mg magnesium stearate, 1 mg talc, and 4 mg polyvinylpyrrolidone (PVP) (as 10% solution in water), are prepared as follows: The bioactive molecules and/or extracts comprising them, starch, and cellulose are passed through a No. 20 mesh U.S. sieve and mixed thoroughly. The solution of PVP is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets. Tablets are scored to provide the ability to create equal half doses.

### Example 6: Capsule Formulation

Capsules, each comprising 500 μg psilocybin, 300 μg psilocin, 2 mg CBD, 2 mg THC, 2 mg coumarin, 16 mg whole Pyropia extract, 119 mg cellulose and/or starch, and 1 mg magnesium stearate are made by blending the bioactive molecules and/or extracts comprising them, together with the cellulose and/or starch and magnesium stearate, passing them through a No. 20 mesh U.S. sieve, and filling the blended mixture into hard or soft gelatin capsules.

### Example 7: Alternate Capsule Formulation, Optionally with Additional Active Agent(s)

Capsules, each comprising 1000 μg psilocybin, 600 μg psilocin, 2 mg CBD, 2 mg THC, 2 mg coumarin, 16 mg whole Pyropia extract, 100 mg cellulose and/or starch, and 1 mg magnesium stearate are made as follows: The bioactive molecules and/or extracts comprising them, cellulose and/or starch, and magnesium stearate are blended, passed through a No. 20 mesh U.S. sieve, and filled into hard or soft gelatin capsules. Optionally, an additional active agent may be added during blending. In some embodiments, the additional active agent is a serotonergic agent, such as an antidepressant or an anxiolytic. In some embodiments, the additional active agent is an antiparkinson agent, such as levodopa, amantadine, a dopaminergic antiparkinsonism agent (e.g., a dopamine agonist), an anticholinergic antiparkinson agent, or another antiparkinson agent, such as an adenosine A2A antagonist, a COMT inhibitor, a MAO-B inhibitor, or another such antiparkinson agent as described herein or generally known in the art.

### Example 8: Suspension Formulation

A suspension, comprising 250 μg psilocybin, 150 μg psilocin, 1 mg CBD, 1 mg THC, 1 mg coumarin, 8 mg whole Pyropia extract, and 25-50 mg levodopa is made as follows: The bioactive molecules and/or extracts comprising them are blended together with 1.75 g sucrose and 4 mg xanthan gum, passed through a No. 10 mesh U.S. sieve, and then mixed with a previously made solution of 50 mg sodium carboxymethyl cellulose (11%) and 50 mg microcrystalline cellulose (89%) in water. Ten mg sodium benzoate and optional flavorant/colorant to taste (quod vide), are diluted with some of the water and added with stirring. Levodopa may be added at 5-10 mg/mL. Sufficient water is then added to produce 5 mL.

### Example 9: Intravenous Solution Formulation

An intravenous formulation, comprising 250 μg psilocybin, 150 μg psilocin, 1 mg CBD, 1 mg THC, 1 mg coumarin, 8 mg whole Pyropia extract, and 1,000 mL isotonic saline, may be prepared as follows: The bioactive molecules and/or extracts comprising them are dissolved in appropriate solvent, such as isotonic saline or another suitable solvent; additional active or inactive ingredients such as solubilizers and preservatives may be added, as otherwise described above, and within the general knowledge of the art. It will be understood that the amount of each bioactive molecule and/or extract can be adjusted accordingly to reach the desired mg/mL.

### Example 10: Injectable Solution Formulation

An injectable formulation, comprising 250 μg psilocybin, 150 μg psilocin, 1 mg CBD, 1 mg THC, 1 mg coumarin, 8 mg whole Pyropia extract, and 5 mL isotonic saline, may be prepared as follows: The bioactive molecules and/or extracts comprising them are dissolved in appropriate solvent, such as isotonic saline or another suitable solvent; additional active or inactive ingredients such as preservatives may be added, as otherwise described above, and within the knowledge in the art. It will be understood that the amount of each bioactive molecule and/or extract can be adjusted accordingly to reach the desired mg/mL.

### Example 11: Topical Formulation for Transdermal Administration

A topical formulation, comprising 400 μg psilocybin, 250 μg psilocin, 2 mg CBD, 2 mg THC, 2 mg coumarin, 8 mg whole Pyropia extract, 30 mg emulsifying wax, 20 mg liquid paraffin, in an amount of white soft paraffin to equal 100 g total, may be prepared as follows: The white soft paraffin is heated until molten. The liquid paraffin and emulsifying wax are incorporated and stirred until dissolved. The bioactive molecules and/or extracts comprising them are added and stirring is continued until dispersed. The mixture is then cooled until solid.

### Example 12: Cut Matrix Sublingual or Buccal Tablet Formulation

Sublingual or buccal tablets, comprising 500 μg psilocybin, 300 μg psilocin, 2 mg CBD, 2 mg THC, 2 mg coumarin, 8 mg whole Pyropia extract, 210.5 mg glycerol, 143 mg water, 4.5 mg sodium citrate, 26.5 mg polyvinyl alcohol, and 15.5 mg polyvinylpyrrolidone (PVP), are made as follows: The glycerol, water, sodium citrate, polyvinyl alcohol, and PVP are admixed by continuous stirring while maintaining a temperature of about 90° C. When the polymers have gone into solution, the solution is cooled to about 50-55° C. and the bioactive molecules and/or extracts comprising them are slowly admixed. The homogenous mixture is poured into forms made of an inert material to produce a drug-containing diffusion matrix having a thickness of about 2-4 mm. The diffusion matrix is then cut to form individual tablets of the appropriate size.

### Example 13: Individually Formed Sublingual or Buccal Lozenge Formulation

Sublingual or buccal lozenges, comprising 500 μg psilocybin, 300 μg psilocin, 2 mg CBD, 2 mg THC, 2 mg coumarin, 8 mg whole Pyropia extract, 350 mg silica gel powder, 400 mg citric acid powder, 600 mg acacia powder, 1 g polyethylene glycol (PEG), and optionally 100 mg flavorant/colorant, are made as follows: The silica gel powder, citric acid powder, acacia powder, optional flavorant/colorant, and PEG are mixed by continuous stirring at a temperature of about 90° C. When the PEG has melted and the other ingredients have gone into solution, the solution is cooled to about 50-55° C. and the bioactive molecules and/or extracts comprising them are slowly admixed. The homogenous mixture is poured into separate molds and allowed to cool. Reference may also be made to U.S. Pat. No. 10,034,832 and the Examples therein.

### Example 14: Intranasal Delivery Formulation

A nasal spray formulation for intranasal delivery, comprising 250 μg psilocybin, 150 μg psilocin, 1 mg CBD, 1 mg THC, 1 mg coumarin, 8 mg whole Pyropia extract, 50 μL DMSO, 5 mL MCT, in saline (1% cremaphor) to a total formulation of 10 mL, may be prepared as follows: The solution at approximately 1 mg/mL of bioactive molecules and/or extracts comprising them in 49.5% MCT, 49.5% saline, .5% DMSO, and .5% cremaphor is prepared for use in nasal spray device. In other embodiments, a nasal formulation can be prepared as a dry powder for inhalation, e.g., by combining the bioactive molecules and/or extracts comprising them with lactose and mixing for use with a dry powder inhaling appliance, or as in U.S. Pub. No. US2015/0367091A1 and references cited therein.

In some exemplary embodiments, a disclosed therapeutic combination or pharmaceutical composition comprises the primary bioactive molecules in the table below, from each listed component of the combination or composition, and such combination or composition may be formulated according to any of the Examples herein, or formulated in many other exemplary formulations according to this disclosure and the general knowledge in the art:

| Component | Cannabis sativa | |
|---|---|---|
| Primary bioactive(s) | D9-THC | Cannabidiol |
| Chemical structure | 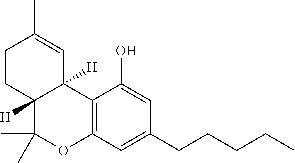 | 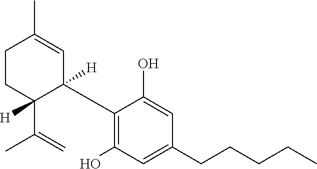 [SMILES: C=C(C)[C@@H]1CCC(C)=C[C@H]1c1c(O)cc(CCCCC)cc1O] |
| Molecular Formula | C₂₁H₃₀O₂ | C₂₁H₃₀O₂ |
| Molecular weight | 314.469 | 314.464 |
| (g/mol) | | |

| | |
|---|---|
| Exemplary Source | National Institute on Drug Abuse, National Institute of Health. |

| Component | Psilocybe cubensis | |
|---|---|---|
| Primary bioactive(s) | Psilocin | Psilocybin |
| Chemical structure | 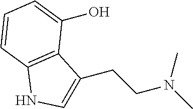 | 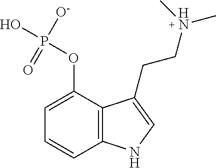 |
| Molecular Formula | C₁₂H₁₆N₂O | C₁₂H₁₇N₂O₄P |
| Molecular weight | 204.27 | 284.25 |
| (g/mol) | | |

| | |
|---|---|
| Exemplary Source | Numinus Wellness |
| | (801-33 Water Street Vancouver, BC V6B 1R4). |

| Component | Dipteryx odorata | Pyropia yezoensis |
|---|---|---|
| Primary bioactive(s) | Coumarin | Porphyran |
| Chemical structure | 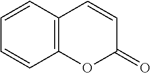 [SMILES: O=c1ccc2ccccc2o1] | 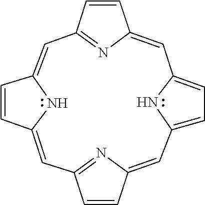 [SMILES: C1=Cc2cc3ccc(cc4nc(cc5ccc(cc1n2)[nH]5)C=C4)[nH]3] |
| Molecular Formula | C₉H₅O₂ | C₂₀H₁₄N₄ |
| Molecular weight | 146.14 | 310.4 |
| (g/mol) | | |
| Exemplary Source | Sunrise Botanicals | Mountain Rose Herbs |
| | (7215 Goreway Dr, Mississauga, | (106 Cleveland St, Eugene OR |
| | ON L4T 0B4) | 97402) |

Note, while certain primary and/or secondary bioactive molecules, as well as certain extracts comprising them, are disclosed above, this should not be construed as limiting the invention, nor should it be construed as limiting the formulation of EXAMPLE 1 to the primary and/or secondary bioactive molecules disclosed above. Thus, in embodiments, the formulation of EXAMPLE 1 (or any other Example herein) contains additional, or fewer, primary and/or secondary bioactive molecules than are disclosed; including any of the primary and/or secondary bioactive molecules disclosed herein for each of the fungal, Cannabis, cumaru, and algal extracts.

In some alternative embodiments of this Example, the formulation comprises at least one bioactive molecule from a fungus, and at least one bioactive molecule from a plant, the bioactive molecule(s) from fungi comprising those from a psilocybin-producing species, such as from the genera Copelandia, Galerina, Gymnopilus, Inocybe, Panaeolus, Pholiotina, and Pluteus, and the bioactive molecule(s) from plant(s) comprising those from a cannabinoid-producing species, such as from the genus Cannabis. In some such alternative embodiments, the formulation comprises no further bioactive molecule(s) from fungi and bioactive molecule(s) from plant(s) except for those from a psilocybin-comprising species and from a cannabinoid-producing species. In some such alternative embodiments, the formulation comprises no further bioactive molecule(s) from fungi and bioactive molecule(s) from plant(s) except for those from the genera Copelandia, Galerina, Gymnopilus, Inocybe, Panaeolus, Pholiotina, and Pluteus, and from the genus Cannabis. In some such alternative embodiments, the formulation comprises no bioactive molecules from the genus Dipteryx. In some such alternative embodiments, the formulation comprises no bioactive molecules from algae, a marine algae, the family Bangiaceae, or the genera Pyropia and Porphyra. In some such alternative embodiments, the only bioactive molecules are psilocybin, psilocin, CBD, and THC. In some such alternative embodiments, the only bioactive molecules are psilocybin, CBD, and THC. In some such alternative embodiments, the only bioactive molecules are psilocybin and CBD. In some such alternative embodiments, the only bioactive molecules are psilocybin and THC. In some such alternative embodiments, the only bioactive molecules are psilocin, CBD, and THC. In some such alternative embodiments, the only bioactive molecules are psilocin and CBD. In some such alternative embodiments, the only bioactive molecules are psilocin and THC.

It will be readily appreciated that the above formulation examples are illustrative only. Any of the primary and/or secondary bioactive molecules disclosed herein may be utilized in the formulation examples above, and in a dosage range applicable for said primary and/or secondary bioactive molecules. It will be understood that reference to a particular primary and/or secondary bioactive molecule is merely illustrative, and bioactive molecules in any Example may be substituted by other primary and/or secondary bioactive molecules disclosed herein.

Moreover, it will be readily appreciated that the disclosed compositions are not limited to combinations of a single primary and/or secondary bioactive molecule, or (when formulated as a pharmaceutical composition) limited to a single carrier, diluent, and/or excipient alone, but may also include combinations of multiple primary and/or secondary bioactive molecules (including additional bioactive molecules), and/or multiple carriers, diluents, and excipients. Pharmaceutical compositions of this invention thus may comprise any of the primary and/or secondary bioactive molecules disclosed above. Meaning, in embodiments, the disclosed composition comprises a primary and/or secondary bioactive molecule from a fungus, a plant, and an algae, optionally together with one or more other bioactive molecules (or their derivatives and analogs) in combination, together with one or more pharmaceutically-acceptable carriers, diluents, and/or excipients, and additionally with one or more other active agents.

In embodiments, a disclosed composition will be prepared so as to increase an existing therapeutic effect, provide an additional therapeutic effect, increase adherence or ease-of-use, increase a desired property such as stability or shelf-life, decrease an unwanted effect or property, alter a property in a desirable way (such as pharmacokinetics or pharmacodynamics), modulate a desired system or pathway (e.g., a neurotransmitter system), or provide synergistic effects.

“Therapeutic effects” that may be increased or added in embodiments of the invention include, but are not limited to, antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, nootropic, empathogenic, psychedelic, sedative, or stimulant effects, as well as such effects as are known to ameliorate symptoms caused by a movement disorder.

The goal of increasing an existing therapeutic effect, providing an additional therapeutic effect, increasing adherence or ease-of-use, increasing a desired property such as stability or shelf-life, decreasing an unwanted effect or property, altering a property in a desirable way (such as pharmacokinetics or pharmacodynamics), modulating a desired system or pathway (e.g, a neurotransmitter system), or otherwise inducing synergy, in embodiments, is achieved by the inclusion of an additional active agent.

The type of formulation employed for the administration of the disclosed primary and/or secondary bioactive molecules employed in the disclosed methods generally may be dictated by the primary and/or secondary bioactive molecule(s) employed, the type of pharmacokinetic profile desired from the route of administration and the primary and/or secondary bioactive molecule(s), and the state of the patient. It will be readily appreciated that any of the above embodiments and classes of embodiments can be combined to form additional embodiments.

a. Routes of Administration

The disclosed pharmaceutical compositions are suitable for administration by a variety of routes. Non-limiting examples of routes of administration include enteral administration, such as oral, sublingual, buccal, and rectal administration, parenteral administration, including bolus injection or continuous infusion, intravenous, intra-arterial, intraperitoneal, intraosseous, intramuscular, intrathecal, intracerebroventricular, vaginal, ocular, nasal, cutaneous, topical, otic, ocular, transdermal, and subcutaneous administration.

In embodiments, a pharmaceutical composition is administered as oral solid and oral liquid dosage forms; sublingually or buccally; as injections, including intravenous, intra-arterial, intraperitoneal, intraosseous, intramuscular, intrathecal, and intracerebroventricular; rectally, vaginally, ocularly, nasally, cutaneously, topically, oticly, transdermally, and subcutaneously.

In embodiments, in which administration is enteral, parenteral, or both, an effective amount of a primary and/or secondary bioactive molecule is systemically administered to a subject. In embodiments, an effective amount of a primary and/or secondary bioactive molecule is administered orally to a subject. In embodiments, an effective amount of a primary and/or secondary bioactive molecule is intravenously administered to a subject. In embodiments, an effective amount of a primary and/or secondary bioactive molecule is administered by inhalation to a subject. In embodiments, an effective amount of a primary and/or secondary bioactive molecule is administered by nasal administration to a subject. In embodiments, an effective amount of a primary and/or secondary bioactive molecule is administered by injection to a subject. In embodiments, an effective amount of a primary and/or secondary bioactive molecule is administered topically (dermally) to a subject. In embodiments, an effective amount of a primary and/or secondary bioactive molecule is administered by ophthalmic administration to a subject. In embodiments, an effective amount of a primary and/or secondary bioactive molecule is administered rectally to a subject. In embodiments, the primary and/or secondary bioactive molecules disclosed herein and employed in the methods described herein are effectively administered to a subject via other means, and prepared as any acceptable composition known to those of skill. In embodiments, such compositions may be prepared in any manner known in the pharmaceutical arts that comprise at least one bioactive molecule (Sheth, 1980).

In embodiments, the primary and/or secondary bioactive molecules disclosed herein are administered by multiple routes, which may differ between subjects, such as a patient, according to subject preferences, comorbidities, side effect profiles, pharmacokinetic and pharmacodynamic considerations, and other factors. In embodiments are the presence of other substances with the primary and/or secondary bioactive molecules, known to those skilled in the art, such as modifications in the preparation to facilitate absorption through various routes (e.g., gastrointestinal, transdermal, etc.), to extend the effect of the drugs, and/or attain higher or more stable serum levels or enhance the therapeutic effect of the primary and/or secondary bioactive molecules disclosed herein.

In embodiments, the pharmaceutical compositions are suitable as oral solid or oral liquid dosage forms, administered sublingually, buccally, rectally, vaginally, ocularly, oticly, nasally, cutaneously, topically, and transdermally; or as intravenous, intra-arterial, intraperitoneal, intraosseous, intramuscular, intrathecal, intracerebroventricular, and subcutaneous injection, wherein such injections comprise physiologically acceptable sterile aqueous or non-aqueous solutions, dispersions, suspensions or emulsions, liposomes, and sterile powders for reconstitution into sterile injectable solutions or dispersions.

In embodiments, a pharmaceutical composition may be administered via enteral or parenteral means, wherein enteral means includes, but is not limited to, oral solid and oral liquid dosage forms, sublingual and buccal administration, and rectal administration; and parenteral administration means includes, but is not limited to, bolus injection or continuous infusion, intravenous, intra-arterial, intraperitoneal, intraosseous, intramuscular, intrathecal, intracerebroventricular, vaginal, ocular, nasal, cutaneous, topical, otic, transdermal, and subcutaneous administration; in addition to other equivalent means known to those of skill.

Enteral administration includes administration involving any part of the gastrointestinal tract. Non-limiting examples include those by mouth (orally), including oral solid and oral liquid dosage forms, and rectal, and in some embodiments may be preferably formulated as tinctures. Parenteral administration refers to administration from any means not involving the gastrointestinal tract, including intravenous (into a vein), intra-arterial (into an artery), intraosseous infusion (into bone marrow), intramuscular (into a muscle), intracerebral (into a brain parenchyma), intracerebroventricular (into a cerebral ventricular system), intrathecal (an injection into a spinal canal), otic (through an ear), ocular (through an eye), vaginal (into a vagina), and subcutaneous (under skin). In embodiments, parenteral administration may include sublingual and/or buccal administration. In embodiments, a pharmaceutical composition may be administered to a subject via injection. In embodiments, a pharmaceutical composition may be administered to a subject via nasal systems or the mouth through, for example, an oral solid and/or oral liquid dosage forms; inhalation, via a nasal spray or oral inhaler; nebulization, from a nebulizer, such as a machine that turns liquid medicine into a mist; or buccally/sublingually. In embodiments, a pharmaceutical composition may be administered to a subject via a combination of administration means. In embodiments, the pharmaceutical composition may be administered to a subject via an enteral administration means. In embodiments, the pharmaceutical composition may be administered to a subject via a parenteral administration means. In embodiments, the composition may be administered to a subject via at least one enteral administration means, and at least one parenteral administration means. In embodiments, an equivalent route of administration known to one of skill is utilized.

In some embodiments, a therapeutic combination may be administered via multiple routes of administration. For example, In embodiments, the fungal portion of a disclosed combination is administered via one route of administration, and the plant portion(s) and algal portion are administered via a different route of administration. In embodiments, the plant portion(s) of a disclosed combination is/are administered via one route of administration, and the fungal and algal portions are administered via a different route of administration. In embodiments, the algal portion of a disclosed combination is administered via one route of administration, and the plant portion(s) and fungal portion are administered via a different route of administration.

Administration of different (fungal, plant, or algal) portions of a disclosed combination to different tissues may maximize the therapeutic effects of the combination, result in reduction of side effects, reduction of patient discomfort, and/or increase of bioavailability. In one illustrative example, a disclosed combination comprises a plant portion comprising a cannabinoid (e.g., THC) or a Cannabis extract, which is administered to the oral mucosa via spray; while remaining ingredients (e.g., fungal, plant, and/or algal portions) are delivered to epithelial cells in the lung via a soft-mist inhaler. In some embodiments, administration according to this exemplary procedure results in reduced throat irritation (e.g., in individuals sensitive to inhaling Cannabis or cannabinoids), maximizes bioavailability for the remaining ingredients (e.g., fungal, plant, and/or algal portions), and improves the pharmacokinetic profiles of the components of the therapeutic combination; for example by expediting the uptake of the longer-lasting plant portion (e.g., the THC or Cannabis extract) while moderating the uptake of shorter-lasting portions.

In another example, a disclosed combination comprises an algal portion comprising Pyropia or a Pyropia extract, which is administered to the oral mucosa via oral spray; while remaining ingredients (e.g., fungal, plant, and/or algal portions) are delivered to epithelial cells in the lungs via soft-mist inhaler. In some embodiments, administration according to this exemplary procedure results in reduced throat irritation, maximizes bioavailability of ingredients (e.g., fungal, plant, and/or algal portions), and improves the pharmacokinetic profiles of the components of the therapeutic combination; for example by expediting the uptake of the long-lasting portions while moderating the uptake of the shorter-lasting algal portion (e.g., the Pyropia or Pyropia extract).

### B. Methods of Administration

In some aspects, provided are methods of administration or methods of administering a primary and/or secondary bioactive molecule disclosed herein. As used herein, the terms “subject,” “user,” “patient,” and “individual” are used interchangeably, and refer to any mammal, preferably a human. Such terms will be understood to include one who has an indication for which the combinations, compositions, or methods described herein may be efficacious, or who otherwise may benefit by the invention. In general, all of the combinations, compositions, and methods of the invention will be appreciated to work for all individuals, although individual variation is to be expected, and will be understood.

The invention provides methods for using therapeutically effective amounts of the pharmaceutical compositions of the invention containing the primary and/or secondary bioactive molecules disclosed herein in a mammal, and preferably a human. Such methods include those for treating a movement disorder, including in a healthy individual.

Administration of pharmaceutical compositions in an “effective amount,” a “therapeutically effective amount,” a “therapeutically effective dose,” or a “pharmacologically effective amount,” refers to an amount of a primary and/or secondary bioactive molecule that is sufficient to provide the desired therapeutic effect, for example, relieving to some extent one or more of the symptoms of the disease or condition being treated. The result can be reduction and/or alleviation of the signs, symptoms, or causes of a disease or disorder, or any other desired alteration of a biological system. “Therapeutically effective amount” includes, for example, a prophylactically effective amount.

An “effective amount” of a primary and/or secondary bioactive molecule disclosed herein is an amount effective to achieve a desired pharmacologic effect or meaningful therapeutic improvement. It is understood that “an effective amount” or “a therapeutically effective amount” can vary from subject to subject due to variation in metabolism of a compound, such as the primary and/or secondary bioactive molecules disclosed herein, of age, weight, general condition of the subject, the condition being treated, the severity of the condition being treated, and the judgment of the prescribing physician. The effective amount will vary depending upon the subject and the disease condition being treated or health benefit sought, the weight and age of the subject, the severity of the disease condition or degree of health benefit sought, the manner of administration, and the like, all of which can readily be determined by one of skill. Additionally, pharmacogenomic (the effect of genotype on the pharmacokinetic, pharmacodynamic or efficacy profile of a therapeutic) information about a particular patient may affect dosage used.

As used herein, “therapeutic effect” or “therapeutic efficacy” means the responses(s) in a mammal, and preferably a human, after treatment that is judged to be desirable and beneficial. Hence, depending on the disorder to be treated, or improvement in physiological or psychological functioning sought, and depending on the particular constituent(s) in the compositions of the invention under consideration, those responses shall differ, but would be readily understood by those of skill. For example, in embodiments, “therapeutic effect” may refer to an effect caused by the disclosed composition, or its use in a method of the invention, such as the treatment of a movement disorder, as disclosed herein.

“Therapeutically effective dose” refers to the dose necessary to elicit a desired result within a patient undergoing treatment. A therapeutically effective dose therefore may, in embodiments, refer to a dose of the pharmaceutical composition or therapeutic combination necessary to deliver measurable patient-specific biologic effects in the treatment or prevention of a condition or disorder. A “therapeutically effective dose” may be used interchangeably with a “therapeutically effective amount” or an “effective amount.”

### H. Dosing

It will be readily appreciated that dosages may vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender, and race of the subject, bioavailability, potential adverse systemic, regional or local side effects, the presence of other disorders or diseases in the subject, and other factors that will be appreciated by the skilled artisan (e.g., medical or familial history).

In embodiments, wherein the combination and/or composition comprises a primary and/or secondary bioactive molecule from a fungus, the primary and/or secondary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In all embodiments herein that include dose amounts of less than about 1 mg, such dose amounts will be understood to include further specific dose amounts of about 0.5 mg or less, about 0.25 mg or less, about 0.1 mg or less, about 0.05 mg or less, about 0.005 mg or less, about 0.001 mg or less, and about 0.0005 mg or less.

In all embodiments herein that include dose amounts of at least about 1 mg or more, up to and including about 75 mg, such dose amounts will be understood to include further specific dose amounts of (with all such milligram dose amounts to be understood also to be preceded by the modifier “about”) 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, as well as amounts within these ranges.

In all embodiments herein that include dose amounts of 75 mg, as well as greater than 75 mg, including 100 mg, 150 mg, 200 mg, or greater than 200 mg, such dose amounts will be understood to include further specific dose amounts of 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, and 200 mg. Such dose amounts additionally will be understood to include amounts within these ranges, and in all such embodiments, a single dose moreover may be greater than 200 mg, including 225 mg, 250 mg, or greater than 250 mg.

In embodiments, wherein the combination and/or composition comprises a primary and/or secondary bioactive molecule from a fungus, the primary and/or secondary bioactive molecule may be present in an amount so that a single dose is (whether or not such dose is present in a unit dosage form), about 100 μg or less, (including 95 μg, 90 μg, 85 μg, 80 μg, 75 μg, 70 μg, 65 μg, 60 μg, 55 μg, 50 μg, 45 μg, 40 μg, 35 μg, 30 μg, 25 μg, 20 μg, 15 μg, 10 μg, 5 μg, and 5 μg or less), or at least about 100 μg, or more, and greater than 1,000 μg, including 1,500 μg, 2,000 μg, up to and including 5,000 μg, and in some embodiments, greater than 5,000 μg.

In all embodiments herein that include dose amounts of about 100 μg or less, such embodiments will be understood to include further specific dose amounts including about 95 μg, 90 μg, 85 μg, 80 μg, 75 μg, 70 μg, 65 μg, 60 μg, 55 μg, 50 μg, 45 μg, 40 μg, 35 μg, 30 μg, 25 μg, 20 μg, 15 μg, 10 μg, 5 μg, and about 5 μg or less.

In all embodiments herein that include dose amounts of at least about 100 μg, or more, and greater than 1,000 μg, including 1,500 μg, 2,000 μg, up to and including 5,000 μg, such embodiments will be understood to include further specific dose amounts including 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 680 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, and 1000 μg, as well as in some embodiments, greater than 5,000 μg.

In some embodiments, wherein the primary bioactive molecule from a fungus is any of psilocybin, psilocin, baeocystin, norbaeocystin, norpsilocin, aeruginascin, or a β-carboline, it may be present in an amount so that a single dose is (whether or not such dose is present in a unit dosage form), about 100 μg or less, at least about 100 μg, or more, and greater than 1,000 μg, including 1,500 μg, 2,000 μg, up to and including 5,000 μg.

In embodiments, wherein the secondary bioactive molecule from a fungus is a polysaccharide, a peptide, a terpene or terpenoid, a phenolic compound, a mineral, a vitamin, an amino acid, a lipid, choline, or a lactone, it may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In embodiments, wherein the combination and/or composition comprises a primary and/or secondary bioactive molecule from a plant, the primary and/or secondary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

For example, in some embodiments, wherein the primary bioactive molecule from a plant is a cannabinoid, THC, CBD, a flavone or a flavonoid, a terpene or a terpenoid, a carbohydrate, a fatty acid or fatty acid ester (FAE), an amide, an amine, a phytosterol, a phenolic compound, coumarin, a compound derived from coumarin, i.e., a coumarin derivative (e.g., phenylpropanoids, coumarins, or coumarinoids), cumaru, an isoflavone, a lupeol derivative, a fatty acid ester, (±)-balanophonin, (−)-lariciresinol, 3′-hydroxyretusin-8-methyl-ether, 5-methoxyxanthocercin A, 6,4′-dihydroxy-3′-methoxyaurone, 7-hydroxychromone, 7,3′-dihydroxy-8,4′-dimethoxyisoflavone, betulin, butin, coumaric-acid-beta-glucoside, dipteryxin, dipteryxic acid, eriodictyol, ferulic-acid, isoliquiritigenin, lupeol, melilotoside, melilotoside-1-p-coumaryl-beta-d-glucose, methyl-linolenate, methyl-oleate, O-coumaricacid, O-hydroxycoumaric-acid, odoratin, P-hydroxy-benzoic-acid, retusin, retusin-8-methyl-ether, sulfuretin, salicylic-acid, afrormisin, castinin, linoleic acid, oleic acid, 3′,4′, 7′-trihydroxyflavone, luteolin, and umbelliferone, it may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In embodiments, wherein the combination and/or composition comprises a primary and/or secondary bioactive molecule from an algae, the primary and/or secondary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

For example, in some embodiments, wherein the primary bioactive molecule from an algae is porphyran or oligo-porphyran, a polysaccharide, an oligo-polysaccharide, a monosaccharide, a peptide, a phycobiliprotein, a mycosporine-like amico acid, an essential amino acid, a nonessential amino acid, a carotene or an intermediate carotenoid, a glycoprotein, an amino sulfonic acid (such as taurine), a mineral, a vitamin, a lipid, a phenolic compound, or a phlorotannin, it may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In some embodiments, a disclosed therapeutic combination (i.e., a disclosed therapeutic combination comprising, inter alia, a bioactive molecule from a fungus and a bioactive molecule from a plant) is administered a total daily dose of between about 50 mg and 1 g. In embodiments, the total daily dose is between about 50 mg and 1 g, 100 mg and 500 mg, and preferably 100 mg and 400 mg. In embodiments, each individual dose of a disclosed therapeutic combination is between 5 mg and 500 mg, or 10 mg and 400 mg, or preferably between 10 mg and 100 mg.

In embodiments, a patient will be administered a therapeutically effective dose of a primary and/or secondary bioactive molecule on a regular or chronic basis, wherein the patient is administered the primary and/or secondary bioactive molecule daily, several times per day (at least one, at least two, at least three, at least four, or greater than four times per day); on a set, repeating schedule, wherein the patient is administered a therapeutically effective dose of the primary and/or secondary bioactive molecule every other day, every three days, every four days, every five days, every six days, every seven days, or more than every seven days; or, in some embodiments, a varying schedule comprised of a plurality of days “on” (wherein the therapeutically effective dose of the primary and/or secondary bioactive molecule is administered), and a plurality of days “off” (wherein no administration occurs), such as one day on two days off, two days on three days off, three days on four days off, or other such schedules as would be apparent to those of skill (see, e.g., what are known as the Paul Stamets or James Fadiman microdosing protocols).

It will be understood that, in embodiments, the dose actually administered will be determined by a physician, in light of the relevant circumstances, the method of delivery, the age of the patient, the weight of the patient, whether the patient has any comorbidities, other medications the patient is taking (routinely or presently), and any patient-specific aspects that could affect the way in which the primary and/or secondary bioactive molecules interact with the patient, such as variations in metabolism, variations in patient response, etc., and therefore any dosage ranges disclosed herein are not intended to limit the scope of the invention. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In such embodiments, the primary and/or secondary bioactive molecules may be administered and dosed in accordance with good medical practice, taking into account the method and scheduling of administration, prior and concomitant medications and medical supplements, the clinical condition of the individual patient and the severity of the underlying disease, the patient's age, sex, body weight, tolerance, and other such factors relevant to medical practitioners, and knowledge of the particular compound(s) used. Dosage levels may differ from patient to patient, for individual patients across time, and for different combinations and formulations, but shall be able to be determined with ordinary skill. Determination of appropriate dosing shall include not only the determination of single dosage amounts, but also the routes of dose administration, determination of the number and timing of doses, and the time(s) of day or time(s) during a psychotherapeutic session preferable for their administration.

In embodiments, a patient may be on a dosing schedule as described above, but may administer the dose to themselves. In embodiments, the combination and/or disclosed composition may be prescribed to a patient, wherein the patient obtains a therapeutically effective dose from a pharmacy or healthcare provider.

Dose amount, frequency, or duration may be increased or reduced, as indicated by the clinical outcome desired, status of the pathology or symptom, any adverse side effects of the treatment or therapy, or concomitant medications. Concentrations or ratios of concentrations of components of disclosed combinations and compositions also may be altered as indicated by the clinical outcome desired, status of the pathology or symptom, any adverse side effects of the treatment or therapy, or concomitant medications. For example, in some embodiments, a disclosed combination comprises a tryptamine (such as, psilocin and/or psilocybin, e.g., as a component of a fungal portion, such as a fungal extract from a psilocin- and/or psilocybin-containing fungus). In some such embodiments, a patient taking a monoamine oxidase inhibitor (MAOI) medication may be administered a composition with a reduced concentration of the tryptamine (e.g., the psilocin and/or psilocybin), as the MAOI would be expected to reduce the metabolic excretion of the tryptamine, thereby affecting the ratios and synergistic effects of bioactive molecules upon administration of the disclosed composition. In another example, the percentage of a component of a disclosed composition may be increased or decreased according to individual tolerance. For example, individuals who frequently consume Cannabis products often develop a tolerance to the effects of cannabinoids. Hence, in some embodiments, an individual with appreciable tolerance for Cannabis or a cannabinoid (e.g., THC) may be administered a composition with an increased concentration of THC to account for such tolerance. In yet another example, individuals with certain medical conditions may be particularly sensitive or tolerant to the effects of a disclosed composition. By way of example only, in some embodiments, an individual with Lewy Body Dementia (LBD) may be administered a disclosed composition comprising a reduced concentration of a cannabinoid (e.g., THC) or a Cannabis extract, for example, if it is reasonably suspected (e.g., per the discretion of a physician or another medical practitioner) that the cannabinoid or Cannabis extract could aggravate cognitive or psychiatric symptoms of LBD, and/or increase the patient's sensitivity to the composition or any of its constituents. In another merely illustrative example, an individual with clinical depression may be administered a disclosed composition with an increased concentration of a tryptamine (such as, psilocin and/or psilocybin, e.g., as a component of a fungal portion, such as a fungal extract from a psilocin- and/or psilocybin-containing fungus), which may provide clinical advantages, such as promoting serotonin regulation, neuroplasticity, and connectivity.

The skilled artisan with the benefit of this disclosure will appreciate the factors that may influence the dosage, frequency, and timing required to provide an amount sufficient or effective for providing a therapeutic effect or benefit, and to do so depending on the type of therapeutic effect desired, as well as to avoid or minimize adverse effects.

In other embodiments, appropriate dosages to achieve a therapeutic effect, including the upper and lower bounds of any dose ranges, can be determined by an individual, including an individual who is not a clinician, by reference to available public information and knowledge, and reference to subjective considerations regarding desired outcomes and effects.

In embodiments, rather than formulated as a single composition, the bioactive molecules in a therapeutic combination are administered to a patient separately, sequentially, or simultaneously. In embodiments, the bioactive molecules in a therapeutic combination can be individually formulated as pharmaceutical compositions that are then administered to a patient separately, sequentially, or simultaneously.

In embodiments, sequential administration refers to administration of one combination drug immediately following administration of another combination drug (e.g., within about 5 minutes of the administration of the first combination drug), simultaneous administration refers to administration of each combination drug at substantially the same time, while separate administration refers to administration with time elapsing between the administrations. In embodiments wherein the combination drugs are administered separately, such administration may include elapsed time between the each administration of between about 5 minutes to about 30 minutes, about 10 minutes to about 60 minutes, about 30 minutes to about 180 minutes, about 180 minutes to about 360 minutes, or more than 360 minutes, such as 8 hours, 12 hours, 16 hours, 20 hours, 24 hours, 36 hours, 48 hours, 72 hours, 5 days, 7 days, 10 days, 14 days, 21 days, 30 days, and any such durations in between, as would be readily understood by one of skill.

The time elapsed between doses will not be confused with the frequency at which dosing occurs. As will be appreciated by one of skill, the frequency of dosing may depend on the disease and symptoms being treated, and by the administration of the primary and/or secondary bioactive molecule disclosed herein. As a non-limiting example, it may be desirable to administer combination drugs between about one and about eight times per day, including once per day, twice per day, three times per day, four times per day, five times per day, six times per day, seven times per day, and eight times per day; in some embodiments, one of the combination drugs (i.e., one of the bioactive compounds in a combination) may be administered twice per day, or three times per day, while another combination drug is administered one time per day, or twice per day, and any further combination drug(s) are administered on appropriate administration schedules as applicable thereto.

Further, the dosing schedule may continue in the same pattern or a modified one for a specific duration of time, including 1-7 days, or greater than 7 days, including 14, 21, 28, 29, 30, 31, 35, 42, 49, 60, 75, 90, 100, 125, 150, 175, 200, 225, 250, 275, 300, 325, 350, 375, 400, 425, 450, 475, 500, 525, 550, 575, 600, 625, 650, 675, and 700 days; and the manner in which administration is completed (i.e., via separate, sequential, or simultaneous administration) may be adjusted as needed, such that the entire dosing schedule may only include sequential, simultaneous, or separate dosing; or may include a combination of two of sequential, simultaneous, or separate dosing; or may include all of sequential, simultaneous, or separate dosing. That said, in embodiments, the frequency of dosing and duration of elapsed time between administrations will depend on the specific primary and/or secondary bioactive molecules utilized and the condition sought to be treated.

In embodiments, administration may comprise separate, sequential, or simultaneous oral administrations, sublingual administrations, buccal administrations, intravenous injections, intra-arterial injections, intraperitoneal injections, intraosseous injections, intramuscular injections, intrathecal injections, intracerebroventricular injections, rectal administrations, vaginal administrations, ocular administrations, nasal administrations, cutaneous administrations, topical administrations, otic administrations, transdermal administrations, or a combination thereof, as would be apparent to one of skill depending on the desired therapeutic effect. Further, as mentioned, in embodiments, a patient is administered the combination drugs through more than one administration, wherein each administration is not the same means of administration. As disclosed herein, such may be advantageous in situations wherein the primary and/or secondary bioactive molecules have varying durations of action and maximum plasma concentrations.

In embodiments, the primary and/or secondary bioactive molecules disclosed herein are administered separately. In embodiments, the primary and/or secondary bioactive molecules disclosed herein are administered sequentially. In embodiments, the primary and/or secondary bioactive molecules disclosed herein are administered simultaneously. In embodiments, the timing of the administration of the primary and/or secondary bioactive molecules disclosed herein (including whether or not more than one administration of the primary and/or secondary bioactive molecules is necessary) will depend on the specific primary and/or secondary bioactive molecules administered to the individual and the indication sought to be treated.

a. Pharmaceutical Kits

In embodiments, especially where a formulation is prepared in single unit dosage form, suggested dosage amounts shall be known by reference to the format of the preparation itself. In other embodiments, suggested dosage amounts may be known by reference to the means of administration or by reference to the packaging and labeling, package insert(s), marketing materials, training materials, or other information and knowledge available to those of skill or the public. Another aspect of this disclosure therefore provides pharmaceutical kits containing a pharmaceutical composition or formulation of the invention, suggested administration guidelines or prescribing information therefore, and a suitable container. Individual unit dosage forms can be included in multi-dose kits or containers. Pharmaceutical compositions also can be packaged in single or multiple unit dosage forms for uniformity of dosage and ease of administration. Accordingly, another aspect of this disclosure provides pharmaceutical kits containing a pharmaceutical composition or formulation of the invention, suggested administration guidelines or prescribing information therefore, and a suitable container. Individual unit dosage forms can be included in multi-dose kits or containers. Pharmaceutical compositions also can be packaged in single or multiple unit dosage forms for uniformity of dosage and ease of administration.

In an exemplary pharmaceutical kit, capsules, tablets, caplets, or other unit dosage forms are packaged in blister packs. “Blister pack” refers to any of several types of pre-formed container, especially plastic packaging, that contains separate receptacles (e.g., cavities or pockets) for single unit doses, where such separate receptacles are individually sealed and can be opened individually.

Blister packs include such pharmaceutical packs known to those of skill, including Aclar® Rx160, Rx20e, SupRx, and UltRx 2000, 3000, 4000, and 6000 (Honeywell). Within the definition of multi-dose containers, and also often referred to as blister packs, are blister trays, blister cards, strip packs, push-through packs, and the like. Preferably, information pertaining to dosing and proper administration (if needed) is printed onto a multi-dose kit directly (e.g., on a blister pack or other interior packaging holding the disclosed combinations or compositions); however, kits of the invention can further contain package inserts and other printed instructions (e.g., on exterior packaging) for administering the disclosed combinations or compositions and for their appropriate therapeutic use.

In embodiments, a patient will have the option of using online software such as a website, or downloadable software such as a mobile application, to assist with compliance or to provide data relating to treatment. Such software can be used to, e.g., keep track of last dose taken and total doses taken, provide reminders and alerts for upcoming doses, provide feedback to discourage taking doses outside of set schedules, and allow for recording of specific subjective effects, or provide means for unstructured journaling. Such data collection can assist with individual patient compliance, can be used to improve or tailor individual patient care plans, and can be anonymized, aggregated, and analyzed (including by AI or natural language processing means) to allow research into the effects of various methods of treatment.

### I. Methods of Treatment

In some aspects are methods of preventing or treating a movement disorder, comprising administering to a patient a disclosed therapeutic combination or pharmaceutical composition.

Without being bound by theory, disclosed combinations, compositions, and methods are in some embodiments useful to treat movement disorders due to their redundant, promiscuous effects on CNS signaling capabilities. In some embodiments, such effects include improvements to the integrity and density of neuronal cell structures, neurotransmitter synthesis and regulation, enhanced neuroprotection, and positive exploitation of plasticity mechanisms (e.g., BDNF, serotonergic muscle control, and glial networks).

In some embodiments, disclosed combinations, compositions, and methods are useful to address failures within these neurological systems, and address multiple contributors to the pathophysiology of movement disorders such as Parkinson's disease. In embodiments, each component of the disclosed combinations and compositions may affect these neurological circuits in multiple ways, for example with differing pharmacodynamics, supported by the privileged access and broad activity afforded to exogenous inputs from natural sources. In one example, the disclosed combinations and compositions affect numerous immunological and mitochondrial interfaces, which in combination with their effects on CNS signaling capabilities, collectively and synergistically creates a fulsome therapeutic for movement disorders, such as Parkinson's disease.

In some aspects, disclosed combinations, compositions, and methods are useful to treat movement disorders due to their redundant, promiscuous negative regulation of pro-inflammatory cycles, for example by reducing cytokines TNF-α and IL-1β through direct and indirect (amplification) interference with NF-κB and AP-1 transduction mechanisms, reduction of oxidative ROS, NO, and MPO, and activation of anti-inflammatory networks PPAR-y and NRf2.

In some aspects, disclosed combinations, compositions, and methods are useful in the treatment of inflammatory and oxidative systems that contribute to the pathophysiology of movement disorders, e.g., Parkinson's disease. For example, neurodegeneration in movement disorders has been proposed to be characterized by metal dyshomeostasis and oxidative stress (Trist et al., 2018). Oxidative stress is well documented in Parkinson's disease and has been attributed to dopamine oxidative metabolism (Ahlskog, 2005). Oxidative mechanisms have been linked to nigral cell death in Parkinson's disease (Jenner, 1998). In embodiments, a disclosed combination synergistically interacts with inflammatory and oxidative systems by, for example, synergistically avoiding receptor-exhaustion, activating self-regulating compensatory measures, and/or inducing immunological reactions. In some embodiments, a disclosed combination possesses antioxidant properties, which, without being bound by theory, directly or indirectly treat or prevent a movement disorder, e.g., by mediating or reducing oxidative stress associated with the disorder.

In some embodiments, a disclosed combination is useful for the treatment of inflammatory and oxidative systems. As one illustrative example, Parkinson's disease typically coincides with a dramatic loss of dopaminergic neurons within the substantia nigra (Meiser et al, 2013). Oxidative stress is implicated in the loss of dopaminergic neurons, and dopamine metabolism itself is considered to be strongly linked to oxidative stress, as its degradation generates reactive oxygen species (ROS) and dopamine oxidation can lead to endogenous neurotoxins (id.). Therefore, without being bound by theory, dopamine metabolism is of special importance for neuronal redox-homeostasis and viability. In embodiments, a disclosed combination exerts antioxidant effects that are useful in treating or preventing Parkinson's disease or another movement disorder. In embodiments, the antioxidant effects of a disclosed combination prevent or reduce the loss of dopaminergic neurons in an individual with Parkinson's disease or another movement disorder, for example, by reducing oxidative stress. In embodiments, reducing oxidative stress comprises reducing the concentration of ROS associated with dopamine metabolism. In embodiments, reducing oxidative stress comprises neutralizing (e.g., chemically reducing) ROS associated with dopamine metabolism. In embodiments, the antioxidant effects of a disclosed combination reduce the concentration of endogenous neurotoxins associated with dopamine oxidation. In embodiments, antioxidant effects of disclosed combinations promote neuronal redox-homeostasis. In embodiments, antioxidant effects of disclosed combinations promote neural viability.

Furthermore, in some embodiments, the antioxidant effects of a disclosed combination may be measured by a change in a biomarker of a movement disorder (e.g, Parkinson's disease), for example, a change (e.g., reduction) in iron levels, and/or a decrease in the concentration of enzymes and substrates associated with oxidative stress. Several biomarkers of oxidative stress have been linked to Parkinson's disease (Frijhoff, 2015). In embodiments, the biomarker is nigral cell death, free iron, glutathione (GSH), glutathione disulfide (GSSG), nuclear factor erythroid 2-related factor 2 (NRF2), advanced glycation end products (AGE), aldehyde 4-hydroxynonenal (HNE), malonaldehyde (MDA), F₂-isoprostanes, isolevuglandins (isoLG), nitrotyrosine (NO₂-Tyr), or a combination thereof. In embodiments, the biomarker is nigral cell death. In embodiments, the biomarker is free iron. In embodiments, the biomarker is glutathione (GSH). In embodiments, the biomarker is glutathione disulfide (GSSG). In embodiments, the biomarker is nuclear factor erythroid 2-related factor 2 (NRF2). In embodiments, the biomarker is advanced glycation end products (AGE). In embodiments, the biomarker is aldehyde 4-hydroxynonenal (HNE). In embodiments, the biomarker is malonaldehyde (MDA). In embodiments, the biomarker is F₂-isoprostanes. In embodiments, the biomarker is isolevuglandins (isoLG). In embodiments, the biomarker is nitrotyrosine (NO₂-Tyr).

In some embodiments, the antioxidant effects of a disclosed combination may result in a decrease of a biomarker of oxidation (e.g., a biomarker of oxidative stress). In embodiments, the concentration of the biomarker is decreased by about at least 99%, 99%, 98%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, or less than 1%, including ranges in between these values. In embodiments, the concentration of the biomarker is decreased by at least 99%. In embodiments, the concentration of the biomarker is decreased by about 99%. In embodiments, the concentration of the biomarker is decreased by about 98%. In embodiments, the concentration of the biomarker is decreased by about 95%. In embodiments, the concentration of the biomarker is decreased by about 90%. In embodiments, the concentration of the biomarker is decreased by about 85%. In embodiments, the concentration of the biomarker is decreased by about 80%. In embodiments, the concentration of the biomarker is decreased by about 75%. In embodiments, the concentration of the biomarker is decreased by about 70%. In embodiments, the concentration of the biomarker is decreased by about 65%. In embodiments, the concentration of the biomarker is decreased by about 60%. In embodiments, the concentration of the biomarker is decreased by about 55%. In embodiments, the concentration of the biomarker is decreased by about 50%. In embodiments, the concentration of the biomarker is decreased by about 45%. In embodiments, the concentration of the biomarker is decreased by about 40%. In embodiments, the concentration of the biomarker is decreased by about 35%. In embodiments, the concentration of the biomarker is decreased by about 30%. In embodiments, the concentration of the biomarker is decreased by about 25%. In embodiments, the concentration of the biomarker is decreased by about 20%. In embodiments, the concentration of the biomarker is decreased by about 15%. In embodiments, the concentration of the biomarker is decreased by about 10%. In embodiments, the concentration of the biomarker is decreased by about 5%. In embodiments, the concentration of the biomarker is decreased by about 4%. In embodiments, the concentration of the biomarker is decreased by about 3%. In embodiments, the concentration of the biomarker is decreased by about 2%. In embodiments, the concentration of the biomarker is decreased by about 1%. In embodiments, the concentration of the biomarker is decreased by less than 1%.

In some embodiments, antioxidant properties and antioxidant effects of disclosed combinations can be measured by a total antioxidant assay, as disclosed in Apak, 2007. In embodiments, the total antioxidant assay is a Folin-Ciocalteu assay. In embodiments, the total antioxidant assay is a 2,2-diphenyl-1-picryl (DPPH) assay. In embodiments, the total antioxidant assay is a Trolox Equivalent Antioxidant Capacity (TEAC) assay. In embodiments, the total antioxidant assay is a Ferric Reducing Antioxidant Power (FRAP) assay. In embodiments, the total antioxidant assay is a Total Antioxidant Capacity (TAC) assay. In embodiments, the total antioxidant assay is a Superoxide Dismutase (SOD) assay. In embodiments, the total antioxidant assay is a Lipid Peroxidation assay. In embodiments, the total antioxidant assay is a Hydroxyl Radical Scavenging assay. In embodiments, the total antioxidant assay is a cupric ion reducing antioxidant capacity (CUORAC) assay.

In some embodiments, antioxidant properties of disclosed combinations result from synergy between the fungal, plant, and algal portions, said synergy being defined In embodiments as an antioxidant effect that is greater than the additive antioxidant effects or antioxidant properties of the portions when administered alone. In embodiments, the synergy is a synergistic effect in, e.g., antioxidant capacity, potency, bioactivity, bioaccessibility, bioavailability, therapeutic effect, or a combination thereof.

“Treatment” covers any treatment of a disorder in a mammal, and particularly in a human, and includes: (a) preventing a disorder from occurring in a subject who may be predisposed to the disorder but has not yet been diagnosed with it: (b) inhibiting a disorder, i.e., arresting its development (including, e.g., prophylaxis); (c) relieving a disorder, i.e., causing regression of the disorder or its clinical symptoms; (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder. One will understand that a therapeutic amount necessary to effect treatment for purposes of this invention will, for example, be an amount that provides for objective indicia of improvement in patients having clinically-diagnosable symptoms. The effect may be prophylactic in terms of completely or partially preventing a disorder or symptom thereof and/or may be therapeutic in terms of a partial or complete cure for a disorder and/or adverse effect attributable to the disorder.

Whether a patient has a movement disorder can be determined according to ordinary skill. Without being bound by theory, movement disorders are generally diagnosed after motor symptoms present. Examples of methods used to diagnose movement disorders include genetic testing to detect known abnormalities that may be indicative of a movement disorder, testing of blood, urine, and cerebrospinal fluid; a trial of condition-specific medication useful in alleviating a symptom of a condition, nuclear imaging, magnetic resonance imaging (MRI), computerized tomography (CT), observance of present symptoms, including observing facial expressions, observing limbs for the presence of tremors and gait for any abnormalities, assessing stiffness of the neck, assessing balance (including the time it takes to regain balance), patient history information, and additional testing to exclude any similar conditions. Because a variety of diseases and conditions have symptoms characteristic of a movement disorder, diagnosis may include a combination of the aforementioned methods, and may include additional methods known to those of skill.

In embodiments, measures of therapeutic effect include outcome measures (primary or secondary), endpoints, effect measures, and measures of effect within clinical or medical practice or research which can be used to assess an effect (positive and/or negative) of an intervention or treatment, whether patient-reported (e.g., questionnaires); based on other patient data (e.g., patient monitoring); gathered through laboratory tests such as from blood or urine; through medical examination by a doctor or other medical professional, or by digital means, such as by using electronic tools such as online tools, smartphones, wireless devices, biosensors, or health apps.

In embodiments, measures of therapeutic effect include (a) the severity of symptoms, (b) motor control, (c) MDS-UPDRS, (d) UPDRS, and (e) UPDRS-8.

In embodiments, measures of therapeutic effect include (a) DaTscan to measure dopamine transporter system dysfunction, and (b) αSyn-SAA (alpha-synuclein seed amplification assay). DaTscan involves injecting technetium-99m (tropane derivative) into the bloodstream, wherein tropane derivative binds to dopamine transporters in the brain. A gamma camera then detects the radiation and creates images which detail the distribution and density of dopamine transporters in the brain. Integrity of dopamine-producing cells in specific regions of the brain can then be evaluated according to known methods. αSyn-SAA investigates the aggregation and propagation of abnormal α-synuclein protein structures, which may play a role in the progression of neurodegenerative disorders such as Parkinson's disease. «Syn-SAA includes seed formation (e.g., creation or isolation of abnormal α-synuclein), propagation, amplification, and detection (e.g., immunohistochemistry, immunofluorescence, or ELISA).

In embodiments, a movement disorder refers to a group of nervous system (neurological) conditions that cause abnormal increased, decreased, reduced, or slowed movements, which may be voluntary or involuntary (Mayo Clinic, 2017). In embodiments, movement disorders include amyotrophic lateral sclerosis (ALS), ataxic disorders, including ataxia, congenital ataxia, hereditary ataxia (e.g., Friedreich ataxia, ataxia due to cerebrotendinous xanthomatosis, ataxia due to refsum disease, ataxia due to abetalipoproteinemia, hereditary episodic ataxia, ataxia due to mitochondrial mutations, and spinocerebellar ataxia), non-hereditary degenerative ataxia (e.g., late onset cerebellar cortical atrophy), acquired ataxia (e.g, ataxia due to alcoholic cerebellar degeneration), cervical dystonia, choreiform disorders, including chorea, benign hereditary chorea, secondary chorea (e.g., Huntington disease, chorea due to Huntington disease-like conditions, chorea due to dentatorubral pallidoluysian atrophy, chorea due to Wilson disease, chorea due to infectious or parainfectious causes, chorea due to systemic lupus erythematosus, drug-induced chorea, and rheumatic chorea), dystonic disorders, including dystonia, primary dystonia, (e.g., benign essential blepharospasm), secondary dystonia (e.g., drug-induced dystonia, dystonia-plus, and dystonia associated with heredodegenerative disorders), paroxysmal dystonia, and functional dystonia or spasms, functional movement disorders, Huntington's disease, multiple system atrophy, myoclonus, Parkinson's disease, including sporadic parkinson disease and familial parkinson disease, Parkinsonism, including atypical parkinsonism (e.g., progressive supranuclear palsy (PSP) and lewy body disease), secondary parkinsonism (e.g., parkinsonism due to heredodegenerative disorders, infectious or postinfectious parkinsonism, vascular parkinsonism, drug-induced parkinsonism, post-traumatic parkinsonism, and parkinsonism due to structural lesions) and functional parkinsonism, atypical Parkinson's, young onset Parkinson's disease (YOPD), restless legs syndrome, tardive dyskinesia, L-dopa induced dyskinesia, disorders associated with tremor, including tremor, essential tremor, enhanced physiological tremor, rest tremor, secondary tremor (e.g., tremor due to metabolic disorders, tremor due to chronic or acute substance use, tremor due to drug withdrawal, tremor due to certain specified central nervous system diseases), and functional tremor, tic disorders, including primary tics or tic disorders (e.g., Tourette syndrome, chronic motor tic disorder, chronic phonic tic disorder, and transient motor tics), secondary tics (e.g., infectious or postinfectious tics and tics associated with developmental disorders), myoclonic disorders (e.g., essential myoclonus, segmental myoclonus, focal myoclonus, such as palatal myoclonus and chronic hiccups), Wilson's disease, hemifacial spasm, certain specified movement disorders (e.g., stereotypies such as primary stereotypy and secondary stereotypy), Rett syndrome, akathisia, and excessive startle reflex, sleep-related movement disorders, including restless legs syndrome, periodic limb movement disorder, sleep-related leg cramps, sleep-related bruxism, sleep-related rhythmic movement disorder, benign sleep myoclonus of infancy, propriospinal myoclonus at sleep onset, sleep-related movement disorder due to a medical condition, sleep-related movement disorder due to a medication or substance, and REM sleep behaviour disorder, and hereditary spastic paraplegia, including autosomal dominant hereditary spastic paraplegia, autosomal recessive hereditary spastic paraplegia, and X-linked hereditary spastic paraplegia.

In some embodiments, the movement disorder is any of ataxia, an ataxic disorder, a certain specified movement disorder, cervical dystonia, chorea, a choreiform disorder, dystonia, a dystonic disorder, essential tremor, Friedreich's ataxia, a functional movement disorder, hemifacial spasm, hereditary spastic paraplegia, Huntington's disease, L-dopa induced dyskinesia, multiple system atrophy (MSA), myoclonus, a myoclonic disorder, Parkinson's disease, atypical Parkinson's, Parkinsonism, Secondary Parkinsonism, progressive supranuclear palsy (PSP), restless legs syndrome, Rett syndrome, a sleep-related movement disorder, spasticity, tardive dyskinesia (TD), tourette syndrome, a tic disorder, a disorder associated with tremor, and Wilson's disease. In some embodiments, the movement disorder is Parkinson's disease (PD).

Parkinson's disease is a disorder primarily affecting the dopamine-producing (dopaminergic) neurons in a specific area of the brain called substantia nigra. PD symptoms generally develop slowly over years (although some aggressive forms of PD are known, and the disclosed invention is suitable for treatment of such aggressive forms as well) (Elkouzi, n.d.).

Without being bound by theory, movement control is accomplished via myriad interactions among various groups of nerve cells in the CNS, one such group being in the substantia nigra (SN). Nigral neurons give rise to an extensive network of axonal processes that innervate the basal ganglia, establishing predominantly symmetrical synapses with dendritic spines and shafts of medium spiny projection neurons (Triarhou, 2002). SN neurons communicate with neurons of the basal ganglia by liberating the neurotransmitter dopamine (DA).

Those with PD generally experience impairment and/or death of the dopamine-producing nerve cells in the basal ganglia, which causes a reduction and cessation in dopamine production, respectively. This prevents the signaling cascade and downstream effects caused by dopamine release and, as a result, fine motor control. The brain cells of those with PD also frequently contain Lewy bodies, which refer to clumps of α-synuclein protein which, while poorly understood, is hypothesized to regulate neurotransmitter release. Those with Lewy bodies may develop Lewy Body Dementia (LBD), which is associated with depletion of acetylcholine (affecting memory and thinking) and dopamine (further affecting movement) in the brain.

Moreover, PD is often characterized by a loss in nerve endings that produce norepinephrine (the main chemical messenger of the sympathetic nervous system) which may explain the non-movement features of PD. As the therapeutic combinations gradually interfere in the inflammation-mediated progression of PD, co-functionalities, including activation of alternative signaling networks afforded by BDNF-mediated plasticity and improved cell viability/energetics, may work in concert to deliver a systemic, low-dose, disease modifying treatment or adjunct to address Parkinsonism.

In embodiments, the therapeutic combinations are effective in increasing endogenous dopamine, serotonin, and norepinephrine. In embodiments, the therapeutic combinations are effective in increasing endogenous dopamine, serotonin, and norepinephrine via any of the activation and mediation of the serotonin system, activation of NPR19, CB₁, CB₂ TRPV1, PPAR-y, rebalancing AchE and AchE activities, increasing dopamine release via PKA and CaMKII, upregulating cAMP and Ca2+, upregulating tyrosine hydroxylase, activating DA transport, and increasing monoamine oxidase (MAO) inhibition.

In embodiments, the therapeutic combinations are effective in repressing co-repressors NF-kβ and AP-1 at NURR1, an orphan nuclear receptor which is severely diminished by α-syn deposits and in many ways central to the dopaminergic collapse characteristic of Parkinson's disease. This may re-enable basal microglial down regulation and create an environment conducive to dopaminergic restoration.

In embodiments, the therapeutic combinations are effective in increasing presynaptic and postsynaptic protein density. In embodiments, the increases in protein density may be observed as increases in synaptic vesicle glycoprotein 2A (SV2A) and postsynaptic density protein 95 (PSD95, also known as synapse-associated protein 90 (SAP-90)).

In embodiments, a patient administered the therapeutic combinations may experience an improvement in any of: (a) the severity of symptoms (e.g., motor symptoms, such as stooped posture, masked facial expression, forward tilt of trunk, flexed elbows and wrists, reduced arm swinging, flexed hips and knees, trembling of extremities, shuffling gait, short-stepped gait, uncoordinated or clumsy balance, altered speech, involuntary limb movements, irregular motor movement, long-lasting contractions, intermittent contractions of neck muscles, causing the head to turn in different ways; repetitive, irregular, involuntary movements involving the face, mouth, trunk, and limbs; twisting, repetitive movements; jerking of muscles or groups of muscles, tremors, stiffness, finger tapping, toe tapping, poor posture, slow, decreased movement or imbalance; difficulties walking, random involuntary eye movement, involuntary blinking, involuntary grimacing, unpleasant, abnormal feelings in limbs which may be relieved by movement; involuntary vocal sounds, and rhythmic shaking of parts of the body, commonly the hands and/or head); (b) motor control (e.g., walking, standing, general movement, and other motor symptoms); (c) MDS-UPDRS; (d) UPDRS; (e) UPDRS-8; (f) biomarkers of Parkinson's disease (e.g., alpha-synuclein, dopamine transporter activity and density, neuroinflammatory markers, olfactory dysfunction, peripheral blood markers DTI and fmRI imaging); and (g) metabolites associated with Parkinson's disease (e.g., oleic acid, arachidonic acid, valeric acid, butyric acid, sphingomyelin, glycosphingolipid, ornithine, glutamine, urate, homovanillic acid, dopamine and metabolites of dopamine, glutathione, malondialdehyde, superoxide dismutase, guanosine, adenosine, inosine phenylalanine, tyrosine, tryptophan, and glycine).

In embodiments, the improvement is an improvement in the severity of at least one symptom of the movement disorder. In embodiments, the improvement is a reduction in at least one symptom of the movement disorder. In embodiments, the improvement is an elimination of at least one symptom of the movement disorder. In embodiments, the reduction is compared to a baseline determination made before such administration. In embodiments, the improvement is durable, e.g., lasts for at least 3 months, 6 months, 9 months, 1 year, or greater than 1 year.

In embodiments, the improvement is an improvement in a symptom which includes stooped posture, masked facial expression, forward tilt of trunk, flexed elbows and wrists, reduced arm swinging, flexed hips and knees (including fully flexed hips and knees, and slightly flexed hips and knees), trembling of extremities, shuffling gait, short-stepped gait, uncoordinated or clumsy balance, altered speech, involuntary limb movements, irregular motor movement, long-lasting contractions (spasms), intermittent contractions of neck muscles, causing the head to turn in different ways; repetitive, irregular, involuntary movements involving the face, mouth, trunk, and limbs; twisting, repetitive movements (motor tics); jerking of muscles or groups of muscles, tremors, stiffness (rigidity), finger tapping, toe tapping, poor posture, slow, decreased movement or imbalance; difficulties walking, random involuntary eye movement, involuntary blinking, involuntary grimacing, unpleasant, abnormal feelings in limbs which may be relieved by movement; involuntary vocal sounds (vocal tics), and rhythmic shaking of parts of the body, commonly the hands and/or head; feelings of depression, anxiety, irritability, mood swings, impaired judgment, loss of empathy, aggression, impulsivity, delusions, and paranoia; cognitive impairment (e.g., cognitive slowing, impaired reasoning, memory loss, and deficits in attention and orientation), hallucinations and psychosis (e.g., hallucinations and illusions), depressed mood (e.g., low mood, sadness, hopelessness, feelings of emptiness, and loss of enjoyment), anxious mood (e.g., nervousness, tense, worry, anxiety), apathy, features of dopamine dysregulation syndrome (e.g., atypical or excessive gambling, such as casinos or lottery tickets; atypical or excessive sexual drive or interests, such as unusual interest in pornography, masturbation, sexual demands on partner; other repetitive activities, including hobbies, dismantling objects, sorting or organizing; or taking extra non-prescribed medication for non-physical reasons, such as addictive behavior); sleep problems (e.g., trouble falling asleep), daytime sleepiness, pain and other sensations (e.g., pain, aches, tingling, or cramps), urinary problems, constipation problems, lightheadedness on standing, and fatigue; issues with speech, overproducing saliva and drooling, problems with chewing and swallowing, difficulty eating, dressing, maintaining proper hygiene, handwriting, doing hobbies and other activities, turning in bed, getting out of bed, a car, or a deep chair; trouble walking and maintaining balance, experiencing tremor, and freezing in place.

In embodiments, the improvement is an improvement in motor symptoms. In embodiments, motor symptoms include stooped posture, masked facial expression, forward tilt of trunk, flexed elbows and wrists, reduced arm swinging, flexed hips and knees (including fully flexed hips and knees, and slightly flexed hips and knees), trembling of extremities, shuffling gait, short-stepped gait, uncoordinated or clumsy balance, altered speech, involuntary limb movements, irregular motor movement, long-lasting contractions (spasms), intermittent contractions of neck muscles, causing the head to turn in different ways; repetitive, irregular, involuntary movements involving the face, mouth, trunk, and limbs; twisting, repetitive movements (motor tics); jerking of muscles or groups of muscles, tremors, stiffness (rigidity), finger tapping, toe tapping, poor posture, slow, decreased movement or imbalance; difficulties walking, random involuntary eye movement, involuntary blinking, involuntary grimacing, unpleasant, abnormal feelings in limbs which may be relieved by movement; involuntary vocal sounds (vocal tics), and rhythmic shaking of parts of the body, commonly the hands and/or head (Cummins, Zandi, and Barker, 2015; and International Parkinson and Movement Disorder Society, 2019). In embodiments, the reduction in symptoms is a reduction in motor symptoms.

In embodiments, the improvement is an improvement in mood symptoms. In embodiments, mood symptoms include depression, anxiety, apathy, irritability, mood swings, impaired judgment, loss of empathy, aggression, impulsivity, delusions, and paranoia (Cummins, Zandi, and Barker, 2015). In embodiments, the reduction in symptoms is a reduction in mood symptoms. In embodiments, the improvement in mood symptoms may be determined via clinician outcome assessments, such as those disclosed herein, as well as those known to be useful for mood assessment, including the Beck Depression Inventory (BDI), the Hospital Anxiety and Depression Scale (HADS), the Hamilton Anxiety Scale (HAM-A or HAS), the Hamilton Depression Scale (HAM-D), the Montgomery-Asberg Depression Rating Scale (MADRS), the Patient Health Questionnaire 9 (PHQ-9), the Generalized Anxiety Disorder 7 (GAD-7), the Obsessive-Compulsive Inventory (OCI), the Personality Disorders Questionnaire (PDQ-IV), Dissociative Experiences Scale (DES), the Mood Disorder Questionnaire (MDQ), and other similar questionnaires. In embodiments, assessment of mood may be completed by assessment prior to, during, and after treatment with the therapeutic combinations.

In embodiments, the improvement in symptoms is an improvement in those outlined in the MDS-UPDRS, which include “non-motor aspects of experiences of daily living” (nM-EDL) table and “motor aspects of experiences of daily living” (M-EDL). In embodiments, nM-EDL include cognitive impairment (e.g., cognitive slowing, impaired reasoning, memory loss, and deficits in attention and orientation), hallucinations and psychosis (e.g., hallucinations and illusions), depressed mood (e.g., low mood, sadness, hopelessness, feelings of emptiness, and loss of enjoyment), anxious mood (e.g., nervousness, tense, worry, anxiety), apathy, features of dopamine dysregulation syndrome (e.g., atypical or excessive gambling, such as casinos or lottery tickets; atypical or excessive sexual drive or interests, such as unusual interest in pornography, masturbation, sexual demands on partner; other repetitive activities, including hobbies, dismantling objects, sorting or organizing; or taking extra non-prescribed medication for non-physical reasons, such as addictive behavior); sleep problems (e.g., trouble falling asleep), daytime sleepiness, pain and other sensations (e.g., pain, aches, tingling, or cramps), urinary problems, constipation problems, lightheadedness on standing, and fatigue. In embodiments, the reduction in symptoms is a reduction in nM-EDL. In embodiments, M-EDL include issues with speech, overproducing saliva and drooling, problems with chewing and swallowing, difficulty eating, dressing, maintaining proper hygiene, handwriting, doing hobbies and other activities, turning in bed, getting out of bed, a car, or a deep chair; trouble walking and maintaining balance, experiencing tremor, and freezing in place (International Parkinson and Movement Disorder Society, 2019). In embodiments, the reduction in symptoms is a reduction in M-EDL.

In embodiments, the time elapsed between administering to the patient in need thereof a therapeutic combination and the reduction in the severity of symptoms is less than about 75 days, including less than 75 days, less than 70 days, less than 65 days, less than 60 days, less than 55 days, less than 50 days, less than 45 days, less than 40 days, less than 35 days, less than 30 days, less than 25 days, less than 20 days, less than 15 days, less than 10 days, less than 5 days, 1 day or less, including 18 hours or less, 12 hours or less, 6 hours or less, 3 hours or less, 2 hours or less, 1 hour or less, 30 mins or less, 15 mins or less, and 1 minute or less, and values in between.

In embodiments, the time elapsed between administering to the patient in need thereof a therapeutic combination and the reduction in the severity of symptoms is greater than about 75 days, but less than about 100 days. In embodiments, the time elapsed between administering to the patient in need thereof the therapeutic combination and the reduction in the severity of symptoms is greater than about 100 days, but less than about 125 days. In embodiments, the time elapsed between administering to the patient in need thereof the therapeutic combination and the reduction in the severity of symptoms is greater than about 125 days, but less than about 150 days. In embodiments, the time elapsed between administering to the patient in need thereof the therapeutic combination and the reduction in the severity of symptoms is greater than about 150 days, but less than about 175 days. In embodiments, the time elapsed between administering to the patient in need thereof the therapeutic combination and the reduction in the severity of symptoms is greater than about 175 days, but less than about 200 days. In embodiments, the time elapsed between administering to the patient in need thereof the therapeutic combination and the reduction in the severity of symptoms is greater than about 200 days, but less than about 225 days. In embodiments, the time elapsed between administering to the patient in need thereof the therapeutic combination and the reduction in the severity of symptoms is greater than about 225 days, but less than about 250 days. In embodiments, the time elapsed between administering to the patient in need thereof the therapeutic combination and the reduction in the severity of symptoms is greater than about 250 days, but less than about 275 days. In embodiments, the time elapsed between administering to the patient in need thereof the therapeutic combination and the reduction in the severity of symptoms is greater than about 275 days, but less than about 300 days. In embodiments, the time elapsed between administering to the patient in need thereof the therapeutic combination and the reduction in the severity of symptoms is greater than about 300 days, but less than about 325 days. In embodiments, the time elapsed between administering to the patient in need thereof the therapeutic combination and the reduction in the severity of symptoms is greater than about 325 days, but less than about 350 days. In embodiments, the time elapsed between administering to the patient in need thereof the therapeutic combination and the reduction in the severity of symptoms is greater than about 350 days, but less than about 1 year. In embodiments, the time elapsed between administering to the patient in need thereof the therapeutic combination and the reduction in the severity of symptoms is greater than about 1 year days, but less than about 2 years. In embodiments, the time elapsed between administering to the patient in need thereof the therapeutic combination and the reduction in the severity of symptoms is greater than about 2 years, but less than about 3 years. In embodiments, the time elapsed between administering to the patient in need thereof the therapeutic combination and the reduction in the severity of symptoms is greater than about 3 years, but less than about 5 years. In embodiments, the time elapsed between administering to the patient in need thereof the therapeutic combination and the reduction in the severity of symptoms is greater than about 5 years, but less than about 10 years. In embodiments, the time elapsed between administering to the patient in need thereof the therapeutic combination and the reduction in the severity of symptoms is greater than about 10 years, but less than about 20 years.

In embodiments, the time elapsed between administering to the patient in need thereof a therapeutic combination and the reduction in the severity of symptoms is less than about 1 year, less than about 2 years, less than about 3 years, less than about 4 years, less than about 5 years, less than about 6 years, less than about 7 years, less than about 8 years, less than about 9 years, less than about 10 years, less than about 15 years, or less than about 20 years.

In embodiments, the reduction in the severity of at least one symptom is a durable reduction, wherein the reduction lasts for at least about 1 week, including about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 18 months, about 24 months, about 36 months, about 48 months, about 60 months, and more than about 60 months.

In embodiments, the improvement is an improvement in motor control. In embodiments, the improvement is compared to a baseline determination made before such administration. In embodiments, motor control may be assessed via analysis of balance, frequency of involuntary movements, amplitude of involuntary movements, strength, endurance, and physical capacity. In embodiments, balance may be assessed via, e.g., a four-stage balance test, wherein a patient is instructed to hold four positions for a predefined period, e.g., 10 seconds. The administrator of the exam may record notes indicating how long the patient was able to hold each pose and, in embodiments, the level of difficulty each pose posed for the patient (CDC.gov, n.d.). In embodiments, balance may additionally be assessed via the Berg Balance Scale (BBS) (Brusse et al., 2005). In embodiments, electrophysiologic assessments, such as those disclosed in Disord, 2009, which is herein incorporated by reference, may be utilized to determine the frequency of involuntary movements and the amplitude of involuntary movements (Park and Kim, 2009). In embodiments, strength may be assessed, e.g., via muscle strength grading, where an examiner applies resistance to key muscles from the upper and lower extremities and assesses strength on a scale of 0-5, wherein 0 indicates no muscle activation, and 5 indicates muscle activation against the examiner's full resistance (full range of motion) (Naqvi and Sherman, 2021). In embodiments, endurance may be assessed via, e.g., the cycle ergometer and ramp protocol endurance tests disclosed by Reuter, Freidwald, and Baas, 1999, which is herein incorporated by reference (1999). In embodiments, physical capacity may be assessed via, e.g., the Forward Functional Reach Test (FFR), the Backward Functional Reach Test (BFR), Timed “Up & Go” Test (TUG), and gait speed (Brusse et al., 2005).

In embodiments, the time elapsed between administering to the patient the therapeutic combinations and the improvement in motor control is less than about 75 days, including less than 75 days, less than 70 days, less than 65 days, less than 60 days, less than 55 days, less than 50 days, less than 45 days, less than 40 days, less than 35 days, less than 30 days, less than 25 days, less than 20 days, less than 15 days, less than 10 days, less than 5 days, 1 day or less, including 18 hours or less, 12 hours or less, 6 hours or less, 3 hours or less, 2 hours or less, 1 hour or less, 30 minutes or less, 15 minutes or less, and 1 minute or less, and values in between.

In embodiments, the improvement in motor control is a durable improvement, wherein the improvement lasts for at least about 1 week, including about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 18 months, about 24 months, about 36 months, about 48 months, about 60 months, and more than about 60 months.

In embodiments, treating or preventing the movement disorder may be assessed by outcome measures, endpoints, effect measures, and measures of effect within clinical or medical practice or research; but, in embodiments, may also be measured by collecting data from the individual themselves, such as but not limited to patient testimonials, a daily activity journal (which may be a physical journal, such as one comprised of paper, or an electronic journal, wherein the individual tracks their activity and changes therein via an electronic device, such as but not limited to a smart watch, a tablet, a computer, and cell phone); the utilization of questionnaires, such as those disclosed herein; and the collection of health measurements, wherein changes thereto illustrate a positive response to treatment with the therapeutic combination.

TABLE 2
Exemplary movement disorder clinical outcome assessments (COAs)

| | |
|---|---|
| 1 | Movement Disorder Society-Unified Parkinson's Disease Rating Scale |
| | (MDS-UPDRS), which assesses the non-motor and motor experiences of daily living and |
| | motor complications in those diagnosed with Parkinson's |
| 2 | Movement Disorder Society Non-Motor Rating Scale (MDS-NMS), which is a |
| | revision of the Non-Motor Symptoms Scale (NMSS) and was developed to improve and |
| | refine the assessment of non-motor symptoms in Parkinson's disease (PD) |
| 3 | Cortical Basal Ganglia Functional Scale (SBFS), which evaluates experiences in daily |
| | living (“EDL”), behavioral, language, and cognitive impairments in patients with 4 repeat |
| | tauopathies (4RTs) |
| 4 | Gastrointestinal Dysfunction Scale for Parkinson's Disease (GIDS-PD), which is a |
| | disease-specific self-report questionnaire to quantitatively assess features of |
| | gastrointestinal dysfunction (GID) symptoms in patients with PD |
| 5 | Global Assessment Scale for Wilson's Disease (GAS for WD), which measures the |
| | severity of the effects of Wilson's disease osseomuscularly, on the liver, cognition, |
| | behavior, and motor skills |
| 6 | Global Dystonia Severity Rating Scale (GDS), which measures global dystonia severity |
| | in 10 body regions, including the eyes and upper face, lower face, jaw and tongue, larynx, |
| | neck, shoulder and proximal arm (left and right), distal arm and elbow (left and right), |
| | proximal leg and pelvis (left and right), distal leg and foot (left and right) and trunk |
| 7 | Modified Bradykinesia Rating Scale (MBRS), which assesses speed, amplitude and |
| | rhythm components of bradykinesia |
| 8 | Non-Motor Symptoms Questionnaire (NMSQ), which is useful in assessing a range of |
| | non-motor symptoms that may occur in all stages of Parkinson's disease |
| 9 | Non-Motor Symptoms Scale for Parkinson's Disease (NMSS), which is a 30-item |
| | rater-based scale to assess a wide range of non-motor symptoms in patients with |
| | Parkinson's disease (PD) |
| 10 | Pantothenate Kinase-Associated Neurodegeneration Disease Rating Scale |
| | (PKAN-DRS), developed to assess domains of cognition, behavior, and disability with |
| | scores based on neurological assessment of dystonia, parkinsonism, and other |
| | neurological signs in patients with PKAN |
| 11 | Progressive Supranuclear Palsy Clinician Deficits Scale (PSP-CDS), which measures |
| | severity of deficits in seven patient-relevant clinical domains: akinesia-rigidity, |
| | bradyphrenia, communication, dysphagia, eye movements, finger dexterity, and gait and |
| | balance |
| 12 | Quality of Life in Essential Tremor Questionnaire, which specifically assesses quality |
| | of life measures in those with essential tremor |
| 13 | Rating Scale for Psychogenic Movement Disorders, which rates 10 phenomena (rest |
| | tremor, action tremor, dystonia, chorea, bradykinesia, myoclonus, tics, athetosis, ballism, |
| | cerebellar incoordination), two functions (gait, speech), and 14 body regions |
| 14 | Rush Dyskinesia Rating Scale (RDRS), to assess the severity of overall dyskinesia |
| | based on interference in activities of daily living, and to distinguish between various |
| | types of dyskinesia |
| 15 | Rush Video-Based Tic Rating Scale (RVBTRS), which utilizes video data gathering |
| | protocols and five domains of tic disability |
| 16 | Scales for Outcome sin Parkinson's Disease - Autonomic Dysfunction |
| | (SCOPA-AUT), which is self-completed by patients and assesses the following domains: |
| | gastrointestinal, urinary, cardiovascular, thermoregulatory, pupillomotor, and sexual |
| 17 | Scales for Outcomes in Parkinson's Disease - Diary Card (SCOPA-DC), which is a |
| | daily diary designed to measure motor impairment in PD patients with motor |
| | complications |
| 18 | Scales for Outcomes in Parkinson's Disease - Psychiatric Complications |
| | (SCOPA-PC), which assesses psychotic and compulsive complications in PD |
| 19 | Scales for Outcomes in Parkinson's Disease - Psychosocial Functioning |
| | (SCOPA-PS), is a self-administered, 11-item questionnaire assessing psychosocial |
| | functioning during the preceding month, on a scale from 0 (not at all) to 3 (very much) |
| 20 | Scales for Outcomes in Parkinson's Disease - Sleep (SCOPA-Sleep; SCOPA-S), |
| | assesses nighttime sleep and daytime sleepiness in patients with Parkinson's disease |
| 21 | Scales for Outcomes in Parkinson's Disease-COGnition (SCOPA-COG), which |
| | assesses cognitive function in PD |
| 22 | Short Parkinson's Evaluation Scale (SPES)/Scales for Outcomes in Parkinson's |
| | Disease - Motor Function (SPES/SCOPA - Motor), which evaluates the motor |
| | function of PD patients and includes motor evaluation, activities of daily living, and |
| | motor complications |
| 23 | Non-Motor Fluctuation Assessment (NoMoFA) Questionnaire, which is an assessment |
| | tool that facilitates the identification and quantification of severity of static and |
| | fluctuating non-motor symptoms that occur in Parkinson's disease |
| 24 | UFMG Sydenham's Chorea Rating Scale (USCRS), which was designed to provide a |
| | quantitative description of the performance of activities of daily living, behavioral |
| | abnormalities, and motor function of patients with SC |
| 25 | Unified Dyskinesia Rating Scale (UDysRS), which captures the essential features of |
| | dyskinesia in Parkinson's disease |
| 26 | Unified Dystonia Rating Scale (UDRS), which was designed to include a more detailed |
| | assessment of the severity of dystonia in individual body areas |
| 27 | Unified Multiple System Atrophy Rating Scale (UMSARS)), which provides a |
| | surrogate measure of disease progression in multiple system atrophy (International |
| | Parkinson and Movement Disorder Society, 2021) |

In embodiments, the improvement is an improvement in the MDS-UPDRS. The MDS-UPDRS is considered a gold standard for Parkinson's assessment. The test includes a self-assessment comprising non-motor aspects of experiences in daily living (nM-EDL), and motor aspects of experiences of daily living (M-EDL), wherein subjects are asked to rate their symptoms on a scale of 0-4, 0 being normal, 1 being slight, 2 being mild, 3 being moderate, and 4 being severe; and motor complications, wherein an examiner provides scores for various observable symptoms, the score being the same 0-4 point scale discussed above, except that dyskinesia impact contains two yes or no questions and Hoehn and Yahr stage is a 0-5 scale, with 5 being the most severe.

In embodiments, the improvement in the MDS-UPDRS is an improvement in nM-EDL, including any of cognitive impairment, hallucinations and psychosis, depressed mood, anxious mood, apathy, features of dopamine dysregulation syndrome, sleep problems, daytime sleepiness, pain and other sensations, urinary problems, constipation problems, lightheadedness on standing, and fatigue.

In embodiments, the improvement in the MDS-UPDRS is an improvement in M-EDL, including any of speech, saliva and drooling, chewing and swallowing, eating tasks, dressing, hygiene, handwriting, doing hobbies and other activities, turning in bed, tremor, getting out of bed, a car, or a deep chair, walking and balance, and freezing. In embodiments, the improvement in the MDS-UPDRS is an improvement in motor examination, including any of speech, facial expression, rigidity, finger tapping, hand movements, pronation-supination movements of hands, toe tapping, leg agility, arising from chair, gait, freezing of gait, postural stability, posture, global spontaneity of movement (body bradykinesia), postural tremor of the hands, kinetic tremor of the hands, rest tremor amplitude, constancy of rest tremor, dyskinesia impact, Hoehn and Yahr stage, time spent with dyskinesias, functional impact of dyskinesias, time spent in the off state, functional impact of fluctuations, complexity of motor fluctuations, and painful off-state dystonia.

In embodiments, the improvement in MDS-UPDRS is a reduction in score. In embodiments, the reduction in score is between 1 and 4 points, including 1, 2, 3, and 4 points.

In embodiments, the improvement is an improvement in the UPDRS. The UPDRS scale is a rating tool used to gauge the course of Parkinson's disease in patients. The UPDRS scale is considered one of the bases of treatment and research in parkinson's clinics. The UPDRS scale includes six assessments: (1) mentation, behavior, and mood, (2) activities of daily living (ADL), (3) motor examination, (4) complications of therapy (in the past week), (5) modified Hoehn and Yahr scale, and (6) the Schwab and England ADL scale. The UPDRS scale is generally a score of 0-4, with four being the most severe; however, in the motor examination assessment, items 35-38, and 40-42 are binary yes and no questions; the modified Hoehn and Yahr staging session contains 8 stages (stage 0, stage 1, stage 1.5, stage 2, stage 2.5, stage 3, stage 4, stage 5), with stage 0 being the lowest, and stage 5 being the most severe; and the Schwab and England ADL scale contains 11 percentages (0%, 10%, 20%, 30%, 40%, 50%, 60%, 70%, 80%, 90%, and 100%), with 100% being completely independent and 0% being in a vegetative state. Some sections of the UPDRS scale require multiple grades assigned to each extremity with a possible maximum of 199 points, indicating total disability, while a score of zero represents no disability (Unified Parkinson Disease Rating Scale, 2022).

In embodiments, the improvement in the UPDRS is an improvement in mentation, behavior, and mood; including any of intellectual impairment, thought disorder (due to dementia or drug intoxication), depression, and motivation/initiative. In embodiments, the improvement in the UPDRS is an improvement in ADL, including any of speech, salivation, swallowing, handwriting, cutting food and handling utensils, dressing, hygiene, turning in bed and adjusting bed clothes, falling (unrelated to freezing), freezing when walking, walking, tremor (symptomatic complaint of tremor in any part of body), and sensory complaints related to parkinsonism. In embodiments, the improvement in the UPDRS is an improvement in motor examination, including speech, facial expression, tremor at rest (head, upper and lower extremities), action or postural tremor of hands, rigidity (judged on passive movement of major joints with patient relaxed in sitting position, cogwheeling to be ignored), finger taps (patient taps thumb with index finger in rapid succession), hand movements (patient opens and closes hands in rapid succession), rapid alternating movements of hands (pronation-supination movements of hands, vertically and horizontally, with as large an amplitude as possible, both hands simultaneously), leg agility (patient taps heel on the ground in rapid succession picking up entire leg; amplitude should be at least 3 inches), arising from chair (patient attempts to rise from a straight backed chair, with arms folded across chest), posture, gait, postural stability (response to sudden, strong posterior displacement produced by pull on shoulders while patient erect with eyes open and feet slightly apart; patient is prepared), and body bradykinesia and hypokinesia (combining slowness, hesitancy, decreased arm swing, small amplitude, and poverty of movement in general). In embodiments, the improvement in the UPDRS is an improvement in complications of therapy, including any of daily duration of dyskinesias (what proportion of the waking day are dyskinesias present), severity of disability from dyskinesias, (how disabling are the dyskinesias), painful dyskinesias (how painful are the dyskinesias), and the proportion of the waking day the patient is “off” on average. In embodiments, the improvement in the UPDRS is a reduction in score. In embodiments, the reduction in score is between 1 and 4 points, including 1, 2, 3, and 4 points.

The modified Hoehn and Yahr staging session contains eight total stages. In embodiments, the improvement in the UPDRS is a reduction in stage of the modified Hoehn and Yahr staging session. In embodiments, the reduction in stage is a reduction in between about 1 and about 7 stages, including 1 stage, 2 stages, 3 stages, 4 stages, 5 stages, 6 stages, and 7 stages.

The Schwab and England ADL scale contains 11 percentages, ranging from 0% to 100%. In embodiments, wherein the improvement is an improvement in the UPDRS, the improvement is a reduction in the Schwab and England ADL scale percentage. In embodiments, the reduction in percentage is between about 10% to about 100%, including 10%, 20%, 30%, 40%, 50%, 60%, 70%, 80%, 90%, and 100%).

Items 35-38, and 40-42 on the UPDRS are binary yes and no questions. In embodiments, the improvement in the UPDRS is a change in said yes or no questions, including presence of early morning dystonia, are “off” periods predictable, are “off” periods unpredictable, do “off” periods come on suddenly, within a few seconds; does the patient have anorexia, nausea, or vomiting; if any sleep disturbances, such as insomnia or hypersomnolence are present; and if the patient has symptomatic orthostasis.

In embodiments, the improvement is an improvement in the UPDRS-8. The UPDRS-8 was developed by the Movement Disorder Society to be more comprehensive than UPDRS, and take less time than the UPDRS to perform (Hauser, Lyons, and Pahwa, 2012). The UPDRS-8 includes eight items from the original UPDRS found to be “key features” of PD, in the categories of mentation, behavior, and mood; motor examination, and complications in therapy.

In embodiments, the improvement in the UPDRS-8 is an improvement in any of mentation, behavior, and mood; including intellectual impairment (UPDRS item 1) and depression (UPDRS item 3); motor examination, including tremor at rest (head, upper, and lower extremities) (UPDRS item 20), finger taps (patient taps thumb with index finger in rapid succession) (UPDRS item 23), and Gait (UPDRS item 29); and complications in therapy, including dyskinesias duration (what proportion of the waking day dyskinesias are present) (UPDRS item 32), dyskinesias disability (how disabling the dyskinesias are) (UPDRS item 33), and the proportion of the waking day the patient is “off” on average (UPDRS item 39). In embodiments, the improvement in the UPDRS-8 is a reduction in score. In embodiments, the reduction in score is a reduction of between about 1 to about 4 points, including 1 point, 2 points, 3 points, and 4 points.

In some embodiments, at least one measurable improvement will be seen within 120 days of initiating treatment with a disclosed combination. In embodiments, the time elapsed between administering to a patient a therapeutic combination and the improvement is less than about 120 days, including less than 120 days, less than 115 days, less than 110 days, less than 105 days, less than 100 days, less than 95 days, less than 90 days, less than 85 days, less than 80 days, less than 75 days, less than 70 days, less than 65 days, less than 60 days, less than 55 days, less than 50 days, less than 45 days, less than 40 days, less than 35 days, less than 30 days, less than 25 days, less than 20 days, less than 15 days, less than 10 days, less than 5 days, 1 day or less, including 18 hours or less, 12 hours or less, 6 hours or less, 3 hours or less, 2 hours or less, 1 hour or less, 30 minutes or less, 15 minutes or less, and 1 minute or less, and values in between.

In embodiments, a patient is administered a therapeutic combination and will experience a durable reduction in at least one symptom of a movement disorder. In embodiments, the durable reduction may be determined based on measures known to ordinary skill, including changes in patient completed self-assessments, and clinical outcome assessment or index scores, wherein a decrease in score of one or more points indicates a decrease in the severity of symptoms; in embodiments, the durable reduction may be determined via methods used to diagnose movement disorders, such as those disclosed herein.

In embodiments, a patient is prescribed a therapeutic combination or may obtain it over-the-counter (OTC). In embodiments, the therapeutic combinations may be obtained from a health-care provider or pharmacy as an OTC medication, and/or may be obtained via purchase at a vendor who is not a health-care provider or pharmacy.

In embodiments, the therapeutic combination will be useful in improving mood, and sleep regulation. In embodiments, the therapeutic combination will be useful in improving fine motor control in between about 5 and about 30 days after beginning treatment, including 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, and 30 days. In embodiments, the therapeutic combination will be useful in improving phenotype-specific gross motor control between about 15 and about 45 days after beginning treatment, including 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, and 45 days. In embodiments, the therapeutic combination will be useful in improving cognitive and behavioral attitudes towards exercise. In embodiments, the therapeutic combination will be useful in reducing the volatility of effects through on-off phases of levodopa therapy, which is utilized in the treatment of movement disorders to increase dopamine concentrations. In embodiments, the therapeutic combination will be useful in returning dopaminergic capacity and reducing the need for levodopa therapy.

In some embodiments, an advantageous effect of a disclosed therapeutic combination, as compared to the current standard of care in the respective indication may include any of: (1) a larger percentage of patients experiencing a clinical response; (2) a larger average clinical response; (3) an earlier onset of the clinical response; (4) a more durable clinical response; (5) a similar or better clinical response with fewer or different side effects and therefore improved compliance; and (6) a similar or better clinical response with a more convenient therapeutic regimen with fewer drug administrations and therefore improved compliance. In some embodiments, a comparison to the current standard of care consists of, or comprises, a comparison to any of the antiparkinson agents known or disclosed herein.

In some embodiments, an advantageous effect of a disclosed therapeutic combination will include delaying levodopa introduction in early-stage patients. In some embodiments, an advantageous effect of a disclosed therapeutic combination will include reducing reliance on levodopa in mid-stage patients.

### J. EXAMPLES

### Example 15: Preliminary Testing

Compositions according to disclosed embodiments were initially tested by individuals with chronic diseases, including Parkinson's disease (PD). Treatment of individuals with PD, who generally had no other therapeutic options, and who reported being afflicted with anxiety, depression, advanced stages of degenerative disease, and other complex disorders, provided initial evidence of visible control of motor damage-related symptoms, including reduction of tremor frequency and intensity, and benefits to behavioral symptoms including mood swing control and apathy reduction. The pattern of recovery was demonstrated across many individuals.

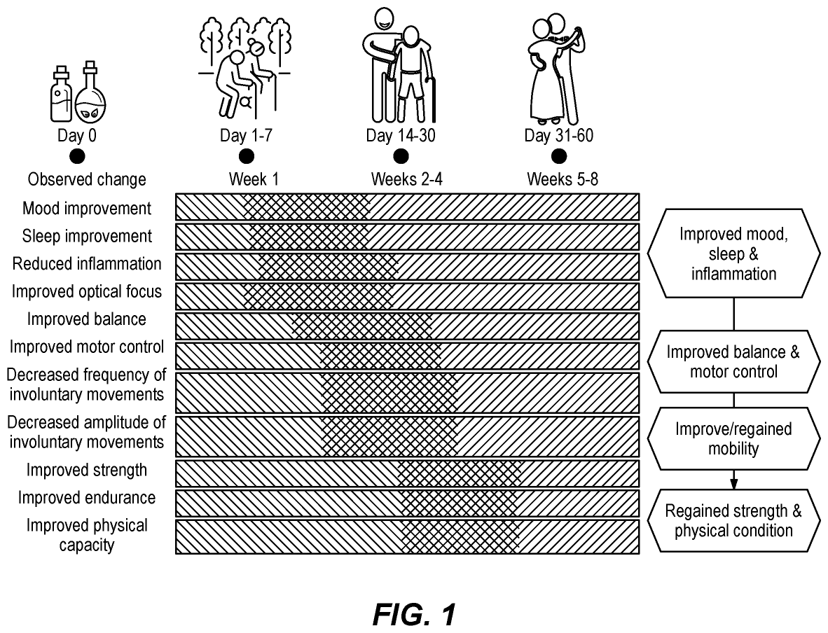

As seen in FIG. 1, repeated patterns of therapeutic activity emerged in those individuals who used a disclosed composition over the course of 8 weeks (1-60 days). Individuals tended to present improvements to mood, sleep, inflammation, and optical focus by the end of week 1, with each bar illustrating the observed change listed on its left hand side. From the left, diagonal hatching in each bar from left downward to the right illustrates the period before the onset of measurable changes. The cross-hatched portion in the middle of each bar illustrates the period from the onset of measurable changes until marked improvements are seen. The right hand portion of each bar, with diagonal hatching from left upward to the right illustrates the period where marked improvements are maintained. During weeks 2-4, individuals tended to show improvements in balance and motor control, in addition to decreases in frequency of involuntary movements and amplitude of involuntary movements. During weeks 5-8, individuals utilizing disclosed compositions displayed improved strength, endurance, and physical capacity. Generally, an improvement to mood, sleep, and inflammation occurred first, followed by improved balance and motor control. Then, mobility was improved or regained, followed by improvements to strength and physical condition. The progression in overall improvements is illustrated by the boxes along the right hand side of the bars, moving from top to bottom.

Dose and Composition: Subjects took 1 drop sublingually/1 puff inhaled, 4 times a day. Each dose of the therapeutic combination was 50 mg total, and contained 48% Psilocybe cubensis extract comprising, among other compounds, psilocin and psilocybin; 16% Cannabis sativa extract comprising, among other compounds, THC and CBD; 4% Dipteryx odorata extract comprising, among other compounds, coumarin; 16% Pyropia yezoensis extract comprising, among other compounds, polysaccharides (porphyran) and taurine; and 16% a ginger ethanol solution. Each dose was compounded to include approximately 250 μg of psilocybin, 150 μg of psilocin, 1 mg of THC, 1 mg of CBD, 1 mg of Coumarin, and 8 mg of whole Pyropia extract.

Evidence was collected by Applicant via text message exchanges from caregivers or subjects, and/or self-shot before and after videos. Confirmed clinical remission was reported in one subject (Case 1 below), after using a disclosed combination for approximately one year.

Case 1: Male, 77 years old with diagnosed late-stage PD. On a wheelchair. Case 1 took the primary and/or secondary bioactive molecules for 54 days. No side effects were reported by a caregiver. Case 1 went under remission after 14 months of stopping using the formulation, which indicates powerful and durable motor and non-motor effects as shown in the following timeline:

Case 1 presented improved balance by day 18 of dosing, reduced tremor within 30 days of dosing with significant strength gains. Subject was able to stop the use of his wheelchair, was able to better take care of himself, and was even able to participate in a walking tour of Paris.

Case 1 began the disclosed therapeutic combination on Jul. 9, 2019 via 1 puff 4 times a day of inhaled formulation. Previous to treatment start, the subject presented limited tremor, inability to stand or walk unassisted, poor communication ability, and depression.

Case 1 showed an improved mood immediately on day 2, and onward stating “feels cheerier.” Subject stated: “Better attitude. Doing the twist,” on day 3. “A slow start this am. Balance not as good. He's back to the twist!” was stated on day 4. “Moving well again after a bit of a slow morning. Easily standing without holding on and walking while carrying stuff,” was stated on day 5. “Feel good today. It is incredible. He did some cooking too. Your dad cleared the table tonight. This is amazing,” was stated on day 6. “Not a great day today. Your dad did not sleep well so it was a rough start,” was stated on day 7.

Improved balance was the next positive outcome of utilizing the disclosed composition. “Feeling better than yesterday and balance is pretty good,” was stated on day 8. “His eyes look good,” was stated on day 9. “Much better today. Mood is good. He's rinsing dishes. Tonight, he was able to easily balance to change into his pajamas (Part 2.5 of the UPDRS). I can't even remember the last time that wasn't a struggle. Wow,” was stated on day 10. “Tremor is bad today, but the balance is ok,” was stated on day 11. On day 12, Subject stated “Parkinson's tremor doesn't look bad and your dad's balance is very good today.”

Case 1 then began showing improved mobility while maintaining wellness. On day 15, the subject stated “Doing well today. Balance and mood are good. Some days are really amazing. And it's more sustained than previous ups. I honestly can't remember when he had that kind of mobility.” “He's still doing well,” and “everything went well” were reported on day 18 and 21 respectively.

Case 1 then began regaining strength, such as on day 30: “He did great today at PT. Remember he needed help to stand. This is with a weighted vest.” “Making amazing progress in PT. His posture is better, his balance is better, he can walk faster and generally get up from a chair without using his hands,” on day 33.

Case 1 additionally began to show regained mobility, such as on day 36: “In 2 walking tests, he improved by 100%. When they did the initial assessment, he was able to go from seated to standing twice in one minute. This time, in a lower chair, he did 9.” On days 39 and 40, subject showed further regaining of mobility: “I joined the YMCA,” “First workout at the YMCA accomplished! Your dad did great.”

Further improvements of Case 1 were observed through neurological examination. Day 47, the subject stated “Neurologist noted a big improvement since she first saw him.”

Following the last day of treatment on day 50, Case 1 noticed continued long-term effects and benefits post-treatment. 9 days post treatment: “Your dad is doing amazingly well on this trip! We did not bring a wheelchair and he's doing a great job of navigating the tours.” 20 days post-treatment, subject stated “Your dad is doing well. We walked about 2 miles.” Long-term effects continued, with the latest update 475 days post-treatment: “Still walking, same status, no reversal despite impact of COVID (14 months after last dose).”

Case 2: Male, 50 years old with diagnosed PD. Case 2 took a disclosed therapeutic combination 4 times a day via inhalation (1 puff every time) for about 28 days. Prior to taking the primary and/or secondary bioactive molecules, Case 2 had a significant tremor and was depressed. After the treatment, Case 2's tremor was dramatically reduced.

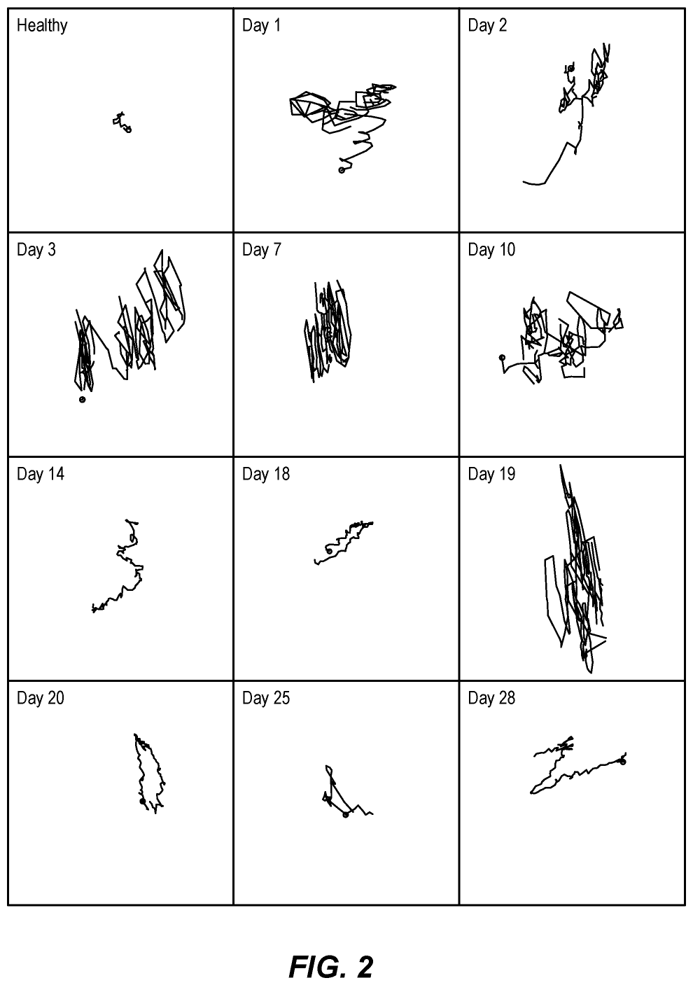

Hand motion improvements were observed by analyzing 6-second videos of the left hand with software-assisted motion tracking (although Day 25 is 3 seconds only) as shown in FIG. 2 (with boxes left to right, and top to bottom, representing healthy, Day 1, Day 2, Day 3, Day 7, Day 10, Day 14, Day 16, Day 19, Day 20, Day 25, and Day 28):

Analyses were for qualitative evaluation. The videos were self-reported. The analysis suggests a transition from uncontrolled, sharp and ample movements to smaller, smoother movements from Day 14 after the start of the treatment. Day 19 represents a “flare” which was short lasting, as from day 20-28 a significant hand motion improvement was observed. Case 2 also reported mood improvements felt after a few days of dosing.

### Example 16: Exemplary Patient Study

Fifteen patients (14 male, one female) diagnosed with Parkinson's between 40 and 84 (median age 74) were administered ABS-108 (i.e., a disclosed composition of EXAMPLE 1, sometimes also referred to as “NIM-01”), for between 30 and 1,142 days. Mood and motor symptoms were assessed, and the date at which measurable response to treatment was identified was graphed. TABLE 3 below illustrates the results for non-motor, motor, and motor complication components of the UPDRS-8 prior to the first administration (labeled as “Pre”) and at the final measured administration of the combinations disclosed herein (labeled as “Post”).

TABLE 3
Non-motor, motor, and motor complication components by patient

| | Non-Motor | | Motor | | Motor Complications | |
|---|---|---|---|---|---|---|
| | Pre | Post | Pre | Post | Pre | Post |
| A2 | 4 | 1 | 11 | 4 | 0 | 0 |
| A3 | 6 | 2 | 7 | 4 | 0 | 0 |
| A4 | 5 | 2 | 7 | 4 | 0 | 0 |
| A5 | 0 | 0 | 7 | 4 | 0 | 0 |
| A6 | 4 | 1 | 8 | 1 | 6 | 3 |
| A9 | 4 | 2 | 12 | 7 | 0 | 0 |
| A10 | 3 | 2 | 8 | 6 | 0 | 0 |
| A11 | 6 | 2 | 12 | 7 | 0 | 0 |
| A12 | 5 | 2 | 12 | 7 | 7 | 3 |
| A13 | 6 | 3 | 15 | 10 | 12 | 9 |
| A14 | 4 | 2 | 6 | 5 | | |
| A15 | ND | ND | ND | ND | ND | ND |
| A16 | 4 | 2 | 4 | 3 | 0 | 0 |
| A17 | 2 | 2 | 7 | 5 | | |
| A18 | 6 | 4 | | | | |

Relevant information about the above patients is provided in the paragraphs that follow. Although the paragraphs that follow in this Example may use the present tense (e.g., in the Clinical Notes sections below), it will be understood that this is merely a consequence of transcribing the unedited clinical observations from the disclosed patient reports, and should not be construed as presenting prophetic results; each of the patient reports provided in this Example represent working examples of the results of administering a disclosed therapeutic combination to an actual patient.

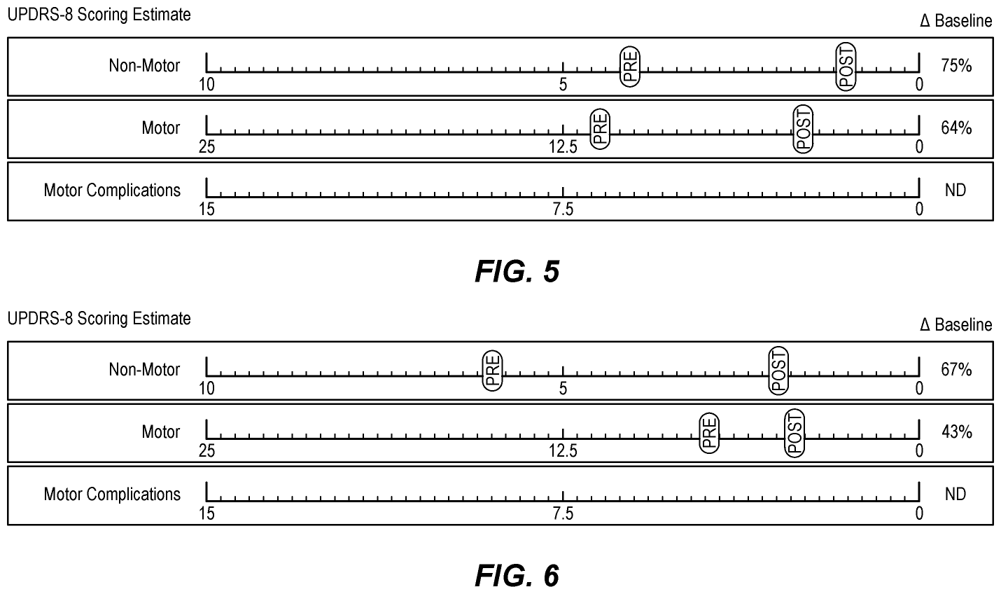

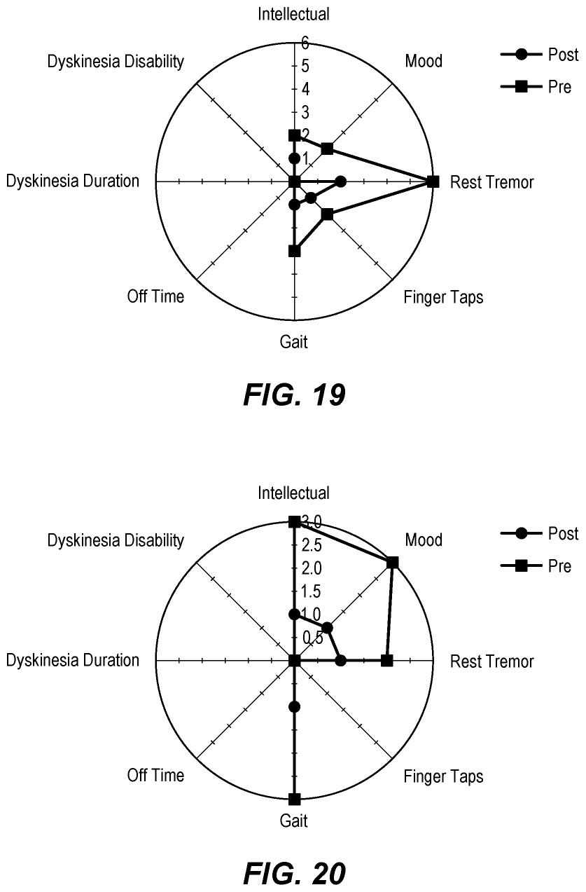

Patient A2: Diagnosed in 2002. Began Treatment with ABS-108 in July 2020, at Age 82.
Patient Background: Male. Background in yoga, alternative medicine, and meditation. Former singer. Currently provides individual therapy and presents as well as teaches to large groups. Very active.
Primary Presentation: Marked tremor in both upper and lower extremities. Unable to walk unassisted. Very rigid. Speech difficult to understand.
Primary Treatment: Sinemet.
Other Treatment: Yoga, meditation, exercise.
Clinical Notes: Caretaker reports rapid (<21 day) changes to mood and endurance. At four months, patient reports continued positive mood, significantly improved endurance (from only short walks to 2-4 mile excursions). Patient and friends report significant changes to voice. At month 6, patient is walking steadily, unassisted, playing piano, meditating again, and generally feels good. Since month 6, patient has been taking ABS-108 intermittently, with a month or two on and a few weeks off, due to surgery and various unrelated illnesses. Update 7/2023, after 3 years of taking ABS-108, is the patient is strong and well. Family and friends continue to express thanks. FIG. 5 details the UPDRS-8 Scoring Estimate of patient A2, including pre- and post-treatment with ABS-108 as a change from a baseline score. The categories measured are non-motor, motor, and motor complications. FIG. 5 depicts an improvement of 75% in non-motor skills and an improvement of 64% in motor skills for patient A2 after beginning treatment. FIG. 19 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None Reported.
   Patient A3: Diagnosed in 2006. Began Treatment with ABS-108 in January 2021, at Age 84.
Patient Background: Male. Former engineer who had previously enjoyed tinkering.
Primary Presentation: Parkinson's with Lewy-body dementia. Unable to stay on task, even for introductory assessment. Very depressed. Moderate tremor and difficulty walking. Falling multiple times a day. Patient and caretakers all very frustrated
Primary Treatment: SINEMET® (carbidopa/levodopa).
Other Treatment: None.
Clinical Notes: Rapid (<14 day) improvement in mood, and the regular falls stop completely. At 60 days, care-takers report notable progress with his ability to complete tasks (namely, fixing a sink he′d been trying to fix for some time). Memory and mood both improved substantially. Spouse and children report regular, continued improvement and remain excited about the protocol. FIG. 6 details the UPDRS-8 Scoring Estimate of patient A3, including pre- and post-treatment with ABS-108 as a change from a baseline score. FIG. 6 depicts an improvement of 67% in non-motor skills and an improvement of 43% in motor skills for patient A3 after beginning treatment. The categories measured are non-motor, motor, and motor complications. FIG. 20 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.

### Patient A4: Diagnosed in 2012. Began ABS-108 Treatment in May 2019, at Age 77.

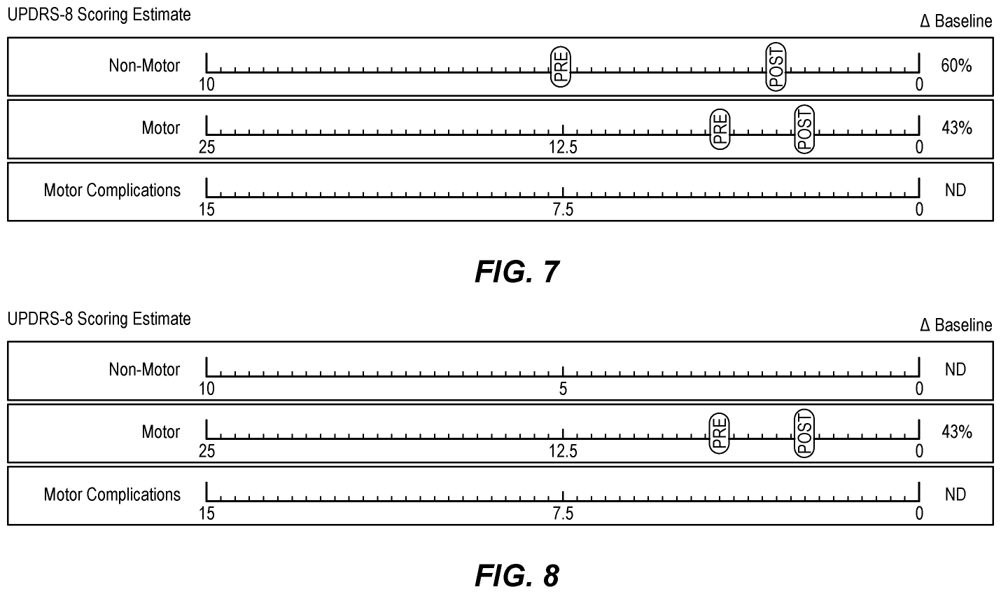

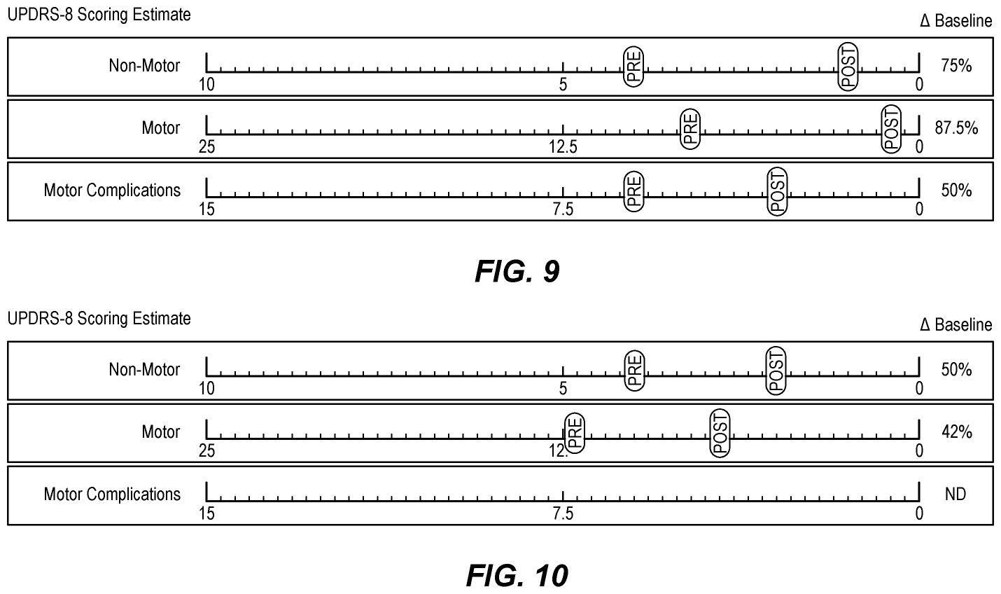

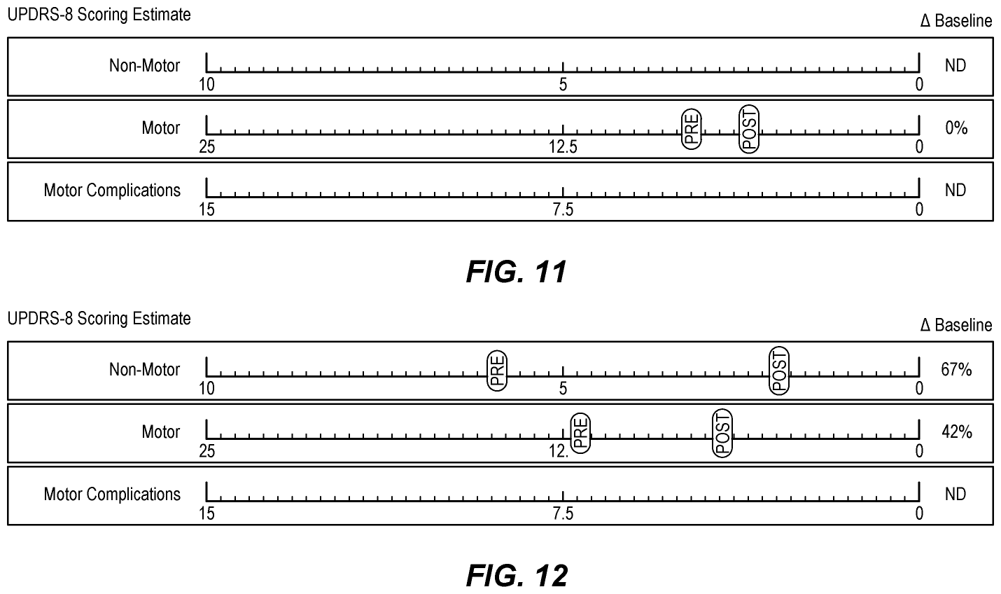

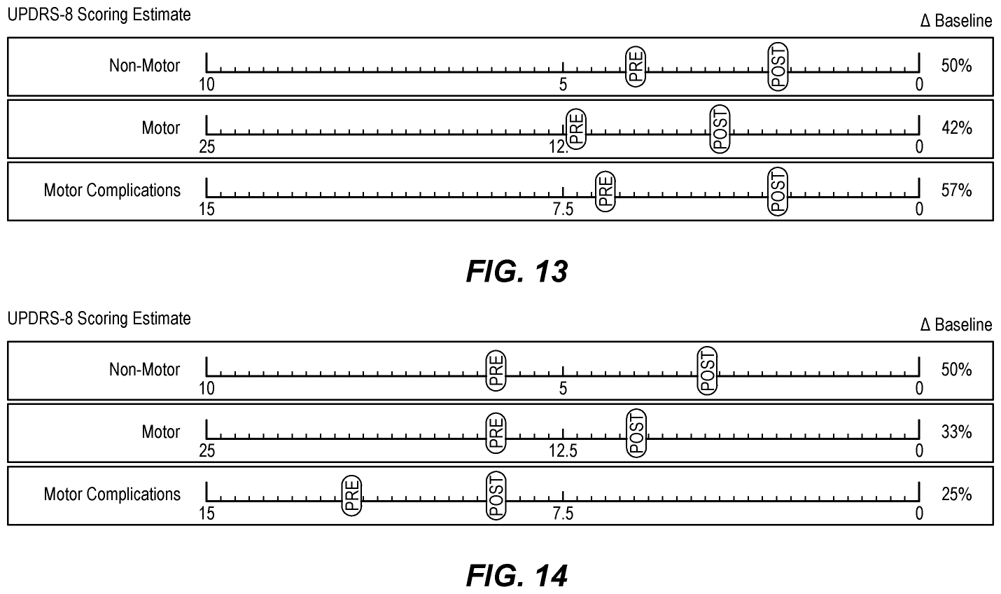

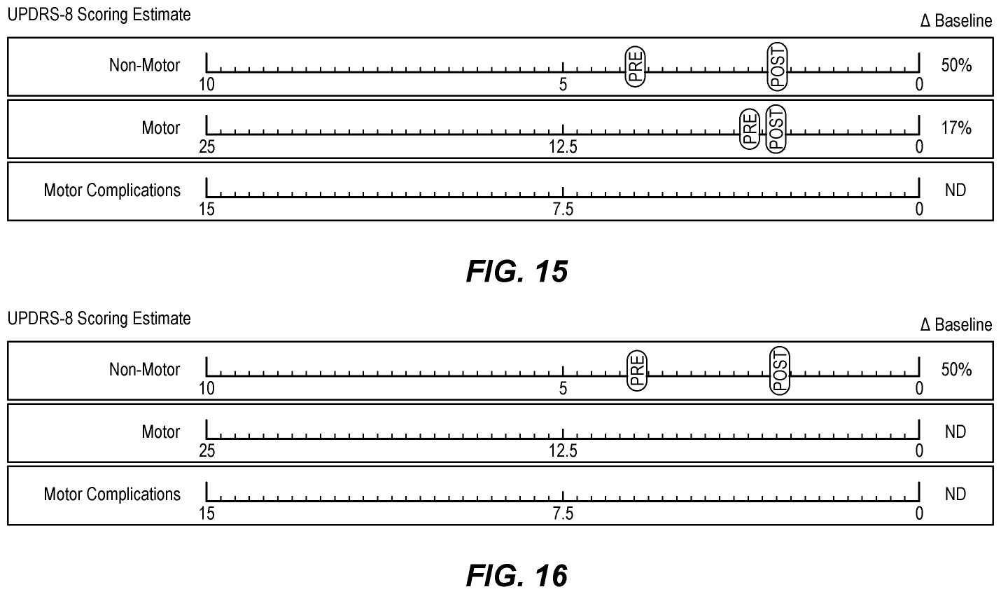

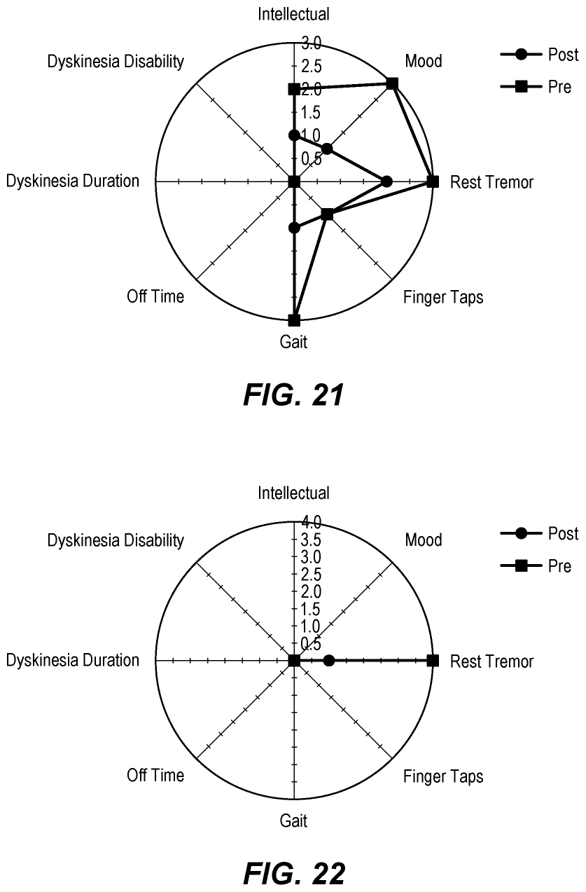

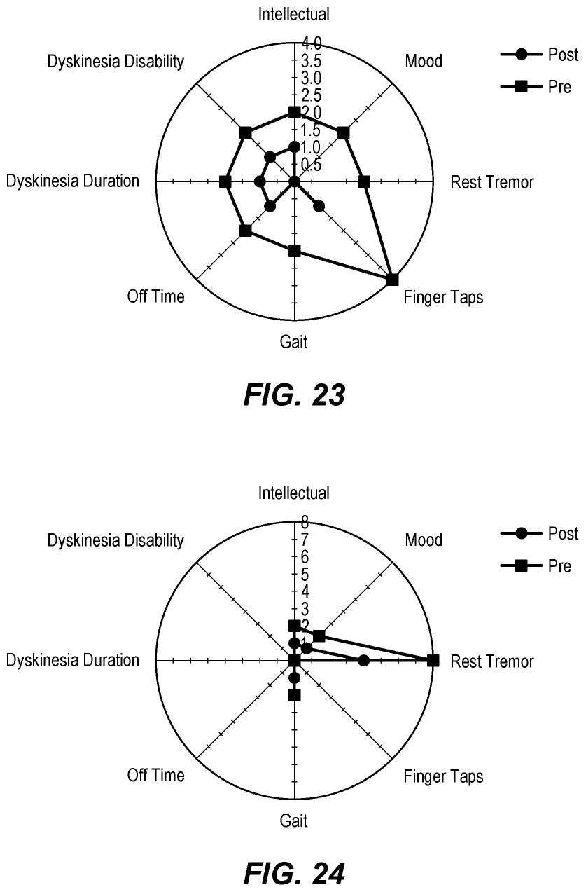

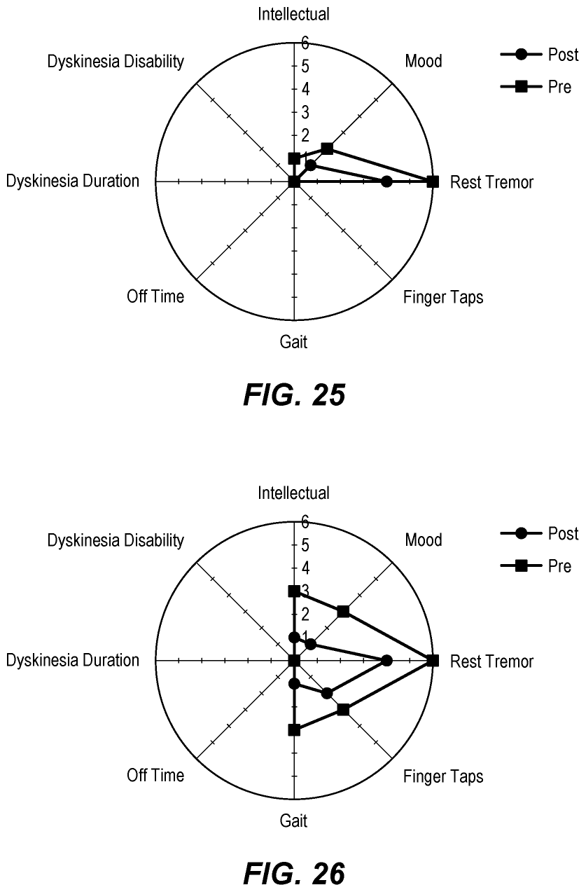

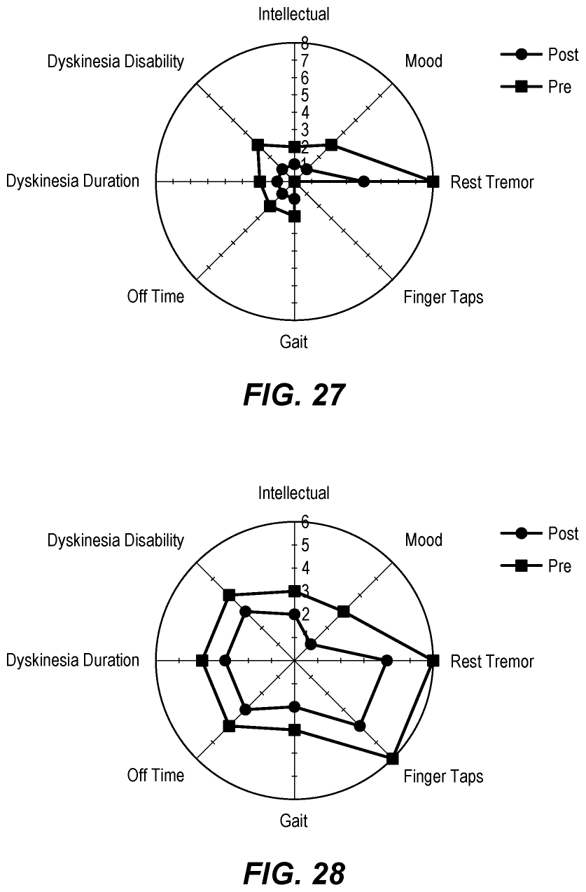

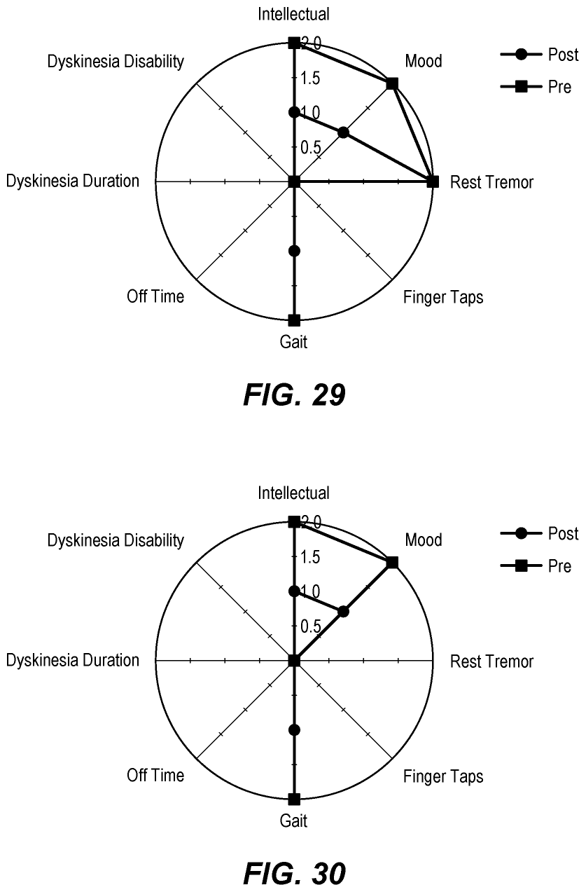

Patient Background: Male. Former USDA employee and farm worker, with significant exposure to pesticides and herbicides
Primary Presentation: Moderate tremor in both hands (one hand may be ET). Requires assistance for walking or any motor tasks (shifting into a chair, for instance). Relies on a wheelchair. Poor vocal ability and eye control.
Primary Treatment: L-Dopa/Carbidopa
Other Treatment: Physical therapy.
Clinical Notes: Patient tried several variations of ABS-108, starting and stopping for the first 6 weeks. Ability to stand and walk unassisted within 30 days of stable formula (70 days total) Caretaker reports significant mood changes in the same period, ability to change clothes independently, will and ability to do basic household chores. At 60 days, patient demonstrates significant improvement to strength and endurance through weighted chair-lift exercises. Patient elects to stop treatment after 95 days for a walking tour in France. Patient's primary symptoms do not return. Summer 2021 check-in highlights some deterioration with the loss of physical therapy during the COVID-19 pandemic, but no return to primary symptoms. FIG. 7 details the UPDRS-8 Scoring Estimate of patient A4, including pre- and post-treatment with ABS-108 as a change from a baseline score. The categories measured are non-motor, motor, and motor complications. FIG. 7 depicts an improvement of 60% in non-motor skills and an improvement of 43% in motor skills for patient A4 after beginning treatment. FIG. 21 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A5: Diagnosed in 2017. Began Treatment with ABS-108 in July 2019, at Age 58.
Patient Background: Male. Attorney with no known familial or environmental predisposition
Primary Presentation: Tremor in both hands, more significantly on the left. Mild confusion/memory lapses, changes to voice, and minor gait issues.
Primary Treatment: Sinemet.
Other Treatment: Parkinson's Boxing.
Clinical Notes: Conducted a 30-day evaluation of ABS-108 effect on his tremor specifically. At day 10, patient's tremor changed amplitude and frequency. At day 17, tremor gets suddenly worse again for ˜24 hours, before dissipating completely. Followup in summer 2019, patient reports that his cohort in PD boxing have degenerated significantly, while he has not. FIG. 8 details the UPDRS-8 Scoring Estimate of patient A5, including pre- and post-treatment with ABS-108 as a change from a baseline score. The categories measured are non-motor, motor, and motor complications. FIG. 8 depicts an improvement of 43% in motor skills for patient A5 after beginning treatment. FIG. 22 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A6: Diagnosed in October 2020. Began Treatment with ABS-18 Apr. 2021 at Age 54.
Patient Background: Male. Very active, athletic. Former software sales manager. Left job on long-term disability after being formally diagnosed.
Primary Presentation: Moderate tremor in right hand, limited tremor in left hand. Struggled extensively and occasionally failed tap test in right hand. Occasional misstep/fall. Erectile dysfunction. Depressed mood
Primary Treatment: Sinemet 25/100× daily
Other Treatment: PD Boxing
Clinical Notes: Rapid response to treatment. Tremor dissipated in right hand within the first 7 days. Speed and quality of tap-test improved within the first 7 days. Sexual function returned within 30 days. Mood improved, as noted by both patient and caretaker. Within 2 months, patient's strength, agility, and endurance improved, as evidenced during PD boxing. Patient stops taking Sinimet at day 45, with no change to condition. Patient continues to progress, and remains on the protocol. In December 2021, patient notes that going multiple days without treatment does cause tremor to return. Patient reports continued health in June 2023, after more than 2 years of taking ABS-108. FIG. 9 details the UPDRS-8 Scoring Estimate of patient A6, including pre- and post-treatment with ABS-108 as a change from a baseline score. The categories measured are non-motor, motor, and motor complications. FIG. 9 depicts an improvement of 75% in non-motor skills, an improvement of 87.5% in motor skills, and an improvement of 50% in motor complications for patient A6 after beginning treatment. FIG. 23 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A9: Diagnosed in 2019. Began Treatment with ABS-108 in June 2021, at Age 62.
Patient Background: Male.
Primary Presentation: Intense, nearly constant tremor in both upper extremities. Difficulty with gait, balance, and strength. Moderate confusion/memory loss.
Primary Treatment: Sinemet.
Other Treatment: Physical therapy.
Clinical Notes: Very rapid (<7 day) loss of tremor at worst part of day (evening). Continued reduction in amplitude and frequency of tremor throughout the day. Some improvement to balance, as recorded by physical therapist. Patient reports improvement to mood and memory. After 60 days, patient feels his progress has plateaued, and elects to stop protocol. FIG. 10 details the UPDRS-8 Scoring Estimate of patient A9, including pre- and post-treatment with ABS-108 as a change from a baseline score. The categories measured are non-motor, motor, and motor complications. FIG. 10 depicts an improvement of 50% in non-motor skills and an improvement of 42% in motor skills for patient A9 after beginning treatment. FIG. 24 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A10: Diagnosed in 2019. Began Treatment with ABS-108 in July 2021, at Age 61.
Patient Background: Male. Plumber. Very rapid onset PD, with complete resistance to all available medications.
Primary Presentation: Tremor throughout whole body, whether resting or active. Reports having tried every medication available, to no effect. Very depressed and overwhelmed.
Primary Treatment: None effective.
Other Treatment: None.
Clinical Notes: No response until ˜40 days. At that point, patient has a sudden quieting of most tremor that lasts approximately one week. Tremor returns, and patient gives up hope that medication will help and elects to stop protocol. FIG. 11 details the UPDRS-8 Scoring Estimate of patient A10, including pre- and post-treatment with ABS-108 as a change from a baseline score. The categories measured are non-motor, motor, and motor complications. FIG. 11 shows no change in motor skills for patient A10 after beginning treatment. FIG. 25 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A11; Diagnosed in 2001. Began Treatment with ABS-108 in September 2021 at Age 73.
Patient Background: Female. Has suffered from PD for 20 years. Formerly quite active, her confusion is too pronounced to take walks safely. Recently had to move in with her daughter and son-in-law for additional support.
Primary Presentation: Tremor in both upper extremities. Confusion, memory loss. Very low endurance. Moderate difficulty with walking and balance. Difficulty sleeping. Cannot function without l-dopa treatment, so taking nearly constantly.
Primary Treatment: Sinemet 10× daily.
Other Treatment: None.
Clinical Notes: Improved mood very quickly (<14 days). Very limited physical response until day 50. At that point, her disorientation improved sufficiently for her to drive and walk alone. Sleep duration improved dramatically. Tremor reduced, and overall motor skills improved. Endurance much improved, with walks going from very short to multiple miles over the course of just 2 weeks. FIG. 12 details the UPDRS-8 Scoring Estimate of patient All, including pre- and post-treatment with ABS-108 as a change from a baseline score. The categories measured are non-motor, motor, and motor complications. FIG. 12 depicts an improvement of 67% in non-motor skills and an improvement of 42% in motor skills for patient All after beginning treatment. FIG. 26 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A12: Diagnosed in 2018. Began Treatment with ABS-108 in June 2021, at Age 40.
Patient Background: Male. Very young. Entrepreneur with very aggressive PD. Friends and coworkers report years of depression, anger, and loss of cognition.
Primary Presentation: Strong, aggressive tremor in upper and lower extremities. Significant depression. Reduced cognitive capabilities. Poor gait.
Primary Treatment: Sinemet, Ropinirole.
Other Treatment: THC/CBD.
Clinical Notes: At 88 days, patient reports increased energy, clearer head, less depression, and a 10-20% reduction in physical symptoms. At that point, patient stops taking roprinirole entirely and begin to wean off sinimet. In another 30 days, patient shows very visible loss of tremor in both upper and lower extremities, with only occasional l-dopa treatment. FIG. 13 details the UPDRS-8 Scoring Estimate of patient A12, including pre- and post-treatment with ABS-108 as a change from a baseline score. The categories measured are non-motor, motor, and motor complications. FIG. 13 depicts an improvement of 50% in non-motor skills, an improvement of 42% in motor skills, and an improvement of 57% in motor complications for patient A12 after beginning treatment. FIG. 27 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A13: Diagnosed in 2006. Began Treatment with ABS-108 in September 2021, at Age 84.
Patient Background: Male. Advanced Parkinson's for some time. Caretakers report struggling to manage his advanced degeneration.
Primary Presentation: Regular, low-amplitude tremors in upper extremities. Walks with a walker and assistance. Very long freezes (up to 10 minutes), occurring every 2 steps. Weak voice, significant depression. Requires help with even very basic tasks
Primary Treatment: Sinemet.
Other Treatment: SEROQUEL® (quetiapine).
Clinical Notes: Within 2 weeks, caretaker reports significant improvement to sleep (sleeping for 7-8 hours uninterrupted) and improved ability to open his hands. By 60 days, caretaker reports he is now able to transfer in and out of bed on his own, and that his physical therapist is very impressed with improvements to his balance. Freezes less frequent, though still occurring. After 2.5 months, caretaker reports “His primary/internal medicine doctor thought he looked the best he has seen in the last several visits!! ” FIG. 14 details the UPDRS-8 Scoring Estimate of patient A13, including pre- and post-treatment with ABS-108 as a change from a baseline score. The categories measured are non-motor, motor, and motor complications. FIG. 14 depicts an improvement of 50% in non-motor skills, an improvement of 33% in motor skills, and an improvement of 25% in motor complications for patient A13 after beginning treatment. FIG. 28 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: Patient reports very low blood pressure on the morning of day 17. Caretaker decides to reduce ABS-108 dosage 2 3× daily, and beings to wean seroquel.
   Patient A14: Diagnosed in 2006. Began Treatment with ABS-108 in October 2021, at Age 78.
Patient Background: Male. Vietnam veteran who began showing symptoms, primarily lethargy and gait problems, roughly 15 years ago.
Primary Presentation: Mild tremor in upper extremities. Very depressed, tired, low energy. Takes multiple naps/day.
Primary Treatment: Sinemet, every 4 hours.
Other Treatment: None.
Clinical Notes: Within 1 week, caretaker reports that patient has more energy, takes 2-3 fewer naps/daily, and is “generally sharper.” Caretaker is ecstatic by patient progress. In early December, Patient's brothers both report remarkable improvements to voice and energy, obvious to them by phone. At the same time, patient feels “plateaued” and is anxious for faster change. FIG. 15 details the UPDRS-8 Scoring Estimate of patient A14, including pre- and post-treatment with ABS-108 as a change from a baseline score. The categories measured are non-motor, motor, and motor complications. FIG. 15 depicts an improvement of 50% in non-motor skills and an improvement of 17% in motor skills for patient A14 after beginning treatment. FIG. 29 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A16: Diagnosed in 2015. Began Treatment with ABS-108 in October 2021, at Age 77.
Patient Background: Male with reasonably aggressive PD.
Primary Presentation: Regular, low-amplitude tremors in upper extremities. Difficulty getting up from a chair unassisted (though possible). Slow, awkward gait. Periods of depression and confusion. Compulsive, possibly resulting from l-dopa treatment or dementia.
Primary Treatment: Sinemet.

### Other Treatment:

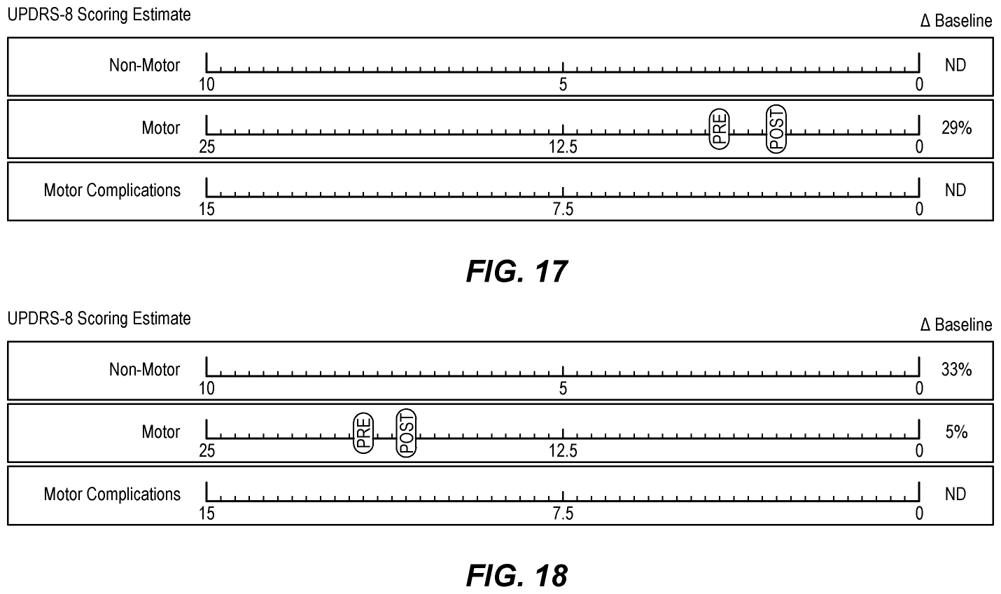

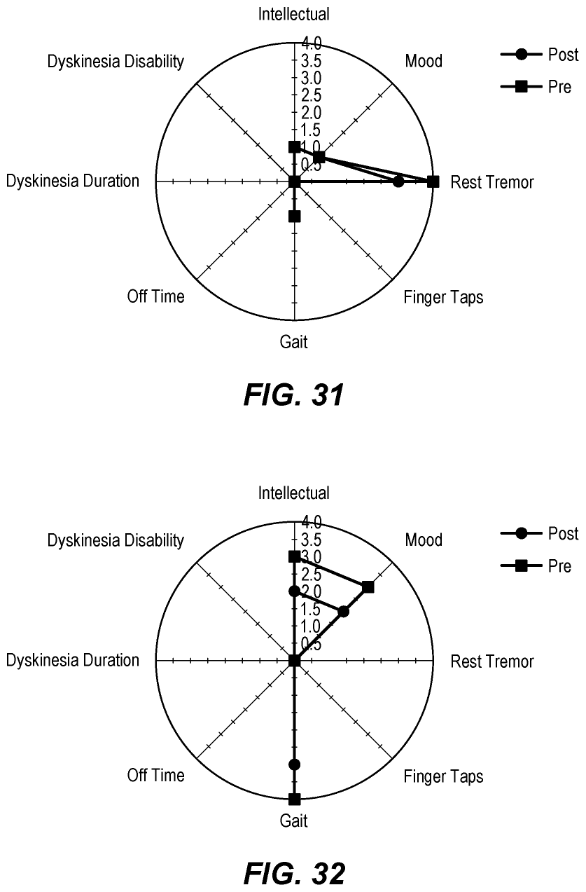

Clinical Notes: Within 45 days, caretaker reports he feels “much much sharper,” and improvements to mood. Patient is anxious to continue treatment and see where it leads him. Improvements continue for the next several check-ins before stabilizing. FIG. 16 details the UPDRS-8 Scoring Estimate of patient A16, including pre- and post-treatment with ABS-108 as a change from a baseline score. The categories measured are non-motor, motor, and motor complications. FIG. 16 depicts an improvement of 50% in non-motor skills for patient A16 after beginning treatment. FIG. 30 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A17: Diagnosed in 2017. Began Treatment with ABS-108 in October 2021 at Age 74.
Patient Background: Male. Incredibly good attitude and supportive wife. Manages PD through yoga, meditation, and l-dopa treatment
Primary Presentation: Regular, low-amplitude tremors in upper extremities. Shuffling, slightly rigid gait. Regular, low-amplitude tremor in mouth/lips
Primary Treatment: Sinemet.
Other Treatment: Yoga, meditation.
Clinical Notes: Rapid change to amplitude of hand tremor. Persists, but much lower amplitude (this is from video evidence--patient doesn't feel as if it has changed much). Patient reports improved mood after one week. By day 30, gait seems to have improved. Increase dosage first week of December 2021, to push through ever-improving tremor. Patient continues to feel healthy and strong nearly 2 years later (August 2023). FIG. 17 details the UPDRS-8 Scoring Estimate of patient A17, including pre- and post-treatment with ABS-108 as a change from a baseline score. The categories measured are non-motor, motor, and motor complications. FIG. 17 depicts an improvement of 29% in motor skills for patient A17 after beginning treatment. FIG. 31 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.
   Patient A18: Diagnosed at Age 2013. Began Treatment with ABS-108 in October 2021, at Age 75.
Patient Background: Male.
Primary Presentation: Generally inactive. Prominent rigidity, tremor. Cannot hold posture. Drools. Severe constipation. Depressed.
Primary Treatment: L-DOPA.
Other Treatment: Rytary, Midodrine, Ritalin, Trulance, Tamulosin, Clonazapam, Gabapentin.
Clinical Notes: Within 45 days, wife and children all report similar progress: (1) Less drooling; (2) Sitting up straight more; (3) Seems more alert; (4) constipation has been better; (5) He has been going out more in the world (lunch on Wednesday of last week, theater on Sunday), and he wasn't as engaged in the world before; (6) He seems to be moving around a bit better. Observations after 4 months show significant improvement in posture and ability to stand from a seated position. FIG. 18 details the UPDRS-8 Scoring Estimate of patient A18, including pre- and post-treatment with ABS-108 as a change from a baseline score. The categories measured are non-motor, motor, and motor complications. FIG. 18 depicts an improvement of 33% in non-motor skills and an improvement of 5% in motor skills for patient A18 after beginning treatment. FIG. 32 further provides improvement in UPDRS-8 scores when comparing pre- and post-treatment with ABS-108 in the categories of Dyskinesia Duration, Dyskinesia Disability, Intellectual, Mood, Rest Tremor, Finger Taps, Gait, and Off-Time.
Adverse Events: None reported.

Summary information about each patient is also provided in TABLE 4, including number of participants, median age, mediant progression, treatment duration, response rate, specific patient age and gender, the beginning and end of treatment, and relative and gross changes in UPDRS-8 scores.

TABLE 4
Patient summary information

| | | | | | Treatment | |
|---|---|---|---|---|---|---|
| | | | | MED | Duration | Response Rate |
| n | | | MED Age | Progression | 30-1142 Days | 100% |
| 14 | | | 74 | 8 Years | UPDRS-8 | UPDRS-8 |
| | Age | Gender | Start Date | End Date | Δ/Relative | Δ/Gross |
| A2 | 82 | M | Jul. 9, 2020 | N/A | −67% | −29% |
| A3 | 84 | M | Jan. 26, 2021 | Sep. 15, 2022 | −54% | −20% |
| A4 | 77 | M | May 9, 2019 | Sep. 1, 2019 | −50% | −17% |
| A5 | 58 | M | Jul. 1, 2019 | Jul. 30, 2019 | −43% | −9% |
| A6 | 54 | M | Apr. 8, 2021 | N/A | −72% | −29% |
| A9 | 62 | M | Jun. 3, 2021 | Aug. 3, 2021 | −44% | −20% |
| A10 | 61 | M | Jul. 13, 2021 | Sep. 21, 2021 | −23% | −9% |
| A11 | 73 | F | Sep. 10, 2021 | Apr. 30, 2022 | −50% | −26% |
| A12 | 40 | M | Jun. 9, 2021 | Dec. 1, 2022 | −50% | −23% |
| A13 | 84 | M | Sep. 17, 2021 | Jul. 5, 2022 | −33% | −23% |
| A14 | 78 | M | Oct. 24, 2021 | Mar. 1, 2022 | −30% | −9% |
| A16 | 77 | M | Oct. 17, 2021 | Apr. 4, 2023 | −38% | −9% |
| A17 | 74 | M | Oct. 19, 2021 | N/A | −22% | −6% |
| A18 | 78 | M | Oct. 22, 2021 | Mar. 1, 2022 | −12% | −9% |

To obtain the relative UPDRS-8 scores, the relative change was calculated by subtracting the sum of the “post” score by the sum of the “pre” score, and dividing that value by the “pre” score. Represented another way, the relative score was calculated as follows: [(sum of post score-sum of pre score)/(the sum of the pre score)]. The gross change was calculated by subtracting the sum of the “post” score by the sum of the “pre” score, and dividing that value by the total possible score. Represented another way, the gross score was calculated as follows: [(sum of post score-sum of pre score)/(the total possible score)].

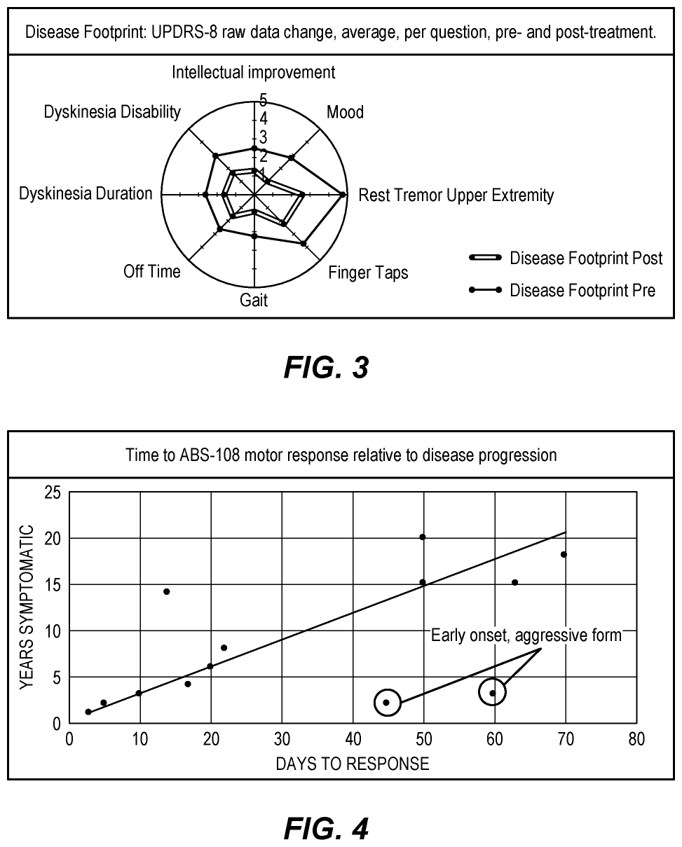

FIG. 3 (“Disease Footprint: UPDRS-8 raw data change, average, per question, pre- and post-treatment”) illustrates the average disease footprint pre- and post-treatment, as measured per question of the UPDRS-8 questionnaire illustrating the disease footprint in all eight assessed categories, including intellectual improvement, mood, rest tremor upper extremity, finger taps, gait, off time, dyskinesia duration, and dyskinesia disability. As is evident by the figure, the disease footprint is less post-treatment than pre-treatment.

FIG. 4 (“Time to ABS-108 motor response relative to disease progression”) illustrates the amount of treatment time (“ABS-108” is a disclosed composition) to realize a motor response generally increased linearly with the years an individual was symptomatic. The main outliers were two cases of early onset, aggressive Parkinson's where the individual in each case had symptoms for less than five years, and it took about 45 and about 60 days, respectively, to measure a motor response to treatment.

TABLE 5 below lists the number of treatment days elapsed (expressed as a range of days, wherein each range is inclusive) prior to a measurable response to treatment in both mood/motor symptoms, and shows that all participants (abbreviated as “Pt” for “patient”) experienced measurable improvements to mood symptoms (abbreviated “MD” for “mood”) within 35 days of beginning treatment, with half of the participants experiencing measurable mood improvements within 10 days. All participants experienced improved motor symptoms (abbreviated “MR” for “motor”) after receiving treatment, with over half of the participants experiencing improvements to motor symptoms within 25 days. A measurable response refers to a change in condition such as mood or motor function. Such measurements were observed through changes in UPDRS scores, questionnaires disclosed herein, physiological indicators (e.g., motor function tests), or other methods known to those of skill.

TABLE 5
Measurable Response Timeline of Patients Undergoing Treatment

| | Treatment Days | | | | | | | | | | | | | |
|---|---|---|---|---|---|---|---|---|---|---|---|---|---|---|
| | 1- | 6- | 11- | 16- | 21- | 26- | 31- | 36- | 41- | 46- | 51- | 56- | 61- | 66- |
| Pt | 5 | 10 | 15 | 20 | 25 | 30 | 35 | 40 | 45 | 50 | 55 | 60 | 65 | 70 |

| | | | | | | | | | | | | |
|---|---|---|---|---|---|---|---|---|---|---|---|---|
| A4 | MD | | | | MR | | | | | | | |
| A5 | MD | MR | | | | | | | | | | |
| A6 | MD | MR | | | | | | | | | | |
| A9 | MD | MR | | | | | | | | | | |
| A16 | | MD | | | MR | | | | | | | |
| A17 | | MD | | | MR | | | | | | | |
| A3 | | MD | | | | | | | | | MR | |
| A14 | | | MD | MR | | | | | | | | |
| A18 | | | MD | | | | MR | | | | | |
| A2 | | | MD | | | | | | | | | MR |
| A10 | | | | MD | | | | MR | | | | |
| A11 | | | | | | MD | | | MR | | | |
| A13 | | | | | | MD | | | MR | | | |
| A12 | | | | | | | MD | | | MR | | |

FIGS. 5-18 illustrate the UPDRS-8 scoring estimate pre- and post-treatment according to the method of EXAMPLE 16 for patients A2-A6, A9-A14 and A16-A18.

FIGS. 19-32 illustrate a spider chart with UPDRS-8 scores pre- and post-treatment for patients A2-A6, A9-A14 and A16-A18.

### Example 17: Exemplary Production of a Disclosed Composition

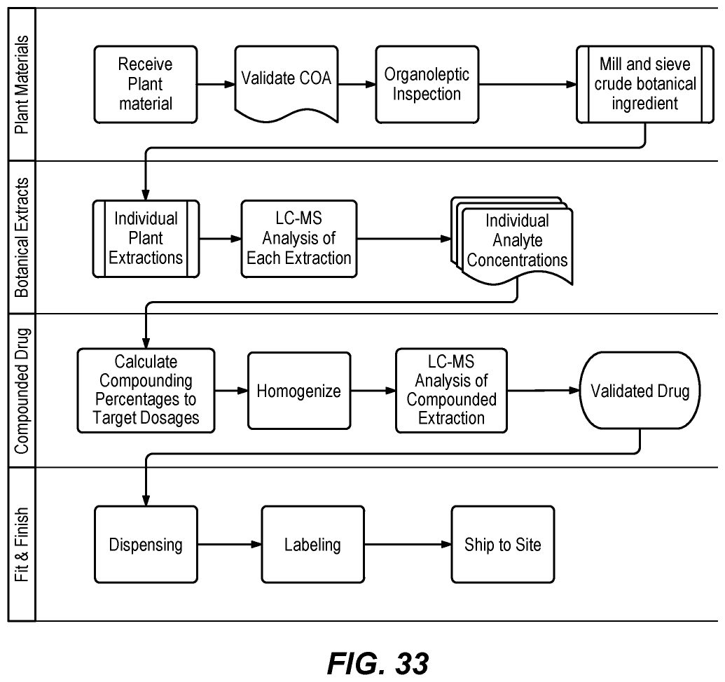

An exemplary process of producing a composition according to the methods of certain described embodiments is depicted in FIG. 33. In some embodiments, when a certain form of plant material is received for purposes of producing a composition, a Certificate of Analysis (COA) is obtained and validated for the plant, fungi, or algae material, and an organoleptic inspection is performed. A COA is a document that communicates the results of certain analyses done on a substance, including the presence of any chemicals used in the substance's manufacturing and testing, and can be created to ensure any applicable regulations are complied with. Organoleptic inspections are performed to detect disease or contamination within a substance. In some embodiments, the plant, fungi, or algae material is thereafter milled and sieved into a crude botanical ingredient.

In some exemplary embodiments, milled and sieved plant, fungi, and/or algae raw material can be prepared, extracted, and filtered as follows: Psilocybe cubensis: 30 grams of milled and sieved P. cubensis powder is first prepared by soaking for 48 hours in a 200 mL solution of 75% ethanol and 25% water. Then, it is extracted ultrasonically for 25 minutes at ¾ power and pulse duration. Next, it is filtered by hot filtration to 0.8 microns. In another embodiment, 40 grams of milled and sieved P. cubensis powder is first prepared for extraction in a Soxhlet apparatus by adding to a solution of 400 mL ethanol and 100 mL water. Then, it is extracted using the Soxhlet apparatus at 84° C. for 7 hours. Next, it is filtered by hot filtration to 0.8 microns (μm). In some preferred embodiments, both such methods of preparation, extraction, and filtration are used, with the results of each combined together. In some such preferred embodiments, where both such methods of preparation, extraction, and filtration are used, and where the results of each are combined together, the resulting combination comprises 80% of the ultrasound extraction, and 20% of the Soxhlet extraction, such as by w/w %. In some other preferred embodiments, the combination is 2:1 ultrasonic to Soxhlet solutions.

Cannabis sativa: 40 grams of milled and sieved C. sativa powder is first prepared for extraction in a Soxhlet apparatus by adding to a solution of 500 mL ethanol. Then, it is extracted using the Soxhlet apparatus at 84° C. for 7 hours. Next, it is filtered by hot filtration to 0.8 μm.

Pyropia yezoensis: 40 grams of milled and sieved P. yezoensis powder is first prepared by soaking for 48 hours in a solution of 200 mL ethanol and 40 mL water. Then, it is extracted ultra-sonically for 1 hour at ¾ power and pulse duration. Next it is filtered by hot filtration to 0.8 μm.

Dipteryx odorata: 50 grams of milled and sieved D. odorata powder is prepared, extracted, and filtered by percolation with 100 mL ethanol through a 0.4 μm filter until complete.

In some embodiments, after each of the milled and sieved plant, fungi, and/or algae raw material is prepared, extracted, and filtered, an LC-MS fingerprint is obtained for each.

In some embodiments, each of the prepared, extracted, and filtered plant, fungi, and/or algae material is blended and adjusted to the dose target, such as by the proportions of each. In some embodiments, a blend includes all of the filtered extracts of P. cubensis, C. sativa, P. yezoensis, and D). odorata, as described above. In some embodiments, a blend includes all of the filtered extracts of P. cubensis, C. sativa, P. yezoensis, and D. odorata, as described above, wherein the filtered extract of P. cubensis comprises a mixture of ultrasound and Soxhlet extraction methods, such as described above. In some embodiments, wherein the filtered extract of P. cubensis comprises a mixture of ultrasound and Soxhlet extraction methods, it comprises a mixture of 80% ultrasound and 20% Soxhlet extraction methods, such as described above. In some embodiments, a blend includes two or more of the filtered extracts of P. cubensis, C. sativa, P. yezoensis, and D. odorata, including a blend of two such extracts, three such extracts, four such extracts, five such extracts, and greater than five such extracts, including multiple extracts of any of P. cubensis, C. sativa, P. yezoensis, and D. odorata, as above or otherwise.

In some embodiments, the blend includes an excipient. In some embodiments, the blend includes water as the excipient. In some embodiments, the blend includes water and a flavorant/colorant as the excipients. In some embodiments, the blend includes ginger and/or bay laurel as the flavorant/colorant. In some embodiments, the blend includes additional excipients. In some embodiments, the blend includes no additional excipients. In some embodiments, the blend includes additional active agents. In some embodiments, the blend includes no additional active agents. In some embodiments, the excipients are added to the extracts before the extracts are blended. In some embodiments, the excipients are added to the extracts while the extracts are blended. In embodiments, the excipients are added to the extracts after the extracts are blended.

In some embodiments, after blending, the blend is homogenized. In some embodiments, homogenization is performed by ultrasonic homogenization for 15 minutes at 25% power.

In some embodiments, the homogenized blend is hot filtered to 0.4 microns. In some embodiments, the hot filtrate is next cooled or let cool, and then filtered to 0.22 microns. In some embodiments, an LC-MS fingerprint is obtained of the homogenized filtrate. In some embodiments, including if the fingerprint demonstrates that the product meets the characterization sought, the product may be administered to patients, including as ABS108.

Through this exemplary embodiment, it will be appreciated that the preparation may be viewed broadly as comprising the steps of (1) milling and sieving the raw material to obtain a crude botanical ingredient; (2) preparation of the crude ingredient to obtain a prepared ingredient; (3) extraction of the prepared ingredient to obtain an extract; (4) filtration of the extract to obtain a filtrate; (5) blending and adjustment of the filtrates to obtain a blended botanical mixture; (6) homogenization and filtration of the blended mixture to obtain a final botanical product.

It will be readily appreciated that steps may be performed in a different order, and steps may be combined, removed, or modified, and further steps may be added. It will be further understood that “botanical” may refer to any plant, fungi, and/or algae ingredients or products.

In embodiments, individual extractions are performed on each resulting formulation of a crude botanical ingredient, according to the U.S. Pharmacopeia (USP) guidelines, and liquid chromatography-mass spectrometry (LC-MS) testing is conducted to establish the primary and/or secondary bioactive molecule concentration for each extraction, for example the concentration of Δ9-THC, CBD, psilocin, psilocybin, coumarin, and porphyran.

In some embodiments, relative ratios of each extraction are calculated according to the desired dosages of the primary and/or secondary bioactive molecules in the formulation, which may be any of the formulations disclosed in Examples 1-14. The primary and/or secondary bioactive molecules are then compounded according to the calculated ratios and homogenized. A final LC-MS test is performed to confirm the concentration of primary and/or secondary bioactive molecules are within the target identified dose range. Excess raw material is then destroyed. The final homogenized compound is thereafter validated as a pharmaceutical to be dispensed, labeled, and shipped to the appropriate site for distribution.

### Example 18: Exemplary Analysis of a Disclosed Composition

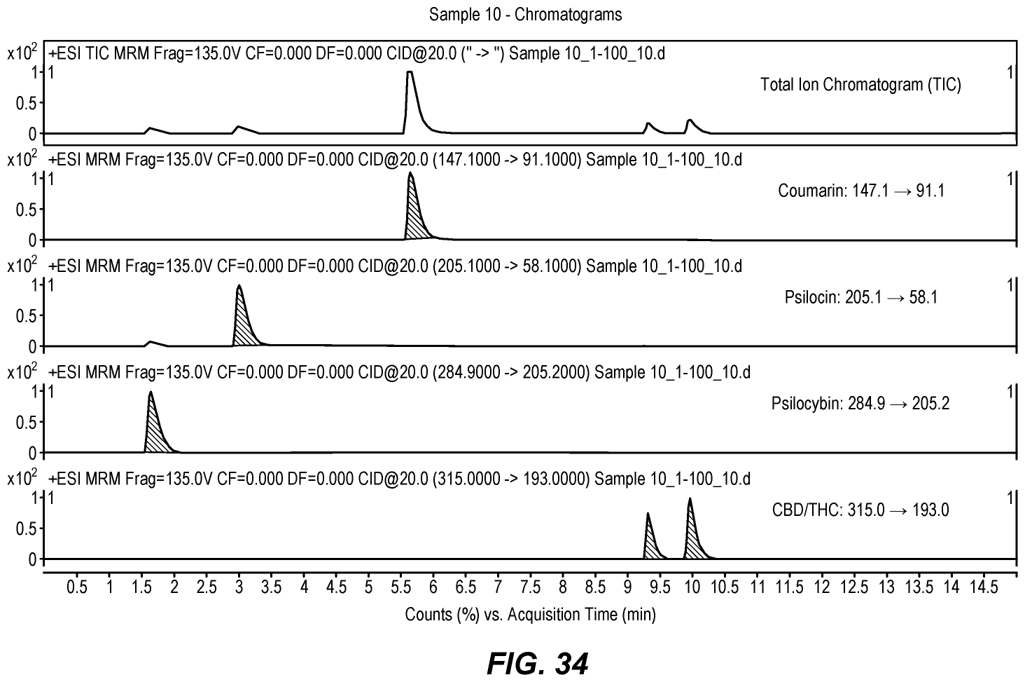

An exemplary composition comprising a therapeutic combination was prepared according to methods disclosed herein, such as in Example 17. This composition was subjected to LC-MS and NMR analysis. Exemplary LC-MS data for the composition (which is labeled “Sample 10”) are provided in FIG. 34, which depicts chromatograms showing the concentrations of constituent bioactive molecules contained in the composition, including the total ion concentration of the formulation, the concentration of coumarin contained in the formulation, the concentration of psilocin contained in the formulation, the concentration of psilocybin contained in the formulation, and the concentration of CBD/THC contained in the formulation.

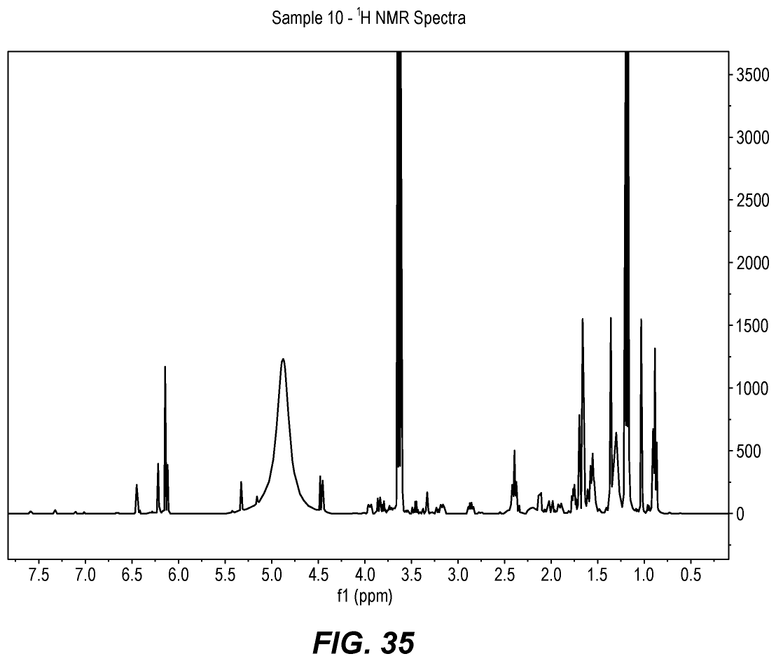

Exemplary proton (1H) nuclear magnetic resonance (NMR) spectroscopy data is provided in FIG. 35, which depicts an 1H NMR spectrum for the composition (which is labeled “Sample 10”). The magnitude of the signal is shown on the ordinate axis (γ-axis), and frequency (i.e., chemical shift) is shown on the horizontal axis (x-axis) in parts per million (ppm).

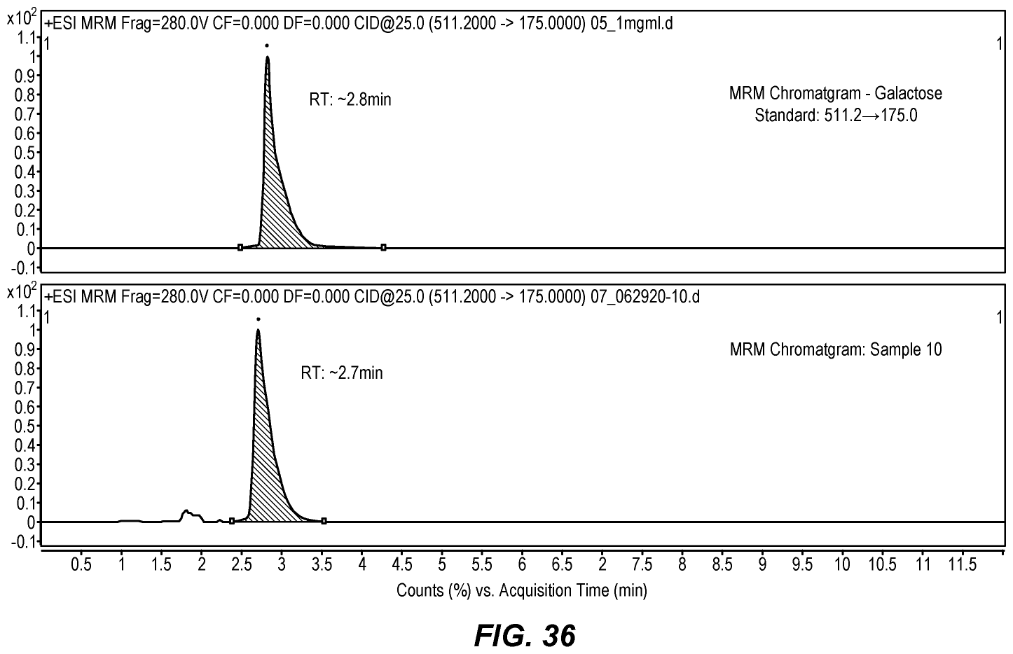

Exemplary multiple reaction monitoring (MRM) LC-MS data are provided in FIG. 36, which shows two MRM chromatograms, including a MRM chromatogram for the composition (which is labeled “Sample 10”) and a MRM chromatogram for a standard sample containing galactose. MRM is a mass spectrometry method that allows for the identification and quantification of fragments of a selected precursor ion. This technique is particularly useful for the analysis of large and complex biomolecules, such as polypeptides and other biopolymers. In some embodiments, MRM is used for quantifying the Pyropia-derived polysaccharide concentration in a disclosed composition. Pyropia contains porphyran, a highly substituted agarose with a linear backbone consisting of 3 linked β-D-galactosyl units alternating with either 4-linked α-L-galactosyl 6-sulfate or 3-6-anhydro-α-L-galactosyl units. The precise composition of porphyran shows seasonal and environmental variations (Jofre et al., 2020). In some embodiments, MRM techniques are used to quantify the amount of galactose (a porphyran fragmentation product) in a sample, thereby providing insight into the composition with regard to porphyran and Pyropia concentration. FIG. 36 shows a retention time (RT) of ˜2.8 minutes for galactose and a RT of ˜2.7 minutes for Sample 10.

Concentrations of the bioactive molecules in the composition, determined according to methods of this Example, are shown below in TABLE 6.

TABLE 6
LC-MS formulation parameters

| | | *Concen- | | | | | |
|---|---|---|---|---|---|---|---|
| Sample | Descrip- | tration | Psilocin | Psilocybin | Coumarin | CBD | THC |
| # | tion | (mg/mL) | (mg/mL) | (mg/mL) | (mg/mL) | (mg/mL) | (mg/mL) |
| 062920- | Full | 7.5 | 1.63 | 12.15 | 39.19 | 56.20 | 52.82 |
| 10 | formula- | Psilocybin | | | | | |
| | tion | 50 | | | | | |
| | | THC/CBD | | | | | |

*Estimates based on Applicant's calculations.
BLD = below limit of detection (~100 mg/mL).

### Example 19: Placebo Controlled Study

A double-blind placebo controlled study will be completed where subjects with a movement disorder are separated into two or more groups. The movement disorder may be Parkinson's disease. Half of the participants will receive one or more doses of the combinations disclosed herein (e.g., the tincture of EXAMPLE 1), while the other half will receive one or more doses of a placebo. A placebo will be that known to those of skill, and can be for example 5 mg niacin in an ethanolic ginger extract. The study will last for between 1 to 10 weeks.

Clinical outcome assessments, such as those disclosed in TABLE 2 (e.g., the MDS-UPDRS, the UPDRS, and the UPDRS-8) will be assessed prior to and after the duration of the study. At the conclusion of the study, it will be determined that the participants receiving one or more doses of the combinations disclosed herein will have equal or greater improvements to one or more symptoms of a movement disorder, as determined by the clinical outcome assessments disclosed in TABLE 2.

### Example 20: Increase in SV2A Density

Purpose: A study will be conducted to determine if the disclosed therapeutic combinations are effective in increasing the presynaptic and postsynaptic protein density of synaptic vesicle glycoprotein 2A (SV2A).

Methods: To complete the study, a movement disorder will be induced by administering a unilateral injection of 6-hydroxydopamine (6-OHDA) to rats according to methods disclosed in Raval, 2021. In one alternative method, mice can be administered 1-methyl-4-phenyl-1,2,3,6-tetrahydropyridine (MPTP) (id.). The 6-OHDA-treated rats will be divided into two treatment groups, one given a disclosed therapeutic combination, and the other given a placebo via intramuscular (IM) injection. In vivo changes in SV2A density will be measured using a PET scan and C-labeled and/or F-labeled SV2A PET tracers. The PET tracers will also be given via IM injection. PET tracers may be injected according to the methods described in Toyonaga, 2022.

In vivo changes in SV2A density may be measured and analyzed according to the methods described in Raval, 2021. All scans will be performed on the Siemens HRRT (High-Resolution Research Tomography), and all rats examined using both [11C]UCB-J and [18F]FDG. All rats will be placed in a custom 2×2 rat holder which enables simultaneous PET scans of four rats. The rats will be anesthetized using 3% isoflurane in oxygen. The rats will be kept warm using an infrared lamp and monitored for respiration during the scans.

Results & Significance: At the conclusion of the study, it will be determined that those given the therapeutic combination will exhibit an increase in SV2A density and/or improved neuronal metabolic function.

### Example 21: Increase in PSD95 Density

Purpose: A study will be conducted to determine if the disclosed therapeutic combinations are effective in increasing postsynaptic density protein 95 (PSD95). Animal studies suggest that PSD-95 disruption is associated with cognitive and learning deficits, such as in Horner, 2021. Reduced expression of PSD-95 has been observed in brain tissue from AD subjects, such as in Gylys, 2004, and in mouse models of AD, such as in Shao, 2011.

Methods: To complete this study, 6-OHDA-treated rats according to the methods described in EXAMPLE 19. The 6-OHDA-treated rats will be divided into two treatment groups, one given a disclosed therapeutic combination, and the other given a placebo via intramuscular (IM) injection.

Cerebrospinal fluid (CSF) will be collected from each group after treatment with the therapeutic combination or placebo respectively. The level of PSD95 will be measured using bead-based digital Simoa immunoassays designed to detect PSD95 in the CSF. PSD95 levels may be measured according to the methods described in Kivisakk, 2022. Increased levels of the synaptic proteins PSD-95, SNAP-25, and neurogranin in the cerebrospinal fluid of patients with Alzheimer's disease (id.).

Results & Significance: At the conclusion of the study, it will be determined that those given the therapeutic combination will exhibit an increase in PSD95 levels in the CSF.

### Example 22: Clinical Mechanical Models

In some further examples, data are collected at baseline (e.g., at the start of treatment, or a selected point before treatment initiation) and at one or more points during and/or following treatment, such as about 1 week, 2 weeks, 3 weeks, 4 weeks, 6 weeks, 2 months, 3 months, 4 months, 5 months, 6 months, 7 months, 8 months, 9 months, 10 months, 11 months, 1 year, 15 months, 18 months, 21 months, 2 years, 2.5 years, 3 years, and more than 3 years following treatment. The data at baseline are compared with the one or more points during and/or following treatment (for a single subject, or between subjects, using statistical methods as will be commonly known) and analyzed to determine the treatment effect. Treatment effect (e.g., post-treatment delta from baseline) will be determined for any of (1) amplitude and frequency of tremor; (2) gait quality; and (3) voice range and amplitude. Assessments may be made using methods known in the art, e.g., using body-worn accelerometers to measure tremor.

### Example 23: Synergistic Effects

Purpose: A study will be conducted to determine if the disclosed therapeutic combinations have synergistic effects in the treatment of movement disorders.

Methods: Subjects having a movement disorder will be selected for treatment during a treatment period and their symptoms will be measured prior to the study to obtain a baseline and in regular intervals throughout the treatment period to monitor their responsiveness to treatment.

During the treatment treatment period, subjects suffering from a movement disorder will be divided into 5 treatment groups, which will receive either a daily oral dose of a placebo; 250 μg of psilocybin, and 150 μg of psilocin; 1 mg of THC and 1 mg of CBD; 250 μg of psilocybin, 150 μg of psilocin, 1 mg of THC and 1 mg of CBD; or 250 μg of psilocybin, 150 μg of psilocin, 1 mg of THC, 1 mg of CBD, 1 mg of Coumarin, and 8 mg of whole Pyropia extract.

Results & Significance: At the conclusion of the study, it will be determined that those given the therapeutic combinations will exhibit a reduction in at least one symptom of a movement disorder. The reduction in the symptom(s) will be observed to be greatest in the treatment group receiving 250 μg of psilocybin, 150 μg of psilocin, 1 mg of THC, 1 mg of CBD, 1 mg of Coumarin, and 8 mg of whole Pyropia extract due to the synergistic effects of the ingredients in this therapeutic combination.

### Example 24: Additional In Vitro and In Vivo Studies

Additional in vitro and in vivo studies may be performed and are expected to confirm the results of the other studies herein as described in the Examples above and/or to provide additional support for the utility and benefits of the disclosed invention.

Non-limiting examples of contemplated in vitro studies include: (1) Inflammatory immune cell activation and profiling of pro- and anti-inflammatory cytokines, restorative growth factors, and neuroendocrine panels; (2) Stem cell effects in vitro lab testing under inflamed culture conditions: screening assays; (3) Neuronal cell models: anti-inflammatory and protective properties; and (4) Stem cell mobilization. Non-limiting examples of contemplated in vitro studies include: (1) Serum pro- and anti-inflammatory cytokines, restorative growth factors; (2) Serum neuroendocrine communication markers; and (3) Mitochondrial resilience testing.

### Example 25: Total Antioxidant Capacity (TPH) Assay

Purpose: A study was conducted to determine the antioxidant capacity of cannabis stock, psilocybe stock, pyropia stock, tonka stock, as well as multi-ingredient combinations.

Methods: The products were tested in the Folin-Ciocalteu assay (also known as the total phenolics assay), according to the methods of Huang 2005 and Kupina 2018. The assay was performed by adding the Folin-Ciocalteu's phenol reagent to serial dilutions of extract, thoroughly mixing, and incubating for 5 minutes. Sodium carbonate was added, starting a chemical reaction producing a color. The reaction was allowed to continue for 30 minutes at 37° C. Optical absorbance was measured at 765 nm in a colorimetric plate reader. Gallic acid was used as a reference standard, and data was reported in Gallic Acid Equivalents (GAE) per gram product. The details of test products utilized are provided in TABLE 7.

TABLE 7
Test products

| Name | Abbreviation | Percentage | Description | Handling |
|---|---|---|---|---|
| Cannabinoid extract | Ca | 15% | CBD, THC | Not |
| | | | | water-soluble* |
| Psilocybe extract | Ps | 45% | Complex extract** | water-soluble |
| Pyropia extract | Py | 15% | Complex extract | water-soluble |
| Tonka bean extract | To | 2.0% | 50% w/v coumarin | water-soluble |

*In the test for total antioxidant capacity, Cannabis was diluted in glycerin, followed by serial dilutions in distilled water. In subsequent tests, Cannabis was emulsified using soy lecithin, followed by serial dilutions in physiological saline. Cannabis blended well with Psilocybe and no emulsifier was needed when blending these two ingredients.
**20 mg/mL, with a 50:50 split between psilocybin and psilocin. Also contains polysaccharides, beta-carbolines, MBAPs, and other complex compounds.

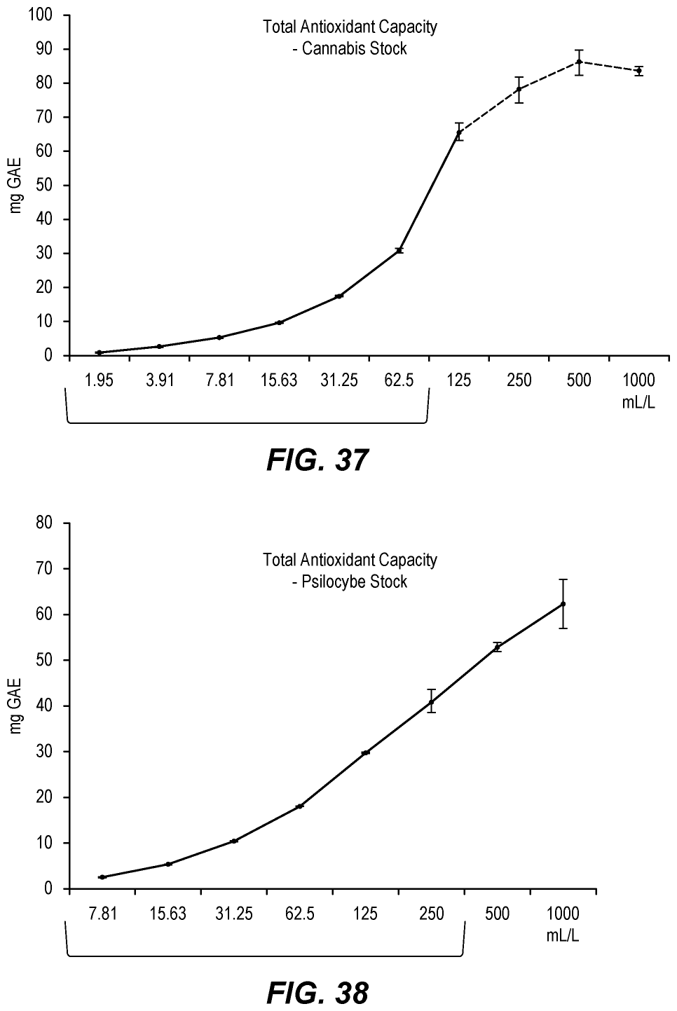

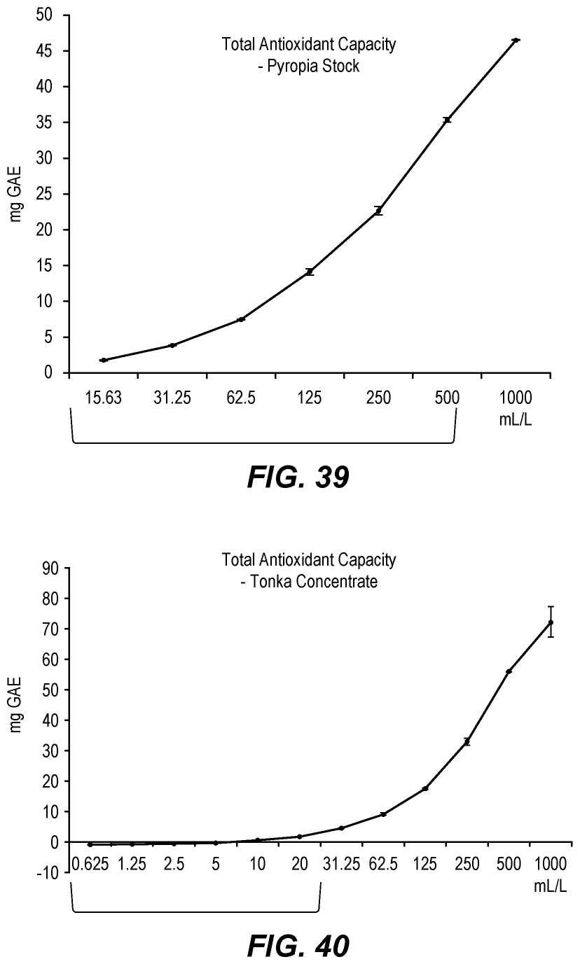

Results & Significance: The antioxidant capacities of disclosed compounds and combinations are provided in TABLE 8 below. The individual total antioxidant capacities obtained via the Folin-Ciocalteu assay for Cannabis Stock, for Psilocybe Stock, for Pyropia Stock, and for Tonka Stock are depicted in FIGS. 37-40, respectively. FIGS. 37-40 show the calculated GAEs for Cannabis Stock, for Psilocybe Stock, for Pyropia Stock, and for Tonka Stock, respectively, in milligrams (mg) per milliliter/liter (mL/L) of solution utilized. The data shown in FIGS. 37-40 includes the average±standard deviation of duplicate data points for each dose, and the bracket located under the abscissa of the graph indicates the dose range used for subsequent assays. The dashed line shown in FIG. 37 indicates that precipitates formed as part of the chemical reaction, and that the light absorbance readings are not reliable. Individually, all ingredients had antioxidant capacity, with cannabis and psilocybe having the most robust antioxidant capacity. Tonka also provided an enhancement of the antioxidant capacity, despite its very low proportion in the blend. Pyropia showed approximately half of the antioxidant capacity seen for Psilocybe. Further details and conclusions are in the Brief Description of the Figures.

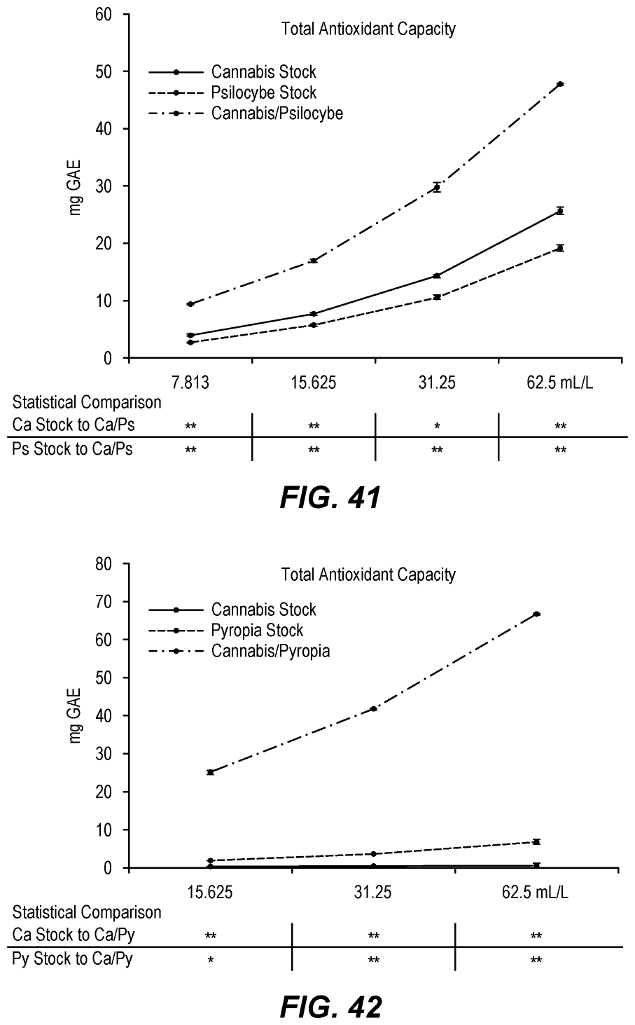

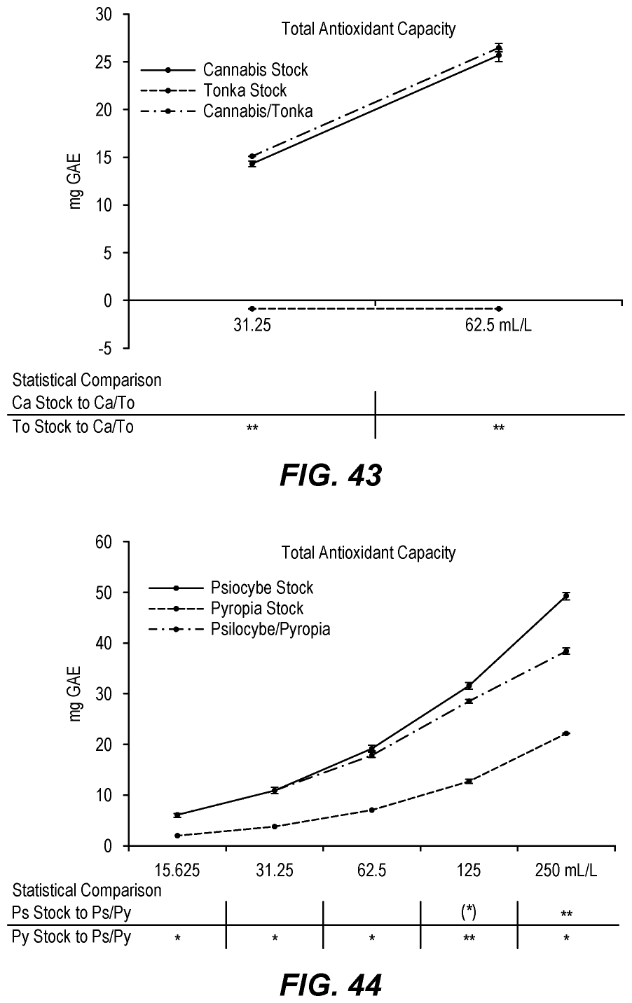

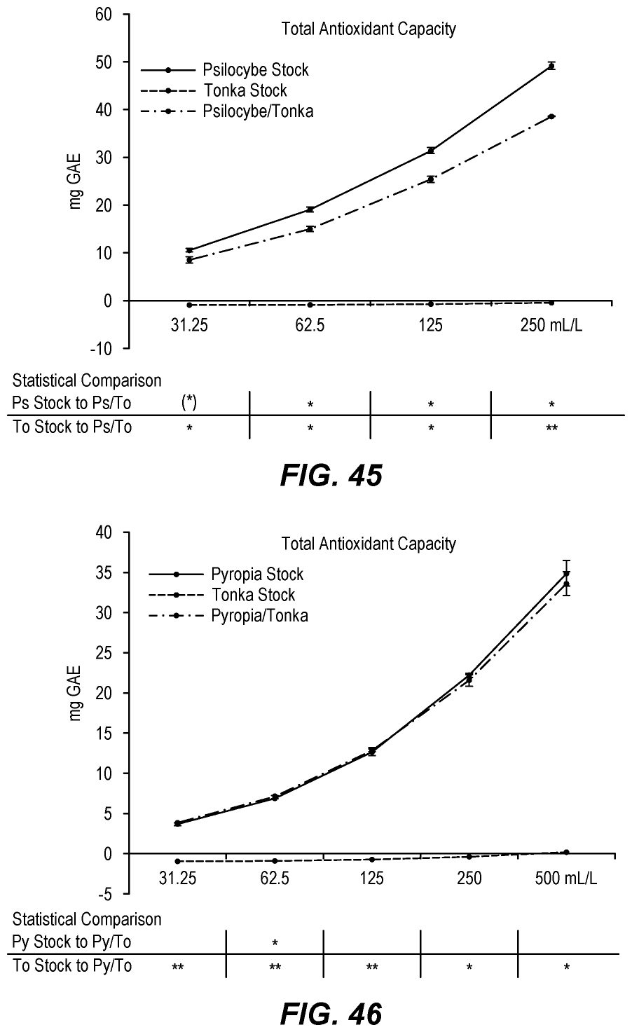

All 2-ingredient blends had antioxidant capacity. The total antioxidant capacities obtained via Folin-Ciocalteu assay for 2-ingredient blends are described in FIGS. 41-46. In FIGS. 41-46, the data is shown as the average±standard deviation of duplicate data points for each dose.

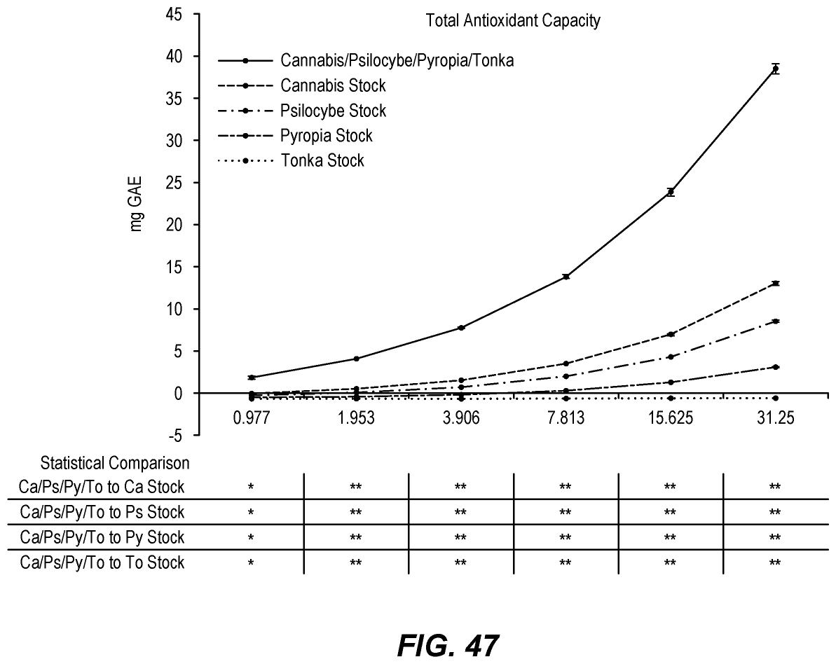

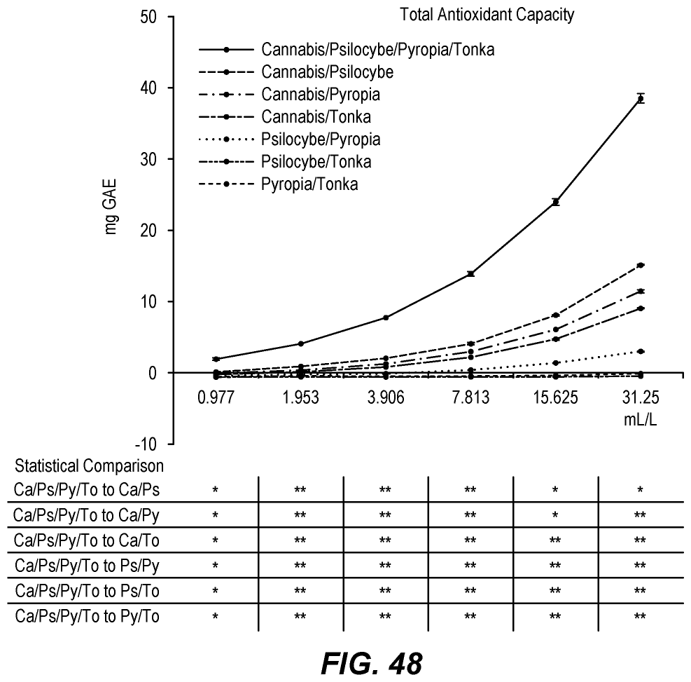

The total antioxidant capacities obtained via the Folin-Ciocalteu assay for the 4-ingredient blends are described in FIGS. 47-48. In FIGS. 47-48, the data is shown as the average±standard deviation of duplicate data points for each dose. The 4-ingredient blend showed a higher antioxidant capacity than what would be expected by adding the contribution of each ingredient together. These data suggest a synergistic effect between the ingredients. The 4-ingredient blend showed a higher antioxidant capacity than what would be expected by adding the contributions of each 2-ingredient blend together. These data suggest a synergistic effect between the ingredients.

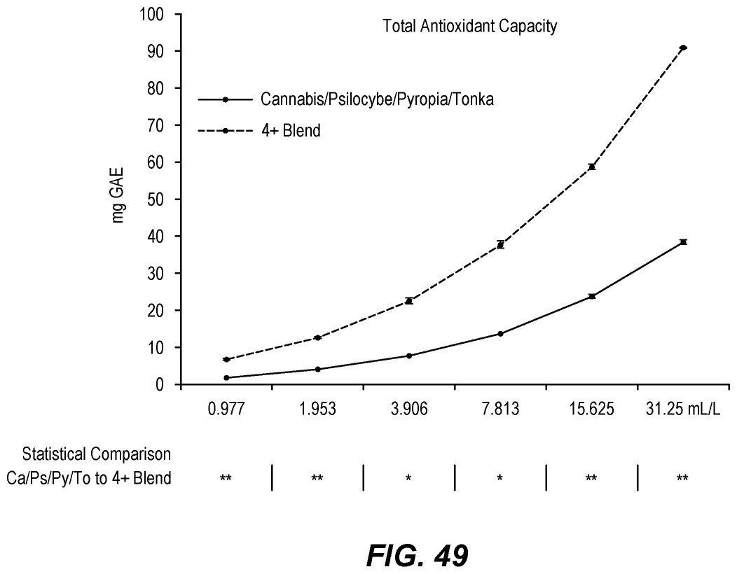

The total antioxidant capacity obtained via the Folin-Ciocalteau assay for the 4+ blend, e.g. Applicant's final product disclosed herein as ABS-108 and NIM-01, and as the formulation of EXAMPLE 1, is described in FIG. 49. FIG. 49 depicts data as the average±standard deviation of duplicate data points for each dose. The 4+ blend additionally comprises a flavorant/colorant, which is not included in the composition of the 4-ingredient blend. The flavorant/colorant is ethanol infused with ginger and bay leaf. The 4+ blend showed advantages above any of the individual ingredients and the 2-ingredient blends. Moreover, as depicted in FIG. 49, the 4+ blend surprisingly showed a higher antioxidant capacity than the 4-ingredient blend. This demonstrates that the additional ingredients in the 4+ blend contribute to the antioxidant properties of the final product. Furthermore, the 4+ blend showed a higher antioxidant capacity than what would be expected by adding the contribution of each ingredient together, which demonstrates an unexpected synergistic effect between the ingredients of the 4+ blend.

TABLE 8
Total antioxidant capacity

| Product | mg GAE per mL of product |
|---|---|
| Cannabis Stock | 0.4 |
| Psilocybe Stock | 0.3 |
| Pyropia Stock | 0.1 |
| Tonka Stock | 0.0 |
| Tonka Concentrate | 0.1 |
| Cannabis/Psilocybe | 0.3 |
| Cannabis/Pyropia | 1.1 |
| Cannabis/Tonka | 0.4 |
| Psilocybe/Pyropia | 0.2 |
| Psilocybe/Tonka | 0.2 |
| Pyropia/Tonka | 0.1 |
| Cannabis/Psilocybe/Pyropia/Tonka | 1.2 |
| 4+ Blend | 2.9 |

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; of course, many modifications and variations are possible in view of the above teachings. The embodiments were chosen and described in order to best explain certain key principles of the invention and its practical applications, through the elucidation of specific examples, and to thereby enable others skilled in the art to best make and utilize the invention and various embodiments with various modifications as are suited to the particular use contemplated, even when such uses are beyond the specific examples disclosed. Accordingly, the scope of the invention shall be defined solely by the following claims and their equivalents.

### REFERENCES

1. Abdelhafez, O. M., et al. (2011). Dopamine release and molecular modeling study of some coumarin derivatives. Neurochemistry international, 59 (6), 906-912. https://doi.org/10.1016/j.neuint.2011.08.004
2. Agca, C. A., Tuzcu, M., Hayirli, A., & Sahin, K. (2014). Taurine ameliorates neuropathy via regulating NF-κB and Nrf2/HO-1 signaling cascades in diabetic rats. Food and chemical toxicology: an international journal published for the British Industrial Biological Research Association, 71, 116-121. https://doi.org/10.1016/j.fct.2014.05.023
3. Ananthakrishnan, R., Chandra, P., Kumar, B., & Rameshkumar, K.B. (2018). Quantification of coumarin and related phenolics in cinnamon samples from south India using UHPLC-ESI-QqQLIT-MS/MS method. International Journal of Food Properties, 21, 50-57
4. Anderson P. Use of Respimat Soft Mist inhaler in COPD patients. Int J Chron Obstruct Pulmon Dis. 2006;1 (3): 251-9. doi: 10.2147/copd.2006.1.3.251. PMID: 18046862; PMCID: PMC2707154.
5. Ansel and Stoklosa, Pharm. Calculations (2001) 11th ed., Lippincott Williams & Wilkins, Baltimore, Md.
6. Anusiya, G., Gowthama Prabu, U., Yamini, N. V., Sivarajasekar, N., Rambabu, K., Bharath, G., & Banat, F. (2021). A review of the therapeutic and biological effects of edible and wild mushrooms. Bioengineered, 12 (2), 11239-11268. https://doi.org/10.1080/21655979.2021.2001183
7. Ahlskog, J. E. (2005). Challenging conventional wisdom: The etiologic role of dopamine oxidative stress in Parkinson's disease. Movement Disorders, 20 (3), 271-282. doi: 10.1002/mds.20362.
8. Apak, R., Güçlü, K., Demirata, B., Özyürek, M., çelik, S., Bektaşoğlu, B., Isil Berker, K., Özyurt, D. (2007). Comparative Evaluation of Various Total Antioxidant Capacity Assays Applied to Phenolic Compounds with the CUPRAC Assay. Molecules, 12 (7). 1496-1547. doi: 10.3390/12071496.
9. Assessment the 4-stage balance test. CDC.gov. (n.d.). Retrieved May 27, 2022, from https://www.cdc.gov/steadi/pdf/STEADI-Assessment-4Stage-508.pdf
10. Bailey, R. (2018 September 12). Pond scum, seaweed and other algae types. ThoughtCo. Retrieved May 4, 2022, from https://www.thoughtco.com/major-types-of-algae-373409
11. Banerjee S P, Snyder S H, Mechoulam R. Cannabinoids: influence on neurotransmitter uptake in rat brain synaptosomes. J Pharmacol Exp Ther. 1975 July; 194 (1): 74-81. PMID: 168349.
12. “Beilstein Handbook of Organic Chemistry,” Beilstein Institute of Organic Chemistry, Frankfurt, Germany.
13. Berge S M, Bighley L D, Monkhouse D C. Pharmaceutical salts. J Pharm Sci. 1977 January;66 (1): 1-19. doi: 10.1002/jps.2600660104. PMID: 833720
14. Bigwood, J., & Beug, M. W. (1982). Variation of psilocybin and psilocin levels with repeated flushes (harvests) of mature sporocarps of psilocybe cubensis (Earle) singer. Journal of Ethnopharmacology, 5 (3), 287-291. https://doi.org/10.1016/0378-8741 (82) 90014-9
15. Bloom, A. S. (1982). Effect of delta9-tetrahydrocannabinol on the synthesis of dopamine and norepinephrine in mouse brain synaptosomes. Journal of Pharmacology and Experimental Therapeutics, 221 (1), 97-103
16. Bonnin, A., de Miguel, R., Castro, J. G. et al. Effects of perinatal exposure to A9-tetrahydrocannabinol on the fetal and early postnatal development of tyrosine hydroxylase-containing neurons in rat brain. J Mol Neurosci 7, 291-308 (1996). https://doi.org/10.1007/BF02737066
17. Boylan, N. J., Finnance, S. S., Yucel, T., and Zale S. E. (Aug. 20, 2020). PCT Pub. No. 2020/167893. “METHODS FOR NON-IRRITATING PULMONARY ADMINISTRATION OF CANNABINOIDS USING SOFT MIST INHALERS”
18. Che, Y., Hou, L., Sun, F., Zhang, C., Liu, X., Piao, F., Zhang, D., Li, H., & Wang, Q. (2018). Taurine protects dopaminergic neurons in a mouse Parkinson's disease model through inhibition of microglial M1 polarization. Cell death & disease, 9 (4), 435. https://doi.org/10.1038/s41419-018-0468-2
19. CHRISTENSEN F. (1964). URICOSURIC EFFECT OF DICOUMAROL. Acta medica Scandinavica, 175, 461-468. https://doi.org/10.1111/j.0954-6820.1964.tb00594.x
20. Chou TC, Talalay P. Quantitative analysis of dose-effect relationships: the combined effects of multiple drugs or enzyme inhibitors. Adv Enzyme Regul. 1984; 22:27-55. doi: 10.1016/0065-2571 (84) 90007-4. PMID: 6382953
21. Chouhan, S., Sharma, K., Zha, J., Guleria, S., & Koffas, M. (2017). Recent Advances in the Recombinant Biosynthesis of Polyphenols. Frontiers in microbiology, 8, 2259. https://doi.org/10.3389/fmich.2017.02259
22. Cummins, G., Zandi, M., & Barker, R. A. (2015). Movement disorders and psychiatry: Five new things. Neurology. Clinical practice, 5 (2), 143-149. https://doi.org/10.1212/CPJ.0000000000000113
23. Dalby RN, Eicher J, Zierenberg B. Development of Respimat (®) Soft Mist™ Inhaler and its clinical utility in respiratory disorders. Med Devices (Auckl). 2011; 4:145-55. doi: 10.2147/MDER.S7409. Epub 2011 Sep. 1. PMID: 22915941; PMCID: PMC3417885.
24. Di Stasi L. C. (2021). Coumarin Derivatives in Inflammatory Bowel Disease. Molecules (Basel, Switzerland), 26 (2), 422. https://doi.org/10.3390/molecules26020422
25. De Backer, B., et al. (2009). Innovative Development and validation of an HPLC/DAD method for the qualitative and quantitative determination of major cannabinoids in cannabis plant material. Journal of Chromatography B, 877 (32), 4115-4124. https://doi.org/10.1016/j.jchromb.2009.11.004
26. Dodd, S., Norman, T., Eyre, H., Stahl, S., Phillips, A., Carvalho, A., & Berk, M. (2022). Psilocybin in neuropsychiatry: A review of its pharmacology, safety, and efficacy. CNS Spectrums, 1-11. doi: 10.1017/S1092852922000888
27. Dos-Santos-Pereira, M., Guimarães, F. S., Del-Bel, E., Raisman-Vozari, R., & Michel, P. P. (2020). Cannabidiol prevents LPS-induced microglial inflammation by inhibiting ROS/NF-κB-dependent signaling and glucose consumption. Glia, 68 (3), 561-573. https://doi.org/10.1002/glia.23738
28. Elkouzi, A. (n.d.). What is parkinson's? Parkinson's Foundation. Retrieved May 26, 2022, from https://www.parkinson.org/understanding-parkinsons/what-is-parkinsons
29. Emad H. M. Hassanein, Ahmed M. Sayed, Omnia E. Hussein, Ayman M. Mahmoud, “Coumarins as Modulators of the Keap1/Nrf2/ARE Signaling Pathway”, Oxidative Medicine and Cellular Longevity, vol. 2020, Article ID 1675957, 25 pages, 2020. https://doi.org/10.1155/2020/1675957
30. Ericson, M., Molander, A., Stomberg, R., & Soderpalm, B. (2006). Taurine elevates dopamine levels in the rat nucleus accumbens; antagonism by strychnine. The European journal of neuroscience, 23 (12), 3225-3229. https://doi.org/10.1111/j.1460-9568.2006.04868.x
31. Feiser et al, “Reagents for Organic Synthesis,” Volumes 1-17, Wiley Interscience (1967-1994).
32. Frijhoff, Jeroen, Paul G. Winyard, Neven Zarkovic, Sean S. Davies, Roland Stocker, David Cheng, Annie R. Knight, Emma Louise Taylor, Jeannette Oettrich, Tatjana Ruskovska, Ana Cipak Gasparovic, Antonio Cuadrado, Daniela Weber, Henrik Enghusen Poulsen, Tilman Grune, Harald H.H.W. Schmidt, & Pietro Ghezzi. (2015). Clinical Relevance of Biomarkers of Oxidative Stress. Antioxidants & Redox Signaling, 23, 14, 1144-1170. DOI: 10.1089/ars.2015. 63/171,144.
33. Gartz J. Analyse der Indolderivate in Fruchtkörpern and Mycelien von Panaeolus subalteatus (Berk. & Br.) Sacc. Biochemie und Physiologie der Pflanzen. 1989; 184 (1-2): 171-178. doi: 10.1016/S0015-3796 (89) 80139-8
34. Gartz, J. Biotransformation of tryptamine derivatives in mycelial cultures of Psilocybe. Journal of Basic Microbiology. 1989; 29 (6): 347-352. https://doi.org/10.1002/jobm.3620290608
35. Gertsch J, Pertwee RG, Di Marzo V. Phytocannabinoids beyond the Cannabis plant-do they exist? Br J Pharmacol. 2010 June; 160 (3): 523-9. doi: 10.1111/j. 1476-5381.2010.00745.x. PMID: 20590562; PMCID: PMC2931553.
36. Gifford, A. N., Bruneus, M., Gatley, S J., & Volkow, N. D. (2000). Cannabinoid receptor-mediated inhibition of acetylcholine release from hippocampal and cortical synaptosomes. British journal of pharmacology, 131 (3), 645-650. https://doi.org/10.1038/sj.bjp.0703599
37. Glennon, R., McKenney, J.D., Lyon, R. & Titeler, M., 5-HT1 and 5-HT2 binding characteristics of 1-(2,5-dimethoxy-4-bromophenyl)-2-aminopropane analogs, Journal of Medicinal Chemistry, 1986; 29 (2), 194-199.
38. Gloss D. (2015). An Overview of Products and Bias in Research. Neurotherapeutics: the journal of the American Society for Experimental Neuro Therapeutics, 12 (4), 731-734. https://doi.org/10.1007/s13311-015-0370-x
39. Gylys KH, Fein JA, Yang F, Wiley DJ, Miller CA, Cole GM. (2004). Synaptic changes in Alzheimer's disease: increased amyloid-beta and gliosis in surviving terminals is accompanied by decreased PSD-95 fluorescence. Am J Pathol. 2004 November; 165 (5): 1809-17. doi: 10.1016/s0002-9440 (10) 63436-0. PMID: 15509549; PMCID: PMC1618663.
40. Gomez-Zavaglia A. Prieto Lage MA, Jimenez-Lopez C, Mejuto JC, Simal-Gandara J. The Potential of Seaweeds as a Source of Functional Ingredients of Prebiotic and Antioxidant Value. Antioxidants (Basel). 2019 Sep. 17;8 (9): 406. doi: 10.3390/antiox8090406. PMID: 31533320; PMCID: PMC6770939.
41. Grandjean, J., Buehlmann, D., Buerge, M., Sigrist, H., Seifritz, E., Vollenweider, F. X., Pryce, C. R., & Rudin, M. (2021). Psilocybin exerts distinct effects on resting state networks associated with serotonin and dopamine in mice. Neurolmage, 225, 117456. https://doi.org/10.1016/j.neuroimage.2020.117456.
42. Greene, T. & Wuts, P., “Protective Groups in Organic Chemistry,” (Wiley, 2nd ed. 1991).
43. Guibourt, N. J. B. G. (1820). Histoire Abrégée des Drogues Simples [Abridged History of Simple Drugs] (in French). Vol. 2. Paris: L. Colas. pp. 160-161.
44. Harford, Robin (n.d.), layer Seaweed: A Foraging Guide to Its Food, Medicine and Other Uses, Retrieved Jun. 1, 2022, from https://www.eatweeds.co.uk/laver-porphyra.
45. Harrison, I., Harrison, S., Hegedus, L., Wade, L., Smith, M. “Compendium of Synthetic Organic Methods,” Vols. 1-8 (John Wiley and Sons, 1971-1996).
46. Hauser R A, Lyons K E, Pahwa R. The UPDRS-8: a brief clinical assessment scale for Parkinson's disease. Int J Neurosci. 2012; 122 (7): 333-337.
47. Hazekamp, Arno & Simons, Ruud & Sengers, Melvin & Zweden, Rianne & Verpoorte, Rob. (2004). Preparative Isolation of Cannabinoids from Cannabis sativa by Centrifugal Partition Chromatography. Journal of Liquid Chromatography & Related Technologies-J LIQ CHROMATOGR RELAT TECHNO. 27. 2421-2439. 10.1081/JLC-200028170.
48. Heravi & Zadsirjan, Recent Advances in the Synthesis of Biologically Active Compounds Containing Benzo [b] Furans as a Framework. Current Organic Synthesis, 2016; 13 (6), 780-833.
49. Hernandez, M. L., Garcia-Gil, L., Berrendero, F. et al. A9-tetrahydrocannabinol increases activity of tyrosine hydroxylase in cultured fetal mesencephalic neurons. J Mol Neurosci 8, 83-91 (1997). https://doi.org/10.1007/BF02736775.
50. Hielscher Ultrasonics. (2022). Fast and Simple Ultrasonic Cannabis Extraction. Hielscher Ultrasound Technology.
51. Holford NH, Sheiner LB. Understanding the dose-effect relationship: clinical application of pharmacokinetic-pharmacodynamic models. Clin Pharmacokinet. 1981 November-Dec;6 (6): 429-53. doi: 10.2165/00003088-198106060-00002. PMID: 7032803.
52. Horner A E, Norris R H, McLaren-Jones R, Alexander L, Komiyama N H, Grant S G N, Nithianantharajah J, Kopanitsa M V. (2021) Learning and reaction times in mouse touchscreen tests are differentially impacted by mutations in genes encoding postsynaptic interacting proteins SYNGAP1, NLGN3, DLGAP1, DLGAP2 and SHANK2. Genes Brain Behav. 2021 January;20 (1): e12723. doi: 10.1111/gbb.12723. Epub 2020 Dec. 29. PMID: 33347690.
53. Huang D, Ou B, Prior R L. The chemistry behind antioxidant capacity assays. J Agric Food Chem. 2005 Mar. 23;53 (6): 1841-56. doi: 10.1021/jf030723c. PMID: 15769103.
54. Huang, M., Xie, S. S., Jiang, N., Lan, J. S., Kong, L. Y., & Wang, X. B. (2015). Multifunctional coumarin derivatives: monoamine oxidase B (MAO-B) inhibition, anti-β-amyloid (AB) aggregation and metal chelation properties against Alzheimer's disease. Bioorganic & medicinal chemistry letters, 25 (3), 508-513. https://doi.org/10.1016/j.bmcl.2014.12.034
55. Huang, Ruo-yue, et al. “ISOBOLOGRAM Analysis: A Comprehensive Review of Methodology and Current Research.” Frontiers in Pharmacol, vol. 10, 2019, https://doi.org/10.3389/fphar.2019.01222.
56. Hydrophilic Interaction Liquid Chromatography. Hydrophilic interaction liquid chromatography. (2022). Retrieved May 1, 2022, from https://www.sigmaaldrich.com/US/en/technical-documents/technical-article/analytical-chemistry/small-molec ule-hplc/hilic? gclid=Cj0KCQjw37iTBhCWARIsACBt1lxwVn7U4M716XDeNBDqDkkTs5Brk7LRw9txgUL6Yid UbZ5zkvJg78aAtBCEALw_wcB
57. Ieri, F., Pinelli, P., & Romani, A. (2012). Simultaneous determination of anthocyanins, coumarins and phenolic acids in fruits, kernels and liqueur of Prunus mahaleb L. Food chemistry, 135 (4), 2157-2162.

https://doi.org/10.1016/j.foodchem.2012.07.083
58. International Parkinson and Movement Disorder Society. (2021). Clinical outcome assessments. Retrieved May 9, 2022, from https://www.movementdisorders.org/MDS/MDS-Clinical-Outcome-Assessment.htm.
59. Jaiswal M, Dudhe R, Sharma PK. Nanoemulsion: an advanced mode of drug delivery system. 3 Biotech. 2015 April;5 (2): 123-127. doi: 10.1007/s13205-014-0214-0. Epub 2014 Apr. 8. PMID: 28324579; PMCID: PMC4362737.
60. Jastrząb A, Gęgotek A, Skrzydlewska E. Cannabidiol Regulates the Expression of Keratinocyte Proteins Involved in the Inflammation Process through Transcriptional Regulation. Cells. 2019; 8 (8): 827.
61. Jenner, P., & Olanow, C. W. (1998). Understanding cell death in parkinson's disease. Annals of Neurology, 44 (S1), S72-S84. https://doi.org/10.1002/ana.410440712
62. Jiang, W., Veress, T., Nagy, J., & Rácz, N. (2020 November 13). Determination of psilocin and psilocybin in magic mushrooms using ihilic@-fusion and MS. Chromatography Online. Retrieved May 1, 2022, from https://www.chromatographyonline.com/view/determination-psilocin- and-psilocybin-magic-mushrooms-usin g-ihilic-fusion- and-ms
63. Jofre J, Celis-Plá P S M, Figueroa F L, Navarro N P. Seasonal Variation of Mycosporine-Like Amino Acids in Three Subantarctic Red Seaweeds. Mar Drugs. 2020 Jan. 24;18 (2): 75. doi: 10.3390/md18020075.
64. J. A. Joule, K. Mills Heterocyclic Chemistry, 4th edition, Blackwell Science, Oxford, U K, 2000.
65. Kedrowski, S., Bower, K., & Dougherty, D. 1-Oxo-5-hydroxytryptamine: A Surprisingly Potent Agonist of the 5-HT3 (Serotonin) Receptor. Organic Letters, 2007; 9 (17), 3205-3207.
66. Keri R S, Chand K, Budagumpi S, Balappa Somappa S, Patil S A, Nagaraja B M. An overview of benzo[b] thiophene-based medicinal chemistry. Eur J Med Chem. 2017 Sep. 29; 138:1002-1033. doi: 10.1016/j.ejmech.2017.07.038. Epub 2017 Jul. 22. PMID: 28759875.
67. Kevin J Brusse, Sandy Zimdars, Kathryn R Zalewski, Teresa M Steffen, Testing Functional Performance in People With Parkinson Disease, Physical Therapy, Volume 85, Issue 2, 1 Feb. 2005, Pages 134-141, https://doi.org/10.1093/ptj/85.2.134
68. Kivisakk P, Carlyle B C, Sweeney T, Quinn J P, Ramirez C E, Trombetta B A, Mendes M, Brock M, Rubel C, Czerkowicz J, Graham D, Arnold SE. (2022). Increased levels of the synaptic proteins PSD-95, SNAP-25, and neurogranin in the cerebrospinal fluid of patients with Alzheimer's disease. Alzheimers Res Ther. 2022 Apr. 23;14 (1): 58. doi: 10.1186/s13195-022-01002-x. PMID: 35461266; PMCID: PMC9034610.
69. Kozela, E., Pietr, M., Juknat, A., Rimmerman, N., Levy, R., & Vogel, Z. (2010). Cannabinoids Delta (9)-tetrahydrocannabinol and cannabidiol differentially inhibit the lipopolysaccharide-activated NF-kappaB and interferon-beta/STAT proinflammatory pathways in BV-2 microglial cells. The Journal of biological chemistry, 285 (3), 1616-1626. https://doi.org/10.1074/jbc.M109.069294
70. Khan, Ikhlas A.; Ehab, Abourashed A. (2010). Leung's Encyclopedia of Common Natural Ingredients Used in Food, Drugs, and Cosmetics (PDF). Hoboken, NJ USA: John Wiley & Sons. pp. 240-242. ISBN 978-9881607416.
71. Kringelbach, M. L., Jenkinson, N., Owen, S. L., & Aziz, T. Z. (2007). Translational principles of deep brain stimulation. Nature reviews. Neuroscience, 8 (8), 623-635. https://doi.org/10.1038/nrn2196
72. Kupina S, Fields C, Roman MC, Brunelle SL. Determination of Total Phenolic Content Using the Folin-C Assay: Single-Laboratory Validation, First Action 2017.13. J AOAC Int. 2018 Sep. 1;101 (5): 1466-1472. doi: 10.5740/jaoacint. 18-0031. Epub 2018 Jun. 12. PMID: 29895350.
73. Laposata, M., Van Cott, E. M., & Lev, M. H. (2007). Case records of the Massachusetts General Hospital. Case 1-2007. A 40-year-old woman with epistaxis, hematemesis, and altered mental status. The New England journal of medicine, 356 (2), 174-182. https://doi.org/10.1056/NEJMcpc069032.
74. Larock, R. “Comprehensive Organic Transformations,” VCH Publishers, 1989.
75. layer seaweed-a foraging guide to its food, medicine and other uses. EATWEEDS. (2022 May 3). Retrieved May 4, 2022, from https://www.eatweeds.co.uk/laver-porphyra
76. Lee, R. E. (2008). Phycology. Cambridge University Press. ISBN 9780521367448.
77. Lenz, C., Wick, J., Braga, D., Garcia-Altares, M., Lackner, G., Hertweck, C., Gressler, M., & Hoffmeister, D. (2020). Injury-Triggered Blueing Reactions of Psilocybe “Magic” Mushrooms. Angewandte Chemie (International Edition English). 59 (4), 1450-1454. doi: 10.1002/anie.201910175.
78. Lenz, C., Dorner, S., Sherwood, A., Hoffmeister, D. (2021). Structure Elucidation and Spectroscopic Analysis of Chromophores Produced by Oxidative Psilocin Dimerization. Chemistry, 27 (47), 12166-12171. doi: 10.1002/chem.202101382.
79. Liu, Y., et al. (2018). Oligo-Porphyran Ameliorates Neurobehavioral Deficits in Parkinsonian Mice by Regulating the PI3K/Akt/Bcl-2 Pathway. Marine drugs, 16 (3), 82. https://doi.org/10.3390/md16030082.
80. Liu, Y., Deng, Z., Geng, L. et al. In vitro evaluation of the neuroprotective effect of oligo-porphyran from Porphyra yezoensis in PC12 cells. J Appl Phycol 31, 2559-2571 (2019).

https://doi.org/10.1007/s10811-018-1713-x.
81. Loewe S., Muischnek H. (1926) Effect of combinations: mathematical basis of problem. Arch. Exp. Pathol. Pharmacol., 114, 313-326.
82. Lowery, T. H., Richardson, K.S.: Mechanism and Theory in Organic Chemistry, 3d edn. Harper Collins Publishers, New York (1987)
83. Ly. C., et al. (2018). Psychedelics Promote Structural and Functional Neural Plasticity. Cell reports, 23 (11). 3170-3182. https://doi.org/10.1016/j.celrep.2018.05.022
84. Mahmood, Z. A. (2013). Bioactive alkaloids from fungi: Psilocybin. Natural Products, 523-552. https://doi.org/10.1007/978-3-642-22144-6_19
85. Maitre L, Staehelin M, Bein HJ (1970). Effect of an extract of cannabis and of some cannabinols on catecholamine metabolism in rat brain and heart. Agents Actions 1:136-143.
86. Mandrake, K. (2021 June 1). Want to measure the magic in your mushrooms? you can now test psilocybin potency at home. DoubleBlind Mag. Retrieved May 1, 2022, from https://doubleblindmag.com/miraculix/.
87. March, J, “Advanced Organic Chemistry,” Wiley Interscience, 1991.
88. Mayo Foundation for Medical Education and Research. (2017 October 19). Movement disorders. Mayo Clinic. Retrieved May 9, 2022, from https://www.mayoclinic.org/diseases-conditions/movement-disorders/symptoms-causes/syc-20363893
89. MDS-Unified Parkinson's Disease Rating Scale (MDS-UPDRS). International Parkinson and Movement Disorder Society. (2019). Retrieved May 25, 2022, from https://www.movementdisorders.org/MDS/MDS-Rating-Scales/MDS-Unified-Parkinsons-Disease-Rating-Scale-MDS-UPDRS.htm
90. Mehta, A. (Dec. 27, 2012). “Principle of Reversed-Phase Chromatography HPLC/UPLC (with Animation)”
91. Meiser, J., Weindl, D. & Hiller, K. (2013). Complexity of dopamine metabolism. Cell Commun Signal 11, 34. https://doi.org/10.1186/1478-811X-11-34
92. Mishraki-Berkowitz. T., Kochelski, E., Kavanagh, P., O'Brien, J., Dunne, C., Talbot, B., Ennis, P., & Wolf, U. E. (2020). The Psilocin (4-hydroxy-N,N-dimethyltryptamine) and Bufotenine (5-hydroxy-N,N-dimethyltryptamine) Case: Ensuring the Correct Isomer has Been Identified. Journal of forensic sciences. 65 (5). 1450-1457. https://doi.org/10.1111/1556-4029.14368
93. Molnár, I., & Horváth, C. (1976). Reverse-phase chromatography of polar biological substances: separation of catechol compounds by HPLC. Clinical chem., 22 (9), 1497-1502.
94. Morishita, T. (2013 August 20). Movement Disorders. International Neuromodulation Society. Retrieved May 9, 2022, from https://www.neuromodulation.com/movement-disorders.
95. Muszyńska, B., Grzywacz-Kisielewska, A., Kała, K., & Gdula-Argasińska, J. (2018). Anti-inflammatory properties of edible mushrooms: A review. Food chemistry, 243, 373-381. https://doi.org/10.1016/j.foodchem.2017.09.149
96. Naqvi U, Sherman A1. Muscle Strength Grading. [Updated 2021 Sep. 2]. In: StatPearls [Internet]. Treasure Island (FL): StatPearls Publishing; 2022 January-. Available from: https://www.ncbi.nlm.nih.gov/books/NBK436008/97. National Center for Biotechnology Information (2022). PubChem Compound Summary for CID 323, Coumarin. Retrieved May 5, 2022 from https://pubchem.ncbi.nlm.nih.gov/compound/Coumarin.
98. Nichols, D., Snyder, S., Oberlender, R., Johnson, M., & Huang, X. 2,3-Dihydrobenzofuran analogs of hallucinogenic phenethylamines. Journal of Medicinal Chemistry, 1991; 34 (1), 276-281.
99. Nichols, J. M., & Kaplan, B. (2020). Immune Responses Regulated by Cannabidiol. Cannabis and cannabinoid research, 5 (1), 12-31. https://doi.org/10.1089/can.2018.0073
100. Nkadimeng, S. M., Steinmann, C., & Eloff, J. N. (2021). Anti-Inflammatory Effects of Four Psilocybin-Containing Magic Mushroom Water Extracts in vitro on 15-Lipoxygenase Activity and on Lipopolysaccharide-Induced Cyclooxygenase-2 and Inflammatory Cytokines in Human U937 Macrophage Cells. Journal of inflammation research, 14, 3729-3738. https://doi.org/10.2147/JIR.S317182
101. Nkadimeng, S. M., Steinmann, C. M. L. & Eloff, J. N. Effects and safety of Psilocybe cubensis and Panaeolus cyanescens magic mushroom extracts on endothelin-1-induced hypertrophy and cell injury in cardiomyocytes. Sci Rep 10, 22314 (2020). https://doi.org/10.1038/s41598-020-79328-5.
102. Oetjen, K. A., Tran, D., Butt, C. M., & Borton, C. (2020). Comparing microflow vs analytical flow high-performance liquid chromatography for the analysis of pesticides.
103. Ostrowska K. (2020). Coumarin-piperazine derivatives as biologically active compounds. Saudi pharmaceutical journal: SPJ: the official publication of the Saudi Pharmaceutical Society, 28 (2), 220-232. https://doi.org/10.1016/j.jsps.2019.11.025.
104. Paquette, L. “Encyclopedia of Reagents for Organic Synthesis,” John Wiley & Sons, 1995.
105. Park, H. D., & Kim, H. T. (2009). Electrophysiologic assessments of involuntary movements: tremor and myoclonus. Journal of movement disorders, 2 (1), 14-17. https://doi.org/10.14802/jmd.09004.
106. Peres, F. F., Lima, A. C., Hallak, J. E. C., Crippa, J. A., Silva, R. H., & Abilio, V.C. (2018). Cannabidiol as a Promising Strategy to Treat and Prevent Movement Disorders?. Frontiers Pharmacology, 9, 482, https://doi.org/10.3389/fphar.2018.00482.
107. Perez-Silanes, S., Martinez-Esparza, J., Oficialdegui, A.M., Villanueva, H., Oruas, L., Monge, A. Synthesis of new 5-substitutedbenzo[b] thiophene derivatives. Journal of Heterocyclic Chemistry. 2001, 38, (5), 1025-1030.
108. Perkin, W. H. (1868). “On the artificial production of coumarin and formation of its homologues”. Journal of the Chemical Society. 21:53-61. doi: 10.1039/js8682100053.
109. Pharm. Principles of Solid Dosage Forms (1993), Technomic Pub. Co., Inc., Lancaster, Pa.
110. Poznansky et al. Drug Delivery Systems (1980), R.L. Juliano, ed., Oxford, N.Y., pp. 253-315.
111. Radwan M M, Chandra S, Gul S, ElSohly M A. Cannabinoids, Phenolics, Terpenes and Alkaloids of Cannabis. Molecules. 2021 May 8;26 (9): 2774. doi: 10.3390/molecules26092774. PMID: 34066753; PMCID: PMC8125862.
112. Raval, N. R., Johansen, A., Donovan, L. L., Ros, N. F., Ozenne, B., Hansen, H. D., & Knudsen, G. M. (2021). A Single Dose of Psilocybin Increases Synaptic Density and Decreases 5-HT2A Receptor Density in the Pig Brain. International journal of molecular sciences, 22 (2). 835.
113. Raval NR, Gudmundsen F. Juhl M. Andersen IV, Speth N, Videbæk A. Petersen IN, Mikkelsen JD, Fisher PM, Herth MM, Plavén-Sigray P. Knudsen GM, Palner M. Synaptic Density and Neuronal Metabolic Function Measured by Positron Emission Tomography in the Unilateral 6-OHDA Rat Model of Parkinson's Disease. Front Synaptic Neurosci. 2021 Nov. 18:13:715811. doi: 10.3389/fnsyn.2021.715811. PMID. 34867258; PMCID: PMC8636601.
114. Remington: Science and Practice of Pharmacy (2005) 21th ed., Mack Pub. Co., Easton, Pa.
115. Reuter, I., Engelhardt, M., Freiwaldt, J., & Baas, H. (1999). Exercise test in Parkinson's disease. Clinical autonomic research: official journal of the Clinical Autonomic Research Society, 9 (3), 129-134. https://doi.org/10.1007/BF02281625 116. Russo EB. Taming THC: potential cannabis synergy and phytocannabinoid-terpenoid entourage effects. Br J Pharmacol. 2011 August: 163 (7): 1344-64. doi: 10.1111/j. 1476-5381.2011.01238.x. PMID: 21749363.
117. Samuelsson, G., & Öquist, G. (1977). A Method for Studying Photosynthetic Capacities of Unicellular Algae Based on in vivo Chlorophyll Fluorescence, Physiologia Plantarum, 40 (4), 315-319. https://doi.org/10.1111/j. 1399-3054.1977.tb04080.x
118. Sakashita, Y., Abe, K., Katagiri, N., Kambe, T., Saitoh, T., Utsunomiya, I., Horiguchi, Y., & Taguchi, K. (2015). Effect of psilocin on extracellular dopamine and serotonin levels in the mesoaccumbens and mesocortical pathway in awake rats. Biological & pharmaceutical bulletin, 38 (1), 134-138. https://doi.org/10.1248/bpb.b14-00315
119. Sasidharan, S., Chen, Y., Saravanan, D., Sundram, K. M., & Yoga Latha, L. (2011). Extraction, isolation and characterization of bioactive compounds from plants' extracts. African journal of traditional, complementary, and alternative medicines: AJTCAM, 8 (1), 1-10.
120. Shao C Y, Mirra S S, Sait H B, Sacktor T C, Sigurdsson EM. (2011) Postsynaptic degeneration as revealed by PSD-95 reduction occurs after advanced AB and tau pathology in transgenic mouse models of Alzheimer's disease. Acta Neuropathol. 2011 September; 122 (3): 285-92. doi: 10.1007/s00401-011-0843-x. Epub 2011 Jun. 1. PMID: 21630115; PMCID: PMC3437675.
121. Sharifi-Rad, J., Cruz-Martins, N., López-Jornet, P., Lopez, E. P., Harun, N., Yeskaliyeva, B., Beyatli, A., Sytar, O., Shaheen, S., Sharopov, F., Taheri, Y., Docea, A. O., Calina, D., & Cho, W. C. (2021). Natural Coumarins: Exploring the Pharmacological Complexity and Underlying Molecular Mechanisms. Oxidative medicine and cellular longevity, 2021, 6492346. https://doi.org/10.1155/2021/6492346.
122. Shulgin & Shulgin, PiHKAL: A Chemical Love Story, Transform Press, Berkeley CA, 1992.
123. Shulgin & Shulgin, TIHKAL: The Continuation, Transform Press, Berkeley CA, 1997.
124. Stamets P., Psilocybin Mushrooms of the World: An Identification Guide, Ten Speed Press, Berkeley CA, 1996.
125. Syah, Y. M., Hakim, E. H., Ghisalberti, E. L., Jayuska, A., Mujahidin, D., & Achmad, S. A. (2009). A modified oligostilbenoid, diptoindonesin C, from Shorea pinanga Scheff. Natural product research, 23 (7), 591-594. https://doi.org/10.1080/14786410600761235.
126. Szabo, B., Siemes, S., & Wallmichrath, I. (2002). SHORT COMMUNICATION Inhibition of GABAergic neurotransmission in the ventral tegmental area by cannabinoids. European Journal of Neuroscience, 15 (12): 2057-2061. https://doi.org/10.1046/j.1460-9568.2002.02041.x.
127. Tahir, M.N., Shahbazi, F., Rondeau-Gagné, S. et al. The biosynthesis of the cannabinoids. J Cannabis Res 3. 7 (2021). https://doi.org/10.1186/s42238-021-00062-4.
128. The Merck Index (1996) 12th ed., Merck Pub. Group, Whitehouse, N.J.
129. “Theilheimer's Synthetic Methods of Organic Chemistry,” Volumes 1-45, Karger, 1991.
130. Thirupathi, A., Pinho, R. A., Baker, J. S., István, B., & Gu, Y. (2020). Taurine Reverses Oxidative Damages and Restores the Muscle Function in Overuse of Exercised Muscle. Frontiers in physiology, 11, 582449. https://doi.org/10.3389/fphys.2020.582449
131. Thomas, B. F., & ElSohly, M. A. (2016). Biosynthesis and pharmacology of phytocannabinoids and related chemical constituents. The Analytical Chemistry of Cannabis, 27-41.
132. Thu, Z. M., Myo, K. K., Aung, H. T., Clericuzio, M., Armijos, C., & Vidari, G. (2020). Bioactive Phytochemical Constituents of Wild Edible Mushrooms from Southeast Asia. Molecules (Basel, Switzerland), 25 (8), 1972. https://doi.org/10.3390/molecules25081972
133. Toyonaga T, Fesharaki-Zadeh A, Strittmatter S M, Carson R E, Cai Z. PET Imaging of Synaptic Density: Challenges and Opportunities of Synaptic Vesicle Glycoprotein 2A PET in Small Animal Imaging. Front Neurosci. 2022 Mar. 8; 16:787404. doi: 10.3389/fnins.2022.787404. PMID: 35345546; PMCID: PMC8957200.
134. Trincone, A. (2019). Enzymatic Techs. for marine polysaccharides. CRC Press, Taylor & Francis Group.
135. Trist, Benjamin Guy, Hare, Dominc James & Double, Kay Lorraine. (2018). A Proposed Mechanism for Neurodegeneration in Movement Disorders Characterized by Metal Dyshomeostasis and Oxidative Stress. Cell Chemical Biology, 25, 807-816, https://doi.org/10.1016/j.chembiol.2018.05.004.
136. Trost, B. & Fleming, I. “Comprehensive Organic Synthesis,” Pergamon Press, 1991.
137. Ultrasonic cold water extraction of psilocybin. Hielscher Ultrasonics. (2022 March 30). Retrieved May 21, 2022, from https://www.hielscher.com/ultrasonic-cold-water-extraction-of-psilocybin.htm
138. Understanding and measuring Magic mushroom potency. Freshminds. (2022 April 5). Retrieved May 1, 2022, from https://frshminds.com/understanding- and-measuring-magic-mushroom-potency/139.
139. Unified Parkinson Disease Rating Scale (UPDRS). (2022). Theracycle Physical Therapy Exercise Bike and Rehabilitation Equipment. https://www.theracycle.com/resources/links- and-additional-resources/updrs-scale/140.
140. Vahdat, M., Hosseini, S.A., Soltani, F. et al. The effects of Taurine supplementation on inflammatory markers and clinical outcomes in patients with traumatic brain injury: a double-blind randomized controlled trial. Nutr J 20, 53 (2021). https://doi.org/10.1186/s12937-021-00712-6
141. Varela-Álvarez, E., Loureiro, J., Paulino, C., & Serrão, E. A. (2018). Polyploid lineages in the genus Porphyra. Scientific reports, 8 (1), 8696. https://doi.org/10.1038/s41598-018-26796-5
142. Venturella, G., Ferraro, V., Cirlincione, F., & Gargano, M. L. (2021). Medicinal Mushrooms: Bioactive Compounds, Use, and Clinical Trials. International journal of molecular sciences, 22 (2), 634.
143. Wan, M., Ding, L., Wang, D., Han, J., & Gao, P. (2020). Serotonin: A potent immune cell modulator in autoimmune diseases. Frontiers in Immunology, 11. https://doi.org/10.3389/fimmu.2020.00186.
144. Wang, Y. H., Avula, B., Nanayakkara, N. P., Zhao, J., & Khan, I. A. (2013). Cassia cinnamon as a source of coumarin in cinnamon-flavored food and food supplements in the United States. Journal of agricultural and food chemistry, 61 (18), 4470-4476. https://doi.org/10.1021/jf4005862
145. Watchel, H., Bickmann, D., and Linz, S. (Aug. 18, 2015). U.S. Pat. No. 9,108,011. “Inhalation device”
146. Yildirim, Z., Kiliç, N., Ozer, C., Babul, A., Take, G., & Erdogan, D. (2007). Effects of taurine in cellular responses to oxidative stress in young and middle-aged rat liver. Annals of the New York Academy of Sciences, 1100, 553-561. https://doi.org/10.1196/annals. 1395.061
147. YingJuan Liu, Y. Liu, Zhenzhen Deng, Z. Deng, Lihua Geng, L. Geng, Jing Wang, J. Wang, & Quanbin Zhang, Q. Zhang. (0000). In vitro evaluation of the neuroprotective effect of oligo-porphyran from Porphyra yezoensis in PC12 cells. Journal of applied phycology, 31, 2559-2571.

The invention claimed is:

1. A therapeutic combination useful to prevent or treat a movement disorder, comprising:
a. a fungal portion;
b. a first plant portion;
c. optionally, a second plant portion; and
d. an algal portion.

2. The therapeutic combination of claim 1, comprising:
a. a fungal portion;
b. a first plant portion;
c. a second plant portion; and
d. an algal portion.

3. The therapeutic combination of claim 2, wherein the fungal portion is from a psilocybin-producing species.

4. The therapeutic combination of claim 3, wherein the psilocybin-producing species is from any of the genera Athelia, Conocybe, Copelandia, Fibularhizoctonia, Galerina, Gymnopilus, Inocybe, Mycena, Panaeolus, Pholiotina, Pluteus, and Psilocybe.

5. The therapeutic combination of claim 4, wherein the psilocybin-producing species is from the genus Psilocybe.

6. The therapeutic combination of claim 5, wherein the psilocybin-producing species from the genus Psilocybe is any of P. azurescens, P. bohemica, P. semilanceata, P. baeocystis, P. cyanescens, P. tampanensis, P. cubensis, P. weilii, P. hoogshagenii, P. stuntzii, P. cyanofibrillosa, and P. liniformans.

7. The therapeutic combination of claim 2, comprising:
a. a fungal portion from a species in the Psilocybe genus;
b. a first plant portion;
c. a second plant portion; and
d. an algal portion.

8. The therapeutic combination of claim 2, wherein the first plant portion is from a species in the Cannabis genus.

9. The therapeutic combination of claim 8, wherein the species in the Cannabis genus is any of Cannabis sativa, Cannabis indica, and Cannabis ruderalis.

10. The therapeutic combination of claim 2, comprising:
a. a fungal portion;
b. a first plant portion from a species in the Cannabis genus;
c. a second plant portion; and
d. an algal portion.

11. The therapeutic combination of claim 10, comprising:
a. a fungal portion from a species in the Psilocybe genus;
b. a first plant portion from a species in the Cannabis genus;
c. a second plant portion; and
d. an algal portion.

12. The therapeutic combination of claim 2, wherein the second plant portion is from a species in the Dipteryx genus.

13. The therapeutic combination of claim 12, wherein the species in the Dipteryx genus is Dipteryx odorata.

14. The therapeutic combination of claim 2, comprising:
a. a fungal portion;
b. a first plant portion;
c. a second plant portion from a species in the Dipteryx genus; and
d. an algal portion.

15. The therapeutic combination of claim 14, comprising:
a. a fungal portion from a species in the Psilocybe genus;
b. a first plant portion from a species in the Cannabis genus;
c. a second plant portion from a species in the Dipteryx genus; and
d. an algal portion.

16. The therapeutic combination of claim 2, wherein the algal portion is from a species of marine algae.

17. The therapeutic combination of claim 16, wherein the species of marine algae is from the family Bangiaceae.

18. The therapeutic combination of claim 17, wherein the species of marine algae is from the genera Pyropia and Porphyra.

19. The therapeutic combination of claim 18, wherein the species of marine algae is any of Pyropia yezoensis, Pyropia perforata, and Porphyra umbilicalis.

20. The therapeutic combination of claim 2, comprising:
a. a fungal portion;
b. a first plant portion;
c. a second plant portion; and
d. an algal portion from a species in the Pyropia or Porphyra genera.

21. The therapeutic combination of claim 20, comprising:
a. a fungal portion from a species in the Psilocybe genus;
b. a first plant portion from a species in the Cannabis genus;
c. a second plant portion from a species in the Dipteryx genus; and
d. an algal portion from a species in the Pyropia or Porphyra genera.

22. The therapeutic combination of claim 3, wherein the fungal portion comprises a fungal extract from the psilocybin-producing species.

23. The therapeutic combination of claim 22, wherein the fungal extract is from P. azurescens, P. bohemica, P. semilanceata, P. baeocystis, P. cyanescens, P. tampanensis, P. cubensis, P. weilii, P. hoogshagenii, P. stuntzii, P. cyanofibrillosa, or P. liniformans.

24. The therapeutic combination of claim 23, wherein the fungal extract is obtained by ultrasonic extraction or Soxhlet extraction.

25. The therapeutic combination of claim 24, wherein the fungal extract comprises a 2:1 mixture of a fungal extract obtained by ultrasonic extraction and a fungal extract obtained by Soxhlet extraction.

26. The therapeutic combination of claim 8, wherein the first plant portion comprises a Cannabis plant extract from a species in the Cannabis genus.

27. The therapeutic combination of claim 26, wherein the Cannabis plant extract is from Cannabis sativa, Cannabis indica, or Cannabis ruderalis.

28. The therapeutic combination of claim 27, wherein the Cannabis plant extract is obtained by Soxhlet extraction.

29. The therapeutic combination of claim 12, wherein the second plant portion comprises a Dipteryx plant extract from a species in the Dipteryx genus.

30. The therapeutic combination of claim 29, wherein the Dipteryx plant extract is from Dipteryx odorata.

31. The therapeutic combination of claim 30, wherein the Dipteryx plant extract is obtained by anhydrous ethanol percolation.

32. The therapeutic combination of claim 16, wherein the algal portion comprises an algal extract from a species of marine algae.

33. The therapeutic combination of claim 32, wherein the algal extract is from Pyropia yezoensis, Pyropia perforata, or Porphyra umbilicalis.

34. The therapeutic combination of claim 33, wherein the algal extract is obtained by ultrasonic extraction.

35. The therapeutic combination of claim 2, comprising:
a. a fungal extract from a species in the Psilocybe genus;
b. a Cannabis plant extract from a species in the Cannabis genus;
c. a Dipteryx plant extract from a species in the Dipteryx genus; and
d. an algal extract from a species in the Pyropia or Porphyra genera.

36. The therapeutic combination of claim 35, comprising:
a. a 2:1 mixture of a fungal extract obtained by ultrasonic extraction and a fungal extract obtained by Soxhlet extraction;
b. a Cannabis plant extract obtained by Soxhlet extraction;
c. a Dipteryx plant extract obtained by anhydrous ethanol percolation; and
d. an algal extract obtained by ultrasonic extraction.

37. The therapeutic combination of either of claim 35 or 36, comprising:
a. a fungal extract comprising psilocybin and psilocin;
b. a Cannabis plant extract comprising Δ⁹-THC (THC) and cannabidiol (CBD);
c. a Dipteryx plant extract comprising coumarin; and
d. an algal extract comprising porphyran.

38. The therapeutic combination of claim 21, comprising:
a. a fungal portion comprising psilocybin and psilocin;
b. a first plant portion comprising Δ⁹-THC (THC) and cannabidiol (CBD);
c. a second plant portion comprising coumarin; and
d. an algal portion comprising porphyran.

39. The therapeutic combination of any of claims 1-36, wherein the fungal portion comprises a bioactive molecule from a fungus.

40. The therapeutic combination of claim 39, wherein the bioactive molecule from a fungus is a primary bioactive molecule from a psilocybin-producing species.

41. The therapeutic combination of claim 40, wherein the primary bioactive molecule from a psilocybin-producing species is one or more tryptamines or one or more beta-carbolines.

42. The therapeutic combination of claim 41, wherein the one or more tryptamines is any of psilocybin, psilocin, baeocystin, norbaeocystin, norpsilocin, and aeruginascin.

43. The therapeutic combination of claim 42, wherein the one or more tryptamines is psilocybin or psilocin.

44. The therapeutic combination of claim 42, wherein the one or more tryptamines is psilocybin and psilocin.

45. The therapeutic combination of claim 44, wherein psilocybin and psilocin are in a weight ratio of about 5:3.

46. The therapeutic combination of claim 41, wherein the one or more beta-carbolines is any of harmane, harmine, harmol, pinoline, harmaline, cordysinin C, cordysinin D, norharmane, and perlolyrine.

47. The therapeutic combination of claim 39, wherein the bioactive molecule from a fungus is a secondary bioactive molecule from a psilocybin-producing species.

48. The therapeutic combination of claim 47, wherein the secondary bioactive molecule from a psilocybin-producing species is any of a polysaccharide, a peptide, a terpene, a phenolic compound, a mineral, a vitamin, an amino acid, a lipid, choline, and a lactone.

49. The therapeutic combination of any of claims 1-36, wherein the first plant portion comprises a bioactive molecule from Cannabis.

50. The therapeutic combination of claim 49, wherein the bioactive molecule from Cannabis is a primary bioactive molecule from a Cannabis species.

51. The therapeutic combination of claim 50, wherein the primary bioactive molecule from a Cannabis species is one or more cannabinoids.

52. The therapeutic combination of claim 51, wherein the one or more cannabinoids is any of a Δ⁹-THC-type cannabinoid, a Δ⁸-THC-type cannabinoid, a CBG-type cannabinoid, a CBD-type cannabinoid, a CBND-type cannabinoid, a CBE-type cannabinoid, a CBL-type cannabinoid, a CBC-type cannabinoid, a CBN-type cannabinoid, a CBT-type cannabinoid, and a miscellaneous-type cannabinoid.

53. The therapeutic combination of claim 52, wherein the one or more cannabinoids is Δ⁹-THC (THC) or cannabidiol (CBD).

54. The therapeutic combination of claim 52, wherein the one or more cannabinoids is THC and CBD.

55. The therapeutic combination of claim 54, wherein THC and CBD are in a weight ratio of about 1:1.

56. The therapeutic combination of claim 49, wherein the bioactive molecule from Cannabis is a secondary bioactive molecule from a Cannabis species.

57. The therapeutic combination of claim 56, wherein the secondary bioactive molecule from a Cannabis species is any of a flavone or flavonoid, a terpene or terpenoid, a carbohydrate, a fatty acid or a fatty acid esters, an amide, an amine, a phytosterol, and a phenolic compound.

58. The therapeutic combination of any of claims 1-36, wherein the second plant portion comprises a bioactive molecule from Dipteryx.

59. The therapeutic combination of claim 58, wherein the bioactive molecule from Dipteryx is a primary bioactive molecule from Dipteryx odorata.

60. The therapeutic combination of claim 59, wherein the primary bioactive molecule from Dipteryx odorata is coumarin.

61. The therapeutic combination of claim 58, wherein the bioactive molecule from Dipteryx is a secondary bioactive molecule from Dipteryx odorata.

62. The therapeutic combination of claim 61, wherein the secondary bioactive molecule from Dipteryx odorata is any of cumaru, a coumarin derivative, an isoflavone, a lupeol derivative, a fatty acid ester, (±)-balanophonin, (−)-lariciresinol, 3′-hydroxyretusin-8-methyl-ether, 5-methoxyxanthocercin A, 6,4′-dihydroxy-3′-methoxyaurone, 7-hydroxychromone, 7,3′-dihydroxy-8,4′-dimethoxyisoflavone, betulin, butin, coumaric-acid-beta-glucoside, dipteryxin, dipteryxic acid, eriodictyol, ferulic-acid, isoliquiritigenin, lupeol, melilotoside, melilotoside-1-p-coumaryl-beta-d-glucose, methyl-linolenate, methyl-oleate, O-coumaricacid, O-hydroxycoumaric-acid, odoratin, P-hydroxy-benzoic-acid, retusin, retusin-8-methyl-ether, sulfuretin, salicylic-acid, afrormisin, castinin, linoleic acid, oleic acid, 3′,4′,7′-trihydroxyflavone, luteolin, and umbelliferone.

63. The therapeutic combination of any of claims 1-36, wherein the algal portion comprises a bioactive molecule from algae.

64. The therapeutic combination of claim 63, wherein the bioactive molecule from algae is a primary bioactive molecule from Pyropia or Porphyra.

65. The therapeutic combination of claim 64, wherein the primary bioactive molecule from Pyropia or Porphyra is any of porphyran, an oligo-porphyran, a polysaccharide, an oligo-polysaccharide, a monosaccharide, a peptide, a phycobiliprotein, a mycosporine-like amino acid, an essential amino acid, a nonessential amino acid, a carotene, an intermediate carotenoid, a glycoprotein, an amino sulfonic acid, and taurine.

66. The therapeutic combination of claim 65, wherein the primary bioactive molecule from Pyropia or Porphyra is porphyran.

67. The therapeutic combination of claim 63, wherein the bioactive molecule from algae is a secondary bioactive molecule from Pyropia or Porphyra.

68. The therapeutic combination of claim 67, wherein the secondary bioactive molecule from Pyropia or Porphyra is any of a mineral, a vitamin, a lipid, a phenolic compound, and a phlorotannin.

69. The therapeutic combination of any one of claims 1-36, further comprising a flavorant or colorant.

70. The therapeutic combination of claim 69, wherein the flavorant is ginger or bay laurel.

71. The therapeutic combination of any one of claims 1-36, further comprising an additional active agent.

72. The therapeutic combination of claim 71, wherein the additional active agent is any of levodopa, carbidopa, carbidopa-levodopa, entacapone, carbidopa-levodopa-entacapone, tolcapone, opicapone, pramipexole, pramipexole, ropinirole, apomorphine, rotigotine, selegiline, rasagiline, safinamide, amantadine, istradefylline, trihexyphenidyl, benztropine, procyclidine, trihexyphenidyl, orphenadrine, and buntanetap.

73. The therapeutic combination of any one of claims 1-36, wherein at least one of the fungal portion, the first plant portion, the second plant portion, or the algal portion further comprises a non-naturally occurring carrier, diluent, or excipient.

74. The therapeutic combination of claim 73, wherein at least two, at least three, or all four of the fungal portion, the first plant portion, the second plant portion, and the algal portion further comprises a non-naturally occurring carrier, diluent, or excipient.

75. The therapeutic combination of claim 21, comprising:
a. a bioactive molecule from a species in the Psilocybe genus;
b. a bioactive molecule from a species in the Cannabis genus;
c. a bioactive molecule from a species in the Dipteryx genus; and
d. a bioactive molecule from a species in the Pyropia or Porphyra genera.

76. The therapeutic combination of claim 75, comprising:
a. a primary bioactive molecule from a species in the Psilocybe genus;
b. a primary bioactive molecule from a species in the Cannabis genus;
c. a primary bioactive molecule from a species in the Dipteryx genus; and
d. a primary bioactive molecule from a species in the Pyropia or Porphyra genera.

77. The therapeutic combination of claim 76, comprising:
a. one or more tryptamines from a species in the Psilocybe genus;
b. one or more cannabinoids from a species in the Cannabis genus;
c. coumarin; and
d. porphyran.

78. The therapeutic combination of claim 77, comprising:
a. psilocybin and psilocin;
b. THC and CBD;
c. coumarin; and
d. porphyran.

79. The therapeutic combination of claim 78, comprising:
a. psilocybin and psilocin in a weight ratio of 5:3;
b. THC and CBD in a weight ratio of 1:1;
c. coumarin; and
d. porphyran.

80. The therapeutic combination of any one of claims 75-79, further comprising:
a. a secondary bioactive molecule from a species in the Psilocybe genus;
b. a secondary bioactive molecule from a species in the Cannabis genus;
c. a secondary bioactive molecule from a species in the Dipteryx genus; and
d. a secondary bioactive molecule from a species in the Pyropia or Porphyra genera.

81. The therapeutic combination of any one of claims 75-79, wherein one of the bioactive molecules, two of the bioactive molecules, three of the bioactive molecules, four of the bioactive molecules, five of the bioactive molecules, six of the bioactive molecules, at least one of the bioactive molecule, at least two of the bioactive molecules, at least three of the bioactive molecules, at least four of the bioactive molecules, at least five of the bioactive molecules, at least six of the bioactive molecules, all of the bioactive molecules, six or fewer of the bioactive molecules, five or fewer of the bioactive molecules, four or fewer of the bioactive molecules, three or fewer of the bioactive molecules, two or fewer of the bioactive molecules, or none of the bioactive molecules are from or are in an extract.

82. The therapeutic combination of any one of claims 75-79, wherein one of the bioactive molecules, two of the bioactive molecules, three of the bioactive molecules, four of the bioactive molecules, five of the bioactive molecules, six of the bioactive molecules, at least one of the bioactive molecule, at least two of the bioactive molecules, at least three of the bioactive molecules, at least four of the bioactive molecules, at least five of the bioactive molecules, at least six of the bioactive molecules, all of the bioactive molecules, six or fewer of the bioactive molecules, five or fewer of the bioactive molecules, four or fewer of the bioactive molecules, three or fewer of the bioactive molecules, two or fewer of the bioactive molecules, or none of the bioactive molecules are isolated molecules.

83. The therapeutic combination of any one of claims 75-79, wherein one of the bioactive molecules, two of the bioactive molecules, three of the bioactive molecules, four of the bioactive molecules, five of the bioactive molecules, six of the bioactive molecules, at least one of the bioactive molecule, at least two of the bioactive molecules, at least three of the bioactive molecules, at least four of the bioactive molecules, at least five of the bioactive molecules, at least six of the bioactive molecules, all of the bioactive molecules, six or fewer of the bioactive molecules, five or fewer of the bioactive molecules, four or fewer of the bioactive molecules, three or fewer of the bioactive molecules, two or fewer of the bioactive molecules, or none of the bioactive molecules are pure or substantially pure molecules.

84. The therapeutic combination of any one of claims 75-79, wherein one of the bioactive molecules, two of the bioactive molecules, three of the bioactive molecules, four of the bioactive molecules, five of the bioactive molecules, six of the bioactive molecules, at least one of the bioactive molecule, at least two of the bioactive molecules, at least three of the bioactive molecules, at least four of the bioactive molecules, at least five of the bioactive molecules, at least six of the bioactive molecules, all of the bioactive molecules, six or fewer of the bioactive molecules, five or fewer of the bioactive molecules, four or fewer of the bioactive molecules, three or fewer of the bioactive molecules, two or fewer of the bioactive molecules, or none of the bioactive molecules are synthetic molecules.

85. The therapeutic combination of claim 79, wherein a single dose comprises:
a. 250 μg of psilocybin;
b. 150 μg of psilocin;
c. 1 mg of CBD;
d. 1 mg of THC;
e. 1 mg of coumarin;
f. 8 mg of an extract of Pyropia;
g. optionally, 8 mg of a flavorant or colorant.

86. The therapeutic combination of claim 85, wherein the flavorant or colorant comprises ginger or bay leaf.

87. The therapeutic combination of claim 85, wherein the flavorant or colorant comprises ginger and bay leaf.

88. The therapeutic combination of claim 85, further comprising a diluent.

89. The therapeutic combination of claim 88, wherein the diluent is water.

90. The therapeutic combination of claim 21, wherein:
a. the fungal portion constitutes about 45% by volume of the total combination;
b. the first plant portion constitutes about 15% by volume of the total combination;
c. the second plant portion constitutes about 2% by volume of the total combination;
d. the algal portion constitutes about 15% by volume of the total combination;
e. a flavorant or colorant constitutes about 15% by volume of the total combination; and
f. a diluent constitutes the remainder of the total combination.

91. The therapeutic combination of either of claim 21 or 90, wherein:
a. the fungal portion comprises a 2:1 mixture of a fungal extract obtained by ultrasonic extraction and a fungal extract obtained by Soxhlet extraction;
b. the first plant portion comprises a Cannabis plant extract obtained by Soxhlet extraction;
c. the second plant portion comprises a Dipteryx plant extract obtained by anhydrous ethanol percolation;
d. the algal portion comprises an algal extract obtained by ultrasonic extraction;
e. the flavorant or colorant comprises ethanol infused with ginger and bay leaf; and
f. the diluent comprises water.

92. The therapeutic combination of claim 91, wherein:
a. the fungal portion is a 2:1 mixture of a fungal extract obtained by ultrasonic extraction and a fungal extract obtained by Soxhlet extraction;
b. the first plant portion is a Cannabis plant extract obtained by Soxhlet extraction;
c. the second plant portion is a Dipteryx plant extract obtained by anhydrous ethanol percolation;
d. the algal portion is an algal extract obtained by ultrasonic extraction;
e. the flavorant or colorant is ethanol infused with ginger and bay leaf; and
f. the diluent is water.

93. The therapeutic combination of claim 91, wherein the diluent comprises water and at least one non-naturally occurring diluent.

94. A method of preparing the therapeutic combination of claim 35, comprising:
a. obtaining the fungal extract by ultrasonic extraction and/or Soxhlet extraction;
b. obtaining the Cannabis plant extract by Soxhlet extraction;
c. obtaining the Dipteryx plant extract by anhydrous ethanol percolation;
d. obtaining the algal extract by ultrasonic extraction;
e. analyzing the concentration of at least one bioactive molecule in each extract;
f. calculating a compounding percentage to achieve a target dose for each of the at least one bioactive molecule in each extract;
g. blending a calculated amount of each extract based on the compounding percentage into a mixture;
h. optionally, homogenizing the mixture;
i. optionally, adding a flavorant or colorant;
j. optionally, adding a diluent to obtain a target volume.

95. The method of preparing the therapeutic combination of claim 94, wherein the target dose for each of the at least one bioactive molecule in each extract comprises:
a. 250 μg of psilocybin;
b. 150 μg of psilocin;
c. 1 mg of CBD;
d. 1 mg of THC; and
e. 1 mg of coumarin.

96. The method of preparing the therapeutic combination of claim 94, wherein the calculated amount of each extract based on the compounding percentage comprises:
a. 45% by volume of the total combination for the fungal extract;
b. 15% by volume of the total combination for the Cannabis plant extract;
c. 2% by volume of the total combination for the Dipteryx plant extract;
d. 15% by volume of the total combination for the algal extract;
e. 15% by volume of the total combination for the flavorant or colorant; and
f. 8% by volume of the total combination for the diluent.

97. A pharmaceutical composition comprising the therapeutic combination of any of claim 1-36 or 85 and a pharmaceutically acceptable carrier, diluent, or excipient.

98. The pharmaceutical composition of claim 97, where the pharmaceutically acceptable carrier, diluent, or excipient is non-naturally occurring.

99. The pharmaceutical composition of claim 97, suitable for enteral or parenteral administration.

100. The pharmaceutical composition of claim 97, prepared as any of a tincture formulation, oral spray formulation, oral mucosal spray formulation, soft mist inhaler formulation, vaporizer formulation, tablet formulation, scorable double-strength tablet formulation, capsule formulation, capsule formulation with additional active agent, suspension formulation, intravenous solution formulation, injectable solution formulation, topical formulation for transdermal administration, cut matrix sublingual or buccal tablet formulation, individually formed sublingual or buccal lozenge formulation, intranasal delivery formulation.

101. The pharmaceutical composition of claim 100, prepared as any of a tincture formulation, oral spray formulation, oral mucosal spray formulation, or soft mist inhaler formulation.

102. The pharmaceutical composition of claim 101, wherein the formulation comprises:
a. 45% by volume of the total combination for the fungal extract;
b. 15% by volume of the total combination for the Cannabis plant extract;
c. 2% by volume of the total combination for the Dipteryx plant extract;
d. 15% by volume of the total combination for the algal extract;
e. 15% by volume of the total combination for the flavorant or colorant; and
f. 8% by volume of the total combination for the diluent.

103. The pharmaceutical composition of claim 101, wherein a single dose comprises:
a. 250 μg of psilocybin;
b. 150 μg of psilocin;
c. 1 mg of CBD;
d. 1 mg of THC;
e. 1 mg of coumarin;
f. 8 mg of an extract of Pyropia;
g. optionally, 8 mg of a flavorant or colorant.

104. The pharmaceutical composition of claim 97, wherein a single dose of psilocybin is between about 0.5 μg to about 200 mg, between about 5 μg to about 5 mg, or between about 100 μg to about 600 μg.

105. The pharmaceutical composition of claim 104, wherein a single dose of psilocybin is 250 μg.

106. The pharmaceutical composition of claim 97, wherein a single dose of psilocin is between about 0.5 μg to about 200 mg, between about 5 μg to about 5 mg, or between about 100 μg to about 600 μg.

107. The pharmaceutical composition of claim 106, wherein a single dose of psilocin is 150 μg.

108. The pharmaceutical composition of claim 97, wherein a single dose of CBD is between about 0.5 μg to about 200 mg, between about 0.01 mg to about 75 mg, or between about 0.5 mg to about 15 mg.

109. The pharmaceutical composition of claim 108, wherein a single dose of CBD is 1 mg.

110. The pharmaceutical composition of claim 97, wherein a single dose of THC is between about 0.5 μg to about 200 mg, between about 0.01 mg to about 75 mg, or between about 0.5 mg to about 15 mg.

111. The pharmaceutical composition of claim 110, wherein a single dose of THC is 1 mg.

112. The pharmaceutical composition of claim 97, wherein a single dose of coumarin is between about 0.5 μg to about 200 mg, between about 0.01 mg to about 75 mg, or between about 0.5 mg to about 15 mg.

113. The pharmaceutical composition of claim 112, wherein a single dose of coumarin is 1 mg.

114. The pharmaceutical composition of claim 97, wherein a single dose includes a Pyropia yezoensis, Pyropia perforata, or Porphyra umbilicalis whole extract, in an amount of between about 0.5 mg to about 100 mg, between about 1 mg to about 50 mg, or between about 5 mg to about 20 mg.

115. The pharmaceutical composition of claim 114, wherein a single dose includes a Pyropia whole extract in an amount of 8 mg.

116. The pharmaceutical composition of claim 97, wherein a single dose includes ethanol infused with ginger and bay leaf, in an amount of between about 0.5 mg to about 100 mg, between about 1 mg to about 50 mg, or between about 5 mg to about 20 mg.

117. The pharmaceutical composition of claim 116, wherein a single dose includes ethanol infused with ginger and bay leaf in an amount of 8 mg.

118. A pharmaceutical kit comprising a first pharmaceutical composition, and a second pharmaceutical composition, wherein:
a. the first pharmaceutical composition comprises at least one portion of the therapeutic combination of claim 21, and a pharmaceutically acceptable carrier, diluent, or excipient; and
b. the second pharmaceutical composition comprises the remaining portions of the therapeutic combination of claim 21, and a pharmaceutically acceptable carrier, diluent, or excipient.

119. The pharmaceutical kit of claim 118, wherein:
a. the first pharmaceutical composition is prepared as a tincture formulation, oral spray formulation, oral mucosal spray formulation, or soft mist inhaler formulation; and
b. the second pharmaceutical composition is prepared as a tincture formulation, oral spray formulation, oral mucosal spray formulation, or soft mist inhaler formulation.

120. A method of preventing or treating a movement disorder, comprising administering to a patient in need thereof, the therapeutic combination of any of claim 1-36 or 85.

121. A method of preventing or treating a movement disorder, comprising administering to a patient in need thereof, the pharmaceutical composition of claim 103.

122. The method of claim 121, wherein the movement disorder is any one or more of ataxia, an ataxic disorder, a certain specified movement disorder, cervical dystonia, chorea, a choreiform disorder, dystonia, a dystonic disorder, essential tremor, Friedreich's ataxia, a functional movement disorder, hemifacial spasm, hereditary spastic paraplegia, Huntington's disease, L-dopa induced dyskinesia, multiple system atrophy (MSA), myoclonus, a myoclonic disorder, Parkinson's disease, atypical Parkinson's, Parkinsonism, Secondary Parkinsonism, progressive supranuclear palsy (PSP), restless legs syndrome, Rett syndrome, a sleep-related movement disorder, spasticity, tardive dyskinesia (TD), tourette syndrome, a tic disorder, a disorder associated with tremor, and Wilson's disease.

123. The method of claim 121, wherein the pharmaceutical composition is administered between 1 and 8 times per day.

124. The method of claim 121, wherein the patient experiences an improvement related to the movement disorder.

125. The method of claim 124, wherein the improvement is a reduction in the severity of at least one symptom of the movement disorder.

126. The method of claim 125, wherein the at least one symptom of the movement disorder is a motor symptom.

127. The method of claim 126, wherein the motor symptom is any of stooped posture, masked facial expression, forward tilt of trunk, flexed elbows and wrists, reduced arm swinging, flexed hips and knees, trembling of extremities, shuffling gait, short-stepped gait, uncoordinated or clumsy balance, altered speech, involuntary limb movements, irregular motor movement, long-lasting contractions, intermittent contractions of neck muscles, causing the head to turn in different ways; repetitive, irregular, involuntary movements involving the face, mouth, trunk, and limbs; twisting, repetitive movements; jerking of muscles or groups of muscles, tremors, stiffness, finger tapping, toe tapping, poor posture, slow, decreased movement or imbalance; difficulties walking, random involuntary eye movement, involuntary blinking, involuntary grimacing, unpleasant, abnormal feelings in limbs which may be relieved by movement; involuntary vocal sounds, and rhythmic shaking of parts of the body, commonly the hands and/or head.

128. The method of claim 126, wherein the motor symptom is any of issues with speech, overproducing saliva and drooling, problems with chewing and swallowing, difficulty eating, dressing, maintaining proper hygiene, handwriting, doing hobbies and other activities, turning in bed, getting out of bed, a car, or a deep chair; trouble walking and maintaining balance, experiencing tremor, and freezing in place.

129. The method of claim 125, wherein the at least one symptom of the movement disorder is a non-motor symptom.

130. The method of claim 129, wherein the non-motor symptom is any of cognitive impairment, hallucinations and psychosis, depressed mood, anxious mood, apathy, features of dopamine dysregulation syndrome, sleep problems, daytime sleepiness, pain and other sensations, urinary problems, constipation problems, lightheadedness on standing, and fatigue.

131. The method of claim 129, wherein the non-motor symptom is a mood symptom.

132. The method of claim 131, wherein the mood symptom is any of feelings of depression, anxiety, irritability, mood swings, impaired judgment, loss of empathy, aggression, impulsivity, delusions, and paranoia.

133. The method of claim 125, wherein the reduction in the severity of at least one symptom of the movement disorder occurs less than about 75 days from the first administration of the pharmaceutical composition.

134. The method of claim 125, wherein the reduction in the severity of at least one symptom of the movement disorder occurs less than about 35 days from the first administration of the pharmaceutical composition.

135. The method of claim 125, wherein the reduction in the severity of at least one symptom of the movement disorder lasts for at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 36 months, at least 48 months, or at least 60 months.

136. The method of claim 124, wherein the improvement is an improvement in motor control.

137. The method of claim 136, wherein the improvement in motor control is an improvement of any of balance, frequency of involuntary movements, amplitude of involuntary movements, strength, endurance, and physical capacity.

138. The method of claim 136, wherein the improvement in motor control occurs less than about 75 days from the first administration of the pharmaceutical composition.

139. The method of claim 136, wherein the improvement in motor control occurs less than about 35 days from the first administration of the pharmaceutical composition.

140. The method of claim 124, wherein the improvement is to a clinical outcome assessment.

141. The method of claim 140, wherein the clinical outcome assessment is any of the Movement Disorder Society-Unified Parkinson's Disease Rating Scale (MDS-UPDRS), the Movement Disorder Society Non-Motor Rating Scale (MDS-NMS), the Cortical Basal Ganglia Functional Scale (SBFS), the Gastrointestinal Dysfunction Scale for Parkinson's Disease (GIDS-PD), the Global Assessment Scale for Wilson's Disease (GAS for WD), the Global Dystonia Severity Rating Scale (GDS), the Modified Bradykinesia Rating Scale (MBRS), the Non-Motor Symptoms Questionnaire (NMSQ), the Non-Motor Symptoms Scale for Parkinson's Disease (NMSS), the Pantothenate Kinase-Associated Neurodegeneration Disease Rating Scale (PKAN-DRS), the Progressive Supranuclear Palsy Clinician Deficits Scale (PSP-CDS), the Quality of Life in Essential Tremor Questionnaire, the Rating Scale for Psychogenic Movement Disorders, the Rush Dyskinesia Rating Scale (RDRS), the Rush Video-Based Tic Rating Scale (RVBTRS), Scales for Outcomes in Parkinson's Disease-Autonomic Dysfunction (SCOPA-AUT), Scales for Outcomes in Parkinson's Disease-Diary Card (SCOPA-DC), Scales for Outcomes in Parkinson's Disease-Psychiatric Complications (SCOPA-PC), Scales for Outcomes in Parkinson's Disease-Psychosocial Functioning (SCOPA-PS), Scales for Outcomes in Parkinson's Disease-Sleep (SCOPA-Sleep; SCOPA-S), Scales for Outcomes in Parkinson's Disease-Cognition (SCOPA-COG), Short Parkinson's Evaluation Scale (SPES)/Scales for Outcomes in Parkinson's Disease-Motor Function (SPES/SCOPA-Motor), The Non-Motor Fluctuation Assessment (NoMoFA) Questionnaire, the UFMG Sydenham's Chorea Rating Scale (USCRS), Unified Dyskinesia Rating Scale (UDysRS), Unified Dystonia Rating Scale (UDRS), Unified Multiple System Atrophy Rating Scale (UMSARS), and the 8-item Unified Parkinson's Disease Rating Scale (UPDRS-8).

142. The method of claim 141, wherein the clinical outcome assessment is the MDS-UPDRS, the UPDRS, or the UPDRS-8.

143. The method of claim 142, wherein the clinical outcome assessment is the MDS-UPDRS.

144. The method of claim 143, wherein the improvement in the MDS-UPDRS is an improvement in the nM-EDL.

145. The method of claim 144, wherein the improvement in the nM-EDL is to any of cognitive impairment, hallucinations and psychosis, depressed mood, anxious mood, apathy, features of dopamine dysregulation syndrome, sleep problems, daytime sleepiness, pain and other sensations, urinary problems, constipation problems, lightheadedness on standing, and fatigue.

146. The method of claim 143, wherein the improvement in the MDS-UPDRS is an improvement in the M-EDL.

147. The method of claim 146, wherein the improvement in the M-EDL is to any of speech, saliva and drooling, chewing and swallowing, eating tasks, dressing, hygiene, handwriting, doing hobbies and other activities, turning in bed, tremor, getting out of bed, a car, or a deep chair; walking and balance, and freezing.

148. The method of claim 143, wherein the improvement in the MDS-UPDRS is an improvement in motor examination.

149. The method of claim 148, wherein the improvement in motor examination is to any of speech, facial expression, rigidity, finger tapping, hand movements, pronation-supination movements of hands, toe tapping, leg agility, arising from chair, gait, freezing of gait, postural stability, posture, global spontaneity of movement (body bradykinesia), postural tremor of the hands, kinetic tremor of the hands, rest tremor amplitude, constancy of rest tremor, Hoehn and Yahr stage, time spent with dyskinesias, functional impact of dyskinesias, time spent in the off state, functional impact of fluctuations, complexity of motor fluctuations, and painful off-state dystonia.

150. The method of any one of claims 143-149, wherein the improvement is a reduction in score.

151. The method of claim 150, wherein the reduction in score is by at least 1 point, at least 2 points, at least 3 points, or at least 4 points.

152. The method of claim 124, wherein the improvement is an improvement in the UPDRS.

153. The method of claim 152, wherein the improvement in the UPDRS is an improvement in mentation, behavior, and mood.

154. The method of claim 153, wherein the improvement in mentation, behavior, and mood is any of intellectual impairment, thought disorder, depression, and motivation/initiative.

155. The method of claim 152, wherein the improvement in the UPDRS is an improvement in ADL.

156. The method of claim 155, wherein the improvement in ADL is to any of speech, salivation, swallowing, handwriting, cutting food and handling utensils, dressing, hygiene, turning in bed and adjusting bed clothes, falling, freezing when walking, walking, tremor, and sensory complaints related to parkinsonism.

157. The method of claim 152, wherein the improvement in the UPDRS is an improvement in motor examination.

158. The method of claim 157, wherein the improvement in motor examination is to any of speech, facial expression, tremor at rest, action or postural tremor of hands, rigidity, finger taps, hand movements, rapid alternating movements of hands, leg agility, arising from chair, posture, gait, postural stability, and body bradykinesia and hypokinesia.

159. The method of claim 152, wherein the improvement in the UPDRS is an improvement in complications of therapy.

160. The method of claim 159, wherein the improvement in complications of therapy is to any of daily duration of dyskinesias, severity of disability from dyskinesias, painful dyskinesias, and the proportion of the waking day the patient is “off” on average.

161. The method of any one of claims 152-160, wherein the improvement is an improvement in score.

162. The method of claim 161, wherein the improvement in score is by at least 1 point, at least 2 points, at least 3 points, or at least 4 points.

163. The method of claim 152, wherein the improvement in the UPDRS is a reduction in stage of the modified Hoehn and Yahr staging session.

164. The method of claim 163, wherein the reduction is a reduction in at least 1 stage.

165. The method of claim 152, wherein the improvement in the UPDRS is a reduction in the Schwab and England ADL scale percentage.

166. The method of claim 165, wherein the reduction is a reduction of between about 10% and about 100%.

167. The method of claim 152, wherein the improvement in the UPDRS is a change in the binary yes and no questions.

168. The method of any one of claims 140-149, wherein the improvement occurs less than about 75 days from the first administration of the pharmaceutical composition.

169. The method of any one of claims 140-149, wherein the improvement occurs less than about 35 days from the first administration of the pharmaceutical composition.

170. The method of any one of claims 140-149, wherein the improvement lasts for at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 36 months, at least 48 months, or at least 60 months.
